SEQUENCE LISTING (40 sequences)

SEQ ID NO: 1 - DNA, 6455 bp, Homo sapiens
aggctgtgacagtcatctgtctggacgcgctgggtggatgcggggggctcctgggaactgtgttggagccgagcaagcgctagccaggcgcaagcgcgcacagactgtagccatccgaggacacccccgcccccccggcccacccggagacacccgcgcagaatcgcctccggatcccctgcagtcggcgggaggtaaggagcagggcttgcaaaccgcccggcgcccagggaagcgacgagcgccggggcaaggcaagccctggacgggattgcgacgtgcgcaccgggcgccctaatatgcccgggggactgtttctgcttccgaaacaaaaccatctctgggttttcccagaaaagccagttccagccccgaaggcatcctggctagaggagacccgccctaatccttttgcagcccttaccggggggagtaatggcttctgcgaaaagaaattccctcggctctagaagatctgtctgtgtttgagctgtcggagagccggtgcgtccccaccccaggctggggttcttctccaaagggtgcccctggaggaagaagagggggggattaggcagggcgaggccgccgcggtcgcaatctgggtcacggctgctccagcttggaggagaggcggctctcccggcgaccctcctcgcgcgggcgcccctgccattcccgggaacaggggctcagcctctccctccctggaagaggacgttgtcgtgggtttggaagagcaggggtgggcttagagagcttccaattaagctattggcaggagtatccctgcagcgggtgaatgccgaggggcgtttgctcaaatttggggaggggaaggatttgtggatatgggtgtctgttgttggtctctgtctagagaaaggcttttttttatttgcaaagttttctaaatcccctgctatcatttgcactcctgaggttgcatttttacaaagggggtagaaggtactccaaataccattcccggtagctgggtcggagagcctggggcttcccctgagcagccggccccacaccgctgcgagtgcggttgtctgcgtgctcgtgagagctagaattctgcagccaggaacagccccctcccccaggcagtgccttgtgtgaatgaaatggcagtttccaaagttgcggagcctcgccaccaccccctgcatctgcatgccccctcccaccccctgtcgtagacagcttgtacacaaaaggagggcgggagggagggagcgagaggcacaacttcctccaccttcgggagcagtgggcagagtggggggcttggagggaagattggggaacctggttagagggggcgcccattgcctatcccctcggtctgccccgtttgcccaccctctccggtgtgtctgtcggttgcagtgttggaggtcggcgccggcccccgccttccgcgccccccacgggaaggaagcacccccggtattaaaacgaacggggcggaaagaagccctcagtcgccggccgggaggcgagccgatgccgagctgctccacgtccaccatgccgggcatgatctgcaagaacccagacctcgagtttgactcgctacagccctgcttctacccggacgaagatgacttctacttcggcggccccgactcgacccccccgggggaggacatctggaagaagtttgagctgctgcccacgcccccgctgtcgcccagccgtggcttcgcggagcacagctccgagcccccgagctgggtcacggagatgctgcttgagaacgagctgtggggcagcccggccgaggaggacgcgttcggcctggggggactgggtggcctcacccccaacccggtcatcctccaggactgcatgtggagcggcttctccgcccgcgagaagctggagcgcgccgtgagcgagaagctgcagcacggccgcgggccgccaaccgccggttccaccgcccagtccccgggagccggcgccgccagccctgcgggtcgcgggcacggcggggctgcgggagccggccgcgccggggccgccctgcccgccgagctcgcccacccggccgccgagtgcgtggatcccgccgtggtcttcccctttcccgtgaacaagcgcgagccagcgcccgtgcccgcagccccggccagtgccccggcggcgggccctgcggtcgcctcgggggcgggtattgccgccccagccggggccccgggggtcgcccctccgcgcccaggcggccgccagaccagcggcggcgaccacaaggccctcagtacctccggagaggacaccctgagcgattcaggtaaagaccgaactcgggtccggctgcctccctggggcactggaccccgggtcgcgtcccctttgttagtgctcgtatgtcttggcctggggagcattttggaggcagtgctaggggcagagaggtcctgtttcccccaagtctctcctcggggtaaagagaaggggctgagagaatgccgttgcaaaaggggtgctctccaattctcgccttcactaaagttccttccaccctctcctggggagccctcctctaggccatcacgggccctcacccggtcccccacctctcttttgcagcgcagtctgaggaataaaattggagaaagttggtggctaaaccgggtgggggtttagggggttgctgggtgcactgcctggacagaaacctgttagcgcaggggtgaaagggactctctggcccaggtcaggggagggaaagacatcccgagaagattcaagggctgtgcaaagccctgtttaaggcgcaggaacttataggagggttgcacagatggctagagccgattttctattctttttctttttctttttttttttttttcaaatgtcggtacctttcccttcccccatcctcggtgggtggtgggctatttgctcctggtgcgtggccagcaggcggcgatatgcgaggccagcaggcgggcccgggatctgaaaggctgggggtggtgggggcaccctccctccctccattcagcagctggctgcaagtgcaacagcagttgtgtacattctcagggggcctcctctttccagtgtgcagtggaaactggctgtagttttgtcttccagcctgaattccaggcctaatttgagatgtgagttgtatctgtaacccagtgcccttgaaggtgagggcaggcactcagcagcctctccaggaaggctcacatcctgggaggactcactgattagttctattgtgttcatttgtctgtgtcttaagctgaagggaagagttaaaaccaagcctttccctgggggtctggatgaacagaactcaacccaaagagtggcattgccttgtccttggagcagggagctgggaccccccttggactttgaaaaccagtgttttcagaatgcaggtggataacaagcctaaatttacttctgggctgaggagagatctttgaggctcctggaaggaaacttggtgataagcctccagtttgaaacggctctgtccctttaatgtctgtgccttgacagcttttggtgaggaagcacttccttccaacagctgtcttcttggcagaaaaccaaaacattggcttaaagggacccacagactggaacagcctcacatttcggctttagaacaaatcccacaattgttcagctttccggtccccttcagatcaagcagaagatatgttttgattttcatgcttgtattttaaacaataattttctaccccagcgtggtagtcaatgaggagagaggggaagaatgcgcacatgatgctacacgtttctgttgttgctgttattattggtggctttgaggagagctgctcccatttgggggtttataccaactgtggattatggctttgtcattaagatttgatctttgttaaatgaaaaactgtttattgtataaaactcaggtttgtggacgaaaagttgttttttttcttcagttaattaaattgttcctcaagtttgtttaaggacttaaaatcaaacacaaccatgtgtaaactgctaaatgaggctcctaaaatgagaggcctcaactctttaagtgtggagctagaaatgtaaataagtccacagggcagactggtgattatgataaaagctaccatttactgagcatctgtctactaggctcagctctatgctaagtctacatgttatctgtcaaagtggtatcatccccatttaatagctgaggaaacagaggcttagaaaggctgggtaacttgaccagggtcatgcaactagtctgcggtggagccaggattctgtctgaccctaaaggccaagttctttatatttatttctaccacctgctaaagtcttgaatggaggctgaaagcacagttggggtatggggaagaaaaatatatatacatacatatatgtatatgtatgtatgtatgtatggggggttgttttgtttttgtttttgataaggagttttgctcttgttgcccaggctggagtgcagtggtatgatctgggctcactgcaacctccgcctcccgggttcaagtcattctcctgcctcagcctcccgagtagctgggattaccggagcatgccaccacacccagcaaagttttgtatttttagtagagacagggtttcaccatgttggccaggctgatcttgaactcctcatctcaggtgatctgcccgcctccgcttcccaaagtgctgggattacaggtgtgagtcaccgcgtccggcctacagatatatttaatttaaagagatctaaaacaaatacaaaactgtccacatctatgttgatggacccataaaaatagcagtctgccagggtctgccggaagagacagataagcatacatattaacatggatatatatgtgaatttcattcaaatggttctcacatgagagtaactagcatctttctctcagatgatgaagatgatgaagaggaagatgaagaggaagaaatcgacgtggtcactgtggagaagcggcgttcctcctccaacaccaaggctgtcaccacattcaccatcactgtgcgtcccaagaacgcagccctgggtcccgggagggctcagtccagcgagctgatcctcaaacgatgccttcccatccaccagcagcacaactatgccgccccctctccctacgtggagagtgaggatgcacccccacagaagaagataaagagcgaggcgtccccacgtccgctcaagagtgtcatccccccaaaggctaagagcttgagcccccgaaactctgactcggaggacagtgagcgtcgcagaaaccacaacatcctggagcgccagcgccgcaacgaccttcggtccagctttctcacgctcagggaccacgtgccggagttggtaaagaatgagaaggccgccaaggtggtcattttgaaaaaggccactgagtatgtccactccctccaggccgaggagcaccagcttttgctggaaaaggaaaaattgcaggcaagacagcagcagttgctaaagaaaattgaacacgctcggacttgctagacgcttctcaaaactggacagtcactgccactttgcacattttgattttttttttaaacaaacattgtgttgacattaagaatgttggtttactttcaaatcggtcccctgtcgagttcggctctgggtgggcagtaggaccaccagtgtggggttctgctgggaccttggagagcctgcatcccaggatgctgggtggccctgcagcctcctccacctcacctccatgacagcgctaaacgttggtgacggttgggagcctctggggctgttgaagtcaccttgtgtgttccaagtttccaaacaacagaaagtcattccttctttttaaaatggtgcttaagttccagcagatgccacataaggggtttgccatttgatacccctggggaacatttctgtaaataccattgacacatccgccttttgtatacatcctgggtaatgagaggtggcttttgcggccagtattagactggaagttcatacctaagtactgtaataatacctcaatgtttgaggagcatgttttgtatacaaatatattgttaatctctgttatgtactgtactaattcttacactgcctgtatactttagtatgacgctgatacataactaaatttgatacttatattttcgtatgaaaatgagttgtgaaagttttgagtagatattactttatcactttttgaactaagaaacttttgtaaagaaatttactatatatatatgcctttttcctagcctgtttcttcctgttaatgtatttgttcatgtttggtgcatagaactgggtaaatgcaaagttctgtgtttaatttcttcaaaatgtatatatttagtgctgcatcttatagcactttgaaatacctcatgtttatgaaaataaatagcttaaaattaaa

SEQ ID NO: 2 - DNA, 30 bp, Artificial Sequence
  An exemplary BCL2 G4 nucleotide sequence
aggggcgggcgcgggaggaagggggcggga

SEQ ID NO: 3 - DNA, 35 bp, Artificial Sequence
  An exemplary KRAS G4 nucleotide sequence
agggcggtgtgggaagagggaagagggggaggcag

SEQ ID NO: 4 - DNA, 27 bp, Artificial Sequence
  An exemplary mTOR G4 nucleotide sequence
ggggaaggcgggcggtggggcaggggg

SEQ ID NO: 5 - RNA, 21 nt, Artificial Sequence
  An exemplary NRAS G4 nucleotide sequence
ugugggaggggcgggucuggg

SEQ ID NO: 6 - DNA, 27 bp, Artificial Sequence
  An exemplary telomeric DNA G4 nucleotide sequence
ttagggttagggttagggttagggtta

SEQ ID NO: 7 - DNA, 34 bp, Artificial Sequence
  An exemplary MYCN G4 wild-type nucleotide sequence
agggggtgggagggggcatgcagatgcagggggt

SEQ ID NO: 8 - DNA, 34 bp, Artificial Sequence
  An exemplary MYCN G4 mutated nucleotide sequence
agggggtgggagggggctttttgatgcagggggt

SEQ ID NO: 9 - DNA, 24 bp, Artificial Sequence
  An exemplary MYCN G4 truncated nucleotide sequence
agggggtgggagggggcagggggt

SEQ ID NO: 10 - DNA, 20 bp, Artificial Sequence
  An exemplary primer for the sense strand of MYCNOS-01
aggctcagtctccctcacta

SEQ ID NO: 11 - DNA, 20 bp, Artificial Sequence
  An exemplary primer for the antisense strand of MYCNOS-01
ttctggaggctgagaagtcc

SEQ ID NO: 12 - DNA, 20 bp, Artificial Sequence
  An exemplary primer for the sense strand of MYCNOS-02
ctcacgagcacgcagacaac

SEQ ID NO: 13 - DNA, 20 bp, Artificial Sequence
  An exemplary primer for the antisense strand of MYCNOS-02
tcccagctttgcagccttct

SEQ ID NO: 14 - DNA, 20 bp, Artificial Sequence
  An exemplary primer for the sense strand of MYCN protein isoform 1
gatctgcaagaacccagacc

SEQ ID NO: 15 - DNA, 20 bp, Artificial Sequence
  An exemplary primer for the antisense strand of MYCN protein isoform 1
cacagctcgttctcaagcag

SEQ ID NO: 16 - DNA, 19 bp, Artificial Sequence
  An exemplary primer for the sense strand of MYCN protein isoform 2
tcctgggaactgtgttgga

SEQ ID NO: 17 - DNA, 20 bp, Artificial Sequence
  An exemplary primer for the antisense strand of MYCN protein isoform 2
cacagtgaccacgtcgattt

SEQ ID NO: 18 - DNA, 34 bp, Homo sapiens
accccctgcatctgcatgccccctcccaccccct

SEQ ID NO: 19 - DNA, 270 bp, Homo sapiens
gaaggtggaggaagttgtgcctctcgctccctccctcccgccctccttttgtgtacaagctgtctacgacagggggtgggagggggcatgcagatgcagggggtggtggcgaggctccgcaactttggaaactgccatttcattcacacaaggcactgcctgggggagggggctgttcctggctgcagaattctagctctcacgagcacgcagacaaccgcactcgcagcggtgtggggccggctgctcaggggaagccccaggctctcc

SEQ ID NO: 20 - DNA, 30 bp, Homo sapiens
gggggtgggagggggcatgcagatgcaggg

SEQ ID NO: 21 - DNA, 13 bp, Homo sapiens
ggtggtggcgagg

SEQ ID NO: 22 - DNA, 11 bp, Homo sapiens
gggggaggggg

SEQ ID NO: 23 - DNA, 13 bp, Homo sapiens
ggtgtggggccgg

SEQ ID NO: 24 - DNA, 29 bp, Homo sapiens
gggggcgggtgggggcgtaagcccggggg

SEQ ID NO: 25 - DNA, 27 bp, Homo sapiens
ggggtccccggaggggcgggtgggggg

SEQ ID NO: 26 - DNA, 57 bp, Homo sapiens
ggggggtggggaaagtgattggcagcagagctcacccaataggggctaggggctggg

SEQ ID NO: 27 - DNA, 48 bp, Homo sapiens
ggggccggggcgccgccacctacgccatccgcgcggcgcgggggcggg

SEQ ID NO: 28 - DNA, 56 bp, Homo sapiens
ggggacgctttgccacggtggtggaggagctcttcagggacggggtgaactggggg

SEQ ID NO: 29 - DNA, 40 bp, Homo sapiens
gggagctaaccaagctgaggcgctgcctggggagggaggg

SEQ ID NO: 30 - DNA, 57 bp, Homo sapiens
gggagggtgaacacacgccggtgagatacaggtagtgagggcaaggggggtgtgggg

SEQ ID NO: 31 - DNA, 46 bp, Artificial Sequence
  An exemplary hTERT G4 oligonucleotide
agggggctgggccggggacccgggaggggtcgggacggggcggggt

SEQ ID NO: 32 - DNA, 29 bp, Artificial Sequence
  An exemplary BCL2 G4 oligonucleotide
cgggcgggagcgcggcgggcgggcgggca

SEQ ID NO: 33 - DNA, 18 bp, Artificial Sequence
  An exemplary RB1 G4 oligonucleotide
cggggggttttgggcggc

SEQ ID NO: 34 - DNA, 22 bp, Artificial Sequence
  An exemplary VEGF G4 oligonucleotide
cggggcgggccgggggcggggt

SEQ ID NO: 35 - DNA, 27 bp, Artificial Sequence
  An exemplary c-MYC G4 oligonucleotide
tggggagggtggggagggtggggaagg

SEQ ID NO: 36 - DNA, 22 bp, Artificial Sequence
  An exemplary c-KIT G4 oligonucleotide
agggagggcgctgggaggaggg

SEQ ID NO: 37 - DNA, 26 bp, Artificial Sequence
  An exemplary dsDNA oligonucleotide
caatcggatcgaattcgatccgattg

SEQ ID NO: 38 - DNA, 19 bp, Artificial Sequence
  An exemplary HIF1-a G4 oligonucleotide
gggagggagagggggcggg

SEQ ID NO: 39 - DNA, 31 bp, Artificial Sequence
  An exemplary ssDNA oligonucleotide
tgtccccacacccctgtccccacacccctgt

SEQ ID NO: 40 - DNA, 6169 bp, Homo sapiens
gcgggcggagaagccgctccacatgcagtcctggaggatgaccgggttgggggtgaggccacccagtccccccaggccgaacgcgtcctcctcggccgggctgccccacagctcgttctcaagcagcatctccgtgacccagctcgggggctcggagctgtgctccgcgaagccacggctgggcgacagcgggggcgtgggcagcagctcaaacttcttccagatgtcctcccccgggggggtcgagtcggggccgccgaagtagaagtcatcttcgtccgggtagaagcagggctgtagcgagtcaaactcgaggtctgggttcttgcagatcatgcccggcatggtggacgtggagcagctcggcatcggctcgcctcccggccggcgactgagggcttctttccgccccgttcgttttaataccgggggtgcttccttcccgtggggggcgcggaaggcgggggccggcgccgacctccaacactgcaaccgacagacacaccggagagggtgggcaaacggggcagaccgaggggataggcaatgggcgccccctctaaccaggttccccaatcttccctccaagccccccactctgcccactgctcccgaaggtggaggaagttgtgcctctcgctccctccctcccgccctccttttgtgtacaagctgtctacgacagggggtgggagggggcatgcagatgcagggggtggtggcgaggctccgcaactttggaaactgccatttcattcacacaaggcactgcctgggggagggggctgttcctggctgcagaattctagctctcacgagcacgcagacaaccgcactcgcagcggtgtggggccggctgctcaggggaagccccaggctctccgacccagctaccgggaatggtatttggagtaccttctaccccctttgtaaaaatgcaacctcaggagtgcaaatgatagcaggggatttagaaaactttgcaaataaaaaaaagcctttctctagacagagaccaacaacagacacccatatccacaaatccttcccctccccaaatttgagcaaacgcccctcggcattcacccgctgcagggatactcctgccaatagcttaattggaagctctctaagcccacccctgctcttccaaacccacgacaacgtcctcttccagggagggagaggctgagcccctgttcccgggaatggcaggggcgcccgcgcgaggagggtcgccgggagagccgcctctcctccaagctggagcagccgtgacccagattgcgaccgcggcggcctcgccctgcctaatccccccctcttcttcctccaggggcaccctttggagaagaaccccagcctggggtggggacgcaccggctctccgacagctcaaacacagacagatcttctagagccgagggaatttcttttcgcagaagccattactccccccggtaagggctgcaaaaggattagggcgggtctcctctagccaggatgccttcggggctggaactggcttttctgggaaaacccagagatggttttgtttcggaagcagaaacagtcccccgggcatattagggcgcccggtgcgcacgtcgcaatcccgtccagggcttgccttgccccggcgctcgtcgcttccctgggcgccgggcggtttgcaagccctgctccttacctcccgccgactgcaggggatccggaggcgattctgcgcgggtgtctccgggtgggccgggggggcgggggtgtcctcggatggctacagtctgtgcgcgcttgcgcctggctagcgcttgctcggctccaacacagttcccaggagccccccgcatccacccagcgcgtccagacagatgactgtcacagcctgcgctttgaagctcgggggatattattaactgaaaaatctgacacacccggggggcggggagagaagggggctgtggcgcagacaagggggagggagaaaggagaggaaagcggctttaccccgccctcctgatttccataaaaatcaggggaggcgccaaggctttcgcgccaaaagccacttgcttttctttgcagagagaaggctgcaaagctgggaagcccagggtgtgctcctcccgcccttttggacccccgggcttgcaccggctgcactctgagaaccagctgcgcgcggagcggtgcaatgcagcacccaccctgcgagcctggcaattgcttgtcattaaaagaaaaaaaaattacggagggctccgggggtgtgtgttggggaggggagaccgatgcttctaacccagcccccgctttgactgcgtgttgtgcagctgagcgcgaggccaacgttgagcaaggccttgcagggaggttgctcctgtgtaattacgaaagaaggctagtccgaaggtgcaaaatagcagggagaggacgcgcccccttaggaacaagacctctggatgtttccagtttcaaattgaaagaagaggggcgccccccttgtttgaaaataaataaataaataagtgcgagctacgagggtggcgccgcggtggtttcttcggccgaaggcgacacctcgcggagactgagcagaaagcggagctgcccggccttggcgcggacctggagtgcggccactccgcaggtctcccgccgcccccgcgggactgtccacgcgtcctcaccactgcggaggaggaggcggcctggctctgcttcctagggggaatgggcgtacgttccctgcagccgtccccagggctcccaagttaaccttttcaaagccaccattctccccagttatgggcaggcgaatctgatttaccgagatggaagtgctgtccacacctcctgagacccagcctcggctcaaacagcttgattccaaactgttgaagggaaaaaggaaagctgctgtcactacttcgtttctttgtgcctcagtttcctcaattgtgaaatggagttcgtagtgcctcctacctgagagttatgaaagctcttataagtccccagcccataatagttgtgtcagctattattatgatgatgaccacgacatgctattcttctggggaaaataacacccctctgtccagcagcagcattggccccttcccttcccgccaccatcaaatcctggctttacttcactctggtctagcttttcttacaggtgtatttgtttgcagggggtcagacattattgcccctgccctccaaactctccaaaaacaaccctgatctcctggacagcccccttcacaaccaaatacacagtgaggtgggattttgccttgaaaaacattccttaggcttgtttggagagaggggaacctggccctgggattggggtgggaaggcttccccgcctctccctacttttttctccaaactggaaatgccattctccatcgcccccacctcttcacctcaccattggatctagttaaggaggaggatgctgagggtcagggggcaacccataaaatgctcacagattagcatttgtgaaggcctttccagccctgaccctggacgcctacagagctctagggagaaagattcccagagttctaggaagaggcccatgaatgcggatgagcagcccttccaagcacctttattattgttatttccctcacaaatgtaaaagccttcatctttgcaaaagccttttacagccccacttctgcttttccctagccccctaatcgccaccaagccctcctgctgaattctcgcctagcatcctgtcctttcctgtccagcactcacctccatttgtaatgatccatgcctgggagtatttgtttagtgctcacttccgtgacactgtggaagacaggctcagtctccctcactaacctgtgacctgggtcagttttgctccagagggaatccttacggcacagcagtgtacctgtatatagtaagtactcatggtattattcaagaccaagatcactgaatgagtccttctaaggtggccagatcttctgtggcaagactgtgaaaataggccaggatgggttcagttgctccgattcaaagtcctacctttgtcctttctcctcagatgaatgaccatcactaggtcattcaccctttgtcgagggacaaggtgtagcagaaaagggggacagagggacaggagacctggttactctacatgcctttgagcaaatcatttatacttcctggacctcaatctctaatctcctgcttttaatgcaagccagtaattactgacaagtctgaatctgaaggttaacagaatgataaaattcaacaaatatttattggttaccttcatttgtaagtagaaagataaaagactttgtacatggagaaaacatgctaatgctgattcttagtctgcttatagagaagcctgaaaatccctcttaaaaatgttgaaatacttcaactatgtgaacacacacaaaacaaatcttcatatggtttcaaagccaaggttttgttcagtactgccatggtttgaatgtgtcctccaaaaaaaaacacgttggaagtttaacccccagtgcaacagtgttggaggggaggtccccaaatgccagtgggacctagaccccagccagtgtccaggctcttgacaccatcaagagaatgaattcaaagaccagttggaaaatagtgaaagtacgatttattgcaaagggaaaagtacacactcaagaaaagggaatatgggtgtactcgagagactcacacgaaagggggtttggggttgctacctttacgggtttctttaaccaagaggcagaatattcatgatcattcctagaaaaagaaggagatttctcagaactgtgacaccacctatttttaaaccaaatatgggtgttctcggaagtgtcatgatgctagtggggtgtgtgatttagtatgttaatgagcgtataatgaggtcctaggagaaacctaggtcaaatccagtgccatgctgggttcagtgggtcttagccagcttggtctacactctgtttttcacggtcttagcccatggcctctgcagccatttcaacagtttccttttgctagtcatgtgaaattgctgcccaaaatcttctattattctgcagccaccttatattattcctgtctcaggacctaatgtgaggtgtttaggtcatgagggctccactcttgtgaatggattaatgctaattataaaagggcttgaggctaggagtttgatctcttgccctctcttcctttttcctgccaggggatggtacgccatgaaggccctcagcaattgctggcaccttgatattggacttctcagcctccagaaacatgagaaatacatttcttttctttataaattacccagtctgtgctattaggttatagcaacacaaaatgaactgagacaagtacccatctattactgtgtctgagcccctgggtgggtttggaacagaggtaagttaaggtacagccgcaaggagctcactgtggaccagggaaggagacccacatctgaaatggatgcctcaatctaatcaccaactgctaggatagagttatacacagcatttggtttataattaaggtgatttttgagagaggagaatattttagtttacaaatctcctctttactaagacacacacacacaaagaaacaacacacacatacacatgttgagaagggcccaaatcatgactggctaggagagcaacagcctctagatggtttttccagattgtgaattaaagatctgctgggttggccaggcatggtggctcacgcctgtaatcctggcacttcaagagtctgaggagggcagatggcttgagcccaggattttgagaccggcctgggcaacatggtgaaaccctgtctctacaaaaaatataaaaattagctgggcgtggtgggtgcgtgcctgtggtcccagctacttgggaggctgaggcaggaagatcacctgagcccagggagatcaaggctgcagtgtgccatcatcacaccactgcactccagcctggatgacagagtgagaccttgtctcaaaagaaaaaaaaaaagatctactgagtctgtcttcagggacaagagcacagtttctcctaggatcttcacacctccctcaaaatgtgtacatttttactgattaaataatttaaaaatc

(19) United States
(12) Patent Application Publication
(10) Pub. No.: US 2024/0287054 A1
(43) Pub. Date: Aug. 29, 2024

(54) COMPOUNDS THAT BIND NON-CANONICAL G-QUADRUPLEX STRUCTURES AND METHODS OF MAKING AND USING THE SAME

(71) Applicant: The U.S.A., as Represented by the Secretary, Department of Health and Human Services, Bethesda, MD (US)

(72) Inventors: John S. Schneekloth, Frederick, MD (US); Mo Yang, Frederick, MD (US); Xiao Liang, Frederick, MD (US); Christopher Fullenkamp, Frederick, MD (US)

(73) Assignee: The U.S.A., as Represented by the Secretary, Department of Health and Human Services, Bethesda, MD (US)

(21) Appl. No.: 18/568,738

(22) PCT Filed: Jun. 9, 2022
(86) PCT No.: PCT/US2022/032798

§ 371 Date: Dec. 8, 2023

Related U.S. Application Data
(60) Provisional application No. 63/208,918, filed on Jun. 9, 2021.

(51) Int. Cl. C07D 413/14; A61K 31/501; A61K 31/5377; A61K 31/55; C07D 413/04

(52) U.S. Cl. CPC C07D 413/14; A61K 31/501; A61K 31/5377; A61K 31/55; C07D 413/04

(57) ABSTRACT

Small molecule compounds that selectively bind to non-canonical G-quadruplex (G4) structures, such as G4s in DNA found in various types of genes described herein, along with methods of using the compounds to reduce or inhibit protein (e.g., N-Myc protein) expression in cells, such as cancer cells. The compounds have a structure according to formulas described herein, or a stereoisomer, tautomer, or pharmaceutically effective salt or ester thereof.

### CROSS REFERENCE TO RELATED APPLICATION

The present application claims the benefit of and priority to the earlier filing date of U.S. Provisional Patent Application No. 63/208,918, filed on Jun. 9, 2021, the entirety of which is incorporated herein by reference.

### ACKNOWLEDGMENT OF GOVERNMENT SUPPORT

This invention was made with government support under project number Z01 ZIA BC011585 07 awarded by the National Institutes of Health, National Cancer Institute—Center for Cancer Research. The government has certain rights in the invention.

### FIELD

This disclosure relates to compounds that selectively bind to non-canonical G-quadruplex DNA structures found in certain genes, as well as methods of using the compounds to reduce or inhibit expression of such genes in cells, such as cancer cells.

### BACKGROUND

The MYC family of genes encode transcription factors that broadly govern and amplify gene expression. Three Myc proteins, c-Myc, N-Myc, and 1-Myc, contain basic helix-loop-helix (bHLH) regions that bind to DNA and directly regulate transcription. Among these genes, MYCN has been shown to be involved in fetal development and is highly expressed in neural tissue. MYCN is often overexpressed or mutated in cancers and is considered an oncogene in cancers, such as neuroblastoma and small cell lung cancers. Embedded within the MYCN locus is a second transcript that is produced through antisense transcription and originally called ncym, which was shown to be imbedded within a longer noncoding RNA (lncRNA) called MYCN opposite-strand (MYCNOS). Expression of MYCNOS has been shown to decrease promoter occupancy of MYCN and to control MYCN transcript stability through sequence complementarity.

More recent studies have shown that NCYM, the variant 2 of the MYCNOS transcript, can also be translated to produce a small protein. This polypeptide, known as NCYM, has been characterized to have diverse regulatory activities, including controlling the stability of N-Myc protein, inhibiting GSK3β, and influencing Wnt/β-catenin signaling.

Molecules that control or inhibit the N-Myc protein and other proteins involved in cancers are of interest as anticancer agents; however, as a transcription factor, the N-Myc protein is classically considered “undruggable” and attempts to develop ligands that target N-Myc itself, and other undruggable proteins (particularly those, that like N-Myc, comprise G-quadruplexes or “G4s”), have not been successful. There exists a need in the art for compounds, such as small molecules, that can bind structures like G4s in disease-relevant genes to achieve selectivity for targeting for such genes, including protein-coding and non-coding gene products.

### SUMMARY

A new class of small molecule compounds that target non-canonical G4 structures, such as hairpin-containing G4s found in the MYCN gene, are described herein. In particular implementations, the compounds are useful, for example, in methods of reducing or inhibiting N-Myc expression in cells (such as cancer cells), as well as in methods of treating or preventing a cancer in a subject, where the cancer is characterized at least in part by N-Myc overexpression.

Described herein are compounds, or a stereoisomer, tautomer, or pharmaceutically acceptable salt or ester thereof, according to Formula IA

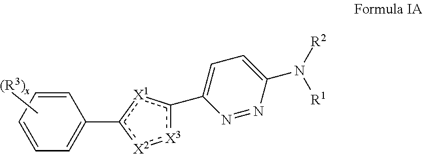

wherein
each of X¹, X², and X³ independently is N or O;
R¹ is -(linker)ₜ-R^{b} wherein R^{b} is aromatic, heteroaliphatic, or aliphatic; the linker is an aliphatic or heteroaliphatic group; and t is 0 or 1;
R² is H or aliphatic;
each R³ independently is heteroaliphatic, hydroxy, aliphatic, haloaliphatic, or halo;
x is an integer selected from 0 to 5; and
   provided that the compound is not 6-(3-(4-methoxyphenyl)-1,2,4-oxadiazol-5-yl)-N-(3-(pyrrolidin-1-yl)propyl)pyridazin-3-amine, N-(3-([1,4′-bipiperidin]-1′-yl)propyl)-6-(3-(4-methoxyphenyl)-1,2,4-oxadiazol-5-yl)pyridazin-3-amine, 6-(3-(4-methoxyphenyl)-1,2,4-oxadiazol-5-yl)-N-(3-(4-methylpiperazin-1-yl)propyl)pyridazin-3-amine, Ni-(6-(3-(4-methoxyphenyl)-1,2,4-oxadiazol-5-yl)pyridazin-3-yl)-N2,N2-dimethylethane-1,2-diamine, N-(3-(azepan-1-yl)propyl)-6-(3-(4-methoxyphenyl)-1,2,4-oxadiazol-5-yl)pyridazin-3-amine, 6-(3-(4-methoxyphenyl)-1,2,4-oxadiazol-5-yl)-N-(3-(4-methylpiperidin-1-yl)propyl)pyridazin-3-amine, or 6-(3-(4-methoxyphenyl)-1,2,4-oxadiazol-5-yl)-N-(2-morpholinoethyl)pyridazin-3-amine.

Also described herein are compounds, or a stereoisomer, tautomer, or pharmaceutically acceptable salt or ester thereof, according to Formula I

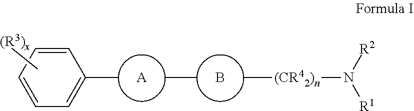

wherein
ring A is a 5-membered heteroaryl ring other than thiophenyl, thiazolyl, furanyl, triazolyl, thiadiazolyl, and 1,3,4-oxadiazolyl;
ring B is a 6-membered heteroaryl ring containing at least two nitrogen atoms;
R¹ is -(linker)ₜ-R^{b} wherein R^{b} is aromatic, heteroaliphatic, or aliphatic; the linker is an aliphatic or heteroaliphatic group; and t is 0 or 1;
R² is H or aliphatic;
each R³ independently is heteroaliphatic, hydroxy, aliphatic, haloaliphatic, or halo;
each R⁴ independently is H, aliphatic, or halo;
x is an integer selected from 0 to 5; and
n is an integer selected from 0 to 10;
   provided that the compound is not 6-(3-(4-methoxyphenyl)-1,2,4-oxadiazol-5-yl)-N-(3-(pyrrolidin-1-yl)propyl)pyridazin-3-amine, N-(3-([1,4′-bipiperidin]-1′-yl)propyl)-6-(3-(4-methoxyphenyl)-1,2,4-oxadiazol-5-yl)pyridazin-3-amine, 6-(3-(4-methoxyphenyl)-1,2,4-oxadiazol-5-yl)-N-(3-(4-methylpiperazin-1-yl)propyl)pyridazin-3-amine, N1-(6-(3-(4-methoxyphenyl)-1,2,4-oxadiazol-5-yl)pyridazin-3-yl)-N2,N2-dimethylethane-1,2-diamine, N-(3-(azepan-1-yl)propyl)-6-(3-(4-methoxyphenyl)-1,2,4-oxadiazol-5-yl)pyridazin-3-amine, 6-(3-(4-methoxyphenyl)-1,2,4-oxadiazol-5-yl)-N-(3-(4-methylpiperidin-1-yl)propyl)pyridazin-3-amine, or 6-(3-(4-methoxyphenyl)-1,2,4-oxadiazol-5-yl)-N-(2-morpholinoethyl)pyridazin-3-amine.

In some implementations, the compound, or a stereoisomer, tautomer, or pharmaceutically acceptable salt or ester thereof, has a structure according to Formula I:

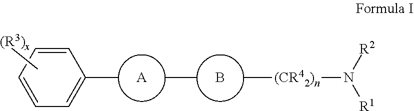

wherein
ring A is a 5-membered heteroaryl ring;
ring B is a 6-membered heteroaryl ring containing at least two nitrogen atoms;
R¹ is —(CR^{a}₂)ₘ—R^{b} where each Ra independently is H, alkyl, or halo, m is 1, 2, 3, 4, or 5, and R^{b} is a nitrogen-containing group;
R² is H or alkyl;
each R³ independently is alkoxy, hydroxy, aliphatic, or halo;
each R⁴ independently is H, alkyl, or halo;
n is 0, 1, 2, or 3; and
x is 0, 1, 2, 3, 4, or 5,
   with the proviso that the compound does not comprise the structure set forth as any one of MY-1, MY-2, MY-10, MY-11, or MY-12.

In some implementations, the compound, or a stereoisomer, tautomer, or pharmaceutically acceptable salt or ester thereof, has a structure according to Formula I:

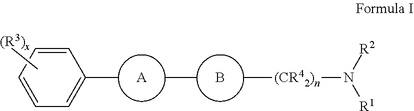

wherein
ring A is a 5-membered heteroaryl ring;
ring B is a 6-membered heteroaryl ring containing at least two nitrogen atoms;
R¹ is —(CR^{a}₂)ₘ—R^{b} or —[(CR^{a}₂)ₘO]ᵣ—(CH₂)ₛ—R^{b}, wherein each R^{a} independently is H, aliphatic, or halo; t is 1, 2, 3, 4, or 5; r is 1, 2, 3, 4, or 5; s is 0 or 1; and R^{b} is an acridinyl group;
R² is H or aliphatic;
each R³ independently is heteroaliphatic, hydroxy, aliphatic, haloaliphatic, or halo;
each R⁴ independently is H, aliphatic, or halo;
x is an integer selected from 0 to 5; and
n is an integer selected from 0 to 10.

Further disclosed are implementations of a pharmaceutical composition comprising a compound according to the present disclosure and at least one pharmaceutically acceptable additive.

Also disclosed is a method of decreasing cancer-relevant protein expression in a cell, comprising contacting the cell with an effective amount of a compound, or a stereoisomer, tautomer, or pharmaceutically acceptable salt or ester thereof, according to the present disclosure. And, use of a compound or a stereoisomer, tautomer, or pharmaceutically acceptable salt or ester thereof, according to the present disclosure for decreasing N-Myc expression in a cell, comprising contacting the cell with an effective amount of the compound is disclosed. In some implementations, the use is for treating or preventing cancer in a subject. In some implementations, the use is for the manufacture of a medicament for treating or preventing cancer in a subject.

The foregoing and other objects and features of the present disclosure will become more apparent from the following detailed description, which proceeds with reference to the accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a table describing five G-quadruplex sequences used for binding selectivity profiling as discussed herein, wherein all the DNA/RNA oligos were labeled with Cy5 fluorescent dye at 5′ ends and guanines that were involved in G4-formation are underlined.

FIG. 2 is a table describing MYCN wild-type/mutated/truncated G4 sequences.

FIG. 3 shows the location of the G4 forming sequence in the MYCN gene.

FIG. 4 is a map showing MYCN G4 forming probability obtained by genome-wide G4-sequence analysis.

FIGS. 5A and 5B show results of Quadruplex Forming G-Rich Sequence (or QGRS) analysis of the G-quadruplex forming sequences in MYCN/MYCNOS, wherein FIG. 5A show the G4-forming sequence (a target of interest) in MYCNOS discovered by G4-seq technology and FIG. 5B shows G-scores of potential G4s in this region.

FIG. 6 shows four hit compounds identified using small molecule microarray (SMM) screening.

FIG. 7 shows fluorescence intensity assay results for 14 hit compounds from the SMM (100 μM in 5% DMSO) and a negative control (compound 15), along with structures for four particular compounds, including compounds 1, 2, and 5, and compound MY-1.

FIG. 8 shows surface plasmon resonance results for the compounds of FIG. 7.

FIG. 9 shows binding affinity measurements by the fluorescence intensity assay for compounds 1, 2, MY-1, and 5.

FIG. 10 shows binding affinity measurements by surface plasmon resonance for compounds 1, 2, MY-1, and 5.

FIGS. 11A-11D show MYCN G4 structure stabilization by small molecule and binding selectivity evaluations, wherein FIG. 11A is circular dichroism (CD) spectra of MYCN G4 DNA in different buffers (10 mM Tris containing 100 mM KCl or 100 mM LiCl) and in water; FIG. 11B is a CD curve recording of 5 μM MYCN G4 during melting in low KCl buffer (10 mM Tris, pH 7.0, 5 mM KCl); FIG. 11C shows MYCN G4 melting with/without compounds 1 or MY-1; FIG. 11D shows CD melting tests of dsDNA incubated with compounds 1 or MY-1.

FIG. 12 shows CD spectra of MYCN G4 after annealing in buffers containing 10 mM Tris, pH 7.0, and 100, 20, or 5 mM KCl.

FIGS. 13A and 13B show the binding selectivity profiles of compounds 1 (FIG. 13A) and MY-1 (FIG. 13B) using tRNA, wherein a 2-aminopurine fluorescence assay was carried out by titrating the compounds into MYCN G4 DNA with the absence/presence of 10×tRNA.

FIGS. 14A-14E are graphs showing binding affinity of compound 1 with six different G4 targets, wherein all of the DNA/RNA G4 oligomers were labeled with fluorophore at 5′ and titration curves were obtained by a fluorescence intensity assay.

FIGS. 15A-15E are graphs showing binding affinity of compound MY-1 with six different G4 targets, wherein all of the DNA/RNA G4 oligomers were labeled with fluorophore at 5′ and titration curves were obtained by a fluorescence intensity assay.

FIGS. 16A-16E are quality control graphs of G4s used in selectivity evaluations described herein, wherein all G4s were tested by a fluorescence intensity assay using a classical G4 binder, TMPyP4 (100 μM), as a positive control.

FIG. 17 is an SPR curve of a competitive binding study between compounds 1 and MY-1.

FIGS. 18A and 18B illustrate results of a competitive assay of compounds 1 and MY-1, wherein FIG. 18A is an SPR curve of continuous injection of compounds 1 and MY-1, and FIG. 18B shows the binding level of compound MY-1 after injecting different concentrations of compound 1.

FIGS. 19A and 19B show results of an SPR binding assay of TMPyP4 with and without compound MY-1 at concentrations of 50 μM (FIG. 19A) and 250 μM (FIG. 19B).

FIGS. 20A and 20B include a CD melting curve of G4 stabilized by an individual compound (MY-1) or compound mixture (1 and MY-1) (FIG. 20A) and the melting temperature of MYCN G4 stabilized by the compound combination (FIG. 20B).

FIG. 21 is a graph showing the stoichiometry of compound MY-1 binding with MYCN G4 determined by Job Plot analysis.

FIG. 22 is a graph showing fluorescence titration of compound MY-1 using 3′-Cy5-labeled MYCN G4 DNA.

FIGS. 23A and 23B summarize results of an microscale thermophoresis (MST) study on compound MY-1 binding with 3′Cy5-MYCN G4.

FIGS. 24A-24F show results from a binding site evaluation using fluorescence quenching-based methods and dimethylsulfate (DMS) footprinting, wherein FIG. 24A is a schematic prediction of the MYCIN G4 folded structure; FIG. 24B is a quenching study by adding 100 μM MY-1 into solutions of 5′/3′-Cy5 labeled MYCN G4s; FIG. 24C is a quenching study by adding 100 μM MY-1 into solutions of 2-AP MYCN G4s labeled at A11, A18 and A24 positions, respectively; FIG. 24D shows fluorescence titration using wild type/mutated/truncated MYCN G4 DNA samples with 5′-Cy5 labeling—fluorescence intensities during titration were recorded and K_{D} values were calculated by fitting the curves; FIG. 24E is a fluorescence replacement assay using minor groove binders (Hoechst 33258 and netropsin); and FIG. 24F shows the DMS footprinting result of MYCN G4-DNA incubated with different concentrations of compound MY-1 (G-tracts involved in quadruplex tetrads are underlined), wherein protected Gs affected by compound MY-1 were labeled with dots (●) (highly affected) or circles (◯) (slightly affected).

FIGS. 25A-25C show quenching fraction determination by 2-AP modification-based titrations, wherein DNA oligomers with 2-AP labeling at the A11 (FIG. 25A), A18 (FIG. 25B), or A24 (FIG. 25C) positions were titrated with a series of concentrations of compound MY-1.

FIGS. 26A-26N show the results of binding assays performed with 14 different compounds of the present disclosure using SPR.

FIGS. 27A-27N show the results of binding assays performed with 14 different compounds of the present disclosure using 2-AP labeled DNA at the A11 position.

FIGS. 28A-28G show the effects of compound MY-8 on MYCN/MYCNOS expression in NBEB cells, wherein FIG. 28A is images of cell confluency after treatment with the compound at different concentrations; FIG. 28B shows time-dependent cell confluence curves at the different concentrations; FIG. 28C is an MTS assay analysis of cell viability; FIGS. 28D-28F are graphs of MYCN (FIG. 28D), MYCNOS001 (FIG. 28E), and MYCNOS002 (FIG. 28F) mRNA expression after MY-8 treatments; and FIG. 28G is a Western blot showing MYCN levels after MY-8 treatments.

FIG. 29 is a table summarizing representative hairpin G4 sequences in cancer-relevant genes.

FIG. 30 is a schematic diagram showing binding activity of a bivalent compound (compound B33) comprising a non-canonical G4-binding component and a G4-stacker component.

FIGS. 31A and 31B are graphs of surface plasmon resonance response as a function of time (FIG. 31A) and concentration (FIG. 31B) showing MYCN hairpin G4 binding affinity for acridine ICR 191.

FIGS. 32A and 32B are graphs of surface plasmon resonance response as a function of time (FIG. 32A) and concentration (FIG. 32B) showing MYCN hairpin G4 binding affinity for a bivalent compound according to the present disclosure (compound B33).

FIGS. 33A and 33B are graphs of surface plasmon resonance response as a function of time showing dsDNA binding affinity of acridine ICR 191 (FIG. 33A) and a bivalent compound according to the present disclosure (compound B33) (FIG. 33B).

FIGS. 34A and 34B are graphs of fluorescent intensity as a function of DNA concentration showing dsDNA binding affinity of acridine ICR 191 (FIG. 34A) and a bivalent compound according to the present disclosure (compound B33) (FIG. 34B) using FIA.

FIGS. 35A-35F show results of binding selectivity profiling using a G4 microarray and three different compounds, including a bivalent compound according to the present disclosure (compound B33), thiazole orange, and amsacrine; wherein FIGS. 35A, 35C, and 35E are optical images showing fluorescence observed with the bivalent compound, thiazole orange, and amsacrine, respectively, and different targets; and FIGS. 35B, 35D, and 35F are bar graphs showing the fluorescence intensity values for the binding selectivity of the bivalent compound, thiazole orange, and amsacrine, respectively, and the different targets.

FIG. 36 is a spectrum of obtained using circular dichroism spectroscopy, which shows results of a thermal melting assay of MYCN G4 DNA exposed to a control (DMSO), acridine ICR, and a bivalent compound according to the present disclosure (compound B33).

FIGS. 37A-37G are images of results obtained from binding selectivity profile testing of a bivalent compound of the present disclosure (compound B33) and thiazole orange using a high-density DNA oligo microarray, wherein FIG. 37A is an optical image showing fluorescence observed with these two different compounds; FIG. 37B is a kernel density estimate (KDE) plot and FIG. 37C is a violin plot representing the distribution of the binding signals throughout the microarray; FIGS. 37D and 37E provide plots summarizing the binding behavior of the two compounds; and FIGS. 37F (thiazole orange) and 37G (bivalent compound) are graphs providing the Gini coefficients for the two different compounds.

FIGS. 38A and 38B provide a DMS footprint of MYCN G4 DNA with and without a bivalent compound (compound B33) (FIG. 38A) and a proposed folded structure of the hairpin-containing G4 (FIG. 38B).

FIGS. 39A and 39B are FIA graphs of results for binding a bivalent compound (compound B33) to MYCN G4s with an unwound hairpin (FIG. 39A) and a truncated hairpin (FIG. 39B).

FIG. 40 is a graph of SPR response as a function of time for binding of a bivalent compound (compound B33) to MANGO II RNA G4, wherein weak binding was observed.

FIGS. 41A and 41B show results of a binding mode evaluation of a bivalent compound (compound B33) and a hairpin G4 having a parallel structure (BCL2).

FIGS. 42A and 42B show results of a binding mode evaluation of a bivalent compound (compound B33) and a hairpin G4 having a (3+1) hybrid structure (HIV).

FIGS. 43A and 43B show results of a binding mode evaluation of a bivalent compound (compound B33) and a hairpin G4 having a (3+1) hybrid structure (PIM1-form 1).

FIGS. 44A and 44B show results of a binding mode evaluation of a bivalent compound (compound B33) and a hairpin G4 having a (2+2) chair-type structure (PIM1-form 2).

FIG. 45 shows a summary of MYCN HP-G4 binding activity results (as assessed using FIA and SPR) for different bivalent compounds (B32, B33, B34, B35, and B38) having different linker groups.

FIGS. 46A-46D are graphs of FIA results for different bivalent compounds (B32, B33, B34, B35, and B38) having different linker groups.

FIGS. 47A-47H are graphs of SPR results for different bivalent compounds (B32, B33, B34, B35, and B38) having different linker groups.

FIGS. 48A and 48B are graphs of SPR results for amsacrine.

FIGS. 49A and 49B are graphs of SPR results for a bivalent compound (B32) (FIG. 49A) and amsacrine (FIG. 49B) with dsDNA.

### SEQUENCES

The nucleic and amino acid sequences listed in the accompanying sequence listing are shown using standard letter abbreviations for nucleotide bases, and three letter code for amino acids, as defined in 37 C.F.R. § 1.822. Only one strand of each nucleic acid sequence is shown, but the complementary strand is understood as included by any reference to the displayed strand. The Sequence Listing is submitted as an ASCII text file in the form of the file named “Sequence.txt” (28,672 bytes), created on Jun. 3, 2022, which is incorporated by reference herein. In the accompanying sequence listing:

SEQ ID NO: 1 is an exemplary genomic DNA sequence encoding human MYCN.

SEQ ID NOs: 2-6 are exemplary nucleic acid sequences of G-quadruplex oligonucleotides for BCL2, KRAS, mTOR, NRAS, and telomeric DNA, respectively.

SEQ ID NO: 7 is an exemplary MYCN G4 wild-type oligonucleotide.

SEQ ID NO: 8 is an exemplary MYCN G4 mutated oligonucleotide.

SEQ ID NO: 9 is an exemplary MYCN G4 truncated oligonucleotide.

SEQ ID NO: 10 is an exemplary nucleic acid sequence of a primer for the sense strand of MYCNOs-01.

SEQ ID NO: 11 is an exemplary nucleic acid sequence of a primer for the antisense strand of MYCNOs-01.

SEQ ID NO: 12 is an exemplary nucleic acid sequence of a primer for the sense strand of MYCNOs-02.

SEQ ID NO: 13 is an exemplary nucleic acid sequence of a primer for the antisense strand of MYCNOs-02.

SEQ ID NO: 14 is an exemplary nucleic acid sequence of a primer for the sense strand of MYCN protein isoform 1.

SEQ ID NO: 15 is an exemplary nucleic acid sequence of a primer for the antisense strand of MYCN protein isoform 1.

SEQ ID NO: 16 is an exemplary nucleic acid sequence of a primer for the sense strand of MYCN protein isoform 2.

SEQ ID NO: 17 is an exemplary nucleic acid sequence of a primer for the antisense strand of MYCN protein isoform 2.

SEQ ID NO: 18 is an exemplary nucleic acid sequence of a MYCN G4 quadruplex forming sequence.

SEQ ID NO: 19 is an exemplary nucleic acid sequence of the G4-forming sequence in MYCNOS.

SEQ ID NOs: 20-23 are exemplary nucleic acid sequences of potential G4s in MYCNOS.

SEQ ID NOs: 24-30 are exemplary nucleic acid sequences of hairpin G4 sequences in FOXA3, KRAS, MYCL, BRD4, BCL2, LINC01018, and SOX12, respectively.

SEQ ID NO: 31 is an exemplary hTERT G4 oligonucleotide.

SEQ ID NO: 32 is an exemplary BCL2 G4 oligonucleotide.

SEQ ID NO: 33 is an exemplary RB1 G4 oligonucleotide.

SEQ ID NO: 34 is an exemplary VEGF G4 oligonucleotide.

SEQ ID NO: 35 is an exemplary c-MYC G4 oligonucleotide.

SEQ ID NO: 36 is an exemplary c-KIT G4 oligonucleotide.

SEQ ID NO: 37 is an exemplary dsDNA oligonucleotide.

SEQ ID NO: 38 is an exemplary HIF1-a G4 oligonucleotide.

SEQ ID NO: 39 is an exemplary ssDNA oligonucleotide.

SEQ ID NO: 40 is an exemplary genomic DNA sequence encoding human MYCNOS.

### DETAILED DESCRIPTION

### I. Definitions and Abbreviations

The following explanations of terms and abbreviations are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise. The term “comprises” means “includes.” Therefore, comprising “A” or “B” refers to including A, including B, or including both A and B.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art. When directly and explicitly distinguishing implementations from discussed prior art, the implementation numbers are not approximates unless the word “about” is recited. It is further to be understood that all base sizes or amino acid sizes, and all molecular weight or molecular mass values, given for nucleic acids or polypeptides are approximate, and are provided for description.

Although there are alternatives for various components, parameters, operating conditions, etc. set forth herein, that does not mean that those alternatives are necessarily equivalent and/or perform equally well. Nor does it mean that the alternatives are listed in a preferred order unless stated otherwise.

A person of ordinary skill in the art would recognize that the definitions provided below are not intended to include impermissible substitution patterns (e.g., methyl substituted with 5 different groups, and the like). Such impermissible substitution patterns are easily recognized by a person of ordinary skill in the art. Any functional group disclosed herein and/or defined above can be substituted or unsubstituted, unless otherwise indicated herein.

Definitions of common terms in chemistry may be found in Richard J. Lewis, Sr. (ed.), Hawley's Condensed Chemical Dictionary, published by John Wiley & Sons, Inc., 2016 (ISBN 978-1-118-13515-0). The presently disclosed compounds also include all isotopes of atoms present in the compounds, which can include, but are not limited to, deuterium, tritium, ¹⁸F, ¹⁴C, etc.

A person of ordinary skill in the art will appreciate that compounds may exhibit the phenomena of tautomerism, conformational isomerism, geometric isomerism, and/or optical isomerism. For example, certain disclosed compounds can include one or more chiral centers and/or double bonds and as a consequence can exist as stereoisomers, such as double-bond isomers (i.e., geometric isomers), enantiomers, diastereomers, and mixtures thereof, such as racemic mixtures. As another example, certain disclosed compounds can exist in several tautomeric forms, including the enol form, the keto form, and mixtures thereof. As the various compound names, formulae and compound drawings within the specification and claims can represent only one of the possible tautomeric, conformational isomeric, optical isomeric, or geometric isomeric forms, a person of ordinary skill in the art will appreciate that the disclosed compounds encompass any tautomeric, conformational isomeric, optical isomeric, and/or geometric isomeric forms of the compounds described herein, as well as mixtures of these various different isomeric forms. Mixtures of different isomeric forms, including mixtures of enantiomers and/or stereoisomers, can be separated to provide each separate enantiomers and/or stereoisomer using techniques known to those of ordinary skill in the art, particularly with the benefit of the present disclosure. In cases of limited rotation, e.g. around the amide bond or between two directly attached rings such as pyridinyl rings, biphenyl groups, and the like, atropisomers are also possible and are also specifically included in the compounds disclosed herein.

In any implementations, any or all hydrogens present in the compound, or in a particular group or moiety within the compound, may be replaced by a deuterium or a tritium. Thus, a recitation of alkyl includes deuterated alkyl, where from one to the maximum number of hydrogens present may be replaced by deuterium. For example, methyl refers to both CH₃ or CH₃ wherein from 1 to 3 hydrogens are replaced by deuterium, such as in CDₓH₃₋ₓ.

As used herein, the term “substituted” refers to all subsequent modifiers in a term, for example in the term “substituted aliphatic-aromatic,” substitution may occur on the “aliphatic” portion, the “aromatic” portion or both portions of the aliphatic-aromatic group.

“Substituted,” when used to modify a specified group or moiety, means that at least one, and perhaps two or more, hydrogen atoms of the specified group or moiety is independently replaced with the same or different substituent groups. In a particular implementation, a group, moiety, or substituent may be substituted or unsubstituted, unless expressly defined as either “unsubstituted” or “substituted.” Accordingly, any of the functional groups specified herein may be unsubstituted or substituted unless the context indicates otherwise or a particular structural formula precludes substitution. In particular implementations, a substituent may or may not be expressly defined as substituted but is still contemplated to be optionally substituted. For example, an “aliphatic” or a “cyclic” moiety may be unsubstituted or substituted, but an “unsubstituted aliphatic” or an “unsubstituted cyclic” is not substituted. In one implementation, a group that is substituted has at least one substituent up to the number of substituents possible for a particular moiety, such as 1 substituent, 2 substituents, 3 substituents, or 4 substituents.

Any group or moiety defined herein can be connected to any other portion of a disclosed structure, such as a parent or core structure, as would be understood by a person of ordinary skill in the art, such as by considering valence rules, comparison to exemplary species, and/or considering functionality, unless the connectivity of the group or moiety to the other portion of the structure is expressly stated, or is implied by context.

In order to facilitate review of the various implementations of the disclosure, the following explanations of specific terms are provided:

Acyl Halide: —C(O)X, wherein X is a halogen, such as Br, F, I, or Cl.

Administration: To provide or give to a subject an agent, for example, a compound (e.g., a small compound) that selectively binds to a non-canonical G4, such as a non-canonical G4 of MYCN (encoding N-Myc), by any effective route. In some implementations, administration can comprise providing or giving to a subject an agent, for example, a small molecule compound that selectively binds to G4 quadruplex DNA in the c-MYC promoter, by any effective route. Exemplary routes of administration include, but are not limited to, oral, injection (such as subcutaneous, intramuscular, intradermal, intraperitoneal, and intravenous), sublingual, rectal, transdermal (for example, topical), intranasal, vaginal, and inhalation routes.

Co-administration or co-administering refers to administration of at least two therapeutic compounds within the same general time period, and does not require administration at the same exact moment in time (although co-administration is inclusive of administering at the same exact moment in time). Thus, co-administration may be on the same day or on different days, or in the same week or in different weeks. The therapeutic compounds disclosed herein may be included in the same composition or they may each individually be included in separate compositions. In certain implementations, the two compounds may be administered during a time frame wherein their respective periods of biological activity overlap. Thus, the term includes sequential as well as coextensive administration of two or more compounds.

“Administration of” and “administering a” compound should be understood to mean providing a compound, a prodrug of a compound, or a pharmaceutical composition as described herein. The compound or composition can be administered by another person to the subject (e.g., intravenously) or it can be self-administered by the subject (e.g., tablets).

Agent: Any substance or any combination of substances that is useful for achieving an end or result; for example, a substance or combination of substances useful for decreasing or reducing tumor growth in a subject. Agents include effector molecules and detectable markers. In some implementations, the agent is a chemotherapeutic agent. The skilled artisan will understand that particular agents may be useful to achieve more than one result; for example, an agent may be useful as both a detectable marker and a chemotherapeutic agent.

Aldehyde: —C(O)H.

Aliphatic: A hydrocarbon group (e.g., a substantially hydrocarbon-based compound) having at least one carbon atom to 50 carbon atoms (C₁₋₅₀), such as one to 25 carbon atoms (C₁₋₂₅), or one to ten carbon atoms (C₁₋₁₀), or one to six carbon atoms (C₁₋₆), or one to four carbon atoms (C₁₋₄), and which includes alkanes (or alkyl), alkenes (or alkenyl), alkynes (or alkynyl), including cyclic versions thereof, and further including straight- and branched-chain arrangements, and all stereo and position isomers as well. Aliphatic groups may be substituted with one or more groups other than hydrogen, such as aliphatic, heteroaliphatic, haloaliphatic, haloheteroaliphatic, aromatic, or an organic functional group. Exemplary substituents include, but are not limited to, alkyl, alkenyl, alkynyl, alkoxy, alkylamino, alkylthio, acyl, aldehyde, amide, amino, aminoalkyl, aryl, arylalkyl, carboxyl, cyano, cycloalkyl, dialkylamino, halo, haloaliphatic, heteroaliphatic, heteroaryl, heterocycloaliphatic, hydroxyl, oxo, sulfonamide, sulfhydryl, thioalkoxy, or other functionality.

Alkenyl: An unsaturated monovalent hydrocarbon having at least two carbon atom to 50 carbon atoms (C₂₋₅₀), such as two to 25 carbon atoms (C₂₋₂₅), or two to ten carbon atoms (C₂₋₁₀), and at least one carbon-carbon double bond, wherein the unsaturated monovalent hydrocarbon can be derived from removing one hydrogen atom from one carbon atom of a parent alkene. An alkenyl group can be branched, straight-chain, cyclic (e.g., cycloalkenyl), cis, or trans (e.g., E or Z). Alkenyl groups may be substituted with one or more groups other than hydrogen, such as aliphatic, heteroaliphatic, haloaliphatic, haloheteroaliphatic, aromatic, or an organic functional group.

Alkoxy: —O-aliphatic, such as —O-alkyl, —O-alkenyl, —O-alkynyl; with exemplary implementations including, but not limited to, methoxy, ethoxy, n-propoxy, isopropoxy, n-butoxy, i-butoxy, t-butoxy, sec-butoxy, n-pentoxy, cyclopropoxy, cyclohexyloxy, and the like (wherein any of the aliphatic components of such groups can comprise no double or triple bonds, or can comprise one or more double and/or triple bonds). Alkoxy groups may be substituted with one or more groups other than hydrogen, such as aliphatic, heteroaliphatic, haloaliphatic, haloheteroaliphatic, aromatic, or an organic functional group.

Alkyl: A saturated monovalent hydrocarbon having at least one carbon atom to 50 carbon atoms (C₁₋₅₀), such as one to 25 carbon atoms (C₁₋₂₅), or one to ten carbon atoms (C₁₋₁₀), wherein the saturated monovalent hydrocarbon can be derived from removing one hydrogen atom from one carbon atom of a parent compound (e.g., alkane). An alkyl group can be branched, straight-chain, or cyclic (e.g., cycloalkyl).

Alkyl groups may be substituted with one or more groups other than hydrogen, such as aliphatic, heteroaliphatic, haloaliphatic, haloheteroaliphatic, aromatic, or an organic functional group. In some implementations, a lower alkyl or (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkanoyl can be acetyl, propanoyl or butanoyl; halo(C₁-C₆)alkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; hydroxy(C₁-C₆)alkyl can be hydroxymethyl, 1-hydroxyethyl, 2-hydroxyethyl, 1-hydroxypropyl, 2-hydroxypropyl, 3-hydroxypropyl, 1-hydroxybutyl, 4-hydroxybutyl, 1-hydroxypentyl, 5-hydroxypentyl, 1-hydroxyhexyl, or 6-hydroxyhexyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₁-C₆)alkylthio can be methylthio, ethylthio, propylthio, isopropylthio, butylthio, isobutylthio, pentylthio, or hexylthio; (C₂-C₆)alkanoyloxy can be acetoxy, propanoyloxy, butanoyloxy, isobutanoyloxy, pentanoyloxy, or hexanoyloxy.

Alkynyl: An unsaturated monovalent hydrocarbon having at least two carbon atom to 50 carbon atoms (C₂₋₅₀), such as two to 25 carbon atoms (C₂₋₂₅), or two to ten carbon atoms (C₂₋₁₀), and at least one carbon-carbon triple bond, wherein the unsaturated monovalent hydrocarbon can be derived from removing one hydrogen atom from one carbon atom of a parent alkyne. An alkynyl group can be branched, straight-chain, or cyclic (e.g., cycloalkynyl). Alkenyl groups may be substituted with one or more groups other than hydrogen, such as aliphatic, heteroaliphatic, haloaliphatic, haloheteroaliphatic, aromatic, or an organic functional group.

Amide: —C(O)NR^{e}R^{f} or —NR^{e}C(O)R^{f} wherein each of Re and R independently is selected from hydrogen, aliphatic, heteroaliphatic, haloaliphatic, haloheteroaliphatic, aromatic, or an organic functional group and can be substituted with one or more groups other than hydrogen, such as aliphatic, heteroaliphatic, haloaliphatic, haloheteroaliphatic, aromatic, or an organic functional group.

Amino: —NR^{e}R^{f}, wherein each of R^{e} and R^{f} independently is selected from hydrogen, aliphatic, heteroaliphatic, haloaliphatic, haloheteroaliphatic, aromatic, or an organic functional group, and can be substituted with one or more groups other than hydrogen, such as aliphatic, heteroaliphatic, haloaliphatic, haloheteroaliphatic, aromatic, or an organic functional group.

Analog: A molecule that differs in chemical structure from a parent compound, for example a homolog (differing by an increment in the chemical structure or mass, such as a difference in the length of an alkyl chain or the inclusion of one of more isotopes), a molecular fragment, a structure that differs by one or more functional groups, or a change in ionization. An analog is not necessarily synthesized from the parent compound. A derivative is a molecule derived from the base structure.

Aromatic: A cyclic, conjugated group or moiety of, unless specified otherwise, from 5 to 15 ring atoms having a single ring (e.g., phenyl) or multiple condensed rings in which at least one ring is aromatic (e.g., naphthyl, indolyl, or pyrazolopyridinyl); that is, at least one ring, and optionally multiple condensed rings, have a continuous, delocalized π-electron system. Typically, the number of out of plane π-electrons corresponds to the Hückel rule (4n+2). The point of attachment to the parent structure typically is through an aromatic portion of the condensed ring system. For example,

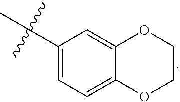 [SMILES: *c1ccc2c(c1)OCCO2]

However, in certain examples, context or express disclosure may indicate that the point of attachment is through a non-aromatic portion of the condensed ring system. For example,

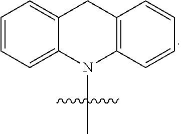 [SMILES: *N1c2ccccc2Cc2ccccc21]

An aromatic group or moiety may comprise only carbon atoms in the ring, such as in an aryl group or moiety, or it may comprise one or more ring carbon atoms and one or more ring heteroatoms comprising a lone pair of electrons (e.g. S, O, N, P, or Si), such as in a heteroaryl group or moiety. Aromatic groups may be substituted with one or more groups other than hydrogen, such as aliphatic, heteroaliphatic, haloaliphatic, haloheteroaliphatic, aromatic, or an organic functional group.

Aryl: An aromatic carbocyclic group comprising at least five carbon atoms, and in some implementations having at least five carbon atoms to 15 carbon atoms (C₅-C₁₅), such as five to ten carbon atoms (C₅-C₁₀), having a single ring or multiple condensed rings, which condensed rings can or may not be aromatic provided that the point of attachment to a remaining position of the compounds disclosed herein is through an atom of the aromatic carbocyclic group. Aryl groups may be substituted with one or more groups other than hydrogen, such as aliphatic, heteroaliphatic, haloaliphatic, haloheteroaliphatic, aromatic, or an organic functional group. In some implementations, The aryl group can be substituted with one or more groups including, but not limited to, alkyl, alkynyl, alkenyl, aryl, halide, nitro, amino, ester, ketone, aldehyde, hydroxy, carboxylic acid, or alkoxy.

Aroxy: —O-aromatic. Aroxy groups may be substituted with one or more groups other than hydrogen, such as aliphatic, heteroaliphatic, haloaliphatic, haloheteroaliphatic, aromatic, or an organic functional group.

Azo: —N═NR^{d} wherein R^{d} is hydrogen, aliphatic, heteroaliphatic, haloaliphatic, haloheteroaliphatic, aromatic, or an organic functional group. Azo groups may be substituted with one or more groups other than hydrogen, such as aliphatic, heteroaliphatic, haloaliphatic, haloheteroaliphatic, aromatic, or an organic functional group.

Cancer: A malignant tumor that has undergone characteristic anaplasia with loss of differentiation, increase rate of growth, invasion of surrounding tissue, and is capable of metastasis. For example, thyroid cancer is a malignant tumor that arises in or from thyroid tissue, and breast cancer is a malignant tumor that arises in or from breast tissue (such as a ductal carcinoma). Residual cancer is cancer that remains in a subject after any form of treatment given to the subject to reduce or eradicate the cancer.

Metastatic cancer is a tumor at one or more sites in the body other than the site of origin of the original (primary) cancer from which the metastatic cancer is derived. Cancer includes, but is not limited to, solid tumors.

Carbamate: —OC(O)NR^{e}R^{f}, wherein each of R^{e} and R^{f} independently is selected from hydrogen, aliphatic, heteroaliphatic, haloaliphatic, haloheteroaliphatic, aromatic, or an organic functional group. Carbamate groups can be substituted with one or more groups other than hydrogen, such as aliphatic, heteroaliphatic, haloaliphatic, haloheteroaliphatic, aromatic, or an organic functional group.

Carbonate: —OC(O)OR^{d}, wherein R^{d} is selected from aliphatic, heteroaliphatic, haloaliphatic, haloheteroaliphatic, aromatic, or an organic functional group. Carbonate groups can be substituted with one or more groups other than hydrogen, such as aliphatic, heteroaliphatic, haloaliphatic, haloheteroaliphatic, aromatic, or an organic functional group. In independent implementations, R^{d} can be hydrogen.

Carboxyl: —C(O)OH.

Carboxylate: —C(O)O⁻ or salts thereof, wherein the negative charge of the carboxylate group may be balanced with an M⁺ counterion, wherein M⁺ may be an alkali ion, such as K⁺, Na⁺, Li⁺; an ammonium ion, such as ⁺N(R^{e})₄ where R^{e} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}.

Chemotherapeutic agent: Any chemical agent with therapeutic usefulness in the treatment of diseases characterized by abnormal cell growth. For example, chemotherapeutic agents are useful for the treatment of neuroblastoma. Particular examples of additional therapeutic agents that can be used include microtubule binding agents, DNA intercalators or cross-linkers, DNA synthesis inhibitors, DNA and RNA transcription inhibitors, antibodies, enzymes, enzyme inhibitors, gene regulators, and angiogenesis inhibitors. In one implementation, a chemotherapeutic agent is a radioactive compound. One of skill in the art can readily identify a chemotherapeutic agent of use (see for example, Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., © 2000 Churchill Livingstone, Inc; Baltzer, L., Berkery, R. (eds): Oncology Pocket Guide to Chemotherapy, 2nd ed. St. Louis, Mosby-Year Book, 1995; Fischer, D. S., Knobf, M. F., Durivage, H. J. (eds): The Cancer Chemotherapy Handbook, 4th ed. St. Louis, Mosby-Year Book, 1993; Chabner and Longo, Cancer Chemotherapy and Biotherapy: Principles and Practice (4th ed.). Philadelphia: Lippincott Willians & Wilkins, 2005; Skeel. Handbook of Cancer Chemotherapy (6th ed.). Lippincott Williams & Wilkins, 2003). Combination chemotherapy is the administration of more than one agent to treat cancer.

Control: A sample or standard used for comparison with an experimental sample. In some implementations, the control is a sample obtained from a healthy patient or a non-tumor tissue sample obtained from a patient diagnosed with cancer. In other implementations, the control is a tumor tissue sample obtained from a patient diagnosed with cancer. In some implementations, the control is a tumor tissue sample obtained from a patient diagnosed with cancer, where the patient has not received treatment with a G4 stabilizing agent as disclosed herein. In still other implementations, the control is a historical control or standard reference value or range of values (such as a previously tested control sample, such as a group of cancer patients with known prognosis or outcome, or group of samples that represent baseline or normal values, such as the expression level of the MYCN or MYC gene in a non-tumor tissue).

Cyano: —CN.

Decrease or Reduce: To reduce the quality, amount, or strength of something; for example a reduction in tumor burden. In one example, a therapy reduces a tumor (such as the size of a tumor, the number of tumors, the metastasis of a tumor, or combinations thereof), or one or more symptoms associated with a tumor, for example as compared to the response in the absence of the therapy. In a particular example, a therapy decreases the size of a tumor, the number of tumors, the metastasis of a tumor, or combinations thereof, subsequent to the therapy, such as a decrease of at least 10%, at least 20%, at least 30%, at least 40%, at least 50%, at least 60%, at least 70%, at least 80%, or at least 90%. Such decreases can be measured using the methods disclosed herein.

Determining or detecting the level of expression of a gene product: Detection of a level of expression in either a qualitative or quantitative manner, for example by detecting nucleic acid molecules or proteins, for instance using routine methods known in the art.

Diagnosis: The process of identifying a disease by its signs, symptoms and results of various tests. The conclusion reached through that process is also called “a diagnosis.” Forms of testing commonly performed include blood tests, medical imaging, urinalysis, and biopsy.

Disulfide: —SSR^{d}, wherein R^{d} is selected from hydrogen, aliphatic, heteroaliphatic, haloaliphatic, haloheteroaliphatic, aromatic, or an organic functional group. Disulfide groups can be substituted with one or more groups other than hydrogen, such as aliphatic, heteroaliphatic, haloaliphatic, haloheteroaliphatic, aromatic, or an organic functional group.

Dithiocarboxylic: —C(S)SR^{d} wherein R^{d} is selected from hydrogen, aliphatic, heteroaliphatic, haloaliphatic, haloheteroaliphatic, aromatic, or an organic functional group. Dithiocarboxylic groups can be substituted with one or more groups other than hydrogen, such as aliphatic, heteroaliphatic, haloaliphatic, haloheteroaliphatic, aromatic, or an organic functional group.

Ester: —C(O)OR^{d} or —OC(O)R^{d}, wherein R^{d} is selected from aliphatic, heteroaliphatic, haloaliphatic, haloheteroaliphatic, aromatic, or an organic functional group. In some implementations, CO₂C₁₋₃alkyl groups are preferred, such as for example, methylester (CO₂Me), ethylester (CO₂Et), and propylester (CO₂Pr) and includes reverse esters thereof (e.g. —OC(O)Me, —OC(O)Et and —OC(O)Pr). Ester groups can be substituted with one or more groups other than hydrogen, such as aliphatic, heteroaliphatic, haloaliphatic, haloheteroaliphatic, aromatic, or an organic functional group.

Ether: -aliphatic-O-aliphatic, -aliphatic-O-aromatic, -aromatic-O-aliphatic, or -aromatic-O-aromatic. Ether groups can be substituted with one or more groups other than hydrogen, such as aliphatic, heteroaliphatic, haloaliphatic, haloheteroaliphatic, aromatic, or an organic functional group.

Hairpin: The term “hairpin” refers to a DNA structure in which two regions of the same strand, usually complementary in sequence when read in opposite directions, base-pair to form a double helix with an unpaired loop at the distal end of the hairpin:

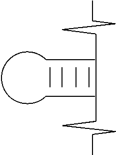

Halo (or halide or halogen): Fluoro, chloro, bromo, or iodo. In some implementations, halo can also include astatine.

Haloaliphatic: An aliphatic group wherein one or more hydrogen atoms, such as one to 10 hydrogen atoms, independently is replaced with a halogen atom, such as fluoro, bromo, chloro, or iodo. Haloaliphatic groups can be substituted with one or more groups other than hydrogen, such as aliphatic, heteroaliphatic, haloaliphatic, haloheteroaliphatic, aromatic, or an organic functional group.

Haloalkyl: An alkyl group wherein one or more hydrogen atoms, such as one to 10 hydrogen atoms, independently is replaced with a halogen atom, such as fluoro, bromo, chloro, or iodo. Haloalkyl groups can be substituted with one or more groups other than hydrogen, such as aliphatic, heteroaliphatic, haloaliphatic, haloheteroaliphatic, aromatic, or an organic functional group. In an independent implementation, haloalkyl can be a CX₃ group, wherein each X independently can be selected from fluoro, bromo, chloro, or iodo.

Haloheteroaliphatic: A heteroaliphatic group wherein one or more hydrogen atoms, such as one to 10 hydrogen atoms, independently is replaced with a halogen atom, such as fluoro, bromo, chloro, or iodo. Haloheteroaliphatic groups can be substituted with one or more groups other than hydrogen, such as aliphatic, heteroaliphatic, haloaliphatic, haloheteroaliphatic, aromatic, or an organic functional group.

Heteroaliphatic: An aliphatic group comprising at least one heteroatom to 20 heteroatoms, such as one to 15 heteroatoms, or one to 5 heteroatoms, which can be selected from, but not limited to oxygen, nitrogen, sulfur, silicon, boron, selenium, phosphorous, and oxidized forms thereof within the group. Alkoxy, ether, amino, disulfide, peroxy, and thioether groups are exemplary (but non-limiting) examples of heteroaliphatic. Heteroaliphatic groups can be substituted with one or more groups other than hydrogen, such as aliphatic, heteroaliphatic, haloaliphatic, haloheteroaliphatic, aromatic, or an organic functional group.

Heteroaryl: An aromatic group (e.g., an aryl group) comprising at least one heteroatom to six heteroatoms, such as one to four heteroatoms, which can be selected from, but not limited to oxygen, nitrogen, sulfur, silicon, boron, selenium, phosphorous, and oxidized forms thereof within the ring. Such heteroaryl groups can have a single ring or multiple condensed rings, wherein the condensed rings may or may not be aromatic and/or contain a heteroatom, provided that the point of attachment is through an atom of the aromatic heteroaryl group. Heteroaryl groups may be substituted with one or more groups other than hydrogen, such as aliphatic, heteroaliphatic, haloaliphatic, haloheteroaliphatic, aromatic, or an organic functional group.

Heteroatom: An atom other than carbon or hydrogen, such as (but not limited to) oxygen, nitrogen, sulfur, silicon, boron, selenium, or phosphorous. In particular disclosed implementations, such as when valency constraints do not permit, a heteroatom does not include a halogen atom.

Heterocycle/heterocycloaliphatic: A cycloaliphatic group having at least one carbon atom and at least one heteroatom, i.e., one or more carbon atoms has been replaced with an atom having at least one lone pair of electrons, typically nitrogen, oxygen, phosphorus, silicon, or sulfur. Heterocyclic groups can be mono-cyclic or bi-cyclic.

Hydroxy: A group represented by the formula —OH.

Isolated or Purified: An biological component is a component that has been substantially separated or purified away from other biological components in the cell of the organism in which the component naturally occurs, i.e., other chromosomal and extra-chromosomal DNA and RNA, proteins, lipids, and organelles. “Isolated” does not require absolute purity. For example, the desired isolated biological component may represent at least 50%, particularly at least about 75%, more particularly at least about 90%, and most particularly at least about 98%, of the total content of the preparation. Isolated biological components as described herein can be isolated by many methods such as salt fractionation, phenol extraction, precipitation with organic solvents (for example, hexadecyltrimethylammonium bromide or ethanol), affinity chromatography, ion-exchange chromatography, hydrophobic chromatography, high performance liquid chromatography, gel filtration, iso-electric focusing, physical separation (e.g., centrifugation or stirring), and the like.

The term purified does not require absolute purity; rather, it is intended as a relative term. Thus, for example, a purified peptide preparation is one in which the peptide or protein is more enriched than the peptide or protein is in its natural environment within a cell. For example, a compound preparation is purified such that the desired polysaccharide protein conjugate represents at least 50%, more particularly at least about 90%, and most particularly at least about 98%, of the total content of the preparation.

Ketone: —C(O)R^{d}, wherein R^{d} is selected from aliphatic, heteroaliphatic, haloaliphatic, haloheteroaliphatic, aromatic, or an organic functional group. Ketone groups can be substituted with one or more groups other than hydrogen, such as aliphatic, heteroaliphatic, haloaliphatic, haloheteroaliphatic, aromatic, or an organic functional group.

Nitrogen-Containing Group: A group that comprises at least one nitrogen atom. In some implementations, the nitrogen-containing group is an amide group or an amino group. In some implementations, the nitrogen-containing group can comprise mono or bicyclic rings, or ring systems, that include at least one nitrogen atom. The rings or ring systems generally include 1 to 9 carbon atoms in addition to the heteroatom(s) and may be saturated, unsaturated or aromatic (including pseudoaromatic). The term “pseudoaromatic” refers to a ring system that is stabilized by means of delocalization of electrons and behaves in a similar manner to aromatic rings. Aromatic includes pseudoaromatic ring systems, such as pyrrolyl rings.

Examples of nitrogen-containing groups include heteroaryl groups and/or cycloheteroaliphatic groups, such as pyrrolyl, H-pyrrolyl, pyrrolinyl, pyrrolidinyl, oxazolyl, oxadiazolyl, (including 1,2,3- and 1,2,4-oxadiazolyls) isoxazolyl, furazanyl, thiazolyl, isothiazolyl, pyrazolyl, pyrazolinyl, pyrazolidinyl, imidazolyl, imidazolinyl, triazolyl (including 1,2,3- and 1,3,4-triazolyls), tetrazolyl, thiadiazolyl (including 1,2,3- and 1,3,4-thiadiazolyls), dithiazolyl, pyridyl, pyrimidinyl, pyridazinyl, pyrazinyl, piperidinyl, morpholinyl, thiomorpholinyl, piperazinyl, triazinyl, 1H thieno[2,3-c]pyrazolyl, indolyl, isoindolyl, benzoxazolyl, benzothiazolyl, benzisoxazolyl, benzisothiazolyl, benzimidazolyl, indazolyl, isoquinolinyl, quinolinyl, quinoxalinyl, purinyl, cinnolinyl, phthalazinyl, quinazolinyl, quinoxalinyl, benzotriazinyl, and the like. Such nitrogen-containing groups can be fused to a carbocyclic ring such as phenyl, naphthyl, indenyl, azulenyl, fluorenyl, and anthracenyl. Unless otherwise defined optionally substituted cyclic nitrogen-containing group include pyridinium salts and the N-oxide form of suitable ring nitrogen atoms. In some implementations, the nitrogen-containing group can be optionally substituted with a broad range of substituents, and preferably with C₁₋₆ alkyl, C₁₋₆ alkoxy, C₂₋₆ alkenyl, C₂₋₆ alkynyl, halo, hydroxy, mercapto, trifluoromethyl, amino, cyano or mono or di(C₁₋₆alkyl)amino.

N-Myc: N-Myc protein is encoded by the MYCN gene. MYCN has been shown to be critical to fetal development and is highly expressed in neural tissue. MYCN is often overexpressed or mutated in cancers and is considered an oncogene, particularly in neuroblastoma and small cell lung cancers. Sequence information for MYCN has been described on public databases, for example, Ensembl (uswest.ensembl.org) gene ID no: ENSG00000134323. MYCN opposite strand (MYCNOS) is located on the antisense strand of MYCN. Expression of MYCNOS has been shown to decrease promoter occupancy of MYCN and control MYCN transcript stability through sequence complementarity. More recent studies have shown that NCYM, the variant 2 of the MYCNOS transcript, can also be translated to produce a small protein. This polypeptide, known as NCYM, has been characterized to have diverse regulatory activities, including controlling the stability of N-Myc protein, inhibiting GSK3β, and influencing Wnt/β-catenin signaling. Sequence information for MYCNOS has been described on public databases, for example, Ensembl (uswest.ensembl.org) gene ID no: ENSG00000233718.

Organic Functional Group: A functional group that may be provided by any combination of aliphatic, heteroaliphatic, aromatic, haloaliphatic, and/or haloheteroaliphatic groups, or that may be selected from, but not limited to, aldehyde; aroxy; acyl halide; halogen; nitro; cyano; azide; carboxyl (or carboxylate); amide; ketone; carbonate; imine; azo; carbamate; hydroxyl; thiol; sulfonyl (or sulfonate); oxime; ester; thiocyanate; thioketone; thiocarboxylic acid; thioester; dithiocarboxylic; phosphonate; phosphate; silyl ether; sulfinyl; sulfonamide; thial; or combinations thereof. Organic functional groups can be substituted with one or more groups other than hydrogen, such as aliphatic, heteroaliphatic, haloaliphatic, haloheteroaliphatic, aromatic, or an organic functional group.

Oxime: —CR^{d}=NOH, wherein R^{d} is hydrogen, aliphatic, heteroaliphatic, haloaliphatic, haloheteroaliphatic, aromatic, or an organic functional group. Oxime groups can be substituted with one or more groups other than hydrogen, such as aliphatic, heteroaliphatic, haloaliphatic, haloheteroaliphatic, aromatic, or an organic functional group.

Peroxy: —O—OR^{d} wherein R^{d} is hydrogen, aliphatic, heteroaliphatic, haloaliphatic, haloheteroaliphatic, aromatic, or an organic functional group. Peroxy groups can be substituted with one or more groups other than hydrogen, such as aliphatic, heteroaliphatic, haloaliphatic, haloheteroaliphatic, aromatic, or an organic functional group.

Pharmaceutical composition: A composition including an amount (for example, a unit dosage) of one or more of the disclosed compounds together with one or more non-toxic pharmaceutically acceptable additives, including carriers, diluents, and/or adjuvants, and optionally other biologically active ingredients. Such pharmaceutical compositions can be prepared by standard pharmaceutical formulation techniques such as those disclosed in Remington's Pharmaceutical Sciences, Mack Publishing Co., Easton, PA (19th Edition).

Pharmaceutically acceptable carrier: The pharmaceutically acceptable carriers of use are conventional. Remington's Pharmaceutical Sciences, by E. W. Martin, Mack Publishing Co., Easton, PA, 19th Edition, 1995, describes compositions and formulations suitable for pharmaceutical delivery of the disclosed immunogens.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate. In particular implementations, suitable for administration to a subject the carrier may be sterile, and/or suspended or otherwise contained in a unit dosage form containing one or more measured doses of the composition suitable to induce the desired tumor response. It may also be accompanied by medications for its use for treatment purposes. The unit dosage form may be, for example, in a sealed vial that contains sterile contents or a syringe for injection into a subject, or lyophilized for subsequent solubilization and administration or in a solid or controlled release dosage.

Pharmaceutically acceptable salt or ester: Salts or esters prepared by conventional means that include salts, e.g., of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid and the like.

Pharmaceutically acceptable salts of the presently disclosed compounds also include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, and tetramethylammonium hydroxide. These salts may be prepared by standard procedures, for example by reacting the free acid with a suitable organic or inorganic base. Any chemical compound recited in this specification may alternatively be administered as a pharmaceutically acceptable salt thereof. “Pharmaceutically acceptable salts” are also inclusive of the free acid, base, and zwitterionic forms. Descriptions of suitable pharmaceutically acceptable salts can be found in Handbook of Pharmaceutical Salts, Properties, Selection and Use, Wiley VCH (2002). When compounds disclosed herein include an acidic function such as a carboxy group, then suitable pharmaceutically acceptable cation pairs for the carboxy group are well known to those skilled in the art and include alkaline, alkaline earth, ammonium, quaternary ammonium cations and the like. Such salts are known to those of skill in the art. For additional examples of pharmacologically acceptable salts, see Berge et al., J. Pharm. Sci. 66:1 (1977).

Pharmaceutically acceptable esters include those derived from compounds described herein that are modified to include a carboxyl group. An in vivo hydrolysable ester is an ester, which is hydrolysed in the human or animal body to produce the parent acid or alcohol. Representative esters thus include carboxylic acid esters in which the non-carbonyl moiety of the carboxylic acid portion of the ester grouping is selected from straight or branched chain alkyl (for example, methyl, n-propyl, t-butyl, or n-butyl), cycloalkyl, alkoxyalkyl (for example, methoxymethyl), aralkyl (for example benzyl), aryloxyalkyl (for example, phenoxymethyl), aryl (for example, phenyl, optionally substituted by, for example, halogen, C.sub.1-4 alkyl, or C.sub.1-4 alkoxy) or amino); sulphonate esters, such as alkyl- or aralkylsulphonyl (for example, methanesulphonyl); or amino acid esters (for example, L-valyl or L-isoleucyl). A “pharmaceutically acceptable ester” also includes inorganic esters such as mono-, di-, or tri-phosphate esters. In such esters, unless otherwise specified, any alkyl moiety present advantageously contains from 1 to 18 carbon atoms, particularly from 1 to 6 carbon atoms, more particularly from 1 to 4 carbon atoms. Any cycloalkyl moiety present in such esters advantageously contains from 3 to 6 carbon atoms. Any aryl moiety present in such esters advantageously comprises a phenyl group, optionally substituted as shown in the definition of carbocycylyl above. Pharmaceutically acceptable esters thus include C₁-C₂₂ fatty acid esters, such as acetyl, t-butyl or long chain straight or branched unsaturated or omega-6 monounsaturated fatty acids such as palmoyl, stearoyl and the like. Alternative aryl or heteroaryl esters include benzoyl, pyridylmethyloyl and the like any of which may be substituted, as defined in carbocyclyl above. Additional pharmaceutically acceptable esters include aliphatic L-amino acid esters such as leucyl, isoleucyl and especially valyl.

For therapeutic use, salts of the compounds are those wherein the counter-ion is pharmaceutically acceptable. However, salts of acids and bases which are non-pharmaceutically acceptable may also find use, for example, in the preparation or purification of a pharmaceutically acceptable compound.

The pharmaceutically acceptable acid and base addition salts as mentioned hereinabove are meant to comprise the therapeutically active non-toxic acid and base addition salt forms which the compounds are able to form. The pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic (i.e. hydroxybutanedioic acid), tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form.

The compounds containing an acidic proton may also be converted into their non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like.

The term addition salt as used hereinabove also comprises the solvates which the compounds described herein are able to form. Such solvates are for example hydrates, alcoholates and the like.

The term “quaternary amine” as used hereinbefore defines the quaternary ammonium salts which the compounds are able to form by reaction between a basic nitrogen of a compound and an appropriate quaternizing agent, such as, for example, an optionally substituted alkylhalide, arylhalide or arylalkylhalide, e.g. methyliodide or benzyliodide. Other reactants with good leaving groups may also be used, such as alkyl trifluoromethanesulfonates, alkyl methanesulfonates, and alkyl p-toluenesulfonates. A quaternary amine has a positively charged nitrogen. Pharmaceutically acceptable counterions include chloro, bromo, iodo, trifluoroacetate and acetate. The counterion of choice can be introduced using ion exchange resins.

Prodrugs of the disclosed compounds also are contemplated herein. A prodrug is an active or inactive compound that is modified chemically through in vivo physiological action, such as hydrolysis, metabolism and the like, into an active compound following administration of the prodrug to a subject. The term “prodrug” as used throughout this text means the pharmacologically acceptable derivatives such as esters, amides and phosphates, such that the resulting in vivo biotransformation product of the derivative is the active drug as defined in the compounds described herein. Prodrugs preferably have excellent aqueous solubility, increased bioavailability and are readily metabolized into the active inhibitors in vivo. Prodrugs of a compounds described herein may be prepared by modifying functional groups present in the compound in such a way that the modifications are cleaved, either by routine manipulation or in vivo, to the parent compound. The suitability and techniques involved in making and using prodrugs are well known by those skilled in the art. For a general discussion of prodrugs involving esters see Svensson and Tunek, Drug Metabolism Reviews 165 (1988) and Bundgaard, Design of Prodrugs, Elsevier (1985).

The term “prodrug” also is intended to include any covalently bonded carriers that release an active parent drug of the present invention in vivo when the prodrug is administered to a subject. Since prodrugs often have enhanced properties relative to the active agent pharmaceutical, such as, solubility and bioavailability, the compounds disclosed herein can be delivered in prodrug form. Thus, also contemplated are prodrugs of the presently disclosed compounds, methods of delivering prodrugs and compositions containing such prodrugs. Prodrugs of the disclosed compounds typically are prepared by modifying one or more functional groups present in the compound in such a way that the modifications are cleaved, either in routine manipulation or in vivo, to yield the parent compound. Prodrugs include compounds having a phosphonate and/or amino group functionalized with any group that is cleaved in vivo to yield the corresponding amino and/or phosphonate group, respectively. Examples of prodrugs include, without limitation, compounds having an acylated amino group and/or a phosphonate ester or phosphonate amide group. In particular examples, a prodrug is a lower alkyl phosphonate ester, such as an isopropyl phosphonate ester.

Protected derivatives of the disclosed compounds also are contemplated. A variety of suitable protecting groups for use with the disclosed compounds are disclosed in Greene and Wuts, Protective Groups in Organic Synthesis; 3rd Ed.; John Wiley & Sons, New York, 1999.

In general, protecting groups are removed under conditions that will not affect the remaining portion of the molecule. These methods are well known in the art and include acid hydrolysis, hydrogenolysis and the like. One preferred method involves the removal of an ester, such as cleavage of a phosphonate ester using Lewis acidic conditions, such as in TMS-Br mediated ester cleavage to yield the free phosphonate. A second preferred method involves removal of a protecting group, such as removal of a benzyl group by hydrogenolysis utilizing palladium on carbon in a suitable solvent system such as an alcohol, acetic acid, and the like or mixtures thereof. A t-butoxy-based group, including t-butoxy carbonyl protecting groups can be removed utilizing an inorganic or organic acid, such as HCl or trifluoroacetic acid, in a suitable solvent system, such as water, dioxane and/or methylene chloride. Another exemplary protecting group, suitable for protecting amino and hydroxy functions amino is trityl. Other conventional protecting groups are known and suitable protecting groups can be selected by those of skill in the art in consultation with Greene and Wuts, Protective Groups in Organic Synthesis; 3rd Ed.; John Wiley & Sons, New York, 1999. When an amine is deprotected, the resulting salt can readily be neutralized to yield the free amine. Similarly, when an acid moiety, such as a phosphonic acid moiety is unveiled, the compound may be isolated as the acid compound or as a salt thereof.

Phosphate: —O—P(O)(OR^{d})₂, wherein each R^{d} independently is hydrogen, aliphatic, heteroaliphatic, haloaliphatic, haloheteroaliphatic, aromatic, or an organic functional group; or wherein one or more R^{d} groups are not present and the phosphate group therefore has at least one negative charge, which can be balanced by a counterion, M+, wherein each M⁺ independently can be an alkali ion, such as K⁺, Na⁺, Li⁺; an ammonium ion, such as ⁺N(R^{e})₄ where R^{e} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}. The R^{d} groups of the phosphate can be substituted with one or more groups other than hydrogen, such as aliphatic, heteroaliphatic, haloaliphatic, haloheteroaliphatic, aromatic, or an organic functional group.

Phosphonate: —P(O)(OR^{d})₂, wherein each R^{d} independently is hydrogen, aliphatic, heteroaliphatic, haloaliphatic, haloheteroaliphatic, aromatic, or an organic functional group; or wherein one or more R^{d} groups are not present and the phosphate group therefore has at least one negative charge, which can be balanced by a counterion, M⁺, wherein each M⁺ independently can be an alkali ion, such as K⁺, Na⁺, Li⁺; an ammonium ion, such as ⁺N(R^{e})₄ where R^{e} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}. The R^{d} groups of the phosphonate group can be substituted with one or more groups other than hydrogen, such as aliphatic, heteroaliphatic, haloaliphatic, haloheteroaliphatic, aromatic, or an organic functional group.

Silyl Ether: —OSiR^{e}R^{f}, wherein each of R^{e} and R^{f} independently is selected from hydrogen, aliphatic, heteroaliphatic, haloaliphatic, haloheteroaliphatic, aromatic, or an organic functional group. Silyl ether groups can be substituted with one or more groups other than hydrogen, such as aliphatic, heteroaliphatic, haloaliphatic, haloheteroaliphatic, aromatic, or an organic functional group.

Small molecule: As used herein, the term “small molecule” refers to a molecule with a molecular weight of 1000 daltons or less (for example 900 daltons or less, 800 daltons or less, 700 daltons or less, 600 daltons or less, 500 daltons or less, 400 daltons or less, 300 daltons or less, 200 daltons or less, or 100 daltons or less). In some examples, a small molecule has a molecular weight of 100-1000 daltons, 200-900 daltons, 200-700 daltons, or 200-500 daltons.

Subject: Includes both human and non-human subjects, including birds and non-human mammals, such as non-human primates, companion animals (such as dogs and cats), livestock (such as pigs, sheep, cows), as well as non-domesticated animals, such as the big cats. The term subject applies regardless of the stage in the organism's life-cycle. Thus, the term subject applies to an organism in utero or in ovo, depending on the organism (that is, whether the organism is a mammal or a bird, such as a domesticated or wild fowl).

Sulfinyl: —S(O)R^{d}, wherein R^{d} is selected from hydrogen, aliphatic, heteroaliphatic, haloaliphatic, haloheteroaliphatic, aromatic, or an organic functional group. Sulfinyl groups can be substituted with one or more groups other than hydrogen, such as aliphatic, heteroaliphatic, haloaliphatic, haloheteroaliphatic, aromatic, or an organic functional group.

Sulfonyl: —SO₂R^{d}, wherein R^{d} is selected from hydrogen, aliphatic, heteroaliphatic, haloaliphatic, haloheteroaliphatic, aromatic, or an organic functional group. Sulfonyl groups can be substituted with one or more groups other than hydrogen, such as aliphatic, heteroaliphatic, haloaliphatic, haloheteroaliphatic, aromatic, or an organic functional group.

Sulfonamide: —SO₂NR^{e}R or —N(R^{e})SO₂R^{f}, wherein each of R^{c} and R independently is selected from hydrogen, aliphatic, heteroaliphatic, haloaliphatic, haloheteroaliphatic, aromatic, or an organic functional group. Sulfonamide groups can be substituted with one or more groups other than hydrogen, such as aliphatic, heteroaliphatic, haloaliphatic, haloheteroaliphatic, aromatic, or an organic functional group.

Sulfonate: —SO₃⁻, wherein the negative charge of the sulfonate group may be balanced with an M⁺ counter ion, wherein M⁺ may be an alkali ion, such as K⁺, Na⁺, Li⁺; an ammonium ion, such as ⁺N(R^{e})₄ where R^{e} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}.

Therapeutically effective amount: The amount of an agent that alone, or together with one or more additional agents, induces the desired response, such as, for example treatment of a tumor in a subject. Ideally, a therapeutically effective amount provides a therapeutic effect without causing a substantial cytotoxic effect in the subject.

In one example, a desired response is to decrease the size, volume, or number (such as metastases) of a tumor in a subject. For example, the agent or agents can decrease the size, volume, or number of tumors by a desired amount, for example by at least 5%, at least 10%, at least 15%, at least 20%, at least 25%, at least 30%, at least 50%, at least 75%, at least 90%, or at least 95% as compared to a response in the absence of the agent.

Several preparations disclosed herein are administered in therapeutically effective amounts. A therapeutically effective amount of a disclosed compound that is administered to a human or veterinary subject will vary depending upon a number of factors associated with that subject, for example the overall health of the subject. A therapeutically effective amount can be determined by varying the dosage and measuring the resulting therapeutic response, such as the regression of a tumor. Therapeutically effective amounts also can be determined through various in vitro, in vivo or in situ immunoassays. The disclosed agents can be administered in a single dose, or in several doses, as needed to obtain the desired response. However, the therapeutically effective amount of can be dependent on the source applied, the subject being treated, the severity and type of the condition being treated, and the manner of administration.

Thial: —C(S)H.

Thiocarboxylic acid: —C(O)SH, or —C(S)OH.

Thiocyanate: —S—CN or —N═C═S.

Thioester: —C(O)SR^{d} or —C(S)OR^{d} wherein R^{d} is selected from hydrogen, aliphatic, heteroaliphatic, haloaliphatic, haloheteroaliphatic, aromatic, or an organic functional group. Thioester groups can be substituted with one or more groups other than hydrogen, such as aliphatic, heteroaliphatic, haloaliphatic, haloheteroaliphatic, aromatic, or an organic functional group.

Thioether: —S-aliphatic or —S-aromatic, such as —S-alkyl, —S-alkenyl, —S-alkynyl, —S-aryl, or —S— heteroaryl; or -aliphatic-S-aliphatic, -aliphatic-S-aromatic, -aromatic-S-aliphatic, or -aromatic-S-aromatic. Thioether groups can be substituted with one or more groups other than hydrogen, such as aliphatic, heteroaliphatic, haloaliphatic, haloheteroaliphatic, aromatic, or an organic functional group.

Thioketone: —C(S)R^{d} wherein R^{d} is selected from hydrogen, aliphatic, heteroaliphatic, haloaliphatic, haloheteroaliphatic, aromatic, or an organic functional group. Thioketone groups can be substituted with one or more groups other than hydrogen, such as aliphatic, heteroaliphatic, haloaliphatic, haloheteroaliphatic, aromatic, or an organic functional group.

Treating or Inhibiting a Disease: A therapeutic intervention that reduces a sign or symptom of a disease or pathological condition related to a disease (such as a tumor). Treatment can also induce remission or cure of a condition, such as a tumor. In particular examples, treatment includes preventing a tumor, for example by inhibiting the full development of a tumor, such as preventing development of a metastasis or the development of a primary tumor. Prevention does not require a total absence of a tumor.

Reducing a sign or symptom of a disease or pathological condition related to a disease, refers to any observable beneficial effect of the treatment. Reducing a sign or symptom associated with a tumor can be evidenced, for example, by a delayed onset of clinical symptoms of the disease in a susceptible subject (such as a subject having a tumor which has not yet metastasized), a reduction in severity of some or all clinical symptoms of the disease, a slower progression of the disease (for example by prolonging the life of a subject having tumor), a reduction in the number of relapses of the disease, an improvement in the overall health or well-being of the subject, or by other parameters well known in the art that are specific to the particular tumor. A “prophylactic” treatment is a treatment administered to a subject who does not exhibit signs of a disease or exhibits only early signs for the purpose of decreasing the risk of developing pathology.

Tumor: An abnormal growth of cells, which can be benign or malignant. Cancer is a malignant tumor, which is characterized by abnormal or uncontrolled cell growth. Other features often associated with malignancy include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. “Metastatic disease” refers to cancer cells that have left the original tumor site and migrate to other parts of the body for example via the bloodstream or lymph system. Thus, a metastatic cancer is a cancer at one or more sites in the body other than the site of origin of the original (primary) cancer from which the metastatic cancer is derived. The amount of a tumor in an individual is the “tumor burden” which can be measured as the number, volume, or weight of the tumor. A tumor that does not metastasize is referred to as “benign.”

The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.” Examples of hematological tumors include leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers, ovarian cancer, prostate cancer, hepatocellular carcinoma, squamous cell carcinoma, basal cell carcinoma, adenocarcinoma, salivary gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyrgioma, ependymoma, pinealoma, hemangioblastoma, acoustic neuroma, oligodendroglioma, meningioma, melanoma, neuroblastoma and retinoblastoma).

An “established” or “existing” tumor is an existing tumor that can be discerned by diagnostic tests. In some implementations, an established tumor can be palpated. In some implementations, an “established tumor” is at least 500 mm³, such as at least 600 mm³, at least 700 mm³, or at least 800 mm³ in size. In other implementations, the tumor is at least 1 cm long. With regard to a solid tumor, an established tumor generally has a robust blood supply, and has induced Tregs and myeloid derived suppressor cells (MDSCs). In several examples, a tumor is neuroblastoma or small-cell lung cancer.

### II. Introduction

Targeting G-quadruplexes (G4s) with small molecules is an attractive strategy to control the expression of undruggable proteins, such as N-Myc and other G4-containing proteins; however, selective binders to G4s are challenging to identify due to the structural similarity of many G4s. Many canonical G4 structures are relatively simple: they contain tetrads of guanines that are stabilized by central potassium ions, and small 1-7 nucleotide loops. Recent examples of more complex G4s (or “non-canonical” G4s), however, have been shown to also contain additional structural elements, such as hairpins and other types of structures (for example, see Onel et al., JACS 2016, 138:2563; 2570; Ngoc Nguyen et al., Nucleic Acids Res. 2020, 48:10567-10575; and Tan et al., Nucleic Acids Res. 2020, 48:11162-11171, which disclose such non-canonical structures that are incorporated herein by reference). Among the reported G4s in MYCN, one example of a non-canonical G4 with such a structure is found in intron 1 of MYCN. Genome-wide probing studies have also experimentally confirmed the existence of a G4 in the MYCN gene, though it does not occur in the promoter region of MYCN like many other regulatory G4s.

New small molecule compounds that target non-canonical G4 structures, such as hairpin-containing G4s, are described herein. In some implementations, the compounds target non-canonical G4s in the MYC gene family, such as MYCN. Pharmaceutical compositions comprising the compounds and methods of making and using the compounds also are disclosed.

Also disclosed are methods of using such compounds for therapeutic treatment, such as in methods of treating diseases and/or disorders, like cancer. In some implementations, treatment with the disclosed compounds results in decreases in both the MYCN and MYCNOS transcripts as well as the N-Myc protein levels.

### III. Compounds

Disclosed herein are compounds for use in novel methods for treating diseases and/or disorders, such as cancer. In particular implementations, the compounds are small molecule compounds (including stereoisomers, tautomers, or pharmaceutically acceptable salts or esters thereof) that can target and bind non-canonical G4s. In particular implementations, the compounds are small molecules that selectively bind directly to structurally complex non-canonical G4s, such as hairpin-containing G4s. In particular implementations, the hairpin-containing G4 is present in DNA. In an independent implementation, the compounds do not bind RNA (or mRNA) that does not comprise a G4 structure. Some compounds, such as the bivalent compounds disclosed herein can bind G4s present in RNA (e.g., mRNA), but they do not bind simple RNA (or mRNA), such as simple RNA (or mRNA) comprising stem-loop structures, bulge structure, or hairpin structures without a G4. Exemplary genes that can be targeted using the compounds disclosed herein can include, but are not limited to, the MYC-family (e.g., MYCN, MYCNOS, MYCL, MYC, and the like), FOXA3, KRAS, BRD4, and any oncogenes and/or non-coding RNAs (e.g., long non-coding RNAs (lncRNA)) thereof, such as BCL2, SOX2, and/or LINC01018. In some implementations, the compounds can bind G4s present in helicases (e.g., DHX15). In some implementations, the compounds selectively bind near a junction at a G4 hairpin found in such genes.

In some implementations, the compound, or a stereoisomer, tautomer, or pharmaceutically acceptable salt or ester thereof, has a structure according to Formula I.

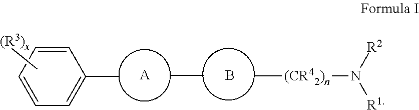

With respect to Formula I, the following variable recitations can apply:
ring A is a 5-membered heteroaryl ring and in particular independent implementations is not a thiophenyl, thiazolyl, furanyl, triazolyl, or thiadiazolyl;
ring B is a 6-membered heteroaryl ring containing at least two nitrogen atoms;
R¹ is -(linker)ₜ-R^{b} wherein R^{b} is aromatic, heteroaliphatic, or aliphatic; the linker is an aliphatic or heteroaliphatic group; and t is 0 or 1;
R² is H or aliphatic;
each R³ independently is heteroaliphatic, hydroxy, aliphatic, haloaliphatic, or halo (e.g., Cl, F, Br, or I) and in an independent implementation, R³ is not fluoro;
each R⁴ independently is H, aliphatic, or halo;
x is an integer selected from 0 to 5, such as 0, 1, 2, 3, 4, or 5; and n is an integer selected from 0 to 10, such as 0, 1, 2, 3, 4, 5, 6, 7, 8, 9, or 10.

In some implementations of Formula I, the following variable recitations can apply:
ring A is oxadiazolyl, and in particular independent implementations is not a 1,3,4-oxadiazolyl;
ring B is selected from diazinyl (e.g., pyridazinyl, pyrimidinyl, or pyrazinyl);
R¹ is -(linker)ₜ-R^{b} wherein R^{b} is aryl, heteroaryl, cycloheteroaliphatic, acycloheteroaliphatic, cycloaliphatic, acycloaliphatic; t is 0 or 1; and the linker is —(CR^{a}₂)^{q}— or —[(CR^{a}₂)_{q}Y]ᵣ—(CR^{a}₂)ₛ—, wherein each R^{a} independently is H, aliphatic, or halo; Y is oxygen, sulfur, or NR′, wherein R′ is H or aliphatic; q is an integer selected from 1 to 10, such as 1, 2, 3, 4, 5, 6, 7, 8, 9, or 10; r is an integer selected from 1 to 10, such as 1, 2, 3, 4, 5, 6, 7, 8, 9, or 10; and s is an integer selected from 0, 1, or 2;
R² is H or lower alkyl;
each R³ independently is alkoxy or hydroxy;
each R⁴ independently is H, alkyl, or halo;
x is an integer selected from 1 to 5; and
n is an integer selected from 0 to 3, such as 0, 1, 2, or 3.

In yet additional implementations of Formula I, the following variable recitations can apply:
ring A is 1,2,4-oxadiazolyl (e.g., N N);

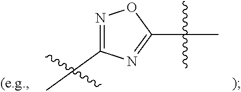 [SMILES: *c1noc(*)n1]

ring B is pyridazinyl

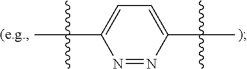 [SMILES: *c1ccc(*)nn1]

R¹ is -(linker)ₜ-R^{b} wherein R^{b} is phenyl, piperazinyl, pyridinyl, azepinyl, cyclopentyl, amino (e.g., NH₂, N(alkyl)₂, and NH(alkyl)), piperidinyl, pyrrolidinyl, oxadiazolyl, triazinyl, amide (e.g., —C(O)NH₂), morpholinyl, acridinyl (e.g., 9-aminoacridine, acridine Cl, and acridine NH₂, acridine ICR 191); t is 0 or 1; and the linker is —(CH₂)_{q}—, —CH(Me)-, —(CH₂)_{q}—CH(Me)-, —[(CH₂)_{q}O]ᵣ—(CH₂)ₛ—; or —(CH₂)_{q}NH—, wherein q is an integer selected from 1 to 4, such as 1, 2, 3, or 4; r is an integer selected from 1 to 4, such as 1, 2, 3, or 4; and s is 0, 1, or 2;
R² is H or Me;
each R³ independently is —OMe or hydroxy; and
n is 0.

In some implementations, the compound has a structure according to Formula I, wherein ring A is a 5-membered heteroaryl ring; ring B is a 6-membered heteroaryl ring containing at least two nitrogen atoms; R¹ is —(CR^{a}₂)ₘ—R^{b} or —[(CR^{a}₂)ₘO]ᵣ—(CH₂)ₛ—R^{b}, where each R^{a} independently is H, alkyl, or halo, m is 1, 2, 3, 4, or 5, r is 1 or 2, s is 2, and R^{b} is a cycloalkyl group, an aryl group, or a nitrogen-containing group (e.g., an N-containing cyclic group, such as a heteroaryl group comprising nitrogen or a cycloheteroaliphatic group comprising a nitrogen atom; an amide group; or an amino group); R² is H or aliphatic; each R³ independently is alkoxy or hydroxy, and x is 0, 1, 2, 3, 4, or 5; each R⁴ independently is H, alkyl, or halo; and, n is 0, 1, 2 or 3. In an independent implementation of such compounds having a structure according to Formula I, the compound does not comprise the following compounds, but can include any pharmaceutically acceptable salt or ester thereof:
6-(3-(4-methoxyphenyl)-1,2,4-oxadiazol-5-yl)-N-(3-(pyrrolidin-1-yl)propyl)pyridazin-3-amine;
N-(3-([1,4′-bipiperidin]-1′-yl)propyl)-6-(3-(4-methoxyphenyl)-1,2,4-oxadiazol-5-yl)pyridazin-3-amine;
6-(3-(4-methoxyphenyl)-1,2,4-oxadiazol-5-yl)-N-(3-(4-methylpiperazin-1-yl)propyl)pyridazin-3-amine;
N1-(6-(3-(4-methoxyphenyl)-1,2,4-oxadiazol-5-yl)pyridazin-3-yl)-N2,N2-dimethylethane-1,2-diamine;
N-(3-(azepan-1-yl)propyl)-6-(3-(4-methoxyphenyl)-1,2,4-oxadiazol-5-yl)pyridazin-3-amine;
6-(3-(4-methoxyphenyl)-1,2,4-oxadiazol-5-yl)-N-(3-(4-methylpiperidin-1-yl)propyl)pyridazin-3-amine; or
6-(3-(4-methoxyphenyl)-1,2,4-oxadiazol-5-yl)-N-(2-morpholinoethyl)pyridazin-3-amine.

In some implementations, the compound can have a structure according to Formula IA

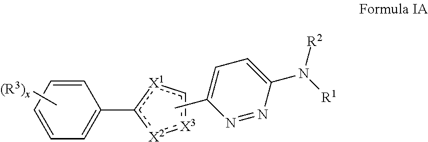

wherein each bond represented by [symbol] is a single or double bond as needed to satisfy valence requirements; each of X¹, X², and X³ independently is N, O, S, or C(R^{c}) where R^{c} is H, alkyl, or halo, provided that at least one of X¹, X², and X³ is other than C(R^{c}); R¹ is -(linker)ₜ-R^{b} wherein R^{b} is aromatic, heteroaliphatic, or aliphatic; the linker is an aliphatic or heteroaliphatic group; and t is 0 or 1; R² is H or aliphatic; each R³ independently is heteroaliphatic, hydroxy, aliphatic, haloaliphatic, or halo (e.g., Cl, F, Br, or I) and in an independent implementation, R³ is not fluoro; each R⁴ independently is H, aliphatic, or halo; x is an integer selected from 0 to 5, such as 0, 1, 2, 3, 4, or 5; and n is an integer selected from 0 to 10, such as 0, 1, 2, 3, 4, 5, 6, 7, 8, 9, or 10. In particular independent implementations, the compound does not comprise a thiophenyl, thiazolyl, furanyl, triazolyl, thiadiazolyl, or a 1,3,4-oxadiazolyl ring. In particular implementations, the compound comprises a 1,2,4-oxadiazolyl ring.

In any of the foregoing or following implementations, ring A may be

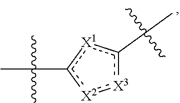

where each bond represented by [symbol] is a single or double bond as needed to satisfy valence requirements; and each of X¹, X², and X³ independently is N, O, S, or C(R^{c}) where R^{c} is H, alkyl, or halo, provided that at least one of X¹, X², and X³ is other than C(R^{c}). In some implementations of Formula I and/or Formula IA, R^{c} is H or alkyl, such as C₁-C₃ alkyl (methyl, ethyl, propyl, or isopropyl). In certain implementations of Formula I and/or Formula IA, R^{c} is H. In some implementations of Formula I and/or Formula IA, at least one of X¹, X², and X³ is N. In certain implementations of Formula I and/or Formula IA, at least one of X¹, X², and X³ is N and one of X¹, X², and X³ is O or S. In some implementations, the five-membered ring of Formula I and/or IA is selected from

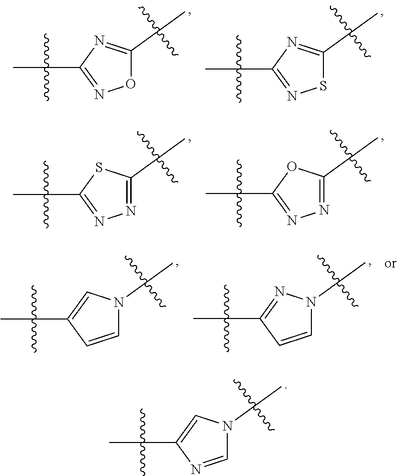

In certain examples, the five-membered ring of Formula I and/or IA is

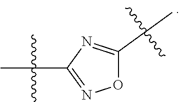 [SMILES: *c1noc(*)n1]

In any of the foregoing or following implementations, ring B of Formula I may be

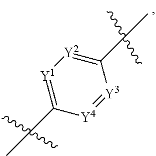

where each of Y¹, Y², Y³, and Y⁴ independently is N or C(R^{c}) where R^{c} is H, alkyl, or halo, provided that at least one of Y¹, Y², Y³, and Y⁴ are N. In some implementations of Formula I, two of Y¹, Y², Y³, and Y⁴ are N, and the other of Y¹, Y², Y³, and Y⁴ are C(R^{c}). Each R^{c} independently may be H, -alkyl, or halo. In particular examples, R^{c} is H. In some implementations of Formula I, ring B is N,

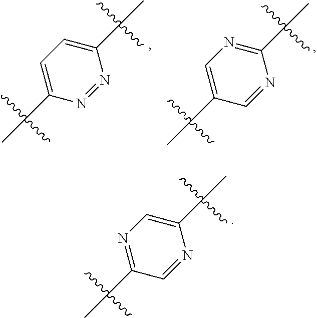 [SMILES: *c1ccc(*)nn1 ; *c1cnc(*)nc1 ; *c1cnc(*)cn1]

In certain implementations of Formula I, ring B is

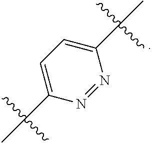 [SMILES: *c1ccc(*)nn1]

The disclosed compounds typically comprise a number of R³ groups (represented as “x” in Formula I), wherein x is 0, 1, 2, 3, 4, or 5, and each R³ independently is alkoxy, hydroxy, haloalkyl, aliphatic, or halo. In an independent implementation, R³ is not fluoro. In some implementations, x is 1, 2, or 3. In certain implementations, x is 1. In some implementations, at least one R³ group is in a para position relative to ring A. In any of the foregoing or following implementations, R³ may be C₁-C₃ alkoxy or hydroxy. In some implementations, R³ is methoxy. In certain implementations, x is 1 and R³ is para to ring A.

In any of the foregoing or following implementations wherein n is an integer other than 0, each R⁴ independently is H, alkyl (e.g., C₁-C₃ alkyl), or halo. In some implementations, R⁴ is H or methyl. In certain implementations, R⁴ is H. In some implementations, n is 0 or 1. In certain implementations, n is 0, and —N(R′)(R²) is attached directly to ring B.

In some implementations, the compound has a structure according to Formula II:

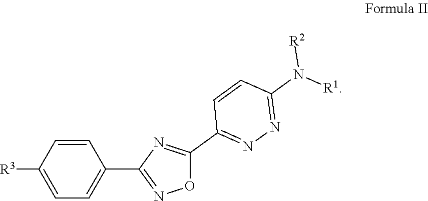

With reference to Formula II, each of R¹, R², and R³ are as defined according to any of the above implementations. In some implementations, R² is H or C₁-C₃ alkyl. In certain implementations, R² is H.

In some implementations, the compound has a structure according to Formula III:

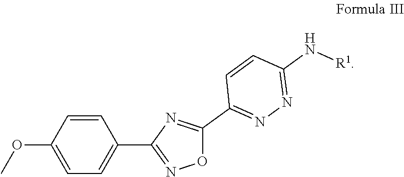

In particular implementations of any one of Formulas I, IA, II, or III, R¹ is —(CR^{a}₂)ₘ—R^{b} or —[(CR^{a}₂)ₘO]ᵣ—(CH₂)₂—R^{b} where each R^{a} independently is H, alkyl, or halo, m is 1, 2, 3, 4, or 5, r is 1, 2, 3, 4, or 5, and R^{b} is a nitrogen-containing group. In some implementations, each R^{a} independently is H or C₁-C₃ alkyl. In certain implementations, each R^{a} is H, and R¹ is —(CH₂)ₘ—R^{b}. In certain implementations, each R^{a} is H, and R¹ is —[(CR^{a}₂)ₘO], —(CH₂)₂—R^{b}, wherein r is 1, 2, or 3. In some implementations, m is 1, 2, or 3, particularly 2 or 3. In any of the foregoing or following implementations, m+n may be 1, 2, 3, 4, or 5. In some implementations, m+n is 2, 3, or 4, particularly 2 or 3. In certain implementations, n is 0 and m is 2 or 3.

In particular implementations of any one of Formulas I, IA, II, or III, R^{b} is a nitrogen-containing group. In some implementations, R^{b} is an N-containing cyclic group, —N(R^{c})₂, or —C(O)N(R^{c})₂ where each R^{c} is H, alkyl, or halo. The N-containing cyclic group may be saturated or unsaturated (including aromatic), and may include from 5 to 15 atoms, such as 5 to 14 atoms, or 5 to 10 atoms, or 5 to 8 atoms. In some implementations, the N-containing cyclic group further comprises one or more substituents, such as substituents selected from halo, heteroaliphatic, or aliphatic. In particular implementations, the N-containing cyclic group is attached to —(CR^{a}₂)ₘ— or —[(CR^{a}₂)ₘO]ᵣ—(CH₂)₂— via a nitrogen atom in the heterocycle. Exemplary N-containing cyclic groups include 1 or 2 nitrogen atoms, and may include another heteroatom, such as a S or O atom. Suitable N-containing cyclic groups include, but are not limited to, acridinyl groups (or other G4 stacking compounds, such as BRACO-19 and the like), hexahydroazepinyl groups, piperazinyl groups, morpholino groups, and piperidinyl groups. In some implementations, R^{b} is —N(R^{c})₂ or —C(O)N(R^{c})₂ where R^{c} is H or alkyl, such as H or C₁-C₃ alkyl; or acridinyl groups, such as acridine ICR 191, 9-aminoacridine, acridine Cl, or acridine NH₂. In implementations comprising such acridinyl groups, the compound can be a “bivalent” compound comprising two G4-responsive groups, including a non-canonical G4-binding component and a G4-stacker component. The non-canonical G4-binding component comprises portion of the compound that includes rings A, B, and/or the R³-bearing phenyl ring; and the G4-stacker component comprises the acridinyl group.

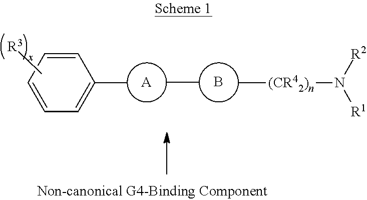

In some implementations of bivalent compounds, the bivalent compound has a structure according to Formula IV:

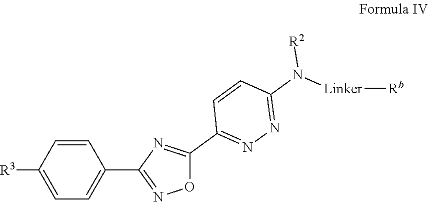

wherein R^{b} is a G4-stacker group, such as an aromatic group (e.g., an acridinyl group); and the linker group is —(CR^{a}₂)ₘ— or —[(CR^{a}₂)ₘO]ᵣ—(CH₂)ₛ— wherein each of R^{a}, m, r, and s are as recited herein, such as provided for Formulas I and/or II.

In some implementations of bivalent compounds, the bivalent compound has a structure according to Formula V:

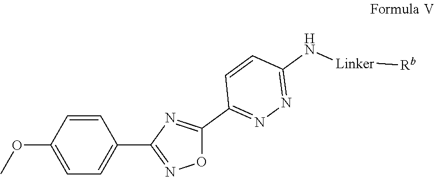

wherein R^{b} is an acridinyl group; and the linker group is —(CH₂)ₘ— or —[(CH^{a}₂)ₘO], —(CH₂)ₛ— wherein each of m, r, and s are as recited herein, such as provided for Formulas I and/or II.

In particular implementations of any one of Formulas I, IA, II, or III, R¹ is selected from the following:

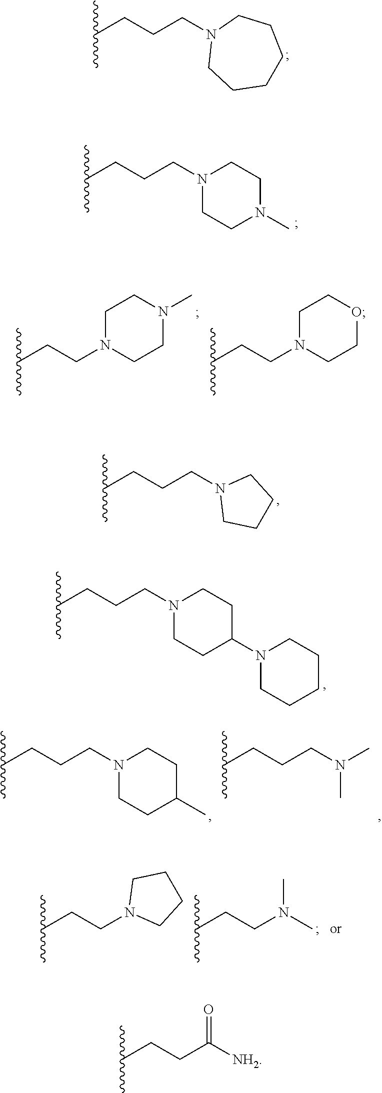

In particular implementations of any one of Formulas I, IA, IV, or V, R¹ is selected from the following:

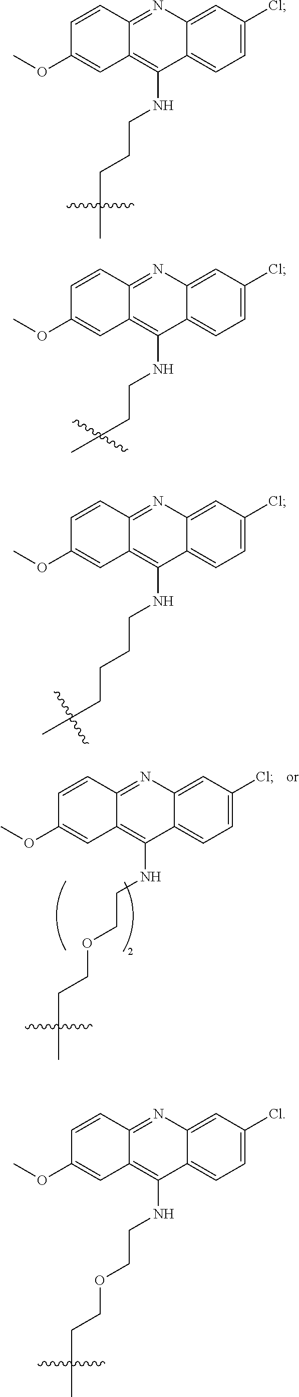

In certain implementations of Formula II, R² is H or C₁-C₃ alkyl and R¹ is as defined according to any of the above implementations. In certain implementations, R² is H.

In certain implementations of Formula III, R² is H, R³ is methoxy, and R¹ is as defined according to any of the above implementations.

Certain compounds according to Formula I are shown in Table 1:

TABLE 1

| | |
|---|---|
| 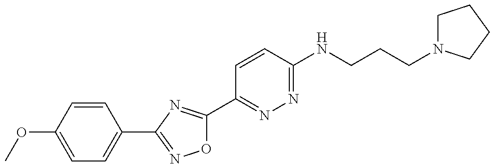 [SMILES: COc1ccc(-c2noc(-c3ccc(NCCCN4CCCC4)nn3)n2)cc1] | MY-1 |
| 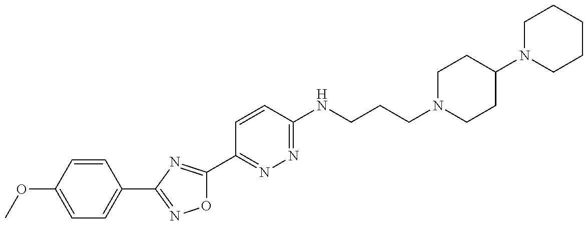 [SMILES: COc1ccc(-c2noc(-c3ccc(NCCCN4CCC(N5CCCCC5)CC4)nn3)n2)cc1] | MY-2 |
| 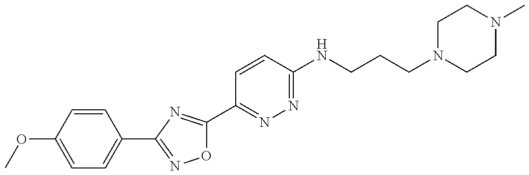 | MY-5 |
| 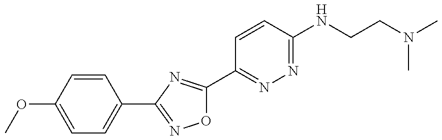 | MY-6 |
| 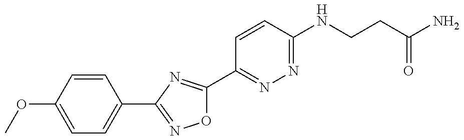 [SMILES: COc1ccc(-c2noc(-c3ccc(NCCC(N)=O)nn3)n2)cc1] | MY-7 |
| 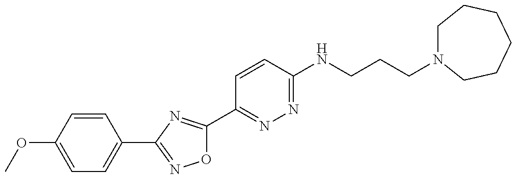 [SMILES: COc1ccc(-c2noc(-c3ccc(NCCCN4CCCCCC4)nn3)n2)cc1] | MY-8 |
| 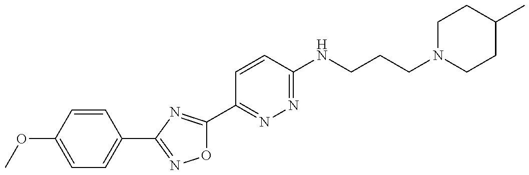 | MY-10 |
| 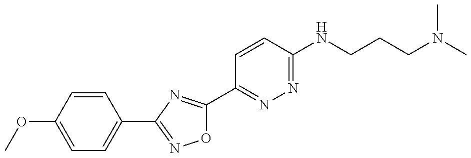 | MY-11 |
| 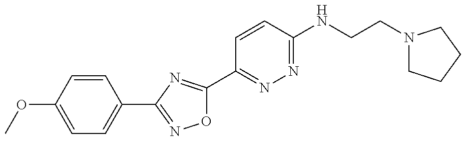 [SMILES: COc1ccc(-c2noc(-c3ccc(NCCN4CCCC4)nn3)n2)cc1] | MY-12 |
| 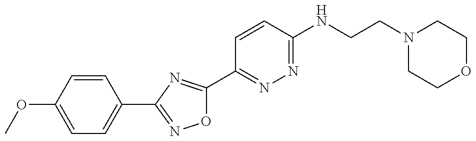 [SMILES: COc1ccc(-c2noc(-c3ccc(NCCN4CCOCC4)nn3)n2)cc1] | MY-13 |
| 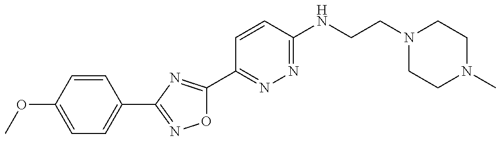 | MY-14 |
| 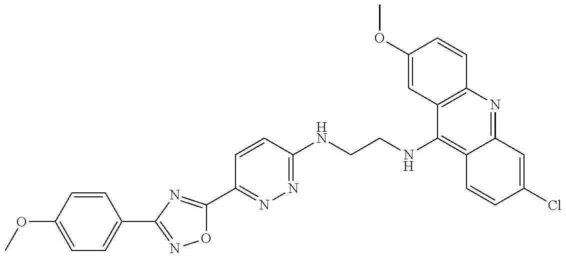 [SMILES: COc1ccc(-c2noc(-c3ccc(NCCNc4c5ccc(Cl)cc5nc5ccc(OC)cc45)nn3)n2)cc1] | B32 |
| 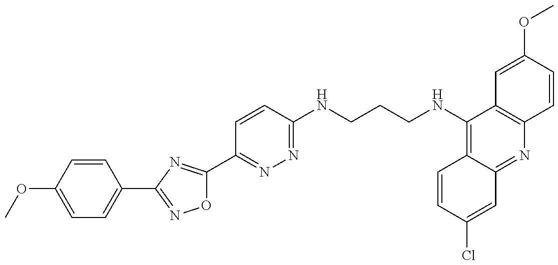 | B33 |
| 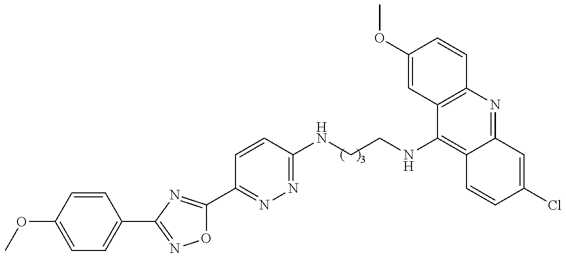 | B34 |
| 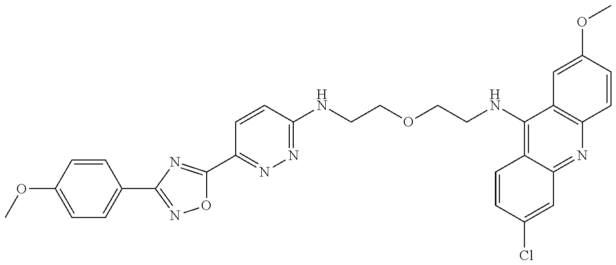 | B35 |
| 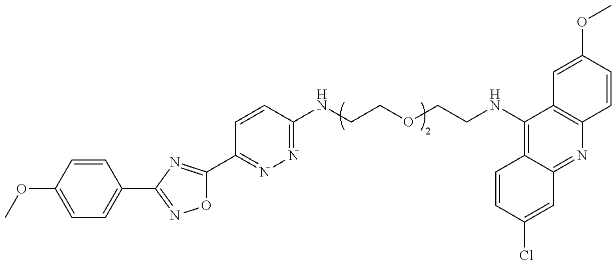 | B38 |

In some implementations, the compound is MY-5, MY-6, MY-7, MY-8, MY-13, MY-14, B32, B33, B34, B35, or B38. In certain examples, the compound is MY-8 or B33.

In an independent implementation, the compound does not comprise, or is not or is other than, the following:
6-(3-(4-methoxyphenyl)-1,2,4-oxadiazol-5-yl)-N-(3-(pyrrolidin-1-yl)propyl)pyridazin-3-amine;
N-(3-([1,4′-bipiperidin]-1′-yl)propyl)-6-(3-(4-methoxyphenyl)-1,2,4-oxadiazol-5-yl)pyridazin-3-amine;
6-(3-(4-methoxyphenyl)-1,2,4-oxadiazol-5-yl)-N-(3-(4-methylpiperazin-1-yl)propyl)pyridazin-3-amine;
N1-(6-(3-(4-methoxyphenyl)-1,2,4-oxadiazol-5-yl)pyridazin-3-yl)-N2,N2-dimethylethane-1,2-diamine;
N-(3-(azepan-1-yl)propyl)-6-(3-(4-methoxyphenyl)-1,2,4-oxadiazol-5-yl)pyridazin-3-amine;
6-(3-(4-methoxyphenyl)-1,2,4-oxadiazol-5-yl)-N-(3-(4-methylpiperidin-1-yl)propyl)pyridazin-3-amine; or
6-(3-(4-methoxyphenyl)-1,2,4-oxadiazol-5-yl)-N-(2-morpholinoethyl)pyridazin-3-amine.

### IV. Method of Making

Disclosed herein are implementations of a method for making compounds according to the present disclosure, such as compounds of Formulas I, IA, II, III, IV, or V. A representative method for making compounds is provided below by Scheme 2, wherein R is used to represent R¹ as described herein.

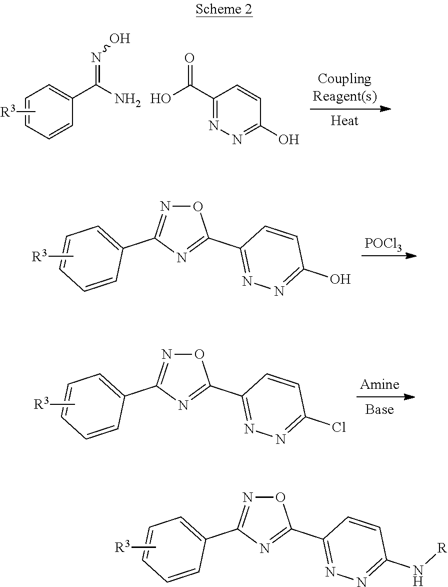

A representative method for making compound embodiments disclosed herein is provided below in Scheme 3.

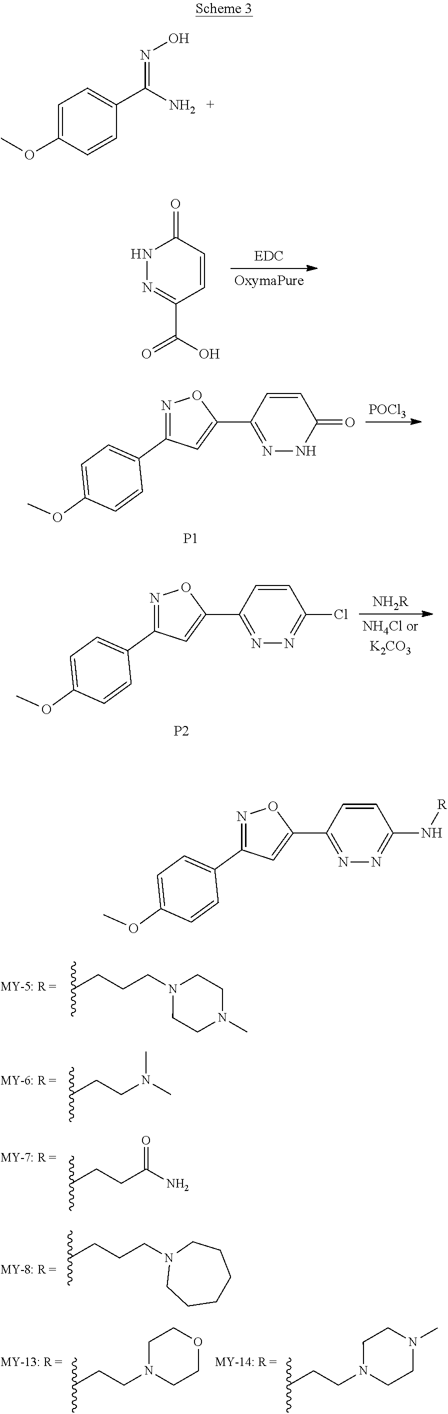

With reference to Scheme 3, the following exemplary procedures can be used (reagent amounts, reaction times, and temperatures described in the Examples Section herein can be used in some implementations):

6-hydroxypyridazine-3-carboxylic acid, EDC·HCl, Oxyma Pure and DMF are combined. The reaction is stirred at room temperature for 10 minutes, followed by the addition of 4-methoxybenzamidoxime. The reaction mixture is then heated to 120° C. and stirred. After 3 hours, the reaction mixture is filtered and washed with DMF. The filtrate is collected and dried to yield the target product P1. A solution of P1 in POCl₃ is then heated to 100° C. and stirred for 4 hours. The reaction crude is poured on ice-cold water and neutralized with 10% NaOH solution. The mixture is then extracted with EtOAc and washed with brine. The combined organic layers are dried over MgSO₄, filtered and concentrated in vacuo. The resulting residue is purified by ISCO flash column chromatography. Then, a mixture of P2, 3-(4-methylpiperazin-1-yl)propan-1-amine and NH₄Cl in EtOH is heated to 80° C. and stirred overnight. The reaction mixture is concentrated in vacuo and the resulting residue is purified by Isco flash column chromatography to afford MY-5;

A mixture of P2, N,N′-dimethylethylenediamine and NH₄Cl in EtOH is heated to 80° C. and stirred overnight. The reaction mixture is concentrated in vacuo and the resulting residue was purified by Isco flash column chromatography to afford MY-6;

A mixture of P2, 3-aminopropanamide hydrochloride and K₂CO₃ in EtOH is heated to 80° C. and stirred overnight. The reaction mixture is concentrated in vacuo and the resulting residue is purified by Isco flash column chromatography to afford MY-7;

A mixture of P2, 3-(1-azepanyl)-1-propanamine and NH₄Cl in EtOH is heated to 80° C. and stirred overnight. The reaction mixture is concentrated in vacuo and the resulting residue is purified by Isco flash column chromatography to afford MY-8. MY-8 was then stirred in TFA for 30 min to yield the corresponding TFA salt;

A mixture of P2, 2-morpholinoethan-1-amine and NH₄Cl in EtOH is heated to 80° C. and stirred overnight. The reaction mixture is concentrated in vacuo and the resulting residue is purified by Isco flash column chromatography to afford MY-13; and

A mixture of P2, 2-(4-methylpiperazin-1-yl)ethan-1-amine and NH₄Cl in EtOH is heated to 80° C. and stirred overnight. The reaction mixture is concentrated in vacuo and the resulting residue is purified by Isco flash column chromatography to afford MY-14.

Bivalent compounds disclosed herein can be made according to the method provided by Scheme 4, below, wherein R is used to represent R¹ as described herein.

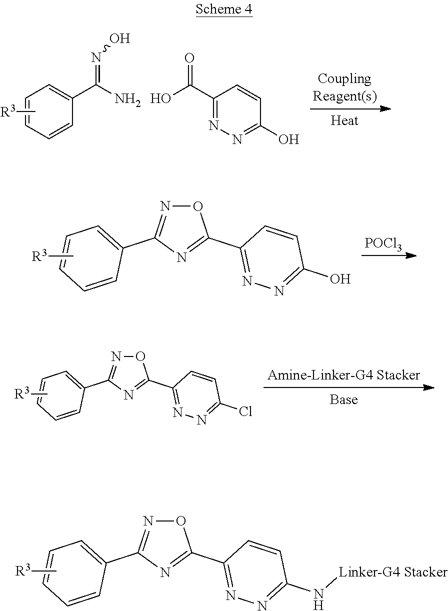

A representative method for making bivalent compound embodiments disclosed herein is provided below in Scheme 5.

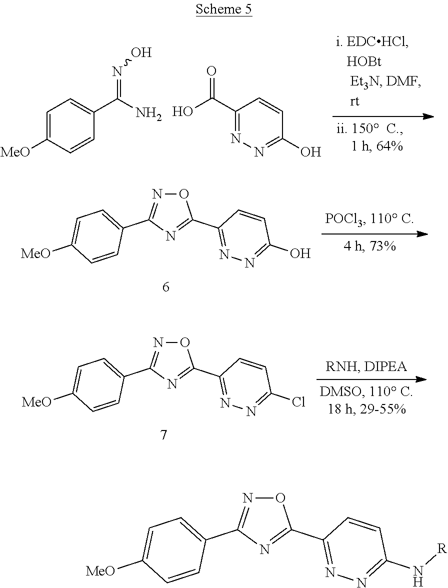

With reference to Scheme 5, the following procedures are used (reagent amounts, reaction times, and temperatures described in the Examples Section herein can be used in some implementations) and R refers to a linker-G4 stacker motif as recited for certain R¹ substituents disclosed herein:
A solution of 7 in DMSO is combined with DIPEA followed by 2. The reaction is heated and maintained at 110° C. for 18 hours. The crude reaction mixture is allowed to cool to room temperature and purified by reverse phase flash chromatography to afford B33 as yellow solid;
A solution of 7 in DMSO is combined with DIPEA followed by 2. The reaction is heated and maintained at 110° C. for 18 hours. The crude reaction mixture is allowed to cool to room temperature and purified by reverse phase flash chromatography to afford B33 as yellow solid;
A solution of 7 in DMSO is combined with DIPEA followed by 3. The reaction is heated and maintained at 110° C. for 18 hours. The crude reaction mixture is allowed to cool to room temperature and is purified by reverse phase flash chromatography to afford B34 as yellow solid;
A solution of 7 in DMSO is combined with DIPEA followed by 4. The reaction is heated and maintained at 110° C. for 18 hours. The crude reaction mixture is allowed to cool to room temperature and purified by reverse phase flash chromatography to afford B35 as a yellow solid; and
A solution of 7 in DMSO is combined with DIPEA followed by 5. The reaction is heated and maintained at 110° C. for 18 hours. The crude reaction mixture is allowed to cool to room temperature and purified by reverse phase flash chromatography to afford B38 as a yellow solid.

Additional details concerning methods for making representative compounds are provided herein in the Examples section.

### V. Method of Use

Disclosed herein are implementations of a method for controlling levels of genes and/or gene expression in a cell wherein the gene(s) comprises at least one region comprising a non-canonical G4, such as a region comprising a hairpin G4. In some implementations, the cell may be a cell present in a subject or in a sample (e.g., a biological sample containing cells, such as cancerous or non-cancerous cells). Controlling a level of a gene and/or gene expression can involve decreasing expression of a gene (e.g., MYCN or MYC), for example, decreasing accumulation of a gene product, such as RNA (e.g., mRNA or lncRNA), associated protein, or combinations thereof. In some implementations of the method, the gene can be selected from the MYC-family (e.g., MYCN, MYCNOS, MYCL, MYC, and the like), FOXA3, KRAS, BRD4, and any related oncogenes.

In some implementations, the method can comprise administering a therapeutically effective amount of a compound according to the present disclosure (or a stereoisomer, tautomer, or pharmaceutically acceptable salt or ester thereof) to a subject with cancer (or a tumor), thereby treating the cancer (or tumor). In other implementations, the method can comprise contacting a cell with an effective amount of a compound according to the present disclosure (or a stereoisomer, tautomer, or pharmaceutically acceptable salt or ester thereof). The cell may be contacted in vitro, ex vivo, or in vivo. In some examples, the compound according to the present disclosure enters the cell upon contact. In some implementations, the method can further comprise detecting a decrease in gene, protein, and/or lncRNA expression after exposing the subject or the sample to the compound.

In particular implementations, a method for decreasing protein expression of N-Myc in a cell is disclosed. The method may include contacting the cell with an effective amount of a disclosed compound, or a stereoisomer, tautomer, or pharmaceutically acceptable salt or ester thereof. The cell may be contacted in vitro, ex vivo, or in vivo. The compound selectively binds to a G4 nucleic acid region in the MYCN gene, which, in particular implementations, is the G4 nucleic acid region comprising a non-canonical G4 structure. In particular implementations, the compound binds such non-canonical G4s in DNA of the gene, but does not bind RNA (e.g., mRNA), of the gene that does not also comprise a G4. In some implementations, the compound selectively binds at or near a G4 hairpin. The cell may be a cell characterized at least in part by overexpression of the MYCN gene. In several implementations, expression of the MYCN gene in the cell is reduced at least 25%, such as at least 50%, at least 75%, at least 80%, at least 85%, or even at least 90% relative to expression in the absence of the compound. The compound also may reduce growth and/or proliferation of the cell. In some implementations, growth and/or proliferation is reduced at least 25%, such as at least 50%, at least 75%, at least 80%, at least 85%, at least 90%, or even at least 95% relative to growth and/or proliferation in the absence of the compound.

In particular implementations, a method for decreasing protein expression of c-Myc in a cell is disclosed. The method may include contacting the cell with an effective amount of a disclosed compound, or a stereoisomer, tautomer, or pharmaceutically acceptable salt or ester thereof. The cell may be contacted in vitro, ex vivo, or in vivo. The compound selectively binds to a G4 nucleic acid region in the MYC gene, which, in particular implementations, is a G4 nucleic acid region comprising a non-canonical G4 structure. In particular implementations, the compound binds such non-canonical G4s in DNA of the gene, but does not bind RNA (e.g., mRNA), of the gene that does not also comprise a G4. The cell may be a cell characterized at least in part by overexpression of the MYC gene. In several implementations, expression of the MYC gene in the cell is reduced at least 25%, such as at least 50%, at least 75%, at least 80%, at least 85%, or even at least 90% relative to expression in the absence of the compound. The compound also may reduce growth and/or proliferation of the cell. In some implementations, growth and/or proliferation is reduced at least 25%, such as at least 50%, at least 75%, at least 80%, at least 85%, at least 90%, or even at least 95% relative to growth and/or proliferation in the absence of the compound.

In any of the foregoing or following implementations, the cell may be a tumor and/or cancer cell in a subject, and the method may further comprise treating or preventing cancer in the subject by administering to a subject in need thereof a therapeutically effective amount of the compound, or the pharmaceutically acceptable salt or ester thereof, to decrease protein (e.g., N-Myc, c-Myc, or the like) expression in the tumor and/or cancer cell, thereby treating or preventing the cancer in the subject. Subjects that can benefit from the disclosed methods include human and veterinary subjects. In some implementations, the cancer cell is a neurological cancer cell or a lung cancer cell. In certain implementations, the cancer is neuroblastoma or small cell lung cancer. In some implementations, treating the cancer in the subject decreases growth and/or proliferation of the cancer cell or a tumor comprising the cancer cell.

Treatment of the cancer is generally initiated after the diagnosis of the cancer, or after the initiation of a precursor condition (such as dysplasia or development of a benign tumor). Treatment can be initiated at the early stages of cancer, for instance, can be initiated before a subject manifests symptoms of a condition, such as during a stage I diagnosis or at the time dysplasia is diagnosed. However, treatment can be initiated during any stage of the disease, such as but not limited to stage I, stage II, stage III and stage IV cancers. In some examples, treatment is administered to these subjects with a benign tumor that can convert into a malignant or even metastatic tumor.

Treatment initiated after the development of a condition, such as malignant cancer, may result in decreasing the severity of one or more symptoms of the condition, or completely removing the symptoms, or reducing metastasis, tumor volume, and/or number of tumors. In some example, the tumor becomes undetectable following treatment. In one aspect of the disclosure, the formation of tumors, such as metastasis, is delayed, prevented or decreased. In another aspect, the size of the primary tumor is decreased. In a further aspect, a symptom of the tumor is decreased. In yet another aspect, tumor volume is decreased.

Subjects can be screened prior to initiating the disclosed therapies, for example to determine whether the subject has a tumor and/or cancer. The presence of a tumor can be determined by methods known in the art, and typically include cytological and morphological evaluation. The tumor can be an established tumor. The cells can be in vivo or ex vivo, including cells obtained from a biopsy. The presence of a tumor indicates that the tumor can be treated using the methods provided herein. In some implementations, a subject with an N-Myc-positive or c-Myc-positive tumor is selected for treatment, for example, by detecting N-Myc or c-Myc expression and/or activity in a biological sample obtained from the subject.

For example, upregulated expression of the MYCN gene (for example, as detected by an increase in mRNA of MYCN, N-Myc protein, or the expression of genes up-regulated by N-Myc compared to a control) can be detected, and in some examples quantified. The MYCN gene expression in the biological sample is compared to a control (such as a normal, non-tumor sample). An increase in the expression of the MYCN gene (such as an increase in mRNA of MYCN, N-Myc protein, or the expression of genes up-regulated by N-Myc) in the biological sample relative to the control indicates the presence of a N-Myc-positive tumor, and can be used to select a subject for treatment with one or more of the compounds or compositions disclosed herein. For example, an increase in the test sample of at least 50%, at least 75%, at least 80%, at least 90%, at least 100%, at least 200% or even greater than 500%, relative to the control, indicates the subject (such as a human subject) is likely to respond favorably to treatment with one or more of the agents disclosed herein. Suitable methods for detecting and/or monitoring a N-Myc-positive tumor in a subject (such as a N-Myc-positive neuroblastoma) can be selected by a treating physician. In one implementation, a sample is obtained from a subject, and the presence of a cell that expresses N-Myc is assessed in vitro.

In yet additional implementations, upregulated expression of the MYC gene (for example, as detected by an increase in mRNA of MYC, c-Myc protein, or the expression of genes up-regulated by c-Myc compared to a control) can be detected, and in some examples quantified. The MYC gene expression in the biological sample is compared to a control (such as a normal, non-tumor sample). An increase in the expression of the MYC gene (such as an increase in mRNA of MYC, c-Myc protein, or the expression of genes up-regulated by c-Myc) in the biological sample relative to the control indicates the presence of a c-Myc-positive tumor, and can be used to select a subject for treatment with one or more of the compounds or compositions disclosed herein. For example, an increase in the test sample of at least 50%, at least 75%, at least 80%, at least 90%, at least 100%, at least 200% or even greater than 500%, relative to the control, indicates the subject (such as a human subject) is likely to respond favorably to treatment with one or more of the agents disclosed herein. Suitable methods for detecting and/or monitoring a c-Myc-positive tumor in a subject (such as a c-Myc-positive neuroblastoma) can be selected by a treating physician. In one implementation, a sample is obtained from a subject, and the presence of a cell that expresses c-Myc is assessed in vitro.

A therapeutically effective amount of a disclosed compound according to any one of Formulas I, IA, II, III, IV, or V, as well as any representative compound species (e.g., MY-1, MY-2, MY-5, MY-6, MY-7, MY-8, MY-10, MY-11, MY-12, MY-13, MY-14, B32, B33, B34, B35, or B38) disclosed herein (or a pharmaceutical composition containing same) can be administered to a subject to treat a tumor and/or cancer in the subject. The subject can be selected for treatment that has, is suspected of having or is at risk of developing a tumor or tumors, such as neuroblastoma or small cell lung cancer.

The administration of a compound according to any one of Formulas I, IA, II, III, IV, or V, as well as any representative compound species (e.g., MY-1, MY-2, MY-5, MY-6, MY-7, MY-8, MY-10, MY-11, MY-12, MY-13, MY-14, B32, B33, B34, B35, or B38) disclosed herein (or a pharmaceutical composition containing same) of the disclosure can be for either prophylactic or therapeutic purpose. When provided prophylactically, the compound is provided in advance of any symptom of an initial occurrence of a tumor and/or cancer or of a recurrence of a previously treated tumor and/or cancer. The prophylactic administration of the compound serves to prevent or ameliorate any subsequent disease process. When provided therapeutically, the compound is provided at (or shortly after) the onset of a symptom of disease or infection.

In some examples, a disclosed compound according to any one of Formulas I, IA, II, III, IV, or V, as well as any representative compound species (e.g., MY-1, MY-2, MY-5, MY-6, MY-7, MY-8, MY-10, MY-11, MY-12, MY-13, MY-14, B32, B33, B34, B35, or B38) disclosed herein (or a pharmaceutical composition containing same) can be administered to a subject to slow or inhibit the growth or metastasis of a tumor and/or cancer. In these applications, a therapeutically effective amount of the compound (or pharmaceutical composition thereof) can be administered to the subject in an amount and under conditions sufficient to bind to a non-canonical G4, such as a non-canonical G4 present in DNA of the MYCN gene, and reduce N-Myc expression, thereby slowing or inhibiting the growth or the metastasis of a tumor, or to inhibit a sign or a symptom of a tumor. Examples of suitable subjects include those diagnosed with or suspecting of having cancer (for example, a subject having a tumor), for example a subject having a neuroblastoma or small cell lung cancer.

In some examples, a disclosed compound according to any one of Formulas I, IA, II, III, IV, or V, as well as any representative compound species (e.g., MY-1, MY-2, MY-5, MY-6, MY-7, MY-8, MY-10, MY-11, MY-12, MY-13, MY-14, B32, B33, B34, B35, or B38) disclosed herein (or a pharmaceutical composition containing same) can be administered to a subject to slow or inhibit the growth or metastasis of a tumor and/or cancer. In these applications, a therapeutically effective amount of the compound (or pharmaceutical composition thereof) can be administered to the subject in an amount and under conditions sufficient to bind to a non-canonical G4, such as a non-canonical G4 present in DNA of the MYC gene, and reduce c-Myc expression, thereby slowing or inhibiting the growth or the metastasis of a tumor, or to inhibit a sign or a symptom of a tumor. Examples of suitable subjects include those diagnosed with or suspecting of having cancer (for example, a subject having a tumor) In some examples, the subject has a cancer that expresses MYCN and/or c-MYC. Non-limiting exemplary cancers include sarcomas, carcinomas, fibrosarcoma, myxosarcoma, liposarcoma, chondrosarcoma, osteosarcoma, and other sarcomas, synovioma, mesothelioma, Ewing's tumor, Kaposi sarcoma, leiomyosarcoma, rhabdomyosarcoma, colon carcinoma, lymphoid malignancy, pancreatic cancer, breast cancer, lung cancers, ovarian cancer, uterine cancer, prostate cancer, hepatocellular carcinoma, squamous cell carcinoma, basal cell carcinoma, adenocarcinoma, adrenal carcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, head and neck cancers, neuroblastoma, retinoblastoma and brain metastasis. Cancer also includes hematological (or hematogenous) cancers, such as leukemia, such as lymphoma, Hodgkin's disease, non-Hodgkin's lymphoma (indolent or high grade forms), multiple myeloma, Waldenstrom's macroglobulinemia, heavy chain disease, myelodysplastic syndrome, hairy cell leukemia or myelodysplasia. In non-limiting examples, the cancer is neuroblastoma, rhabdomyosarcoma, prostate cancer, or small cell lung cancer.

The therapeutically effective amount will depend upon the severity of the disease and the general state of the subject's health. A therapeutically effective amount is that which provides either subjective relief of one or more symptoms or an objectively identifiable improvement as noted by the clinician or other qualified observer. In one implementation, a therapeutically effective amount is the amount necessary to inhibit tumor growth, or the amount that is effective at reducing a sign or a symptom of the tumor. The therapeutically effective amount of the agents administered can vary depending upon the desired effects and the subject to be treated. In some examples, therapeutic amounts are amounts which eliminate or reduce the patient's tumor burden, or which prevent or reduce the proliferation of metastatic cells.

The actual dosage of the compound can vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the compound for eliciting the desired activity or biological response in the subject. Dosage regimens can be adjusted to provide an optimum prophylactic or therapeutic response. A therapeutically effective amount is also one in which any toxic or detrimental side effects of the compound and/or other biologically active agent is outweighed in clinical terms by therapeutically beneficial effects. A non-limiting range for a therapeutically effective amount of a compound and/or other biologically active agent within the methods and formulations of the disclosure is about 0.01 mg/kg body weight to about 20 mg/kg body weight, such as about 0.05 mg/kg to about 5 mg/kg body weight, or about 0.2 mg/kg to about 2 mg/kg body weight.

Dosage can be varied by the attending clinician to maintain a desired concentration at a target site (for example, the lungs or systemic circulation). Higher or lower concentrations can be selected based on the mode of delivery, for example, trans-epidermal, rectal, oral, pulmonary, intraosseous, or intranasal delivery versus intravenous or subcutaneous or intramuscular delivery. Dosage can also be adjusted based on the release rate of the administered formulation, for example, of an intrapulmonary spray versus powder, sustained release oral versus injected particulate or transdermal delivery formulations, and so forth.

Any method of administration can be used for the disclosed therapeutic agents, including local and systemic administration. For example topical, oral, intravascular such as intravenous, intramuscular, intraperitoneal, intranasal, intradermal, intrathecal and subcutaneous administration can be used. The particular mode of administration and the dosage regimen will be selected by the attending clinician, taking into account the particulars of the case (for example the subject, the disease, the disease state involved, and whether the treatment is prophylactic). In cases in which more than one agent or composition is being administered, one or more routes of administration may be used. In some implementations, administration is oral, intravascular such as intravenous, intramuscular, intraperitoneal, intranasal, or intrathecal.

For prophylactic and therapeutic purposes, the compound can be administered to the subject by the oral route or in a single bolus delivery, via continuous delivery (for example, continuous intravenous delivery) over an extended time period, or in a repeated administration protocol (for example, by an hourly, daily or weekly, repeated administration protocol). The therapeutically effective dosage of the compound can be provided as repeated doses within a prolonged prophylaxis or treatment regimen that will yield clinically significant results to alleviate one or more symptoms or detectable conditions associated with a targeted disease or condition as set forth herein. Determination of effective dosages in this context is typically based on animal model studies followed up by human clinical trials and is guided by administration protocols that significantly reduce the occurrence or severity of targeted disease symptoms or conditions in the subject. Suitable models in this regard include, for example, murine, rat, avian, dog, sheep, porcine, feline, non-human primate, and other accepted animal model subjects known in the art. Alternatively, effective dosages can be determined using in vitro models. Using such models, only ordinary calculations and adjustments are required to determine an appropriate concentration and dose to administer a therapeutically effective amount of the compound (for example, amounts that are effective to alleviate one or more symptoms of a targeted disease). In alternative implementations, an effective amount or effective dose of the compound may simply inhibit or enhance one or more selected biological activities correlated with a disease or condition, as set forth herein, for either therapeutic or diagnostic purposes.

In some implementations, local administration of the disclosed compounds can be used, for instance by applying a disclosed compound to a region of tissue from which a tumor has been removed, or a region suspected of being prone to tumor development. In some implementations, sustained intra-tumoral (or near-tumoral) release of the pharmaceutical preparation that includes a therapeutically effective amount of a disclosed compound may be beneficial.

The disclosed compounds can be formulated in unit dosage form suitable for individual administration of precise dosages. In addition, the disclosed compounds may be administered in a single dose or in a multiple dose schedule. A multiple dose schedule is one in which a primary course of treatment may be with more than one separate dose, for instance 1-10 doses, followed by other doses given at subsequent time intervals as needed to maintain or reinforce the action of the compositions. Treatment can involve daily or multi-daily doses of compound(s) over a period of a few days to months, or even years. Thus, the dosage regimen will also, at least in part, be determined based on the particular needs of the subject to be treated and will be dependent upon the judgment of the administering practitioner.

In particular examples, the subject is administered a therapeutic composition that includes one or more of the disclosed compounds on a multiple daily dosing schedule, such as at least two consecutive days, 10 consecutive days, and so forth, for example for a period of weeks, months, or years. In one example, the subject is administered the composition for a period of at least 30 days, such as at least 2 months, at least 4 months, at least 6 months, at least 12 months, at least 24 months, or at least 36 months.

In some implementations, the disclosed methods include providing surgery, radiation therapy, and/or chemotherapeutics to the subject in combination with administration of a disclosed compound or composition containing same. Methods and therapeutic dosages of such agents and treatments are known to those skilled in the art, and can be determined by a skilled clinician. Preparation and dosing schedules for the additional agent may be used according to manufacturer's instructions or as determined empirically by the skilled practitioner. Preparation and dosing schedules for such chemotherapy are also described in Chemotherapy Service, (1992) Ed., M. C. Perry, Williams & Wilkins, Baltimore, Md.

Non-limiting examples of additional therapeutic agents that can be used with the combination therapy include microtubule binding agents, DNA intercalators or cross-linkers, DNA synthesis inhibitors, DNA and RNA transcription inhibitors, antibodies, enzymes, enzyme inhibitors, gene regulators, angiogenesis inhibitors, and proteosome inhibitors (such as bortezomib or carfilzomib). These agents (which are administered at a therapeutically effective amount) and treatments can be used alone or in combination. For example, any suitable anti-cancer or anti-angiogenic agent can be administered in combination with the compounds disclosed herein. Methods and therapeutic dosages of such agents are known to those skilled in the art, and can be determined by a skilled clinician.

Additional chemotherapeutic agents include, but are not limited to alkylating agents, such as nitrogen mustards (for example, chlorambucil, chlormethine, cyclophosphamide, ifosfamide, and melphalan), nitrosoureas (for example, carmustine, fotemustine, lomustine, and streptozocin), platinum compounds (for example, carboplatin, cisplatin, oxaliplatin, and BBR3464), busulfan, dacarbazine, mechlorethamine, procarbazine, temozolomide, thiotepa, and uramustine; antimetabolites, such as folic acid (for example, methotrexate, pemetrexed, and raltitrexed), purine (for example, cladribine, clofarabine, fludarabine, mercaptopurine, and tioguanine), pyrimidine (for example, capecitabine), cytarabine, fluorouracil, and gemcitabine; plant alkaloids, such as podophyllum (for example, etoposide, and teniposide), taxane (for example, docetaxel and paclitaxel), vinca (for example, vinblastine, vincristine, vindesine, and vinorelbine); cytotoxic/antitumor antibiotics, such as anthracycline family members (for example, daunorubicin, doxorubicin, epirubicin, idarubicin, mitoxantrone, and valrubicin), bleomycin, rifampicin, hydroxyurea, and mitomycin; topoisomerase inhibitors, such as topotecan and irinotecan; monoclonal antibodies, such as alemtuzumab, bevacizumab, cetuximab, gemtuzumab, rituximab, panitumumab, pertuzumab, and trastuzumab; photosensitizers, such as aminolevulinic acid, methyl aminolevulinate, porfimer sodium, and verteporfin; and other agents, such as alitretinoin, altretamine, amsacrine, anagrelide, arsenic trioxide, asparaginase, axitinib, bexarotene, bevacizumab, bortezomib, celecoxib, denileukin diftitox, erlotinib, estramustine, gefitinib, hydroxycarbamide, imatinib, lapatinib, pazopanib, pentostatin, masoprocol, mitotane, pegaspargase, tamoxifen, sorafenib, sunitinib, vemurafinib, vandetanib, and tretinoin. Selection and therapeutic dosages of such agents are known to those skilled in the art, and can be determined by a skilled clinician.

The combination therapy may provide synergy and prove synergistic, that is, the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation, a synergistic effect may be attained when the compounds are administered or delivered sequentially, for example by different injections in separate syringes. In general, during alternation, an effective dosage of each active ingredient is administered sequentially, i.e. serially, whereas in combination therapy, effective dosages of two or more active ingredients are administered together.

In any method implementations of the present disclosure, the compound that is used in the method can be any compound according to the present disclosure and including 6-(3-(4-methoxyphenyl)-1,2,4-oxadiazol-5-yl)-N-(3-(pyrrolidin-1-yl)propyl)pyridazin-3-amine; N-(3-([1,4′-bipiperidin]-1′-yl)propyl)-6-(3-(4-methoxyphenyl)-1,2,4-oxadiazol-5-yl)pyridazin-3-amine; 6-(3-(4-methoxyphenyl)-1,2,4-oxadiazol-5-yl)-N-(3-(4-methylpiperazin-1-yl)propyl)pyridazin-3-amine; Ni-(6-(3-(4-methoxyphenyl)-1,2,4-oxadiazol-5-yl)pyridazin-3-yl)-N2,N2-dimethylethane-1,2-diamine; N-(3-(azepan-1-yl)propyl)-6-(3-(4-methoxyphenyl)-1,2,4-oxadiazol-5-yl)pyridazin-3-amine; 6-(3-(4-methoxyphenyl)-1,2,4-oxadiazol-5-yl)-N-(3-(4-methylpiperidin-1-yl)propyl)pyridazin-3-amine; and 6-(3-(4-methoxyphenyl)-1,2,4-oxadiazol-5-yl)-N-(2-morpholinoethyl)pyridazin-3-amine.

### VI. Pharmaceutical Compositions

Another aspect of the disclosure includes pharmaceutical compositions prepared for administration to a cell, such as an ex vivo, in vivo, or in vitro cell, and which include a therapeutically effective amount of one or more of the compounds disclosed herein. The therapeutically effective amount of a disclosed compound will depend on the route of administration, the species of subject and the physical characteristics of the subject being treated. Specific factors that can be taken into account include disease severity and stage, weight, diet and concurrent medications. The relationship of these factors to determining a therapeutically effective amount of the disclosed compounds is understood by those of skill in the art.

Pharmaceutical compositions for administration to a subject can include at least one further pharmaceutically acceptable additive such as carriers, thickeners, diluents, buffers, preservatives, surface active agents and the like in addition to the molecule of choice. Pharmaceutical compositions can also include one or more additional active ingredients such as antimicrobial agents, anti-inflammatory agents, anesthetics, and the like. The pharmaceutically acceptable carriers useful for these formulations are conventional. Remington's Pharmaceutical Sciences, by E. W. Martin, Mack Publishing Co., Easton, PA, 19th Edition (1995), describes compositions and formulations suitable for pharmaceutical delivery of the compounds herein disclosed.

In general, the nature of the carrier will depend on the particular mode of administration being employed. For instance, parenteral formulations usually contain injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Pharmaceutical compositions disclosed herein include those formed from pharmaceutically acceptable salts and/or solvates of the disclosed compounds. Pharmaceutically acceptable salts include those derived from pharmaceutically acceptable inorganic or organic bases and acids. Particular disclosed compounds possess at least one basic group that can form acid-base salts with acids. Examples of basic groups include, but are not limited to, amino and imino groups. Examples of inorganic acids that can form salts with such basic groups include, but are not limited to, mineral acids such as hydrochloric acid, hydrobromic acid, sulfuric acid or phosphoric acid. Basic groups also can form salts with organic carboxylic acids, sulfonic acids, sulfo acids or phospho acids or N-substituted sulfamic acid, for example acetic acid, propionic acid, glycolic acid, succinic acid, maleic acid, hydroxymaleic acid, methylmaleic acid, fumaric acid, malic acid, tartaric acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid, and, in addition, with amino acids, for example with α-amino acids, and also with methanesulfonic acid, ethanesulfonic acid, 2-hydroxymethanesulfonic acid, ethane-1,2-disulfonic acid, benzenedisulfonic acid, 4-methylbenzenesulfonic acid, naphthalene-2-sulfonic acid, 2- or 3-phosphoglycerate, glucose-6-phosphate or N-cyclohexylsulfamic acid (with formation of the cyclamates) or with other acidic organic compounds, such as ascorbic acid. In particular, suitable salts include those derived from alkali metals such as potassium and sodium, alkaline earth metals such as calcium and magnesium, among numerous other acids well known in the pharmaceutical art.

Certain compounds include at least one acidic group that can form an acid-base salt with an inorganic or organic base. Examples of salts formed from inorganic bases include salts of the presently disclosed compounds with alkali metals such as potassium and sodium, alkaline earth metals, including calcium and magnesium and the like. Similarly, salts of acidic compounds with an organic base, such as an amine (as used herein terms that refer to amines should be understood to include their conjugate acids unless the context clearly indicates that the free amine is intended) are contemplated, including salts formed with basic amino acids, aliphatic amines, heterocyclic amines, aromatic amines, pyridines, guanidines and amidines. Of the aliphatic amines, the acyclic aliphatic amines, and cyclic and acyclic di- and tri-alkyl amines are particularly suitable for use in the disclosed compounds. In addition, quaternary ammonium counterions also can be used.

Particular examples of suitable amine bases (and their corresponding ammonium ions) for use in the present compounds include, without limitation, pyridine, N,N-dimethylaminopyridine, diazabicyclononane, diazabicycloundecene, N-methyl-N-ethylamine, diethylamine, triethylamine, diisopropylethylamine, mono-, bis- or tris-(2-hydroxyethyl)amine, 2-hydroxy-tert-butylamine, tris(hydroxymethyl)methylamine, N,N-dimethyl-N-(2-hydroxyethyl)amine, tri-(2-hydroxyethyl)amine and N-methyl-D-glucamine. For additional examples of “pharmacologically acceptable salts,” see Berge et al., J. Pharm. Sci. 66:1 (1977).

Compounds disclosed herein can be crystallized and can be provided in a single crystalline form or as a combination of different crystal polymorphs. As such, the compounds can be provided in one or more physical form, such as different crystal forms, crystalline, liquid crystalline or non-crystalline (amorphous) forms. Such different physical forms of the compounds can be prepared using, for example different solvents or different mixtures of solvents for recrystallization. Alternatively or additionally, different polymorphs can be prepared, for example, by performing recrystallizations at different temperatures and/or by altering cooling rates during recrystallization. The presence of polymorphs can be determined by X-ray crystallography, or in some cases by another spectroscopic technique, such as solid phase NMR spectroscopy, IR spectroscopy, or by differential scanning calorimetry.

The pharmaceutical compositions can be administered to subjects by a variety of mucosal administration modes, including by oral, rectal, intranasal, intrapulmonary, or transdermal delivery, or by topical delivery to other surfaces. Optionally, the compositions can be administered by non-mucosal routes, including by intramuscular, subcutaneous, intravenous, intra-arterial, intra-articular, intraperitoneal, intrathecal, intracerebroventricular, or parenteral routes. In other alternative implementations, the compound can be administered ex vivo by direct exposure to cells, tissues or organs originating from a subject.

To formulate the pharmaceutical compositions, the compound can be combined with various pharmaceutically acceptable additives, as well as a base or vehicle for dispersion of the compound. Desired additives include, but are not limited to, pH control agents, such as arginine, sodium hydroxide, glycine, hydrochloric acid, citric acid, and the like. In addition, local anesthetics (for example, benzyl alcohol), isotonizing agents (for example, sodium chloride, mannitol, sorbitol), adsorption inhibitors (for example, Tween 80 or Miglyol 812), solubility enhancing agents (for example, cyclodextrins and derivatives thereof), stabilizers (for example, serum albumin), and reducing agents (for example, glutathione) can be included. Adjuvants, such as aluminum hydroxide (for example, Amphogel, Wyeth Laboratories, Madison, NJ), Freund's adjuvant, MPL™ (3-O-deacylated monophosphoryl lipid A; Corixa, Hamilton, IN) and IL-12 (Genetics Institute, Cambridge, MA), among many other suitable adjuvants well known in the art, can be included in the compositions. When the composition is a liquid, the tonicity of the formulation, as measured with reference to the tonicity of 0.9% (w/v) physiological saline solution taken as unity, is typically adjusted to a value at which no substantial, irreversible tissue damage will be induced at the site of administration. Generally, the tonicity of the solution is adjusted to a value of about 0.3 to about 3.0, such as about 0.5 to about 2.0, or about 0.8 to about 1.7.

The compound can be dispersed in a base or vehicle, which can include a hydrophilic compound having a capacity to disperse the compound, and any desired additives. The base can be selected from a wide range of suitable compounds, including but not limited to, copolymers of polycarboxylic acids or salts thereof, carboxylic anhydrides (for example, maleic anhydride) with other monomers (for example, methyl (meth)acrylate, acrylic acid and the like), hydrophilic vinyl polymers, such as polyvinyl acetate, polyvinyl alcohol, polyvinylpyrrolidone, cellulose derivatives, such as hydroxymethylcellulose, hydroxypropylcellulose and the like, and natural polymers, such as chitosan, collagen, sodium alginate, gelatin, hyaluronic acid, and nontoxic metal salts thereof. Often, a biodegradable polymer is selected as a base or vehicle, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters and the like can be employed as vehicles. Hydrophilic polymers and other vehicles can be used alone or in combination, and enhanced structural integrity can be imparted to the vehicle by partial crystallization, ionic bonding, cross-linking and the like. The vehicle can be provided in a variety of forms, including fluid or viscous solutions, gels, pastes, powders, microspheres and films for direct application to a mucosal surface.

The compound can be combined with the base or vehicle according to a variety of methods, and release of the compound can be by diffusion, disintegration of the vehicle, or associated formation of water channels. In some circumstances, the compound is dispersed in microcapsules (microspheres) or nanocapsules (nanospheres) prepared from a suitable polymer, for example, isobutyl 2-cyanoacrylate (see, for example, Michael et al., J. Pharmacy Pharmacol. 43:1-5, 1991), and dispersed in a biocompatible dispersing medium, which yields sustained delivery and biological activity over a protracted time.

The compositions of the disclosure can alternatively contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate. For solid compositions, conventional nontoxic pharmaceutically acceptable vehicles can be used which include, for example, pharmaceutical grades of mannitol, lactose, starch, magnesium stearate, sodium saccharin, talcum, cellulose, glucose, sucrose, magnesium carbonate, and the like.

Pharmaceutical compositions for administering the compound can also be formulated as a solution, microemulsion, or other ordered structure suitable for high concentration of active ingredients. The vehicle can be a solvent or dispersion medium containing, for example, water, ethanol, polyol (for example, glycerol, propylene glycol, liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants. In many cases, it will be desirable to include isotonic agents, for example, sugars, polyalcohols, such as mannitol and sorbitol, or sodium chloride in the composition. Prolonged absorption of the compound can be brought about by including in the composition an agent which delays absorption, for example, monostearate salts and gelatin.

In certain implementations, the compound can be administered in a time release formulation, for example in a composition which includes a slow release polymer. These compositions can be prepared with vehicles that will protect against rapid release, for example a controlled release vehicle such as a polymer, microencapsulated delivery system or bioadhesive gel. Prolonged delivery in various compositions of the disclosure can be brought about by including in the composition agents that delay absorption, for example, aluminum monostearate hydrogels and gelatin. When controlled release formulations are desired, controlled release binders suitable for use in accordance with the disclosure include any biocompatible controlled release material which is inert to the active agent and which is capable of incorporating the compound and/or other biologically active agent. Numerous such materials are known in the art. Useful controlled-release binders are materials that are metabolized slowly under physiological conditions following their delivery (for example, at a mucosal surface, or in the presence of bodily fluids). Appropriate binders include, but are not limited to, biocompatible polymers and copolymers well known in the art for use in sustained release formulations. Such biocompatible compounds are non-toxic and inert to surrounding tissues, and do not trigger significant adverse side effects, such as nasal irritation, immune response, inflammation, or the like. They are metabolized into metabolic products that are also biocompatible and easily eliminated from the body.

Exemplary polymeric materials for use in the present disclosure include, but are not limited to, polymeric matrices derived from copolymeric and homopolymeric polyesters having hydrolyzable ester linkages. A number of these are known in the art to be biodegradable and to lead to degradation products having no or low toxicity. Exemplary polymers include polyglycolic acids and polylactic acids, poly(DL-lactic acid-co-glycolic acid), poly(D-lactic acid-co-glycolic acid), and poly(L-lactic acid-co-glycolic acid). Other useful biodegradable or bioerodable polymers include, but are not limited to, such polymers as poly(epsilon-caprolactone), poly(epsilon-aprolactone-CO-lactic acid), poly(epsilon.-aprolactone-CO-glycolic acid), poly(beta-hydroxy butyric acid), poly(alkyl-2-cyanoacrilate), hydrogels, such as poly(hydroxyethyl methacrylate), polyamides, poly(amino acids) (for example, L-leucine, glutamic acid, L-aspartic acid and the like), poly(ester urea), poly(2-hydroxyethyl DL-aspartamide), polyacetal polymers, polyorthoesters, polycarbonate, polymaleamides, polysaccharides, and copolymers thereof. Many methods for preparing such formulations are well known to those skilled in the art (see, for example, Sustained and Controlled Release Drug Delivery Systems, J. R. Robinson, ed., Marcel Dekker, Inc., New York, 1978). Other useful formulations include controlled-release microcapsules (U.S. Pat. Nos. 4,652,441 and 4,917,893), lactic acid-glycolic acid copolymers useful in making microcapsules and other formulations (U.S. Pat. Nos. 4,677,191 and 4,728,721) and sustained-release compositions for water-soluble peptides (U.S. Pat. No. 4,675,189).

The pharmaceutical compositions of the disclosure typically are sterile and stable under conditions of manufacture, storage and use. Sterile solutions can be prepared by incorporating the compound in the required amount in an appropriate solvent with one or a combination of ingredients enumerated herein, as required, followed by filtered sterilization. Generally, dispersions are prepared by incorporating the compound and/or other biologically active agent into a sterile vehicle that contains a basic dispersion medium and the required other ingredients from those enumerated herein. In the case of sterile powders, methods of preparation include vacuum drying and freeze-drying which yields a powder of the compound plus any additional desired ingredient from a previously sterile-filtered solution thereof. The prevention of the action of microorganisms can be accomplished by various antibacterial and antifungal agents, for example, parabens, chlorobutanol, phenol, sorbic acid, thimerosal, and the like.

The instant disclosure also includes kits, packages and multi-container units containing the herein described pharmaceutical compositions, active ingredients, and/or means for administering the same for use in the prevention and treatment of diseases and other conditions in mammalian subjects. Kits for diagnostic use are also provided. In one implementation, these kits include a container or formulation that contains one or more of the compounds described herein. In one example, this component is formulated in a pharmaceutical preparation for delivery to a subject. The compound is optionally contained in a bulk dispensing container or unit or multi-unit dosage form. Optional dispensing means can be provided, for example a pulmonary or intranasal spray applicator. Packaging materials optionally include a label or instruction indicating for what treatment purposes and/or in what manner the pharmaceutical agent packaged therewith can be used.

In any composition implementations of the present disclosure, the compound implementation that is used in the composition can be any compound according to the present disclosure and including 6-(3-(4-methoxyphenyl)-1,2,4-oxadiazol-5-yl)-N-(3-(pyrrolidin-1-yl)propyl)pyridazin-3-amine; N-(3-([1,4′-bipiperidin]-1′-yl)propyl)-6-(3-(4-methoxyphenyl)-1,2,4-oxadiazol-5-yl)pyridazin-3-amine; 6-(3-(4-methoxyphenyl)-1,2,4-oxadiazol-5-yl)-N-(3-(4-methylpiperazin-1-yl)propyl)pyridazin-3-amine; Ni-(6-(3-(4-methoxyphenyl)-1,2,4-oxadiazol-5-yl)pyridazin-3-yl)-N2,N2-dimethylethane-1,2-diamine; N-(3-(azepan-1-yl)propyl)-6-(3-(4-methoxyphenyl)-1,2,4-oxadiazol-5-yl)pyridazin-3-amine; 6-(3-(4-methoxyphenyl)-1,2,4-oxadiazol-5-yl)-N-(3-(4-methylpiperidin-1-yl)propyl)pyridazin-3-amine; and 6-(3-(4-methoxyphenyl)-1,2,4-oxadiazol-5-yl)-N-(2-morpholinoethyl)pyridazin-3-amine.

### VII. Representative Implementations

Disclosed herein are compounds, or a stereoisomer, tautomer, or pharmaceutically acceptable salt or ester thereof, according to Formula IA

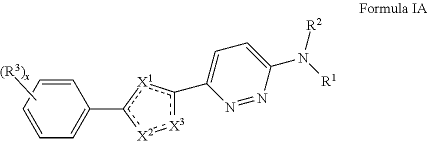

wherein
each of X¹, X², and X³ independently is N or O;
R¹ is -(linker)ₜ-R^{b} wherein R^{b} is aromatic, heteroaliphatic, or aliphatic; the linker is an aliphatic or heteroaliphatic group; and t is 0 or 1;
R² is H or aliphatic;
each R³ independently is heteroaliphatic, hydroxy, aliphatic, haloaliphatic, or halo;
x is an integer selected from 0 to 5; and
   provided that the compound is not 6-(3-(4-methoxyphenyl)-1,2,4-oxadiazol-5-yl)-N-(3-(pyrrolidin-1-yl)propyl)pyridazin-3-amine, N-(3-([1,4′-bipiperidin]-1′-yl)propyl)-6-(3-(4-methoxyphenyl)-1,2,4-oxadiazol-5-yl)pyridazin-3-amine, 6-(3-(4-methoxyphenyl)-1,2,4-oxadiazol-5-yl)-N-(3-(4-methylpiperazin-1-yl)propyl)pyridazin-3-amine, N1-(6-(3-(4-methoxyphenyl)-1,2,4-oxadiazol-5-yl)pyridazin-3-yl)-N2,N2-dimethylethane-1,2-diamine, N-(3-(azepan-1-yl)propyl)-6-(3-(4-methoxyphenyl)-1,2,4-oxadiazol-5-yl)pyridazin-3-amine, 6-(3-(4-methoxyphenyl)-1,2,4-oxadiazol-5-yl)-N-(3-(4-methylpiperidin-1-yl)propyl)pyridazin-3-amine, or 6-(3-(4-methoxyphenyl)-1,2,4-oxadiazol-5-yl)-N-(2-morpholinoethyl)pyridazin-3-amine.

Also disclosed herein are compounds, or a stereoisomer, tautomer, or pharmaceutically acceptable salt or ester thereof, according to Formula I

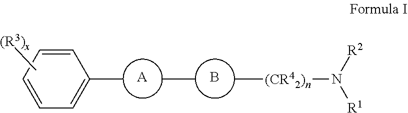

wherein
ring A is a 5-membered heteroaryl ring other than thiophenyl, thiazolyl, furanyl, triazolyl, thiadiazolyl, and 1,3,4-oxadiazolyl;
ring B is a 6-membered heteroaryl ring containing at least two nitrogen atoms;
R¹ is -(linker)ₜ-R^{b} wherein R^{b} is aromatic, heteroaliphatic, or aliphatic; the linker is an aliphatic or heteroaliphatic group; and t is 0 or 1;
R² is H or aliphatic;
each R³ independently is heteroaliphatic, hydroxy, aliphatic, haloaliphatic, or halo;
each R⁴ independently is H, aliphatic, or halo;
x is an integer selected from 0 to 5; and
n is an integer selected from 0 to 10;
   provided that the compound is not 6-(3-(4-methoxyphenyl)-1,2,4-oxadiazol-5-yl)-N-(3-(pyrrolidin-1-yl)propyl)pyridazin-3-amine, N-(3-([1,4′-bipiperidin]-1′-yl)propyl)-6-(3-(4-methoxyphenyl)-1,2,4-oxadiazol-5-yl)pyridazin-3-amine, 6-(3-(4-methoxyphenyl)-1,2,4-oxadiazol-5-yl)-N-(3-(4-methylpiperazin-1-yl)propyl)pyridazin-3-amine, Ni-(6-(3-(4-methoxyphenyl)-1,2,4-oxadiazol-5-yl)pyridazin-3-yl)-N2,N2-dimethylethane-1,2-diamine, N-(3-(azepan-1-yl)propyl)-6-(3-(4-methoxyphenyl)-1,2,4-oxadiazol-5-yl)pyridazin-3-amine, 6-(3-(4-methoxyphenyl)-1,2,4-oxadiazol-5-yl)-N-(3-(4-methylpiperidin-1-yl)propyl)pyridazin-3-amine, or 6-(3-(4-methoxyphenyl)-1,2,4-oxadiazol-5-yl)-N-(2-morpholinoethyl)pyridazin-3-amine.

Also disclosed herein are compounds, a stereoisomer, tautomer, or pharmaceutically acceptable salt or ester thereof, according to Formula I:

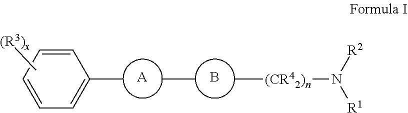

wherein
ring A is a 5-membered heteroaryl ring;
ring B is a 6-membered heteroaryl ring containing at least two nitrogen atoms;
R¹ is —(CR^{a}₂)ₘ—R^{b} where each R^{a} independently is H, alkyl, or halo, m is 1, 2, 3, 4, or 5, and R^{b} is a nitrogen-containing group;
R² is H or alkyl;
each R³ independently is alkoxy, hydroxy, aliphatic, or halo;
each R⁴ independently is H, alkyl, or halo;
n is 0, 1, 2, or 3; and
x is 0, 1, 2, 3, 4, or 5,
   with the proviso that the compound does not comprise the structure set forth as any one of MY-1, MY-2, MY-10, MY-11, or MY-12.

Also disclosed herein are compounds, or a stereoisomer, tautomer, or pharmaceutically acceptable salt or ester thereof, according to Formula I

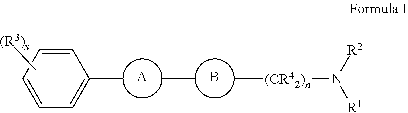

wherein
ring A is a 5-membered heteroaryl ring;
ring B is a 6-membered heteroaryl ring containing at least two nitrogen atoms;
R¹ is —(CR^{a}₂)ₘ—R^{b} or —[(CR^{a}₂)ₘO]ᵣ—(CH₂)ₛ—R^{b}, wherein each R^{a} independently is H, aliphatic, or halo; t is 1, 2, 3, 4, or 5; r is 1, 2, 3, 4, or 5; s is 0 or 1; and R is an acridinyl group;
R² is H or aliphatic;
each R³ independently is heteroaliphatic, hydroxy, aliphatic, haloaliphatic, or halo;
each R⁴ independently is H, aliphatic, or halo;
x is an integer selected from 0 to 5; and
n is an integer selected from 0 to 10.

In any or all of these representative implementations, ring A is

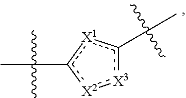

where:
each bond represented by [symbol] is a single or double bond as needed to satisfy valence requirements; and
each of X¹, X², and X³ independently is N, O, S, or C(R^{c}) where R^{c} is H, alkyl, or halo, provided that at least one of X¹, X², and X³ is other than C(R^{c}).

In any or all of these representative implementations, ring A is

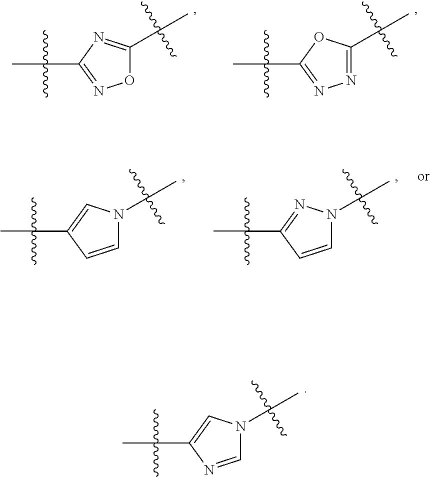 [SMILES: *c1noc(*)n1 ; *c1nnc(*)o1 ; *c1ccn(*)c1 ; *c1ccn(*)n1 ; *c1cn(*)cn1]

In any or all of these representative implementations, ring A is

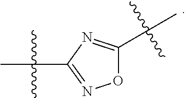 [SMILES: *c1noc(*)n1]

In any or all of these representative implementations, ring B is

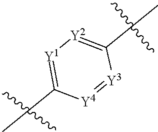

where:
each of Y¹, Y², Y³, and Y⁴ independently is N or C(R^{c}) where R^{c} is H, alkyl, or halo, provided that at least two of Y¹, Y², Y³, and Y⁴ are N.

In any or all of these representative implementations, ring B is

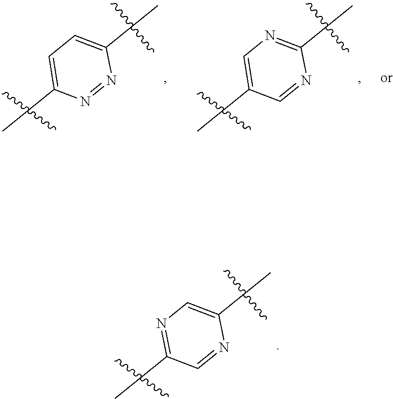 [SMILES: *c1ccc(*)nn1 ; *c1cnc(*)nc1 ; *c1cnc(*)cn1]

In any or all of these representative implementations, ring B is

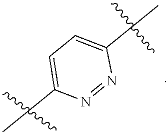 [SMILES: *c1ccc(*)nn1]

In any or all of these representative implementations, (i) x is 1, 2, or 3; or (ii) one R³ is in a para position to ring A; or (iii) both (i) and (ii).

In any or all of these representative implementations, n is 0.

In any or all of these representative implementations, each R³ independently is C₁-C₃ alkoxy or hydroxy.

In any or all of these representative implementations, x is 1 and R³ is methoxy.

In any or all of these representative implementations, the compound has a structure according to Formula II

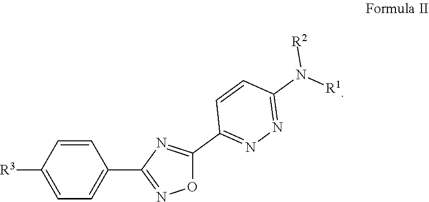

In any or all of these representative implementations, R² is H.

In any or all of these representative implementations, the compound has a structure according to Formula III

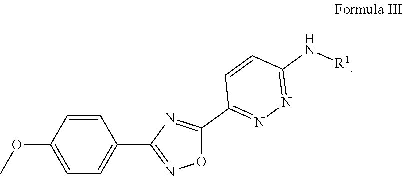

In any or all of these representative implementations, R¹ is —(CH₂)ₘ—R^{b}.

In any or all of these representative implementations, m is 2 or 3.

In any or all of these representative implementations, R^{b} is an N-containing cyclic group —N(R^{c})₂, or —C(O)N(R^{c})₂ where each R^{c} is H or alkyl.

In any or all of these representative implementations, R¹ is:

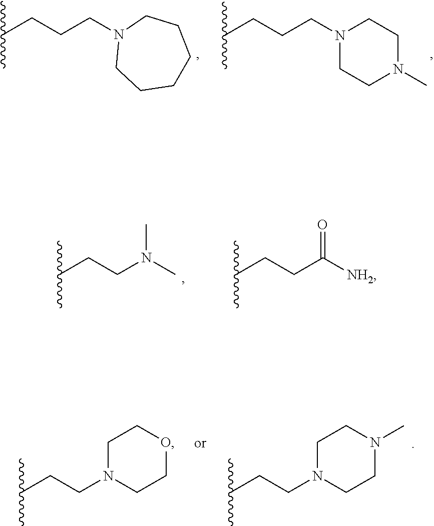

In any or all of these representative implementations, the compound is:

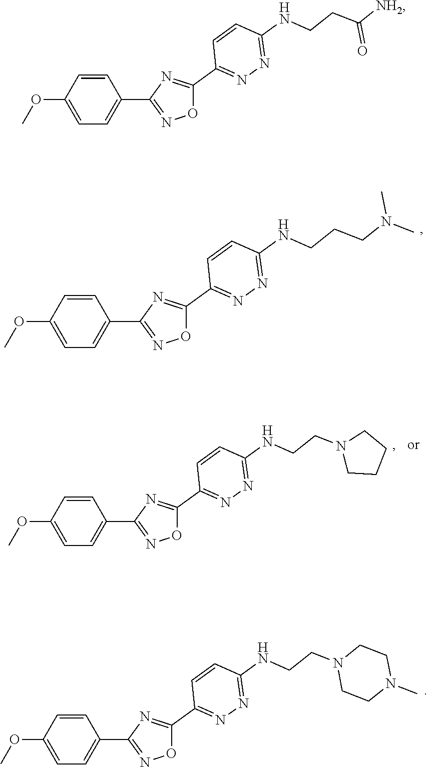

In any or all of these representative implementations, the compound has a structure according to Formula IV

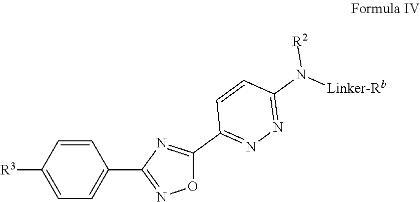

wherein the linker is —(CR^{a}₂)ₘ— or —[(CR^{a}₂)ₘO]ᵣ—(CR^{a}₂)ₛ— wherein each R^{a} independently is H, aliphatic, or halo; m is 1, 2, 3, 4, or 5; r is 1, 2, 3, 4, or 5; and s is 0 or 1.

In any or all of these representative implementations, the compound has a structure according to Formula V

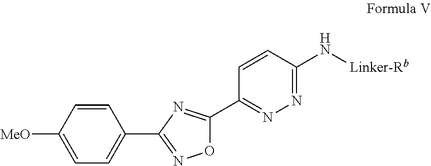

wherein the linker is —(CH₂)ₘ— or —[(CH₂)ₘO]ᵣ—(CH₂)ₛ— wherein m is 1, 2, 3, 4, or 5; r is 1, 2, 3, 4, or 5; and s is 0 or 1.

In any or all of these representative implementations, R¹ is:

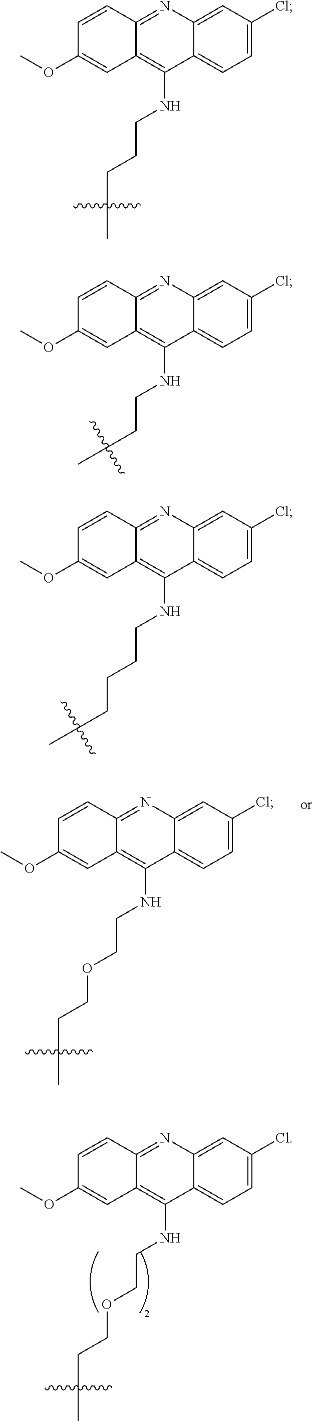

In any or all of these representative implementations, the compound is:

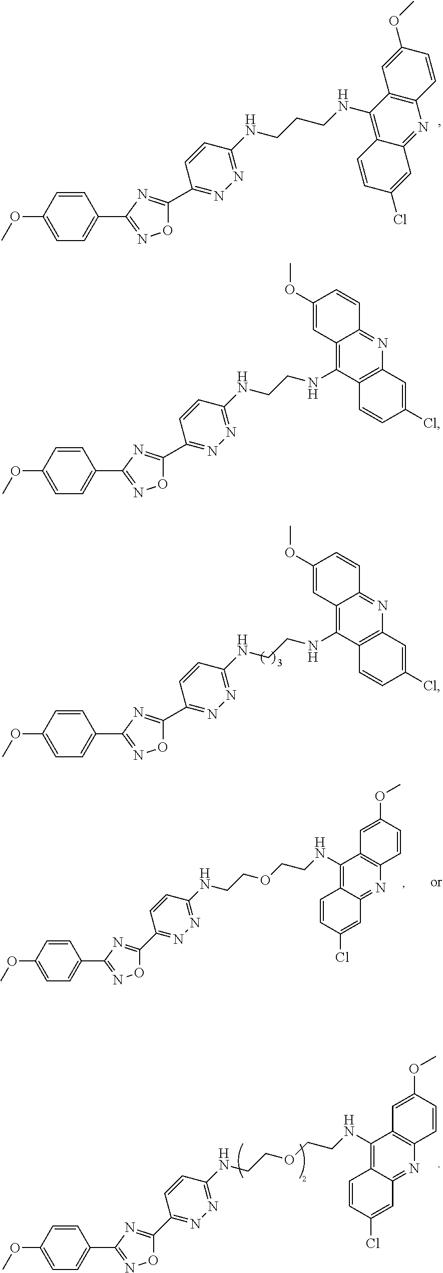

Also disclosed herein is a pharmaceutical composition comprising a compound according to any or all of the representative implementations described above and at least one pharmaceutically acceptable additive.

In any or all of these representative implementations, the pharmaceutical composition comprises a unit dosage form of a therapeutic amount of the compound.

In any or all of these representative implementations, the pharmaceutical composition further comprises an anticancer agent.

Also disclosed herein is a method of decreasing cancer-relevant protein expression in a cell, comprising contacting the cell with an effective amount of a compound, or a stereoisomer, tautomer, or pharmaceutically acceptable salt or ester thereof, according to Formula IA

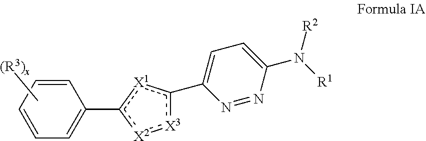

wherein
each of X¹, X², and X³ independently is N or O;
R¹ is -(linker)ₜ-R^{b} wherein R^{b} is aromatic, heteroaliphatic, or aliphatic; the linker is an aliphatic or heteroaliphatic group; and t is 0 or 1;
R² is H or aliphatic;
each R³ independently is heteroaliphatic, hydroxy, aliphatic, haloaliphatic, or halo;
x is an integer selected from 0 to 5; and
   provided that the compound is not 6-(3-(4-methoxyphenyl)-1,2,4-oxadiazol-5-yl)-N-(3-(pyrrolidin-1-yl)propyl)pyridazin-3-amine, N-(3-([1,4′-bipiperidin]-1′-yl)propyl)-6-(3-(4-methoxyphenyl)-1,2,4-oxadiazol-5-yl)pyridazin-3-amine, 6-(3-(4-methoxyphenyl)-1,2,4-oxadiazol-5-yl)-N-(3-(4-methylpiperazin-1-yl)propyl)pyridazin-3-amine, Ni-(6-(3-(4-methoxyphenyl)-1,2,4-oxadiazol-5-yl)pyridazin-3-yl)-N2,N2-dimethylethane-1,2-diamine, N-(3-(azepan-1-yl)propyl)-6-(3-(4-methoxyphenyl)-1,2,4-oxadiazol-5-yl)pyridazin-3-amine, 6-(3-(4-methoxyphenyl)-1,2,4-oxadiazol-5-yl)-N-(3-(4-methylpiperidin-1-yl)propyl)pyridazin-3-amine, or 6-(3-(4-methoxyphenyl)-1,2,4-oxadiazol-5-yl)-N-(2-morpholinoethyl)pyridazin-3-amine.

Also disclosed is a method of decreasing cancer-relevant protein expression in a cell, comprising contacting the cell with an effective amount of a compound, or a stereoisomer, tautomer, or pharmaceutically acceptable salt or ester thereof, according to any or all of the above listed representative implementations concerning Formulas, I, II, III, IV, and V.

In any or all representative implementations of these methods,
(i) ring A is

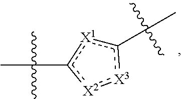

where each bond represented by [symbol] is a single or double bond as needed to satisfy valence requirements, and each of X¹, X², and X³ independently is N, O, S, or C(R^{c}) where R^{c} is H or alkyl, provided that at least one of X¹, X², and X³ is other than C(R^{c}); or
(ii) ring B is

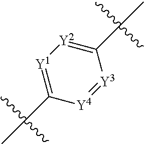

where each of Y¹, Y², Y³, and Y⁴ independently is N or C(R^{c}) where R^{c} is H or alkyl, provided that at least two of Y¹, Y², Y³, and Y⁴ are N; or
(iii) both (i) and (ii).

In any or all representative implementations of these methods,
(i) ring A is

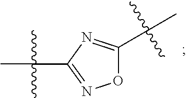 [SMILES: *c1noc(*)n1]

or
(ii) ring B is

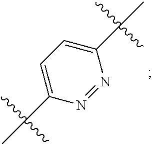 [SMILES: *c1ccc(*)nn1]

or
(iii) both (i) and (ii).

In any or all representative implementations of these methods,
(i) R² is H; or
(ii) R³ is C₁-C₃ alkoxy or hydroxy; or
(iii) x is 1, 2, or 3; or
(iv) one R³ is in a para position to ring A;
(v) n is 0; or
(vi) any combination of (i), (ii), (iii), (iv), and (v).

In any or all representative implementations of these methods, the compound has a structure according to any one of Formulas II, III, IV, or V

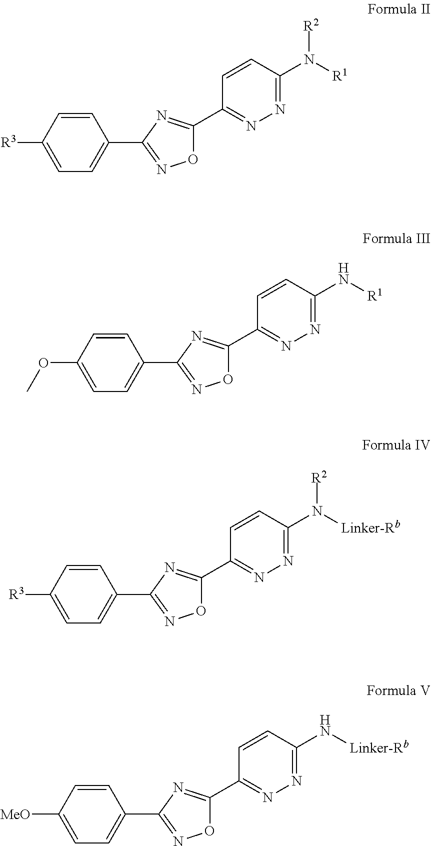

wherein for Formulas VI and V, the linker is —(CR^{a}₂)ₘ— or —[(CR^{a}₂)ₘO]ᵣ—(CR^{a}₂)ₛ— wherein each R^{a} independently is H, aliphatic, or halo; m is 1, 2, 3, 4, or 5; r is 1, 2, 3, 4, or 5; and s is 0 or 1.

In any or all representative implementations of these methods, the compound is:

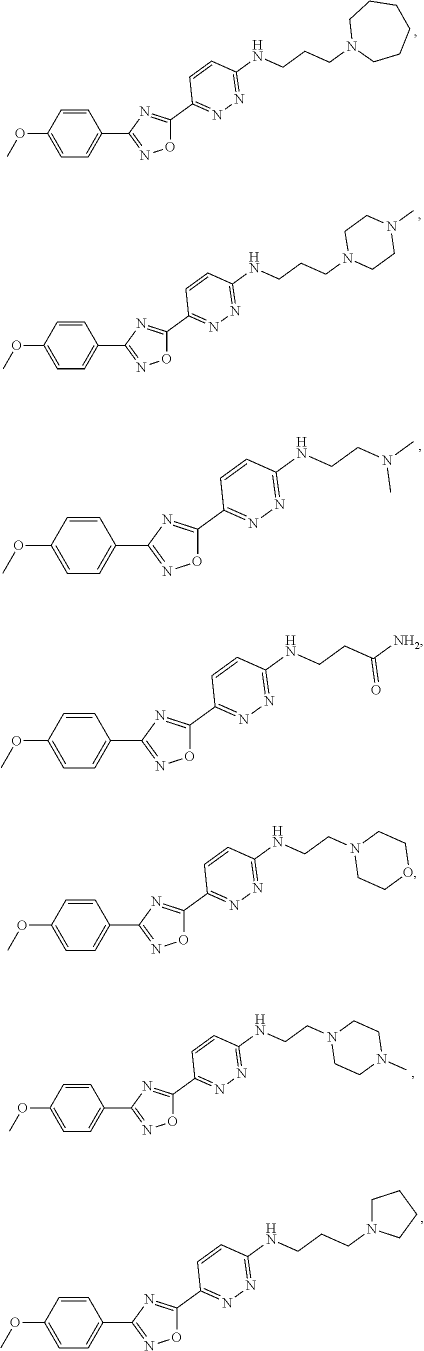

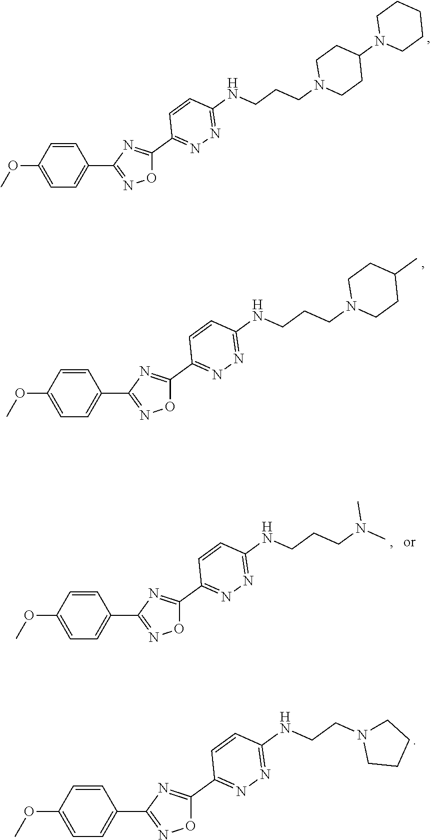

In any or all representative implementations of these methods, the compound is

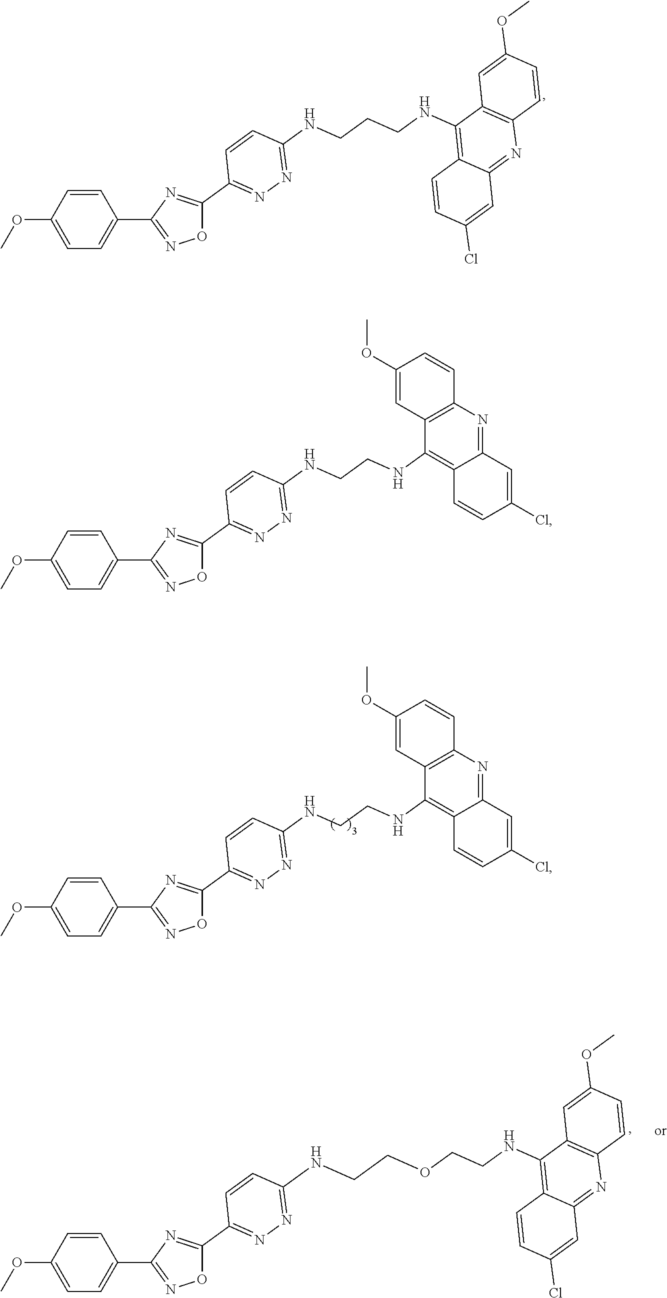

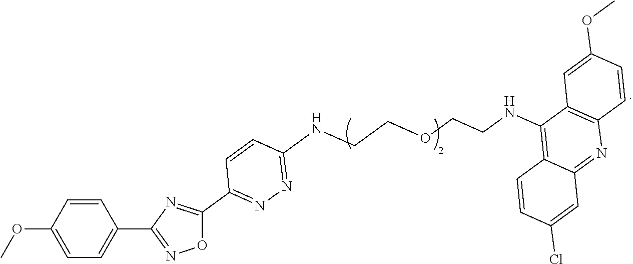

In any or all representative implementations of these methods, the compound is

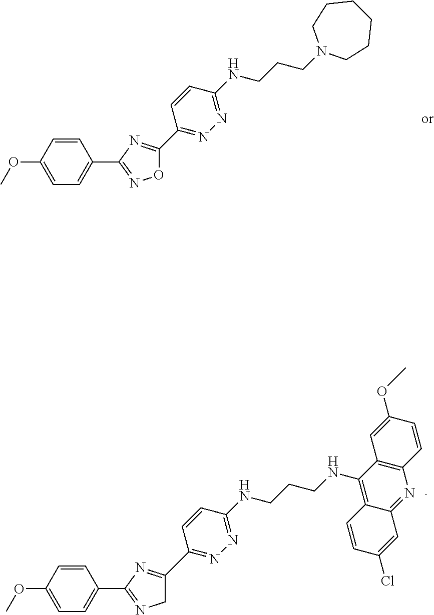 [SMILES: COc1ccc(-c2noc(-c3ccc(NCCCN4CCCCCC4)nn3)n2)cc1 ; COc1ccc(C2=NCC(c3ccc(NCCCNc4c5ccc(Cl)cc5nc5ccc(OC)cc45)nn3)=N2)cc1]

In any or all representative implementations of these methods, the cell is in vitro.

In any or all representative implementations of these methods, the cell is in vivo.

In any or all representative implementations of these methods, decreasing cancer-relevant protein expression in the cell decreases growth and/or proliferation of the cell.

In any or all representative implementations of these methods, the cell is a cell with overexpression of the MYCN gene.

In any or all representative implementations of these methods, the compound selectively binds to a non-canonical G4 quadruplex nucleic acid region in the MYCN gene.

In any or all representative implementations of these methods, the non-canonical G4 quadruplex nucleic acid region comprises a hairpin structure.

In any or all representative implementations of these methods, the cell is a cancer cell in a subject, and wherein the method further comprising treating or preventing cancer in the subject, comprising the step of administering to a subject in need thereof a therapeutically effective amount of the compound, or the pharmaceutically acceptable salt or ester thereof, to decrease N-Myc expression in the cancer cell, thereby treating or preventing the cancer in the subject.

In any or all representative implementations of these methods, the cancer cell is a neurological cancer cell or a lung cancer cell.

In any or all representative implementations of these methods, the cancer is neuroblastoma, rhabdomyosarcoma, prostate cancer, or small cell lung cancer.

In any or all representative implementations of these methods, treating the cancer comprises decreasing tumor volume, decreasing the number or size of metastases, or lessening a symptom of the cancer.

In any or all representative implementations of these methods, the method further comprises administering a therapeutically effective amount of an additional anticancer agent to the subject.

Also disclosed is a use of a compound or a stereoisomer, tautomer, or pharmaceutically acceptable salt or ester thereof, according to Formula IA, for decreasing N-Myc expression in a cell, comprising contacting the cell with an effective amount of the compound according to Formula IA

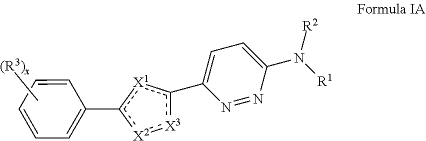

wherein
each of X¹, X², and X³ independently is N or O;
R¹ is -(linker)ₜ-R^{b} wherein R^{b} is aromatic, heteroaliphatic, or aliphatic; the linker is an aliphatic or heteroaliphatic group; and t is 0 or 1;
R² is H or aliphatic;
each R³ independently is heteroaliphatic, hydroxy, aliphatic, haloaliphatic, or halo;
x is an integer selected from 0 to 5; and
   provided that the compound is not 6-(3-(4-methoxyphenyl)-1,2,4-oxadiazol-5-yl)-N-(3-(pyrrolidin-1-yl)propyl)pyridazin-3-amine, N-(3-([1,4′-bipiperidin]-1′-yl)propyl)-6-(3-(4-methoxyphenyl)-1,2,4-oxadiazol-5-yl)pyridazin-3-amine, 6-(3-(4-methoxyphenyl)-1,2,4-oxadiazol-5-yl)-N-(3-(4-methylpiperazin-1-yl)propyl)pyridazin-3-amine, Ni-(6-(3-(4-methoxyphenyl)-1,2,4-oxadiazol-5-yl)pyridazin-3-yl)-N2,N2-dimethylethane-1,2-diamine, N-(3-(azepan-1-yl)propyl)-6-(3-(4-methoxyphenyl)-1,2,4-oxadiazol-5-yl)pyridazin-3-amine, 6-(3-(4-methoxyphenyl)-1,2,4-oxadiazol-5-yl)-N-(3-(4-methylpiperidin-1-yl)propyl)pyridazin-3-amine, or 6-(3-(4-methoxyphenyl)-1,2,4-oxadiazol-5-yl)-N-(2-morpholinoethyl)pyridazin-3-amine.

Further disclosed is a use of a compound or a stereoisomer, tautomer, or pharmaceutically acceptable salt or ester thereof, according to any or all representative implementations described herein for Formulas I, II, III, IV, and V for decreasing N-Myc expression in a cell, comprising contacting the cell with an effective amount of the compound according any or all representative implementations described herein for Formulas I, II, III, IV, and V.

Also disclosed is a use of a compound or a stereoisomer, tautomer, or pharmaceutically acceptable salt or ester thereof, according to Formula IA, for treating or preventing cancer in a subject, wherein the compound has a structure according to Formula IA:

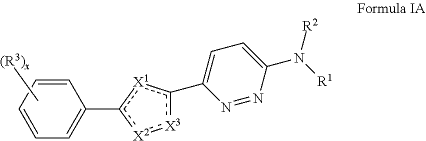

wherein
each of X¹, X², and X³ independently is N or O;
R¹ is -(linker)ₜ-R^{b} wherein R^{b} is aromatic, heteroaliphatic, or aliphatic; the linker is an aliphatic or heteroaliphatic group; and t is 0 or 1;
R² is H or aliphatic;
each R³ independently is heteroaliphatic, hydroxy, aliphatic, haloaliphatic, or halo;
x is an integer selected from 0 to 5; and
   provided that the compound is not 6-(3-(4-methoxyphenyl)-1,2,4-oxadiazol-5-yl)-N-(3-(pyrrolidin-1-yl)propyl)pyridazin-3-amine, N-(3-([1,4′-bipiperidin]-1′-yl)propyl)-6-(3-(4-methoxyphenyl)-1,2,4-oxadiazol-5-yl)pyridazin-3-amine, 6-(3-(4-methoxyphenyl)-1,2,4-oxadiazol-5-yl)-N-(3-(4-methylpiperazin-1-yl)propyl)pyridazin-3-amine, Ni-(6-(3-(4-methoxyphenyl)-1,2,4-oxadiazol-5-yl)pyridazin-3-yl)-N2,N2-dimethylethane-1,2-diamine, N-(3-(azepan-1-yl)propyl)-6-(3-(4-methoxyphenyl)-1,2,4-oxadiazol-5-yl)pyridazin-3-amine, 6-(3-(4-methoxyphenyl)-1,2,4-oxadiazol-5-yl)-N-(3-(4-methylpiperidin-1-yl)propyl)pyridazin-3-amine, or 6-(3-(4-methoxyphenyl)-1,2,4-oxadiazol-5-yl)-N-(2-morpholinoethyl)pyridazin-3-amine.

Also disclosed is a use of a compound or a stereoisomer, tautomer, or pharmaceutically acceptable salt or ester thereof, according to any or all representative implementations described herein for Formulas I, II, III, IV, and V for treating or preventing cancer in a subject, wherein the compound has a structure according to any or all representative implementations described herein for Formulas I, II, III, IV, and V.

Also disclosed is a use of a compound or a stereoisomer, tautomer, or pharmaceutically acceptable salt or ester thereof, according to Formula IA, in the manufacture of a medicament for treating or preventing cancer in a subject, wherein the compound has a structure according to Formula IA

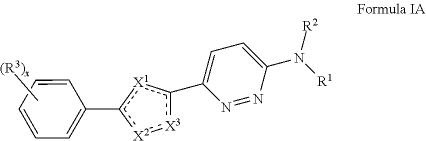

wherein
each of X¹, X², and X³ independently is N or O;
R¹ is -(linker)ₜ-R^{b} wherein R^{b} is aromatic, heteroaliphatic, or aliphatic; the linker is an aliphatic or heteroaliphatic group; and t is 0 or 1;
R² is H or aliphatic;
each R³ independently is heteroaliphatic, hydroxy, aliphatic, haloaliphatic, or halo;
x is an integer selected from 0 to 5; and
   provided that the compound is not 6-(3-(4-methoxyphenyl)-1,2,4-oxadiazol-5-yl)-N-(3-(pyrrolidin-1-yl)propyl)pyridazin-3-amine, N-(3-([1,4′-bipiperidin]-1′-yl)propyl)-6-(3-(4-methoxyphenyl)-1,2,4-oxadiazol-5-yl)pyridazin-3-amine, 6-(3-(4-methoxyphenyl)-1,2,4-oxadiazol-5-yl)-N-(3-(4-methylpiperazin-1-yl)propyl)pyridazin-3-amine, Ni-(6-(3-(4-methoxyphenyl)-1,2,4-oxadiazol-5-yl)pyridazin-3-yl)-N2,N2-dimethylethane-1,2-diamine, N-(3-(azepan-1-yl)propyl)-6-(3-(4-methoxyphenyl)-1,2,4-oxadiazol-5-yl)pyridazin-3-amine, 6-(3-(4-methoxyphenyl)-1,2,4-oxadiazol-5-yl)-N-(3-(4-methylpiperidin-1-yl)propyl)pyridazin-3-amine, or 6-(3-(4-methoxyphenyl)-1,2,4-oxadiazol-5-yl)-N-(2-morpholinoethyl)pyridazin-3-amine.

Further disclosed is a use of a compound or a stereoisomer, tautomer, or pharmaceutically acceptable salt or ester thereof, according to any or all representative implementations described herein for Formulas I, II, III, IV, and V in the manufacture of a medicament for treating or preventing cancer in a subject, wherein the compound has a structure according to any or all representative implementations described herein for Formulas I, II, III, IV, and V.

### VIII. Examples

The following examples are provided to illustrate particular features of certain implementations, but the scope of the claims should not be limited to those features exemplified.

### Materials and Methods

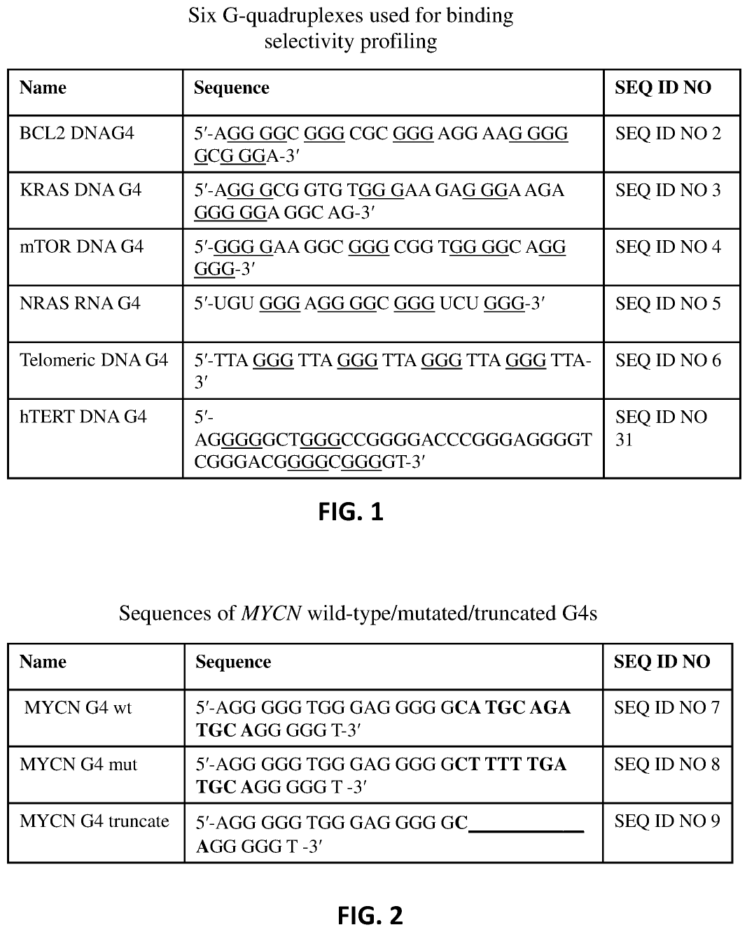

MYCN G4 oligonucleotides (5′-AGG GGG TGG GAG GGG GCA TGC AGA TGC AGG GGG T-3′, SEQ ID NO: 7) with or without labeling were purchased from Integrated DNA Technology. Other sequences of DNA/RNA samples used for binding specificity study are summarized in FIGS. 1-2:

Hit compounds for binding validation were purchased from ChemDiv and Chembridge. Analogs of compound MY-1 were synthesized in house or purchased. The RNA extraction kit RNeasy Plus Mini kit (#74134) was purchased from Qiagen, CA. The cDNA synthesis kit High-Capacity RNA-to-cDNA™ Kit (#4387406) was purchased from Life Technologies (Invitrogen), New York. The Fast SYBR™ Green Master Mix was purchased from Life Technologies (Invitrogen), NY. The anti-N-Myc (B8.4.B): sc-53993 antibody (1:4000 dilution; Santa Cruz Biotechnology (SCB), USA), the anti-GAPDH (0411): sc-47724 antibody (1:2000 dilution; SCB, USA), and the goat anti-mouse IgG-HRP: sc-2005 antibody (1:1000 dilution; SCB, USA) were used for immunoblots.

General chemistry methods. All chemical reagents were obtained from commercial suppliers and used without further purification. In these examples, compounds for SAR study were either purchased from ChemDiv (MY-1, MY-2, MY-3, MY-4, MY-9, MY-10, MY-11, MY-12) or synthesized in the lab (MY-5, MY-6, MY-7, MY-8, MY-13, MY-14). Solvents were removed using a Buchi rotary evaporator under reduced pressure. Flash column chromatography was performed using a Teledyne ISCO CombiFlash Rf automated chromatography system. ¹H and ¹³C NMR spectra were recorded on a Bruker spectrometer at 500 MHz or at 125 MHz, respectively and are reported relative to deuterated solvent signals. Data for ¹H NMR spectra are reported as follows: chemical shift (δ ppm), multiplicity (s=singlet, d=doublet, t=triplet, q=quartet, quin=quintet m=multiplet), coupling constants (Hz), and integration. Data for ¹³C NMR spectra are reported in terms of chemical shift.

High resolution mass spectrometry data were acquired on an Agilent 6520 Accurate-Mass Q-TOF LC/MS System, (Agilent Technologies, Inc.) equipped with a dual electro-spray source, operated in the positiveion mode. Separation was performed on Zorbax 300SB-C18 Poroshell column (2.1 mm×150 mm; particle size 5 μm). The analytes were eluted using a water/acetonitrile gradient with 0.1% formic acid. Data were acquired at high resolution (1,700 m/z), 4 GHz. To maintain mass accuracy during the run time, an internal mass calibration sample was infused continuously during the LC/MS runs. Data acquisition and analysis were performed using MassHunter Workstation Data Software, LCMS Data Acquisition (version B.06.01) and Qualitative Analysis (version B.07.00).

Small molecule microarray screening. Small molecule microarrays (SMMs) were prepared on 2D epoxy glass slides (Schott). Briefly, ˜15,000 compounds (10 mM in DMSO) from ChemDiv and Chembridge libraries as well as control dyes (Alexa Fluor 647 and 488, 10 μM in DMSO) were prepared in 384 well plates (Arrayjet Jetstar™). Each of the compounds was printed in duplicate blocks by Arrayjet robotic microarray printer. After printing, glass slides were incubated in the arrayer overnight for immobilization, followed by vacuum drying for 24 h. To quench the unreacted epoxy groups, the slides were incubated in 1 M ethanolamine aqueous solution (pH 8.5) for 2 h, then thoroughly rinsed with DMF and ddH₂O, followed by N₂ drying.

To begin with the screening against the target of interest, 5′Cy5-labeled MYCN G4 DNA sample was first prepared in the annealing buffer (10 mM Tris, pH 7.0, 100 mM KCl) at 10 μM concentration. The DNA sample was incubated in a heating block at 95° C. for 5 min, then slowly cooled down to room temperature for more than 1 h. Then the folded DNA was diluted into the screening buffer (10 mM Tris, pH 7.0, 100 mM KCl, 0.005% Tween 20) to meet the final concentration of 50 nM. A SMM slide was placed into a 4-well slide holder and incubated with 3 mL 10×tRNA (500 nM in screening buffer) for 2 h. Then, the slide was carefully washed by screening buffer and incubated with 3 mL MYCN G4 DNA solution (including 10×tRNA) for another 2 h. After incubation, the slide was transferred into a 50 mL conical tube and gently washed with PBST and ddH₂O for 3 times in each buffer solution. Then, the slide was dried by centrifuging at 1,700 g for 2 minutes. Finally, the slide with SMM was imaged by a fluorescence scanner (InnoScan 1100 AL) at 647 nm, with a resolution of 5 μm. The fluorescent intensity of each spot was quantified by Innopsys Mapix software and the hits were identified based on the criteria reported previously (Connelly et al., ACS Chemical Biology 2017, 12:435-443; Connelly et al., Nature Communications 2019, 10:1501). To identify hits, another slide only incubated with screening buffer was also tested and imaged using the same method as a negative control.

Fluorescence intensity assay. In this disclosure, fluorescence intensity assay (FIA) was used for both validation of hit binding at a single dose and binding affinity determination. For validating hit compounds from SMM screening, 100 μM solution of each compound was prepared in triplicate in a 96 well-plate (Costar, black side clear bottom), resulting in 5% DMSO concentration. 5′Cy5-labeled MYCN G4 DNA was annealed as described above, then added into the well plate resulting in a final concentration of 100 nM. The plate was incubated shaking for 30 min, followed by centrifuging at 500 rpm for 2 minutes. The fluorescence intensity was then measured on a Synergy Mx microplate reader (BioTek) at Ex 649 nm/Em 670 nm. TMPyP4/DMSO were used as positive and negative controls, respectively. In certain examples, compounds were considered binders when a change of >10% change in fluorescence intensity was observed compared to that of negative control (DMSO solution). For binding affinity measurement, small molecule solutions were prepared as serial dilutions in DMSO. Then a final test plate was prepared by adding 5 μL small molecule solutions and 10 μL annealed DNA samples into 85 μL buffer solutions, resulting in the final small molecule concentrations from 0 to 250 μM (5% final DMSO concentration). The fluorescence intensities were normalized, and the binding affinity was calculated by fitting the curve using one-site total model in GraphPad Prism 8.3.1 software. The fluorescence titration using 3′-labeled oligonucleotides was also performed the same way.

Fluorescence displacement assay. Fluorescence displacement assays were carried out by using two classical minor groove binders (Hoechst 33258 and netropsin). Hoechst 33258, as a fluorophore, was prepared at 5 μM concentration in the buffer (10 mM Tris, pH7.0, 100 mM KCl), and then titrated with different concentrations of unlabeled MYCN G4 oligonucleotides. For displacement, either compound MY-1 or netropsin was added into the solution resulting in a final concentration of 5 μM or 50 μM. Fluorescence signals were obtained with an excitation wavelength of 352 nm and an emission wavelength of 500 nm.

Surface plasmon resonance (SPR) analysis. The SPR binding assays were performed with a BIAcore 3000 (GE Healthcare) instrument. A CM5 SPR biochip was loaded into the system and primed with running buffer (10 mM Tris, pH7.0, 100 mM KCl, 0.005% Tween 20, 5% DMSO) with a flow rate of 5 μL/min. Then both Flow Cell (Fc) 1 and 2 were activated by EDC/NHS (0.4 M/0.1 M) aqueous solution for 15 min, followed with an injection of streptavidin (SA) solution (0.2 mg/mL in 10 mM sodium acetate buffer, pH4.5) for 30 minutes. After the immobilization amount of SA reached 8,000-10,000 RU, the surface was deactivated by flowing 1 M Ethanolamine aqueous solution (pH 8.5) for 10 min and regenerated with 10 mM NaOH for 2 min to remove the unbound SA. In the meantime, biotinylated MYCN G4 DNA was prepared at 5 μM in the annealing buffer and heated up to 95° C. for 5 min and slowly cooled down. After annealing, a total of 150 μL solution was injected in the Fc 2 of SPR system for 30 min to immobilize DNA onto the chip surface. The small molecule solutions were tested once the baseline was stable.

To detect the binding signal as well as the binding affinity, a higher flowing rate (25 μL/min) was used in both Fc 1 (reference) and Fc 2 (DNA). Each of the compound solution was prepared at 20× designed the concentrations in DMSO, and then diluted into non-DMSO running buffer, resulting in a final concentration of 5% DMSO. Then, a total of 50 μL compound solution was injected in Fc 1-2 flow path for 120 s for association, followed with 200 s running of buffer for dissociation. An injection of 50 μL regeneration buffer (1 M KCl) could be performed between two samples if necessary. The final binding curve was obtained by reference subtraction. To determine the binding affinity (K_{D}), a series of diluted compound solutions were injected and K_{D} was calculated by BIAevaluation 4.0 software (GE Healthcare) using Langmuir 1:1 binding model.

Circular dichroism (CD) characterization and thermal melting assay. The folded G-quadruplex structure was characterized by circular dichroism using a J-1500 circular dichroism spectrometer (Jasco). Unlabeled MYCN oligonucleotide (5′-AGG GGG TGG GAG GGG GCA TGC AGA TGC AGG GGG T-3′, SEQ ID NO: 7) was prepared in annealing buffer (10 mM Tris, pH7.0, 100 mM KCl) at 5 μM concentration. As a contrast, DNA samples diluted in Li⁺-containing buffer (10 mM Tris, pH7.0, 100 mM LiCl) and H₂O were also used as negative controls. The annealing procedure was the same as mentioned above. CD spectra were recorded from 320 nm to 200 nm at 25° C. with a step of 1 nm. Each spectrum was obtained by averaging the signals of three replicate samples.

For thermal melt assay, MYCN oligonucleotides were prepared in a low KCl buffer (10 mM Tris, pH7.0, 5 mM KCl) at 5 μM concentration. To test the stabilization effect, G4 samples were incubated with and without 20 μM compound (final solution containing 5% DMSO). Then a total of 300 μL solution was added in a cuvette and heated from 20° C. to 95° C. in a CD spectrometer with an interval of 1° C. To calculate the melting temperature (Tm), the peak of CD spectrum at 263 nm was tracked, and ellipsometry vs temperature was plotted and fitted using a nonlinear sigmoidal dose-response model with a variable slope in GraphPad Prism 8 software. The shifting of melting temperature (ΔTₘ) was calculated using Tₘ (compound)−Tₘ (DMSO).

2-Aminopurine (2-AP) fluorescence titration. Fluorescence titrations based on 2-aminopurine labeling were performed using the protocol reported previously. Briefly, MYCN G4 oligonucleotide with a 2-AP substitution at either A11 or A18 position was annealed by heating the sample to 95° C. for 5 min and cooling down to RT slowly. The oligonucleotide with folded structure was diluted to 10 μM in Tris buffer (10 mM Tris, pH7.0, 100 mM KCl, 0.005% Tween 20) and prepared in a black 96-well plate (Costar) in triplicate as designed. Small molecules were diluted in DMSO to obtain a series of solutions with concentrations ranging from 0.1 to 5 mM, and then diluted by 20 times added into the plate (5% DMSO final concentration). The final concentration of 2-AP DNA was 1 μM in the well plate. To obtain the background fluorescence, small molecule solutions without DNA were also prepared in the same plate. After incubation for 30 min at RT, the plate was briefly centrifuged and scanned in Synergy Mx microplate reader (BioTek) with Ex 310 nm/Em 365 nm. Fluorescence signals were calculated by averaging the intensities in triplicate wells after subtracting the references. Finally, the binding signals were normalized and the K_{D} value was determined by fitting the curve using a nonlinear sigmoidal dose-response model with a variable slope in GraphPad Prism 8.3.1 software.

Job plot analysis. To determine the stoichiometry of binding, a continuous variation method was used by changing the faction of the compound in the solution (Renny et al., Angewandte Chemie 2013, 52:11998-12013). Briefly, stock solutions (5% DMSO containing) of 5 μM annealed A11 2-AP labeled MYCN G4 DNA sample and 5 μM compound were prepared, respectively. Two series of solutions were then prepared in 96 black well plates for the experiment: one with a varying fraction of small molecules by mixing the DNA samples with compound stocks, keeping the total concentration constant (5 μM); the other with a varying concentration of DNA samples diluted with buffer, resulting in a track of samples as references. After scanning, the difference between two series of solutions in fluorescence intensity is calculated to generate a Job plot. Then, linear regression analysis was performed using GraphPad Prism 8.3.1 software.

Microscale thermophoresis (MST). MST experiments were carried out on a Monolith NT.115 system (NanoTemper Technologies). The 3′-Cy5 MYCN G4 DNA solutions were prepared in 10 mM Tris (pH 7.5), 100 mM KCl, 0.005% T20, annealed (according to the above-mentioned method), and diluted to 100 nM (2×). The DMSO solutions of small molecules were prepared in the buffer with a series 1:1 dilution, resulting in 2× of designed concentrations (10% DMSO). Then, DNA samples were 1:1 (v/v) mixed with corresponding small molecule solutions, resulting in 50 nM DNA and 5% DMSO. Finally, MST signals were detected in triplicate capillaries, and the dissociation constant was determined by fitting the curve using a single-site model (MO.Affinity Analysis v2.3).

DMS footprinting. Footprinting of 5′Cy5-labelled DNA was performed as described in the literature (Zhang et al., JACS 2014, 136:1381-1390; Li et al., PNAS 2015, 112:14581-14586). MYCN G4 DNA samples were prepared in appropriate buffer (10 mM Tris, pH7.0, 5 mM KCl, 0.005% T20) with 5 μM concentration, followed by annealing using above-mentioned method. Then, DNA solutions were diluted to 50 nM in corresponding buffers (2 mL final volume in each tube) and compound stock solutions (in DMSO) were added resulting in solutions with designed concentrations (resulting in 5% DMSO). After incubation for 30 min, the folded DNA samples were treated with 0.5% DMS for 10 min at RT and the reaction was stopped by adding 200 μL stop buffer (2.5 M NH₄OAc, 0.1 M β-mercaptoethanol, 1 mg/mL calf thymus DNA). After phenol/chloroform/isoamyl alcohol extraction and ethanol precipitation, the DNAs were dissolved in 50 μL of nuclease free water. An equal volume of 10% piperidine was added to each tube, and the mixed solutions were heated at 90° C. for 30 min, followed by quick chilling on ice. The DNAs were again subjected to phenol/chloroform/isoamyl alcohol extraction and ethanol precipitation. The precipitated DNAs were dissolved in 10 μL of nuclease free water, denatured at 95° C. for 5 min, and resolved on a 17% denaturing polyacrylamide gel. After gel running, Cy5-labeled DNA fragments were visualized on a Typhoon Imager (Amersham) and digitized using Image J software.

Cell lines and cell culture. The neuroblastoma cell line NBEB (Single Copy MYCN) cells were used to evaluate the efficacy of the MYCN/MYCNOs G-quadruplex binding molecules. NBEB came from stocks in Pediatric Oncology Branch, National Cancer Institute, National Institutes of Health, Bethesda, MD and have been STS verified. Cells were cultured in RPMI-1640, supplemented with 10% FBS (Atlanta Biologicals, Atlanta), 2 mM glutamine (Life Technologies, New York), and antibiotics (penicillin 100 μg/mL, streptomycin 100 μg/mL; Life Technologies, New York) at 37° C. in a 5% CO₂ incubator.

To examine the effects of G4-binding compounds on NBEB cells, live-cell imaging was performed using an Incucyte interface (described below). NBEB (5K/well), cells were seeded into 96-well plates in triplicate, treated with compound MY-8 at various concentrations (0-45 μM), and cultured until control wells reached confluence. For RT-qPCR and western blotting, NBEB (300K) cells were treated with MY-8 in 6-well dish for 48 h. To examine the time-dependent decrease in MYCNOs expression, cells were treated with 22.5 μM and 45 μM MY-8 and analyzed at various times (0, 4, 24, 48, 72 and 96 h). To validate cell viability, a Trypan Blue exclusion test was performed following treatment at 48 h and 96 h post treatment and the proportion of live versus dead cells was calculated using a hemocytometer.

Cell survival. Following live cell image analysis, a 3-(4, 5-dimethylthiazol-2-yl)-5-(3-carboxymethoxyphenyl)-2-(4-sulfophenyl)-2H-tetrazolium inner salt assay was performed to examine cell survival. (MTS; inner salt assay; Promega, America). Absorbance was measured with a microplate reader (BIO-RAD, America) at 490 nm wavelength. To calculate the proportion of viable cells, MY-8 treated cells were divided by the control samples from each group.

Incucyte live cell imaging system. Live cell imaging was performed using the Incucyte Zoom Live-cell Imaging System from Essen Bioscience (Ann Arbor, MI, USA). Incucyte measured cell confluence from pre-defined processing definitions for NBEB NB cells. The Incucyte Zoom Live-cell Imaging System scanned three phase contrast images per well every 6 h for the duration of treatment (0-120 h).

Quantitative RT-PCR. Total RNA was extracted from NBEB cells according to manufacturer's instructions (RNeasy Plus Mini Extraction kit (#74134) (Qiagen, CA). RNA (1 μg) was reverse transcribed into cDNA using the cDNA synthesis High-Capacity RNA-to-cDNA™ Kit. Quantitative PCR was performed using Fast SYBR™ Green Master Mix according to the manufacturer's protocol. Beta-actin was used as a housekeeping gene. Relative expression was calculated using the 2(−ΔCₜ) method. The qPCR primer sequences can be found in supplementary materials.

Protein assay. Forty-eight hours post-MY-8 treatment in 6-well plates, single copy MYCN cells were lysed directly in the plate with Radioimmunoprecipitation assay buffer (Beyotime, China), pelleted, and chilled at −80° C. Attributed to poor cellular adhesion, NBEB cells were collected into 1.5 mL microcentrifuge tubes, washed with Phosphate Buffer Solution (Sigma-Aldrich, USA) twice, pelleted, and chilled at −80° C. Total protein concentrations were determined using a Bradford Assay using the Bradford reagent (Beyotime, China). Each protein sample (10 μg) was loaded onto a 12% gel (Bio-Rad, USA), electrophoresed at 90 V for 90 min and transferred to a nitrocellulose membrane (Immobilon-P, Millipore, Bedford, MA, USA) using a Bio Rad Trans-Blot Turbo Transfer System (Bio-Rad, USA). Nitrocellulose membranes were incubated in 5% milk in Tris-buffered saline supplemented with 0.5% Tween 20 for 1 h at room temperature and incubated with anti-N-Myc: sc-53993 (1:4000) and anti-GAPDH: sc-47724 (1:2000) antibodies overnight at 4° C. Following incubation, the membranes were washed with Tris-buffered saline supplemented with 0.5% Tween 20 and probed with a goat anti-mouse (1:1000) antibody conjugated to peroxidase and incubated for 1 h at room temperature. Chemiluminescent signal was detected using Clarity™ Western ECL Substrate (Bio-Rad, USA) or Supersignal™ West Femto Maximum Sensitivity Substrate (ThermoFisher Scientific, USA) with a ChemiDoc gel imaging system (Bio-Rad, USA).

Bioinformatics study. Two files were downloaded from the Gene Expression Omnibus (GSE63874) pertaining to the experimental G4 data (files named “Na_K_PDS_minus_hits_intersect.bed.gz” and “Na_K_PDS_plus_hits_intersect.bed.gz”) (1). The genomic sequences of the bed files were extracted from GRCh37 (hg19) reference genome using bedtools getfasta (bedtools 2.30.0) (2). In the case of the “Na_K_PDS_plus_hits_intersect.bed” locations, the complementary strand sequence was extracted due to the G4 sequences existing on the negative strand of the genome assembly. G4-forming sequences were identified from the extracted unique OQs using the regular expression formula: ((G₃₋₆(N13₃)₃G₃-6).

The potential for hairpin-forming loops within the G4 sequences was then calculated. UNAFold 4.0 was used to calculate hairpin formation from sequences flanked by two G-tracts of length 3 nt in the G4s. UNAFold was run using “hybrid-ss-min—NA DNA” setting to output probabilities of each nt position forming a single strand at 37° C. under DNA energy rules (3). Sequences with more than 50% of their nucleotides base paired were identified as hairpin-forming sequences. Then, the hairpin-forming G4s were identified and traced back to the OQs they were extracted from. Coordinates of the G4-hairpin-containing OQs were then converted to GRCh38 (hg38) coordinates using the UCSC Lift Genome Annotations tool (liftOver) (4).

The resulting hg38 coordinates were annotated with the GENCODE, hg38 version 36 comprehensive gene annotation GFF3 file (5). Promoters were also added to the annotation defined as regions 1000 nt upstream of TSS sites of GENCODE genes. Introns were also added to the GENCODE annotation using the Genome Tools package (genometools 1.6.1) “gt gff3-addintrons” command (6). Overlapping genomic features with GQ-hairpin-containing OQs were identified using bedtools intersect command. Python 3.8 was used for computational operations not performed by the mentioned software packages.

qPCR experiments. The primer sequences for qPCR experiments are detailed as follows:

MYCNOs-01 (ENST00000641263.1 - 1,313, bps)
Sense

SEQ. ID NO 10

5′ - AGGCTCAGTCTCCCTCACTA 3′,
Antisense

SEQ ID NO: 11

5′ TTCTGGAGGCTGAGAAGTCC3′,
MYCNOs-02 (ENST00000419083.5 - 770 bps)
Sense

SEQ ID NO: 12

5′ CTCACGAGCACGCAGACAAC 3′,
Antisense

SEQ ID NO: 13

5′ TCCCAGCTTTGCAGCCTTCT 3′,
MYCN Protein Isoform 1 (ENST00000281043.4 -
464 AA)
Sense

SEQ ID NO: 14

5′ GATCTGCAAGAACCCAGACC 3′,
Antisense

SEQ ID NO: 15

5′ CACAGCTCGTTCTCAAGCAG 3′,
MYCN Protein Isoform 2 (ENST00000638417.1 -
253 AA)
Sense

SEQ ID NO: 16

5′ TCCTGGGAACTGTGTTGGA 3′,
Antisense

SEQ ID NO: 17

5′ CACAGTGACCACGTCGATTT 3′,

### Compound Synthesis—Monovalent Compounds

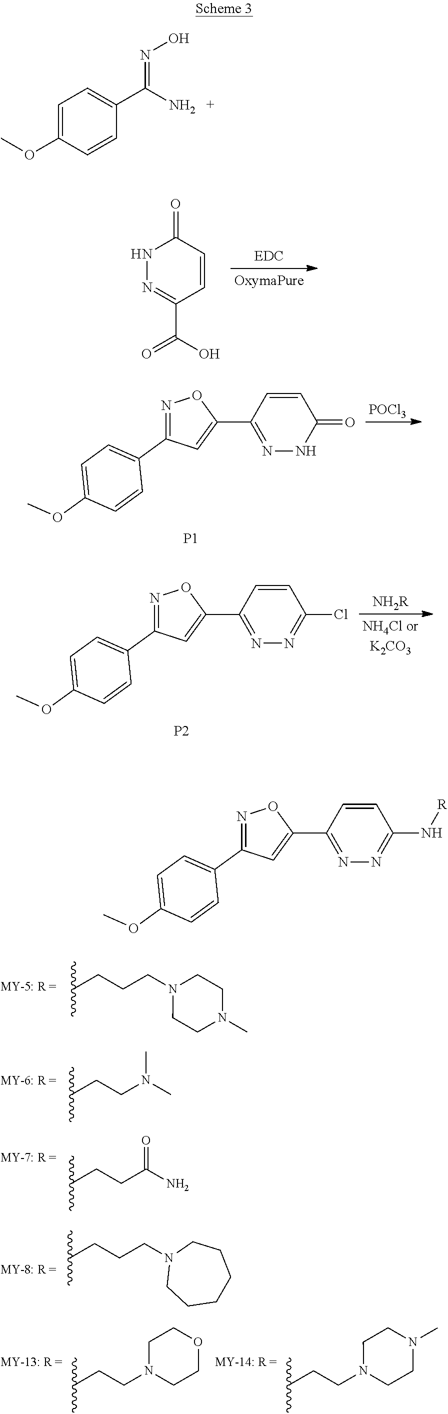

To a reaction flask was added 6-hydroxypyridazine-3-carboxylic acid (392 mg, 2.80 mmol), EDC·HCl (589 mg, 3.08 mmol), Oxyma Pure (438 mg, 3.08 mmol) and DMF (8 mL). The reaction was stirred at room temperature for 10 min, followed by the addition of 4-methoxybenzamidoxime (605 mg, 3.64 mmol). The reaction mixture was then heated to 120° C. and stirred. After 3 h, the reaction mixture was filtered and washed with DMF (3 mL×3). The filtrate was collected and dried to yield the target product P1 (344 mg, 46%). ¹H NMR (500 MHz, DMSO-d₆): δ 13.85 (s, 1H), 8.08 (d, J=9.9 Hz, 1H), 8.04-8.01 (m, 2H), 7.17-7.14 (m, 1H), 7.12 (d, J=9.9 Hz, 1H), 3.85 (s, 3H); ¹³C NMR (125 MHz, DMSO-d₆): δ 171.2, 167.9, 162.0, 160.3, 132.4, 131.9, 130.4, 128.9, 118.0, 114.8, 55.5; HRMS: (ESI+) m/z calculated for C₁₃H₁₁N₄O₃ [M+H]⁺: 271.0826, found: 271.0833.

A solution of P1 (970 mg, 3.59 mmol) in POCl₃ (6 mL) was heated to 100° C. and stirred for 4 h. The reaction crude was poured on ice-cold water and neutralized with 10% NaOH solution. The mixture was then extracted with EtOAc (10 mL×3) and washed with brine. The combined organic layers were dried over MgSO₄, filtered and concentrated in vacuo. The resulting residue was purified by ISCO flash column chromatography (0-25% MeOH in CH₂Cl₂) to yield P2 (246 mg, 72%) as a light pink solid. ¹H NMR (500 MHz, DMSO-d₆) δ 8.57 (d, J=8.9 Hz, 1H), 8.26 (d, J=8.9 Hz, 1H), 8.09-8.06 (m, 2H), 7.19-7.16 (m, 2H), 3.87 (s, 3H); ¹³C NMR (125 MHz, DMSO-d₆) δ 172.0, 168.3, 162.1, 158.4, 147.1, 130.4, 130.2, 129.0, 117.8, 114.9, 55.5; HRMS: (ESI+) m/z calculated for C₁₃H₁₀³⁵ClN₄O₂[M+H]⁺: 289.0487, found: 289.0493.

A mixture of P2 (20 mg, 0.07 mmol), 3-(4-methylpiperazin-1-yl)propan-1-amine (22 mg, 0.14 mmol) and NH₄Cl (4 mg, 0.07 mmol) in EtOH (3 mL) was heated to 80° C. and stirred overnight. The reaction mixture was concentrated in vacuo and the resulting residue was purified by Isco flash column chromatography (0-25% MeOH in CH₂Cl₂) to afford MY-5 (13 mg, 45%) as a white solid. ¹H NMR (500 MHz, DMSO-d₆) δ 8.04-8.01 (m, 2H), 7.98 (d, J=9.3 Hz, 1H), 7.83 (s, 1H), 7.16-7.13 (m, 2H), 6.98 (d, J=9.3 Hz, 1H), 3.85 (s, 3H), 3.50-3.43 (m, 2H), 2.56-2.25 (m, 10H), 2.19 (s, 3H), 1.77 (quin, J=7.0 Hz, 2H); ¹³C NMR (125 MHz, DMSO-d₆) δ 173.4, 167.7, 161.8, 159.4, 138.3, 128.8, 127.4, 118.4, 114.7, 55.5, 55.3, 54.6, 52.4, 45.5, 25.7; HRMS: (ESI+) m/z calculated for C₂₁H₂₈N₇O₂[M+H]⁺: 410.2299, found: 410.2305.

A mixture of P2 (30 mg, 0.10 mmol), N,N′-dimethylethylenediamine (18 mg, 0.20 mmol) and NH₄Cl (5 mg, 0.10 mmol) in EtOH (3 mL) was heated to 80° C. and stirred overnight. The reaction mixture was concentrated in vacuo and the resulting residue was purified by Isco flash column chromatography (0-25% MeOH in CH₂Cl₂) to afford MY-6 (15 mg, 44%) as a white solid. ¹H NMR (500 MHz, DMSO-d₆) δ 8.17-8.14 (m, 1H), 8.05-8.03 (m, 2H), 7.35-7.32 (m, 1H), 7.17-7.14 (m, 2H), 4.08 (t, J=6.4 Hz, 2H), 3.86 (s, 3H), 3.24-3.20 (m, 5H), 2.59-2.58 (m, 3H); ¹³C NMR (125 MHz, DMSO-d₆) δ 173.3, 167.8, 161.9, 159.6, 138.2, 128.8, 127.8, 118.3, 114.7, 111.8, 55.5, 46.1, 46.0, 36.5, 33.0; HRMS: (ESI+) m/z calculated for C₁₇H₂₁N₆O₂ [M+H]⁺: 341.1721, found: 341.1725.

A mixture of P2 (20 mg, 0.07 mmol), 3-aminopropanamide hydrochloride (17 mg, 0.14 mmol) and K₂CO₃ (37 mg, 0.28 mmol) in EtOH (3 mL) was heated to 80° C. and stirred overnight. The reaction mixture was concentrated in vacuo and the resulting residue was purified by Isco flash column chromatography (0-25% MeOH in CH₂Cl₂) to afford MY-7 (12 mg, 50%) as a white solid. ¹H NMR (500 MHz, DMSO-d₆) δ 8.04-8.01 (m, 2H), 7.99 (d, J=9.6 Hz, 1H), 7.84 (t, J=5.9 Hz, 1H), 7.39 (s, 1H), 7.16-7.13 (m, 2H), 7.01 (d, J=9.6 Hz, 1H), 6.88 (s, 1H), 3.85 (s, 3H), 3.68-3.66 (m, 2H), 2.45 (t, J=6.7 Hz, 2H); ¹³C NMR (125 MHz, DMSO-d₆) δ 173.4, 172.5, 167.7, 161.8, 159.2, 138.4, 128.8, 127.4, 118.4, 114.7, 55.4, 37.3, 34.3; HRMS: (ESI+) m/z calculated for C₁₆H₁₇N₆O₃ [M+H]⁺: 341.1357, found: 341.1359.

A mixture of 2 (32.91 mg, 0.11 mmol), 3-(1-azepanyl)-1-propanamine (36 mg, 0.23 mmol) and NH₄Cl (6 mg, 0.11 mmol) in EtOH (3 mL) was heated to 80° C. and stirred overnight. The reaction mixture was concentrated in vacuo and the resulting residue was purified by Isco flash column chromatography (0-25% MeOH in CH₂Cl₂) to afford MY-8 (31 mg, 66%) as a white solid. ¹H NMR (500 MHz, DMSO-d₆) δ 8.04-7.99 (m, 4H), 7.16-7.14 (m, 2H), 7.04 (d, J=9.5 Hz, 1H), 3.85 (s, 3H), 3.55-3.54 (m, 2H), 3.15-3.07 (m, 6H), 2.08-2.00 (m, 2H), 1.83-1.77 (m, 4H), 1.67-1.57 (m, 4H); ¹³C NMR (125 MHz, DMSO-d₆) δ 173.3, 167.8, 161.8, 159.5, 138.6, 128.8, 127.5, 118.4, 114.7, 55.5, 54.4, 53.8, 38.3, 26.0, 23.7, 23.3; HRMS: (ESI+) m/z calculated for C₂₂H₂₉N₆O₂ [M+H]⁺: 409.2347, found: 409.2354. MY-8 (5 mg) was then stirred in TFA (1 mL) for 30 min to yield the corresponding TFA salt.

A mixture of 2 (30 mg, 0.10 mmol), 2-morpholinoethan-1-amine (14 mg, 0.20 mmol) and NH₄Cl (5 mg, 0.10 mmol) in EtOH (3 mL) was heated to 80° C. and stirred overnight. The reaction mixture was concentrated in vacuo and the resulting residue was purified by Isco flash column chromatography (0-25% MeOH in CH₂C₂) to afford MY-13 (22.5 mg, 59%) as a white solid. ¹H NMR (500 MHz, DMSO-d₆) δ 8.05-8.02 (m, 2H), 7.99 (d, J=9.3 Hz, 1H), 7.70 (t, J=5.5 Hz, 1H), 7.17-7.14 (m, 2H), 7.04 (d, J=9.4 Hz, 1H), 3.85 (s, 3H), 3.61-3.58 (m, 6H), 2.56 (t, J=6.6 Hz, 2H), 2.46-2.44 (m, 4H); ¹³C NMR (125 MHz, DMSO-d₆) δ 173.4, 167.7, 161.8, 138.4, 128.8, 127.4, 118.4, 114.7, 66.2, 56.9, 55.4, 53.4, 38.0; HRMS: (ESI+) m/z calculated for C₁₉H₂₃N₆O₃ [M+H]⁺: 383.1826, found: 383.1831.

A mixture of 2 (20 mg, 0.07 mmol), 2-(4-methylpiperazin-1-yl)ethan-1-amine (20 mg, 0.14 mmol) and NH₄Cl (4 mg, 0.07 mmol) in EtOH (3 mL) was heated to 80° C. and stirred overnight. The reaction mixture was concentrated in vacuo and the resulting residue was purified by Isco flash column chromatography (0-25% MeOH in CH₂C₂) to afford MY-14 (16 mg, 58%) as a white solid. ¹H NMR (500 MHz, DMSO-d₆) δ 8.04-8.02 (m, 2H), 7.99 (d, J=9.5 Hz, 1H), 7.68 (t, J=5.3 Hz, 1H), 7.16-7.14 (m, 2H), 7.03 (d, J=9.5 Hz, 1H), 3.85 (s, 3H), 3.62-3.56 (m, 2H), 2.56 (t, J=6.7 Hz, 2H), 2.46-2.26 (m, 8H), 2.17 (s, 3H); ¹³C NMR (125 MHz, DMSO-d₆) δ 173.4, 167.7, 161.8, 138.4, 128.8, 127.4, 118.4, 114.7, 56.4, 55.4, 54.6, 52.6, 38.3; HRMS: (ESI+) m/z calculated for C₂₀H₂₆N₇O₂ [M+H]⁺: 396.2142, found: 396.2141.

### Compound Synthesis—Bivalent Compounds

Bivalent compounds disclosed herein were made according to Scheme 5 below.

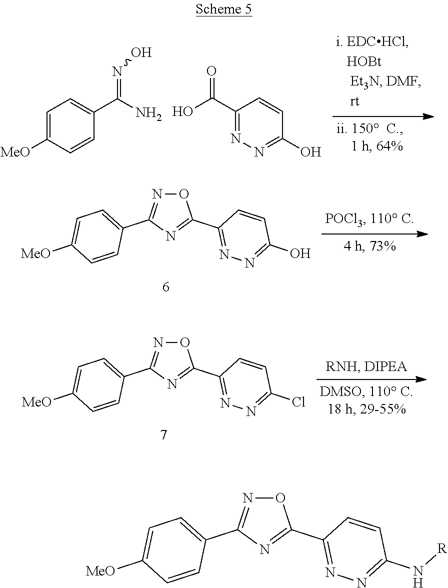

The R¹ groups for the bivalent compounds (referred to as “R” in Scheme 5 above), were made as indicated below in Scheme 6 and then combined with compound 7 in Scheme 5 using the conditions also described below.

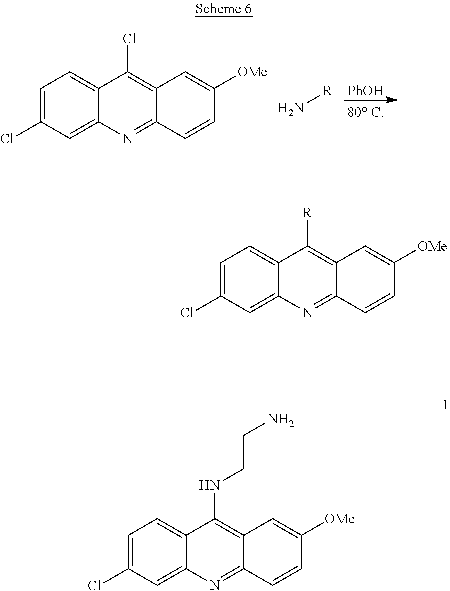

### N¹-(6-chloro-2-methoxyacridin-9-yl)ethane-1,2-diamine (1)

Combine 6,9-dichloro-2-methoxycridine (99.2 mg, 0.357 mmol, 1 equiv), phenol (33.6 mg, 0.357 mmol, 1 equiv) and 1,2-diaminoethane (214 mg, 3.57 mmol, 10 equiv) in a sealed vial and heat at 110° C. for 8 h in the dark. Upon completion by LCMS, the reaction was allowed to cool to room temperature and concentrated onto celite. The reaction mixture was purified by reverse phase flash chromatography (0-100% acetonitrile/water with 0.1% TFA as a modifier) to afford 66.3 mg (62%) of 1 as a yellow solid: ¹H NMR (500 MHz, CD₃OD) δ 8.47 (dd, J=9.5, 2.6 Hz, 1H), 7.86 (q, J=2.9, 2.4 Hz, 2H), 7.81 (dd, J=9.3, 2.0 Hz, 1H), 7.74-7.67 (m, 1H), 7.54 (dt, J=9.3, 2.1 Hz, 1H), 4.52 (t, J=6.2 Hz, 2H), 4.03 (d, J=2.0 Hz, 3H), 3.58 (t, J=6.1 Hz, 2H); ¹³C NMR (126 MHz, CD₃OD) δ 158.72, 158.55, 142.20, 141.78, 136.27, 129.37, 129.19, 125.48, 121.60, 118.79, 116.08, 111.61, 103.92, 56.75, 47.44, 39.78; LRMS calculated for C₁₆H₁₆ClN₃O⁺ [M+H]+m/z 301.09, measured LC/MS (ESI)R, 1.18 min, m/z 302.20 [M+H]⁺.

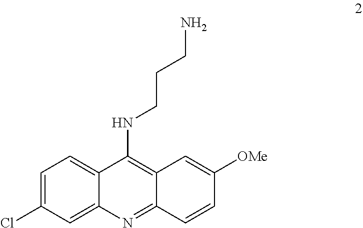 [SMILES: COc1ccc2nc3cc(Cl)ccc3c(NCCCN)c2c1]

### N¹-(6-chloro-2-methoxyacridin-9-yl)propane-1,3-diamine (2)

Combine 6,9-dichloro-2-methoxycridine (200 mg, 0.719 mmol, 1 equiv), phenol (67.7 mg, 0.719 mmol, 1 equiv) and 1,3-diaminopropane (533 mg, 7.19 mmol. 10 equiv) in a sealed vial and heat at 110° C. for 8 h in the dark. Upon completion by LCMS, the reaction was allowed to cool to room temperature and concentrated onto celite. The reaction mixture was purified by reverse phase flash chromatography (0-100% acetonitrile/water with 0.1% TFA as a modifier) to afford 125 mg (57%) of 2 as a yellow solid: ¹H NMR (500 MHz, CD₃OD) δ 8.47 (dd, J=9.5, 3.0 Hz, 1H), 7.82 (dd, J=9.8, 2.4 Hz, 2H), 7.77 (dd, J=9.1, 3.1 Hz, 1H), 7.66 (dd, J=9.2, 2.6 Hz, 1H), 7.50 (dt, J=9.4, 2.2 Hz, 1H), 4.27 (t, J=7.0 Hz, 2H), 4.00 (d, J=4.2 Hz, 2H), 3.13 (t, J=7.6 Hz, 2H), 2.38-2.31 (m, 2H); ¹³C NMR (125 MHz, CD₃OD) δ 158.47, 158.33, 142.05, 141.73, 136.08, 129.34, 128.99, 125.29, 121.59, 118.67, 115.88, 111.59, 103.86, 56.71, 47.30, 38.11, 28.66; LRMS calculated for C₁₇H₁₈ClN₃O⁺ [M+H]+m/z 315.11, measured LC/MS (ESI)R, 1.17 min, m/z 316.20 [M+H]⁺.

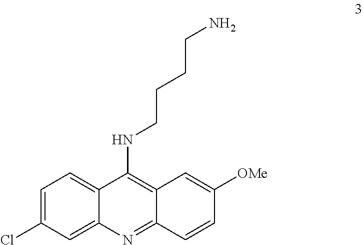 [SMILES: COc1ccc2nc3cc(Cl)ccc3c(NCCCCN)c2c1]

### N¹-(6-chloro-2-methoxyacridin-9-yl)butane-1,4-diamine (3)

Combine 6,9-dichloro-2-methoxycridine (250 mg, 0.899 mmol, 1 equiv), phenol (84.6 mg, 0.889 mmol, 1 equiv) and 1,4-diaminobutane (792 mg, 8.99 mmol. 10 equiv) in a sealed vial and heat at 110° C. for 8 h in the dark. Upon completion by LCMS, the reaction was allowed to cool to room temperature and concentrated onto celite. The reaction mixture was purified by reverse phase flash chromatography (0-100% acetonitrile/water with 0.1% TFA as a modifier) to afford 424 mg (85%) of 3 as a yellow-orange solid: ¹H NMR (500 MHz, CD3OD) δ 8.20 (dd, J=9.4, 2.5 Hz, 1H), 7.80 (t, J=2.0 Hz, 1H), 7.78-7.75 (m, 1H), 7.46 (d, J=2.7 Hz, 1H), 7.39 (dt, J=9.3, 2.2 Hz, 1H), 7.25 (dt, J=9.3, 2.1 Hz, 1H), 3.95 (s, 3H), 3.83 (t, J=7.1 Hz, 2H), 2.83 (dd, J=8.9, 6.0 Hz, 2H), 1.83 (p, J=8.4, 7.6 Hz, 2H), 1.70-1.62 (m, 2H); LRMS calculated for C₁₈H₂₀ClN₃O⁺[M+H]+m/z 329.13, measured LC/MS (ESI)R, 1.56 min, m/z 330.25 [M+H]⁺.

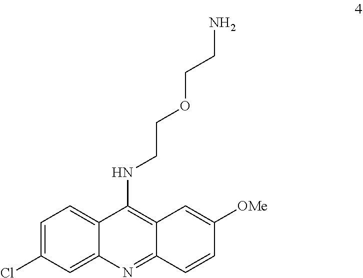 [SMILES: COc1ccc2nc3cc(Cl)ccc3c(NCCOCCN)c2c1]

### N-(2-(2-aminoethoxy)ethyl)-6-chloro-2-methoxyacridin-9-amine (4)

Combine 6,9-dichloro-2-methoxycridine (250 mg, 0.899 mmol, 1 equiv), phenol (84.6 mg, 0.889 mmol, 1 equiv) and 2,2′-oxybis(ethan-1-amine) (936 mg, 8.99 mmol. 10 equiv) in a sealed vial and heat at 110° C. for 8 h in the dark. Upon completion by LCMS, the reaction was allowed to cool to room temperature and concentrated onto celite. The reaction mixture was purified by reverse-phase flash chromatography (0-100% acetonitrile/water with 0.1% TFA as a modifier) to afford 441 mg (86%) of 4 as a yellow solid: ¹H NMR (500 MHz, CD₃OD) δ 8.15 (d, J=9.4 Hz, 1H), 7.80 (s, 1H), 7.78 (d, J=9.3 Hz, 1H), 7.38 (s, 2H), 7.33 (d, J=9.0 Hz, 1H), 7.21 (d, J=9.3 Hz, 1H), 3.91 (s, 3H), 3.85 (t, J=5.1 Hz, 2H), 3.63 (m, 2H), 3.43 (m, 2H), 2.70 (m, 2H); ¹³C NMR (126 MHz, CD₃OD) δ 155.89, 151.58, 147.51, 145.60, 134.99, 129.02, 125.70, 125.37, 124.81, 123.31, 117.87, 115.40, 99.69, 71.75, 70.15, 54.76, 49.38, 40.63; LRMS calculated for C₁₈H₂₀ClN₃O₂⁺ [M+H]+m/z 345.12, measured LC/MS (ESI) Rₜ 1.74 min, m/z 346.25 [M+H]⁺.

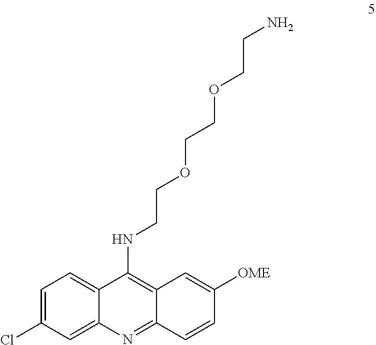

### N-(2-(2-(2-aminoethoxy)ethoxy)ethyl)-6-chloro-2-methoxyacridin-9-amine (5)

Combine 6,9-dichloro-2-methoxycridine (250 mg, 0.899 mmol, 1 equiv), phenol (84.6 mg, 0.889 mmol, 1 equiv) 2,2′-(ethane-1,2-diylbis(oxy))bis(ethan-1-amine) (1.33 g, 8.99 mmol. 10 equiv) in a sealed vial and heat at 110° C. for 8 h in the dark. Upon completion by LCMS, the reaction was allowed to cool to room temperature and concentrated onto celite. The reaction mixture was purified by reverse phase flash chromatography (0-100% acetonitrile/water with 10 mM NH₄OH as a modifier) to afford 200 mg (57%) of 5 as a yellow solid: ¹H NMR (500 MHz, DMSO-D6) δ 8.37 (d, J=9.2 Hz, 1H), 7.86 (s, 1H), 7.63 (d, J=2.7 Hz, 1H), 7.41 (d, J=9.3 Hz, 1H), 7.34 (d, J=10.5 Hz, 1H), 6.78-6.55 (m, 1H), 3.93 (s, 3H), 3.86 (d, J=7.9 Hz, 2H), 3.68 (t, J=5.6 Hz, 2H), 3.49 (dd, J=5.7, 3.9 Hz, 2H), 3.40 (dd, J=5.9, 3.9 Hz, 2H), 3.25 (t, J=5.8 Hz, 2H), 2.56 (t, J=5.8 Hz, 2H); ¹³C NMR (126 MHz, DMSO-D6) δ 155.15, 150.62, 147.98, 146.23, 133.44, 130.81, 127.23, 126.34, 126.18, 117.69, 115.38, 100.54, 73.06, 69.79, 69.50, 55.59, 41.28.

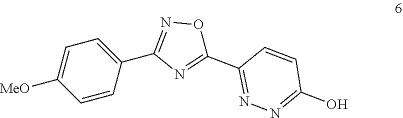 [SMILES: COc1ccc(-c2noc(-c3ccc(O)nn3)n2)cc1]

### 6-(3-(4-methoxyphenyl)-1,2,4-oxadiazol-5-yl)pyridazin-3-ol (6)

To a solution of 6-hydroxypyridazine-3-carboxylic acid (1.50 g, 11 mmol, 1 equiv) in DMF (48 mL) was added triethylamine (3.0 mL, 21 mmol, 2 equiv), HOBt (1.60 g, 11 mmol, 1 equiv) and EDC·HCl (2.40 g, 11 mmol, 1 equiv). The solution was allowed to stir at room temperature for 30 minutes before the addition of N′-hydroxy-4-methoxybenzimidamide (1.80 g, 11 mmol, 1 equiv). The reaction was maintained at room temperature for 1 hour, then heated and maintained at 150° C. for 1 h or until judged complete by TLC. The solution was allowed to cool to room temperature, at which point the product precipitated from the solution. Water (˜300 mL) was added, and the solid was triturated at room temperature for 30 minutes. The product was isolated by vacuum filtration and dried in vacuo to afford 1.84 g of 6 (64%) as a white solid: ¹H NMR (500 MHz, DMSO-D6) δ 13.85 (s, 1H, exchanges with D₂O), 8.08 (d, J=9.9 Hz, 1H), 8.02 (d, J=8.9 Hz, 2H), 7.15 (d, J=8.9 Hz, 2H), 7.12 (d, J=9.9 Hz, 1H), 3.85 (s, 3H); ¹³C NMR (125 MHz, DMSO-D6) δ 171.2, 167.9, 162.0, 160.3, 132.3, 131.8, 130.4, 128.9, 117.9, 114.8, 55.4. NMR data is consistent with literature values.^{[1]}

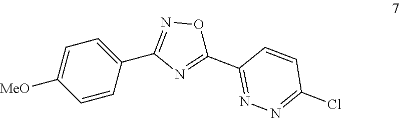 [SMILES: COc1ccc(-c2noc(-c3ccc(Cl)nn3)n2)cc1]

### 5-(6-chloropyridazin-3-yl)-3-(4-methoxyphenyl)-1,2,4-oxadiazole (7)

A vial of 1 (500 mg, 1.85 mmol, 1 equiv) and POCl₃ (3.14 mL, 0.59 M) was sealed and heated to 100° C. for 4 hours. Once judged complete by TLC, the reaction mixture was allowed to cool to room temperature and concentrated in vacuo. The crude mixture was purified by flash column chromatography (0-100% EtOAc/hexanes) to afford 388 mg (72%) of 7 as a tan solid: ¹H NMR (500 MHz, DMSO-D6) δ ¹H NMR (500 MHz, DMSO-D6) δ 8.56 (d, J=8.9 Hz, 1H), 8.26 (d, J=9.0 Hz, 1H), 8.07 (d, J=8.8 Hz, 2H), 7.17 (d, J=8.8 Hz, 2H), 3.86 (s, 3H); ¹³C NMR (125 MHz, DMSO-D6) δ 172.0, 168.3, 162.1, 158.4, 147.0, 130.3, 130.2, 129.0, 117.8, 114.8, 55.5.

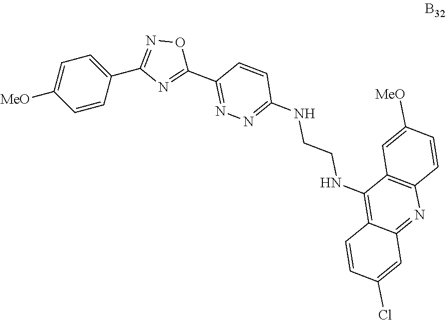 [SMILES: COc1ccc(-c2noc(-c3ccc(NCCNc4c5ccc(Cl)cc5nc5ccc(OC)cc45)nn3)n2)cc1]

### N1-(6-chloro-2-methoxyacridin-9-yl)-N2-(6-(3-(4-methoxyphenyl)-1,2,4-oxadiazol-5-yl)pyridazin-3-yl)ethane-1,2-diamine (B32)

To a solution of 7 (21.3 mg, 0.07 mmol, 1 equiv) in DMSO (1 mL) was added DIPEA (64.3 μL, 0.370 mmol, 5 equiv) followed by 2 (44.4 mg, 0.148 mmol, 2 equiv). The reaction was heated and maintained at 110° C. for 18 hours. The crude reaction mixture was allowed to cool to room temperature and purified by reverse phase flash chromatography (0-100% acetonitrile/water with 0.1% TFA as a modifier) to afford 3.26 mg (7%) of B32 as yellow solid: ¹H NMR (500 MHz, DMSO-D6) δ 8.68 (d, J=9.3 Hz, 1H), 8.04 (d, J=8.9 Hz, 2H), 7.96 (d, J=9.3 Hz, 1H), 7.87 (d, J=2.1 Hz, 1H), 7.86-7.73 (m, 2H), 7.67 (dd, J=9.2, 2.5 Hz, 1H), 7.50 (dd, J=9.2, 2.2 Hz, 1H), 7.16 (d, J=8.9 Hz, 2H), 6.91 (d, J=9.4 Hz, 1H), 4.42 (d, J=5.9 Hz, 2H), 4.00 (s, 3H), 3.86 (s, 3H), 3.75 (m, 2H).

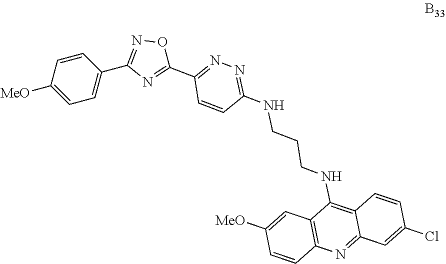 [SMILES: COc1ccc(-c2noc(-c3ccc(NCCCNc4c5ccc(Cl)cc5nc5ccc(OC)cc45)nn3)n2)cc1]

### N¹-(6-chloro-2-methoxyacridin-9-yl)-N³-(6-(3-(4-methoxyphenyl)-1,2,4-oxadiazol-5-yl)pyridazin-3-yl)propane-1,3-diamine (B33)

To a solution of 7 (30 mg, 0.1 mmol, 1 equiv) in DMSO (1 mL) was added DIPEA (91 μL, 0.52 mmol, 5 equiv) followed by 2 (65 mg, 21 μmol, 2 equiv). The reaction was heated and maintained at 110° C. for 18 hours. The crude reaction mixture was allowed to cool to room temperature and purified by reverse phase flash chromatography (0-100% acetonitrile/water with 0.1% TFA as a modifier) to afford 17 mg (29%) of B33 as yellow solid: ¹H NMR (500 MHz, CD₃OD) δ 8.49 (d, J=9.2 Hz, 1H), 8.11 (dd, J=8.9, 2.1 Hz, 2H), 7.88 (d, J=9.4 Hz, 1H), 7.78 (d, J=2.6 Hz, 1H), 7.73 (d, J=2.2 Hz, 1H), 7.70 (d, J=9.4 Hz, 1H), 7.58 (dd, J=9.3, 2.5 Hz, 1H), 7.44 (dd, J=9.3, 2.3 Hz, 1H), 7.10 (d, J=8.8 Hz, 2H), 6.81 (d, J=9.4 Hz, 1H), 4.26 (t, J=6.2 Hz, 2H), 4.02 (s, 3H), 3.90 (s, 3H), 3.75 (t, J=6.4 Hz, 2H), 2.31 (t, J=6.4 Hz, 2H); LRMS calculated for C₃₀H₂₆ClN₇O₃⁺ [M+H]+ m/z 567.18, measured LC/MS (ESI) Rₜ 4.14 min, m/z 568.35 [M+H]⁺.

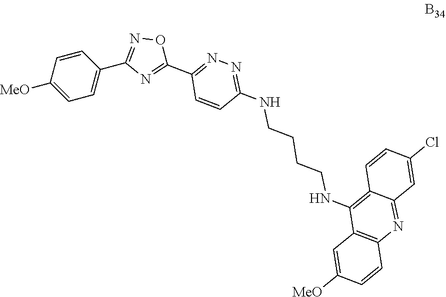 [SMILES: COc1ccc(-c2noc(-c3ccc(NCCCCNc4c5ccc(Cl)cc5nc5ccc(OC)cc45)nn3)n2)cc1]

### N¹-(6-chloro-2-methoxyacridin-9-yl)-N⁴-(6-(3-(4-methoxyphenyl)-1,2,4-oxadiazol-5-yl)pyridazin-3-yl)butane-1,4-diamine (B34)

To a solution of 7 (30 mg, 0.1 mmol, 1 equiv) in DMSO (1.0 mL) was added DIPEA (91 μL, 0.52 mmol) followed by 3 (51 mg, 0.16 mmol, 1.5 equiv). The reaction was heated and maintained at 110° C. for 18 h. The crude reaction mixture was allowed to cool to room temperature and was purified by reverse phase flash chromatography (0-100% acetonitrile/water with 0.1% TFA as a modifier) to afford 19 mg (31%) of B34 as yellow solid: ¹H NMR (500 MHz, CD₃OD) δ 8.36 (d, J=9.3 Hz, 1H), 8.09 (d, J=8.9 Hz, 2H), 7.92 (d, J=9.4 Hz, 1H), 7.79 (d, J=2.2 Hz, 1H), 7.75 (d, J=9.3 Hz, 1H), 7.64 (d, J=2.7 Hz, 1H), 7.47 (dd, J=9.3, 2.5 Hz, 1H), 7.35 (dd, J=9.3, 2.2 Hz, 1H), 7.09 (d, J=8.9 Hz, 2H), 6.78 (d, J=9.4 Hz, 1H), 4.08 (t, J=7.0 Hz, 2H), 3.96 (s, 3H), 3.89 (s, 3H), 3.55 (t, J=6.9 Hz, 2H), 2.03-1.97 (m, 2H), 1.83 (t, J=7.3 Hz, 2H); LRMS calculated for C31H28ClN7O3⁺ [M+H]+ m/z 581.19, measured LC/MS (ESI) Rₜ 4.10 min, m/z 582.40 [M+H]⁺.

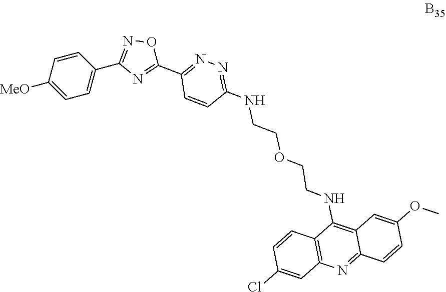 [SMILES: COc1ccc(-c2noc(-c3ccc(NCCOCCNc4c5ccc(Cl)cc5nc5ccc(OC)cc45)nn3)n2)cc1]

### 6-chloro-2-methoxy-N-(2-(2-((6-(3-(4-methoxyphenyl)-1,2,4-oxadiazol-5-yl)pyridazin-3-yl)amino)ethoxy)ethyl)acridin-9-amine (B35)

To a solution of 7 (42 mg, 0.15 mmol, 1 equiv) in DMSO (2 mL) was added DIPEA (130 μL, 0.73 mmol, 5 equiv) followed by 4 (75 mg, 0.22 mmol, 1.5 equiv). The reaction was heated and maintained at 110° C. for 18 h. The crude reaction mixture was allowed to cool to room temperature and purified by reverse phase flash chromatography (0-100% acetonitrile/water with 0.1% TFA as a modifier) to afford 48 mg (55%) of B35 as a yellow solid: ¹H NMR (500 MHz, DMSO) δ 8.58 (d, J=9.3 Hz, 1H), 8.03 (d, J=8.8 Hz, 2H), 7.89 (d, J=2.6 Hz, 1H), 7.81-7.71 (m, 3H), 7.64 (dd, J=9.3, 2.5 Hz, 1H), 7.45 (dd, J=9.2, 2.1 Hz, 1H), 7.17 (d, J=9.0 Hz, 2H), 6.73 (d, J=9.4 Hz, 1H), 4.26 (m, 2H), 4.01 (t, J=5.1 Hz, 2H), 3.93 (s, 3H), 3.87 (s, 3H), 3.75 (t, J=5.3 Hz, 2H), 3.63 (m, 2H); LRMS calculated for C31H28ClN7O4+[M+H]+m/z 597.18, measured LC/MS (ESI) Rₜ 3.89 min, m/z 598.35 [M+H]⁺.

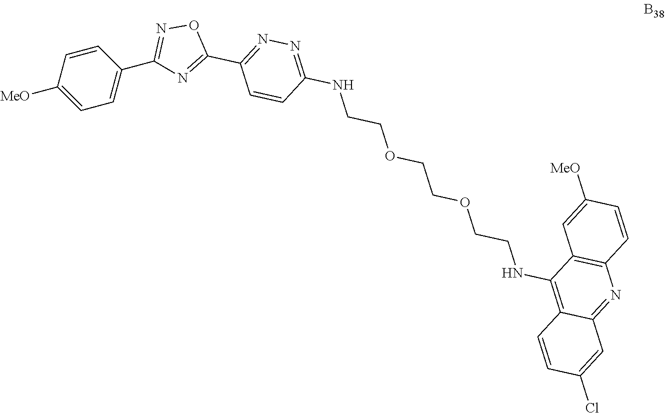 [SMILES: COc1ccc(-c2noc(-c3ccc(NCCOCCOCCNc4c5ccc(Cl)cc5nc5ccc(OC)cc45)nn3)n2)cc1]

### 6-chloro-2-methoxy-N-(2-(2-(2-((6-(3-(4-methoxyphenyl)-1,2,4-oxadiazol-5-yl)pyridazin-3-yl)amino)ethoxy)ethoxy)ethyl)acridin-9-amine (B38)

To a solution of 7 (100 mg, 0.35 mmol, 1 equiv) in DMSO (2 mL) was added DIPEA (302 μL, 1.73 mmol, 5 equiv) followed by 5 (168 mg, 0.43 mmol, 1.5 equiv). The reaction was heated and maintained at 110° C. for 18 h. The crude reaction mixture was allowed to cool to room temperature and purified by reverse phase flash chromatography (0-100% acetonitrile/water with 0.1% TFA as a modifier) to afford 50 mg (23%) of B38 as a yellow solid: ¹H NMR (500 MHz, DMSO) δ 8.60 (d, J=9.3 Hz, 1H), 8.01 (d, J=8.8 Hz, 2H), 7.92 (d, J=2.6 Hz, 1H), 7.88 (d, J=9.4 Hz, 1H), 7.81-7.75 (m, 2H), 7.68 (dd, J=9.3, 2.5 Hz, 1H), 7.51 (dd, J=9.3, 2.2 Hz, 1H), 7.15 (d, J=8.9 Hz, 2H), 6.90 (d, J=9.4 Hz, 1H), 4.24 (q, J=5.6 Hz, 2H), 3.94 (m, 5H), 3.86 (s, 3H), 3.64 (m, 2H), 3.59-3.53 (m, 4H), 3.51 (m, 2H); LRMS calculated for C33H32ClN7O5⁺[M+H]+m/z 641.21, measured LC/MS (ESI) R₁ 3.87 min, m/z 642.50 [M+H]⁺.

### Example 1

### Analysis of G-Quadruplexes in the MYCN Gene

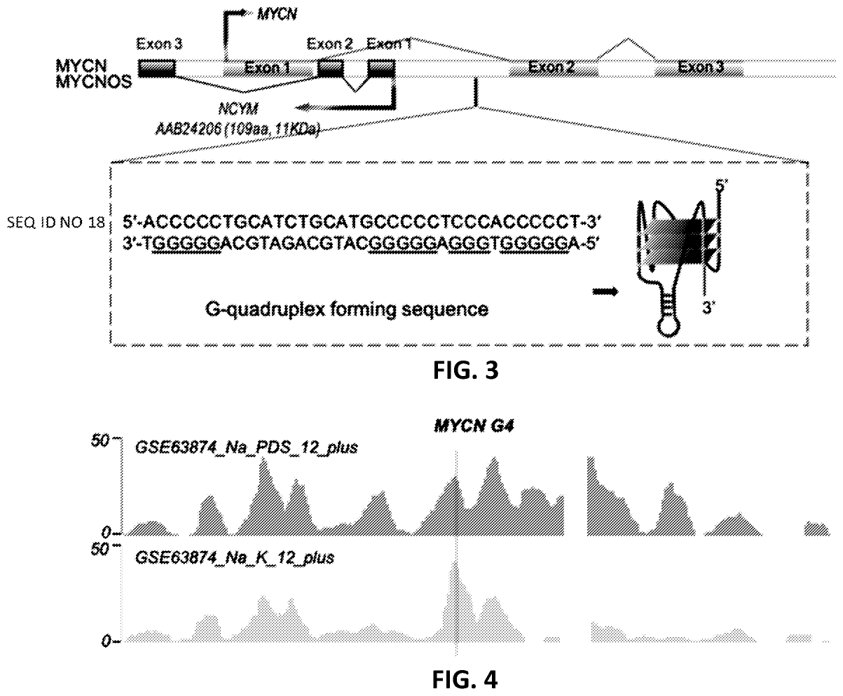

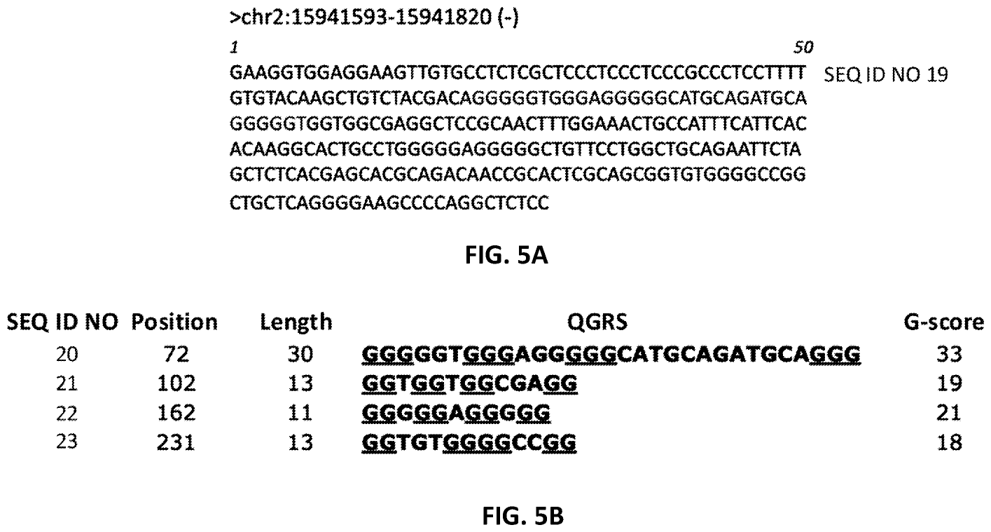

As shown in FIG. 3, the MYCN G4 discussed herein is located in the negative strand and near the transcription start site (TSS) of MYCNOS. However, unlike simple G4s, the MYCN G4 contains a hairpin flanking the tetrads (Benabou et al., Biochim. et Biophys. Acta 2014, 1840(41-52). This class of hybrid G4 structures have been recently characterized by both genomic data mining and experimental approaches (Onel et al., JACS 2016, 138:2563-2570; Ngoc Nguyen et al., Nucleic Acids Res. 2020, 48:10567-10575; Lim et al., Nucleic Acids Res. 2015, 43:5630-5646), and represent intriguing targets for small molecules due to their unique structures Kang et al., JACS 2016, 138:13673-13692). Although the MYCN G4 has been structurally characterized in vitro, questions frequently arise about the existence of G4s in biological contexts. In order to confirm that the MYCN gene contains sequences that fold into G4s, the genome-wide G4-seq data reported by the Balasubramanian group (Chambers et al., Nature Biotechnology 2015; 33:877-881) was analyzed. This approach consisted of two rounds of sequencing before and after stabilizing the G4s. A higher mismatch rate between the experiments represented a higher chance to form G4 structure in the corresponding region. Within these data, the G4 sequence of interest to this work was found to have a much higher mismatch rate in both K⁺ and PDS-stabilized data sets, confirming the presence of the G4 in MYCN gene (FIG. 4). Further, by calculating the G-scores within this sequence using QGRS mapper (Kikin et al., Nucleic Acids Res. 2006, 34:W676-682), the target of interest was found to show a promising G-score of 33 (FIGS. 5A-5B).

### Example 2

### Small Molecule Microarray Screening and Hit Identification

To identify a small molecule binder of the MYCN G4, a small molecule microarray (SMM) screening approach was used. In this study, glass slides modified with epoxy groups were prepared according to an analogous protocol reported previously (Abulwerdi et al., Methods 2016, 103:188-195). Compounds were printed by a robotic arrayer (Arrayjet™) and covalently immobilized on the glass surfaces. Then the slides were incubated with 50 nM MYCN G4 DNA with Cy5 labeling at 5′ and buffer in parallel. After washing and drying, the slides were imaged by a microarray scanner. For each compound, a composite Z-score was calculated, and hits were identified (Connelly et al., ACS Chemical Biology 2017, 12:435-443). Compounds that lacked promiscuous binding to other nucleic acids in other SMM screens were prioritized. In total, 14 hits were identified (Table 2).

TABLE 2

| | |
|---|---|
| 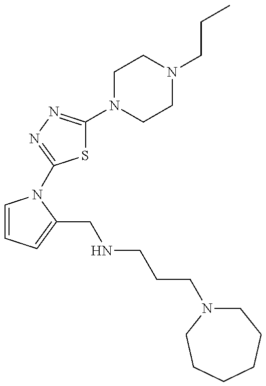 [SMILES: CCCN1CCN(c2nnc(-n3cccc3CNCCCN3CCCCCC3)s2)CC1] | 1 |
| 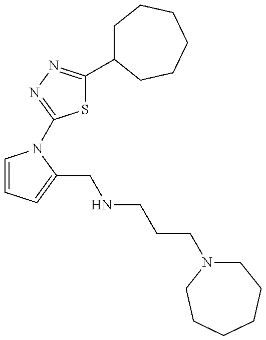 [SMILES: c1cc(CNCCCN2CCCCCC2)n(-c2nnc(C3CCCCCC3)s2)c1] | 2 |
| 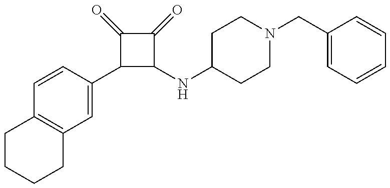 [SMILES: O=C1C(=O)C(c2ccc3c(c2)CCCC3)C1NC1CCN(Cc2ccccc2)CC1] | 3 |
| 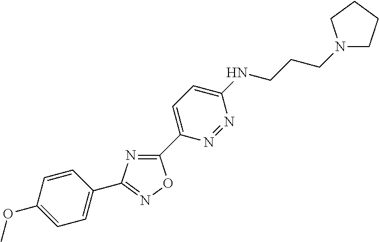 [SMILES: COc1ccc(-c2noc(-c3ccc(NCCCN4CCCC4)nn3)n2)cc1] | MY-1 |
| 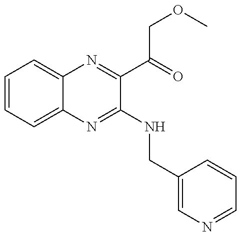 [SMILES: COCC(=O)c1nc2ccccc2nc1NCc1cccnc1] | 5 |
| 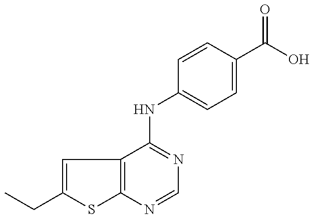 [SMILES: CCc1cc2c(Nc3ccc(C(=O)O)cc3)ncnc2s1] | 6 |
| 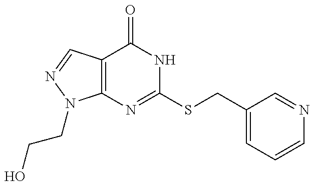 [SMILES: O=c1[nH]c(SCc2cccnc2)nc2c1cnn2CCO] | 7 |
| 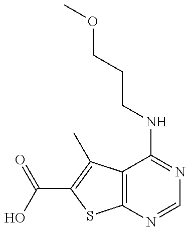 [SMILES: COCCCNc1ncnc2sc(C(=O)O)c(C)c12] | 8 |
| 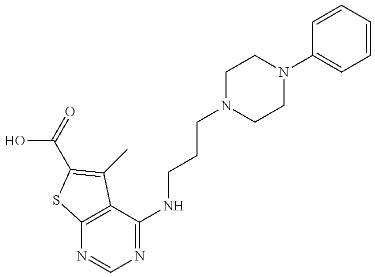 [SMILES: Cc1c(C(=O)O)sc2ncnc(NCCCN3CCN(c4ccccc4)CC3)c12] | 9 |
| 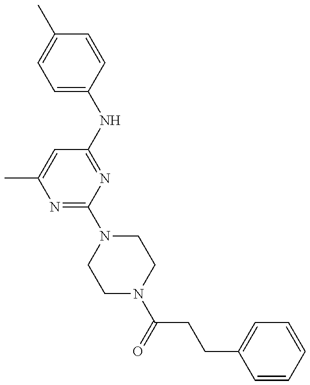 [SMILES: Cc1ccc(Nc2cc(C)nc(N3CCN(C(=O)CCc4ccccc4)CC3)n2)cc1] | 10 |
| 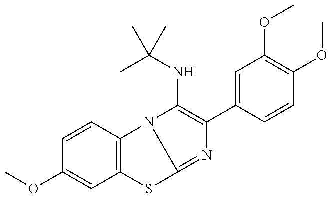 | 11 |
| 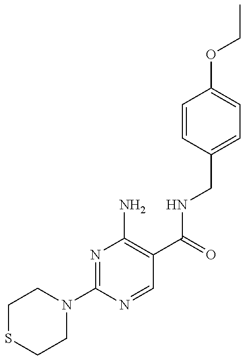 [SMILES: CCOc1ccc(CNC(=O)c2cnc(N3CCSCC3)nc2N)cc1] | 12 |
| 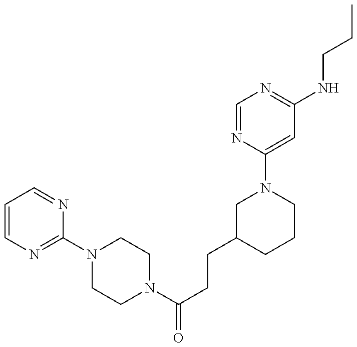 [SMILES: CCCNc1cc(N2CCCC(CCC(=O)N3CCN(c4ncccn4)CC3)C2)ncn1] | 13 |
| 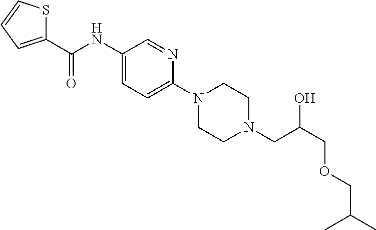 [SMILES: CC(C)COCC(O)CN1CCN(c2ccc(NC(=O)c3cccs3)cn2)CC1] | 14 |

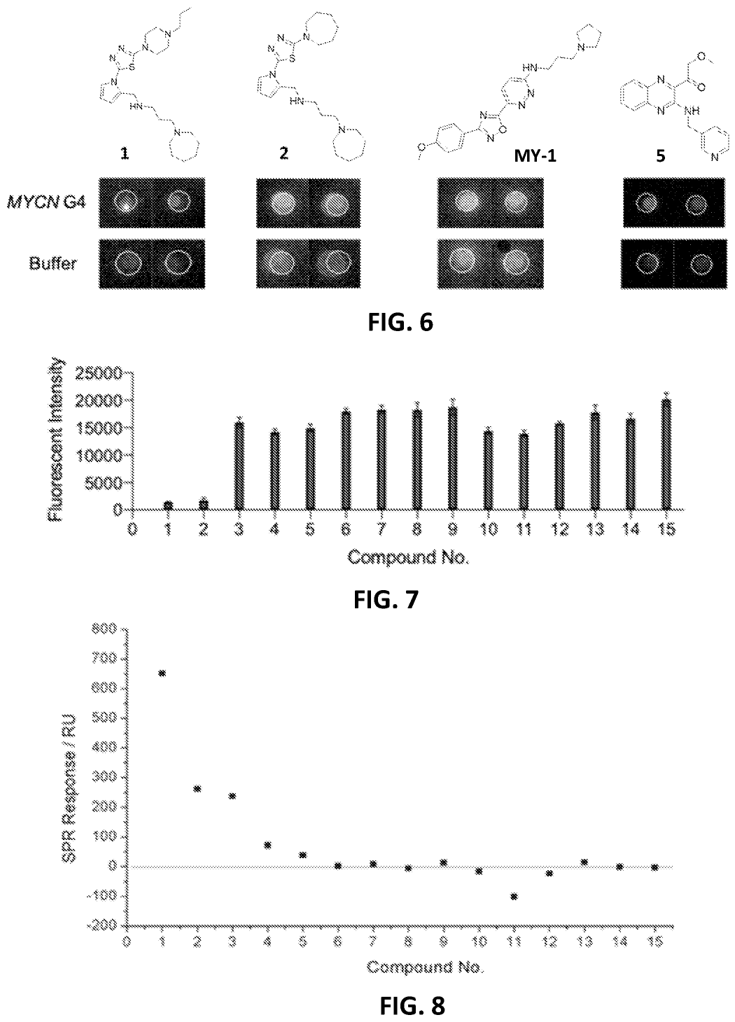

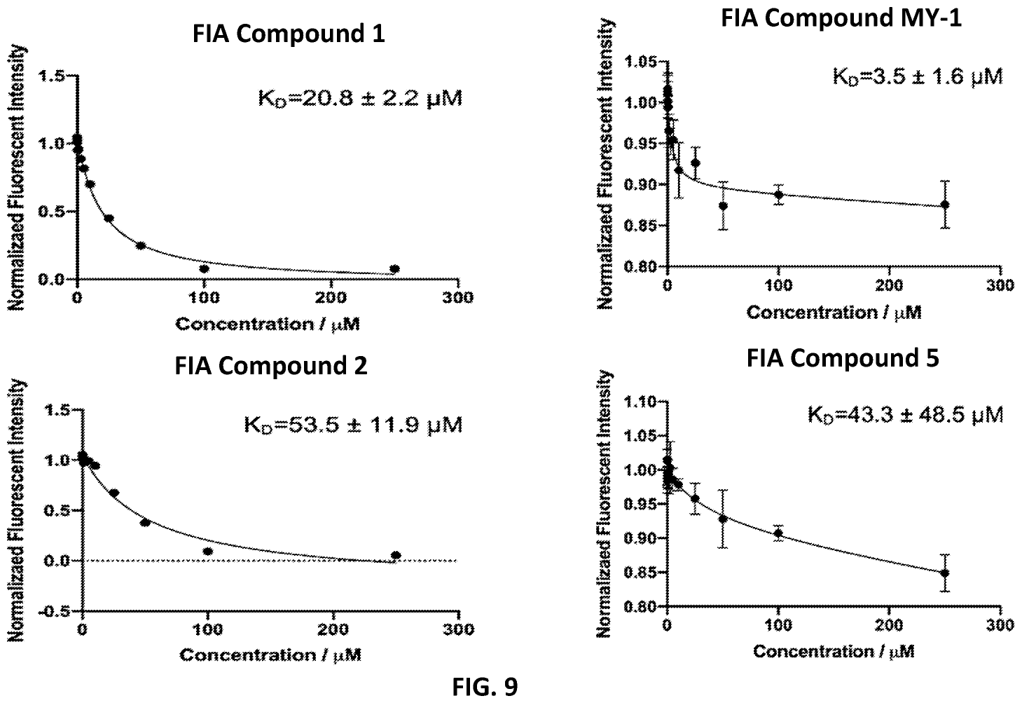

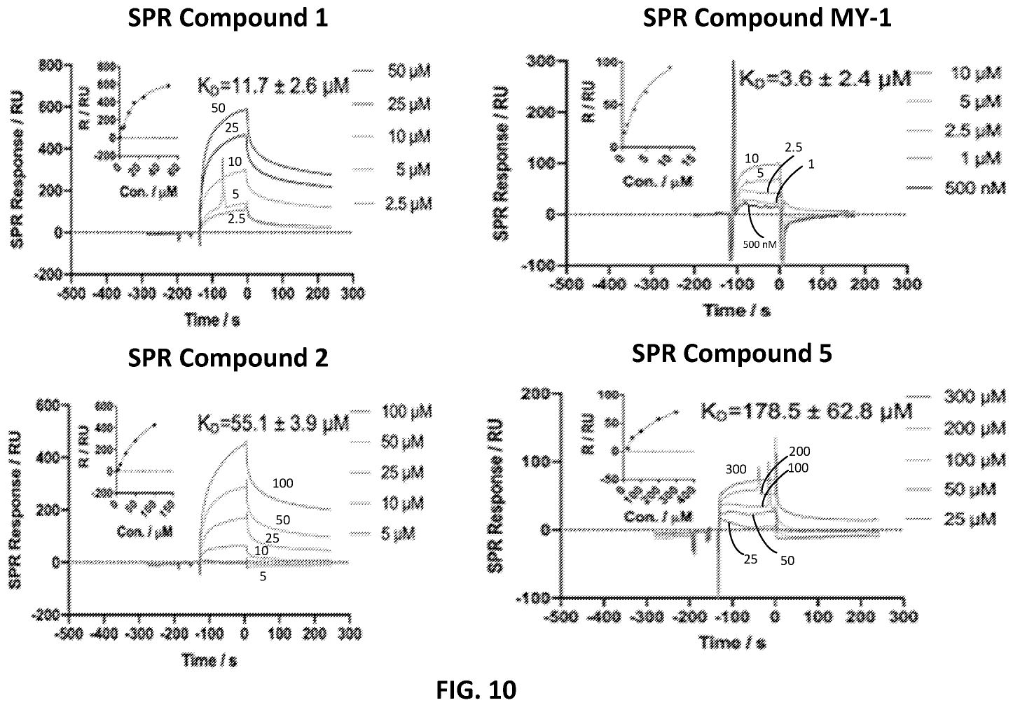

To validate the 14 hit compounds as genuine binders, single-concentration binding assays were performed by fluorescence intensity assay (FIA) and surface plasmon resonance (SPR). Compounds were evaluated at 100 μM concentration in each assay. In this example, a positive binding was defined with a criteria of >10% quenching in FLA study, while SNR>3 was used as the cut-off in SPR experiments. Compounds 1, 2, MY-1, 5, 10, 11, 12, and 14 were hits in the FIA study; compounds 1-3, MY-1, 5, 9 and 13 were hits in the SPR study. From these assays four compounds (1, 2, MY-1, and 5) were identified that showed positive responses in both binding assays (FIGS. 6-8). Equilibrium dissociation binding constant (K_{D}) values were measured by FIA and SPR (FIGS. 9 and 10). By titrating MYCN G4 DNA sample with the gradient concentrations of compounds, compound MY-1 was found to show the best binding among the 4 candidates (K_{D}=3.5±1.6 μM by FIA, and K_{D}=3.6±2.4 μM by SPR). Compound 1 shared a similar structure with 2 but had a better binding affinity (20.8±2.2 μM by FIA, and 11.7±2.6 μM by SPR).

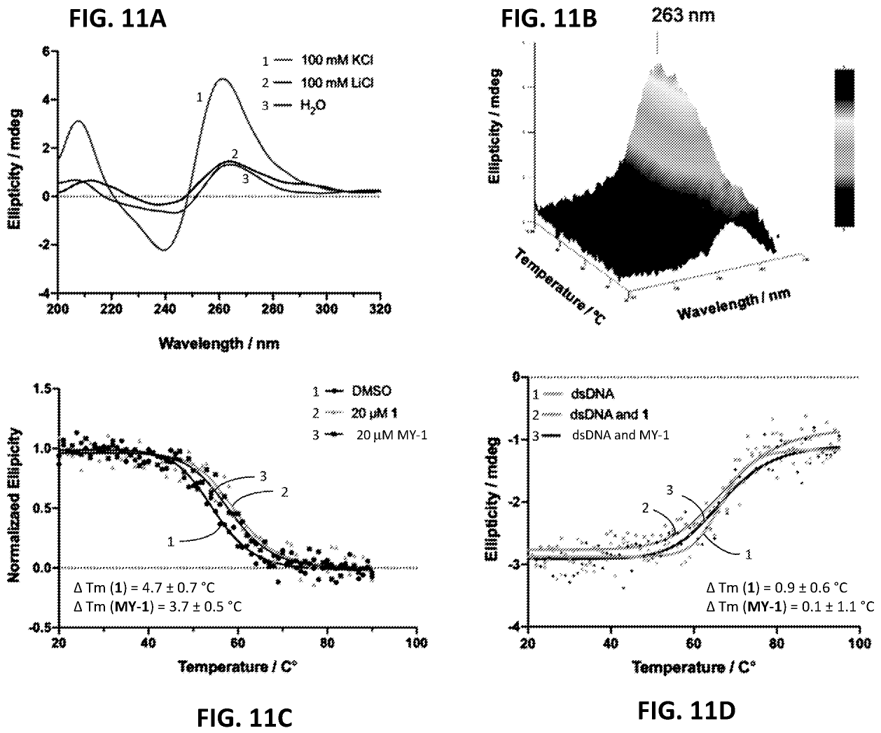

The effect of MY-1 on MYCN G4 thermal stability was evaluated by circular dichroism (CD). First, G4 formation was confirmed by CD spectroscopy. Samples of MYCN G4 DNA were prepared in 3 different buffer conditions (10 mM Tris with 100 mM KCl, 10 mM Tris with 100 mM LiCl, and H₂O). As shown in FIG. 11A, the MYCN G4 DNA exhibited a positive peak at 263 nm and a negative peak at 240 nm, characteristic of a properly folded parallel G-quadruplex structure in KCl buffer. In contrast, spectra of DNA samples in LiCl buffer and H₂O showed a much weaker peak at 263 nm while the peak at 240 nm was negligible.

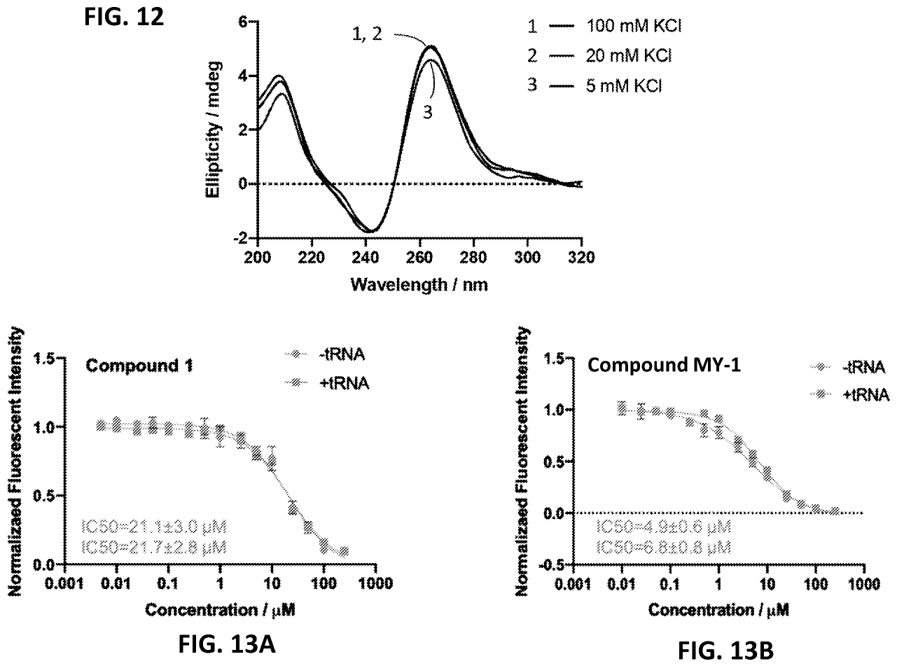

Next, a melting assay was demonstrated on the CD instrument using MYCN G4 DNA incubated without/with small molecule binders that were validated. The 263 nm peak in the CD spectrum decreased gradually during heating (FIG. 11B), and Tₘ was determined by fitting the temperature-dependent intensities. The MYCN G4 was first melted in 10 mM Tris buffer (pH7.0) with 100 mM KCl, and Tₘ was determined as 78.0±1.9° C., which was consistent with a previously reported value (Benabou et al., Biochim. et Biophys. Acta 2014; 1840:41-52). To evaluate the impact of molecules on thermal unfolding, assays were performed using reduced KCl concentration (Kumari et al., Nature Chemical Biology 2007, 3:218-221), after confirming the folding of G4 was not affected by lower KCl (FIG. 12). In 5 mM KCl buffer, the melting curve of MYCN G4 was better fitted, with a Tₘ of 54.6±0.4° C. The Tₘ was not affected by the addition of 5% DMSO into the buffer. 20 μM 1 or MY-1 was added into MYCN G4 solution and incubated for 15 min before unfolding. As a result, both of the compounds induced significant shift in melting curves, increasing Tₘ by 4.7±0.7° C. (for 1) and 3.7±0.5° C. (for MY-1), respectively (FIG. 11C). Neither of the compounds showed a measurable effect on dsDNA melting (FIG. 11D), indicating that those hit compounds were not B-DNA binders.

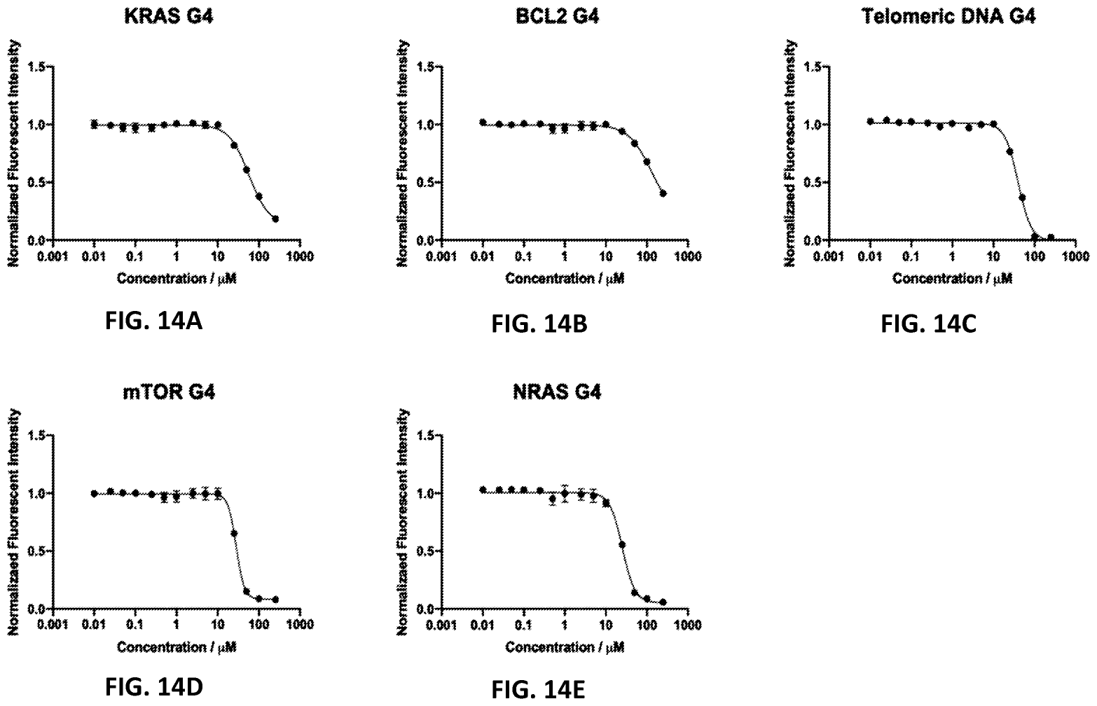

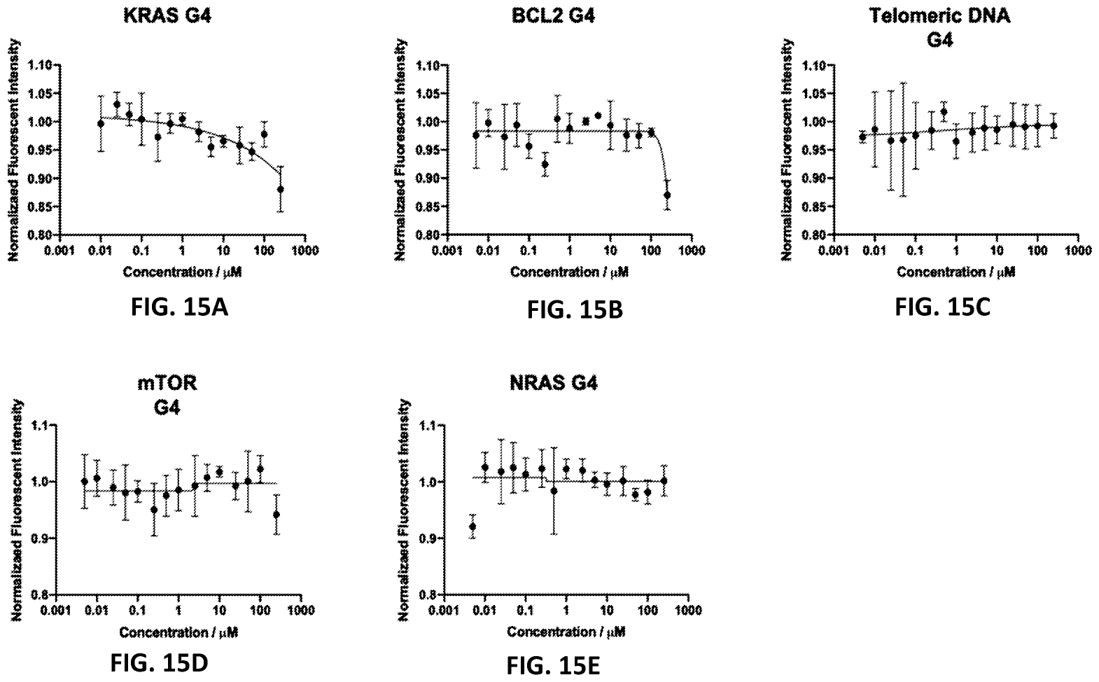

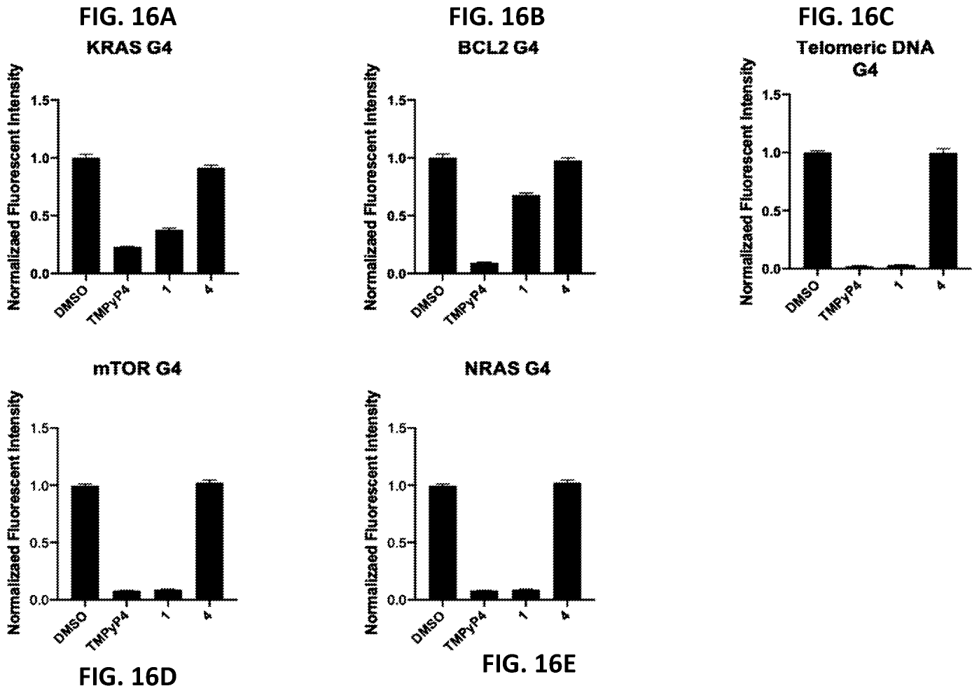

The binding selectivities of compounds 1 and MY-1 were also evaluated. In a separate experiment, fluorescence-based assays were performed by titrating the compounds into G4 solutions with the presence/absence of excess tRNA. As with dsDNA, excess tRNA had no effect on the binding affinity of 1 or MY-1, again suggesting a specific mode of interaction (FIGS. 13A and 13B). In addition, another five 5′Cy5-labeled G4s from cancer-relevant genes (FIG. 1) were also introduced in selectivity profiling. By titrating the two hit compounds, compound 1 was observed to show some binding to all the G4s with binding affinities ranging from 25.2 to 127.9 μM, but weaker than that of MYCN G4 (FIGS. 14A-14E). However, MY-1 showed no significant quenching behavior and K_{D} values could not be measured for any G4 except MYCN (FIGS. 15A-15E, 16A-16E). This result indicated that MY-1 bound to MYCN G4 via its unique structure.

### Example 3

### Identification of Compound MY-1 as a Non-Canonical G4 Binder

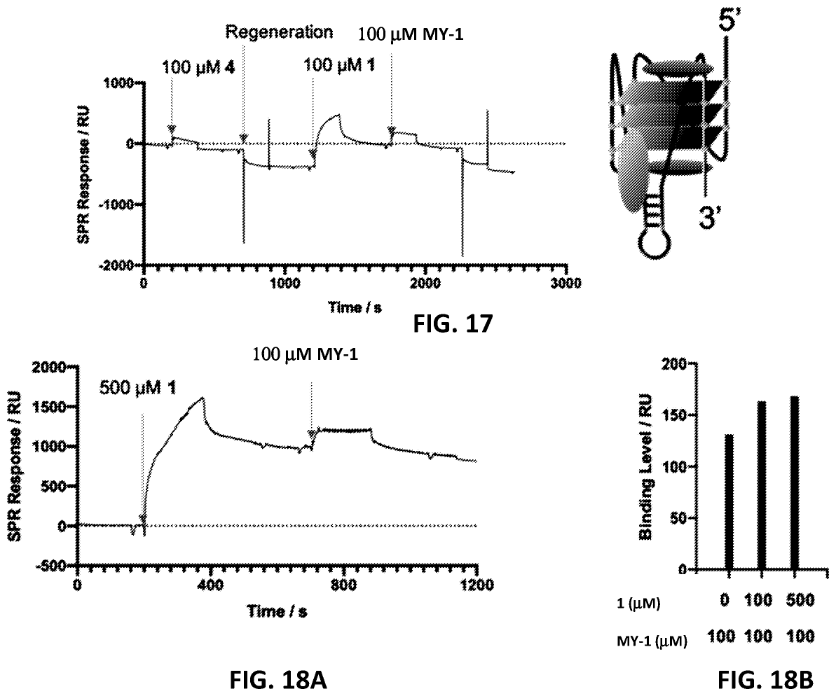

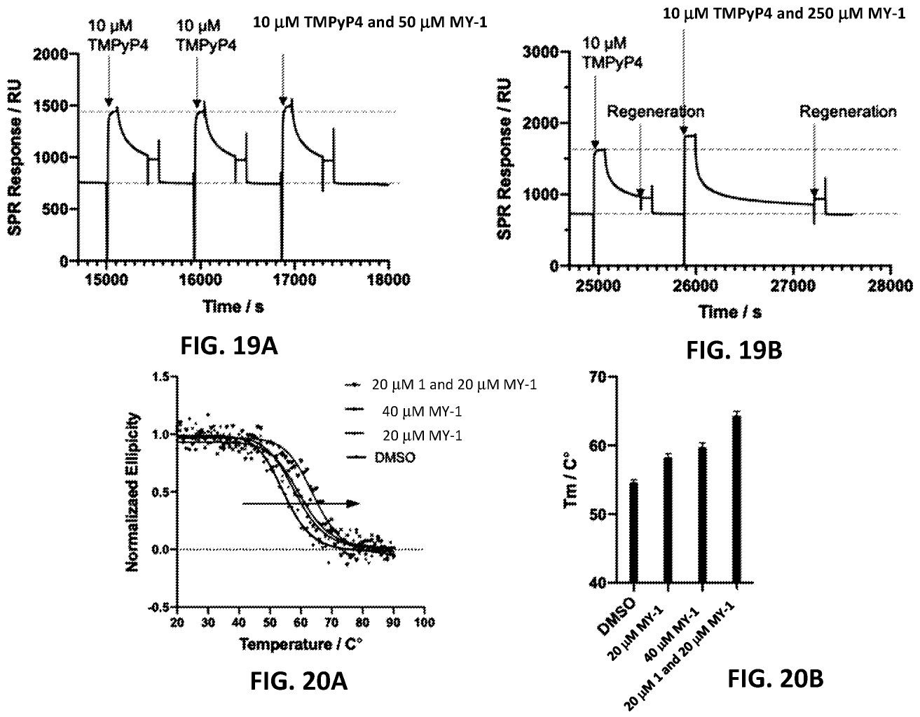

By analyzing SPR sensorgrams, significantly different binding levels (Rₘₐₓ) between compounds 1 and MY-1 were observed, which suggested that the binding modes of the two compounds might be different. Based on this unexpected observation, and without being bound to a single theory, it currently is believed that the two compounds might bind to MYCN G4 on different sites. To investigate, a competitive assay on SPR was performed by injecting the compounds step-wise in a designated order or injecting a mixture (Brooks et al., Drug Discovery Today 2014, 19:1040-1044; Spurny et al., PNAS 112:E2543-2552). By injecting 100 μM of 1 followed by100 μM of MY-1 injection, the observed binding level of MY-1 was not significantly changed, compared with that of individual injection (FIG. 17). Even at a higher concentration (500 μM) of 1, the level of MY-1 binding was maintained (FIGS. 18A and 18B), suggesting that 1 does not compete with MY-1. Next, TMPyP4, a classical stacking G4-binder (Seenisamy, JACS 2004, 126:8702-8709), was also tested together with MY-1. The addition of MY-1 (both 100 μM and 250 μM) in TMPyP4 solution resulted in an increase of SPR binding signals (FIGS. 19A and 19B), suggesting that MY-1 bound to G4 in a distinct mode. In addition, a CD melting assay was also performed using either MY-1 or a mixture of two hits (1 and MY-1). A mixture of solution containing 20 μM 1 and 20 μM MY-1 increased Tₘ of MYCN G4 by 9.7±0.7° C., which was significantly higher than that of 20 μM MY-1 only (FIGS. 20A and 20B), suggesting non-overlapping modes of interaction. In contrast, the ΔTₘ of MYCN G4 with 40 μM MY-1 addition was not significantly increased, presumably due to the saturation of binding sites.

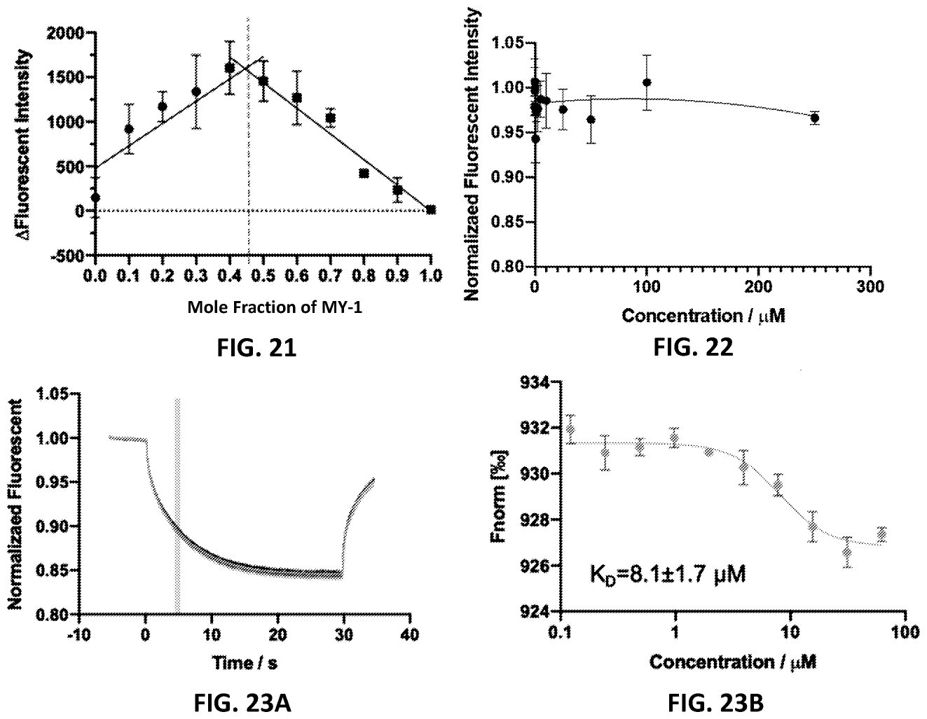

To evaluate the binding stoichiometry of MY-1 with MYCN G4, a Job plot analysis was performed. Briefly, by varying the component fraction during fluorescent titration, the changes in fluorescent intensity were plotted and fitted, resulting in a maximum at 0.47, indicating a 1:1 binding stoichiometry (FIG. 21). The binding between MY-1 and MYCN G4 was studied by microscale thermophoresis (MST). 3′-Cy5 labeled DNA sample was used since there was no quenching observed (FIG. 22). The microscale thermophoresis (MST) curve was well fitted with 1:1 binding model, and the binding affinity was calculated as 8.1±1.7 μM which agreed with the results of FIA and SPR experiments (FIGS. 23A and 23B). Consequently, unlike a majority of other reported G4-stackers, and distinct from 1, MY-1 was identified as a non-canonical G4-binder.

### Example 4

### Binding Site Identification Using FIA and DMS-Footprinting

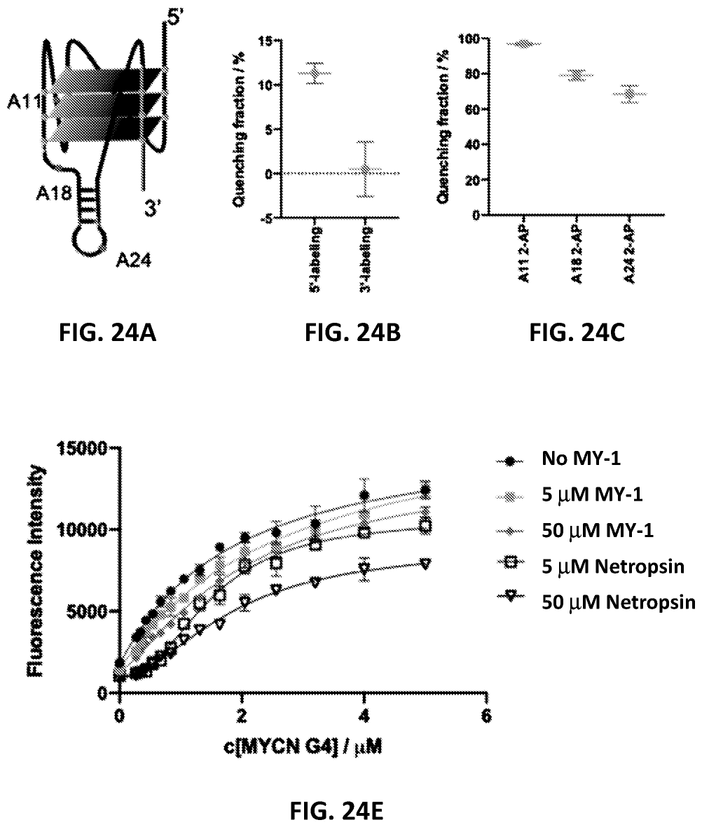

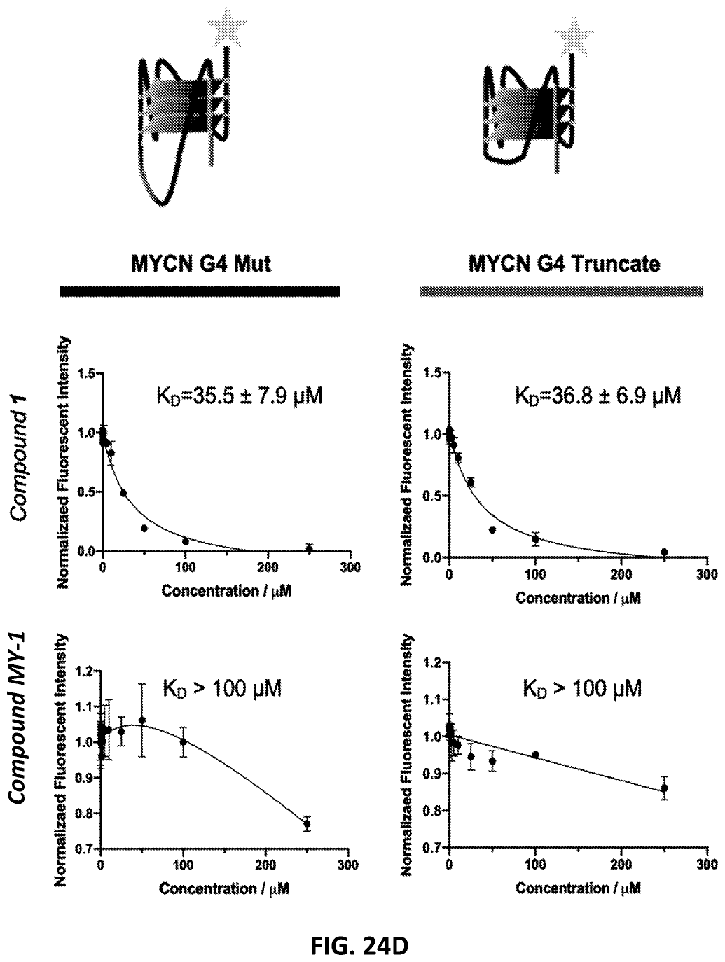

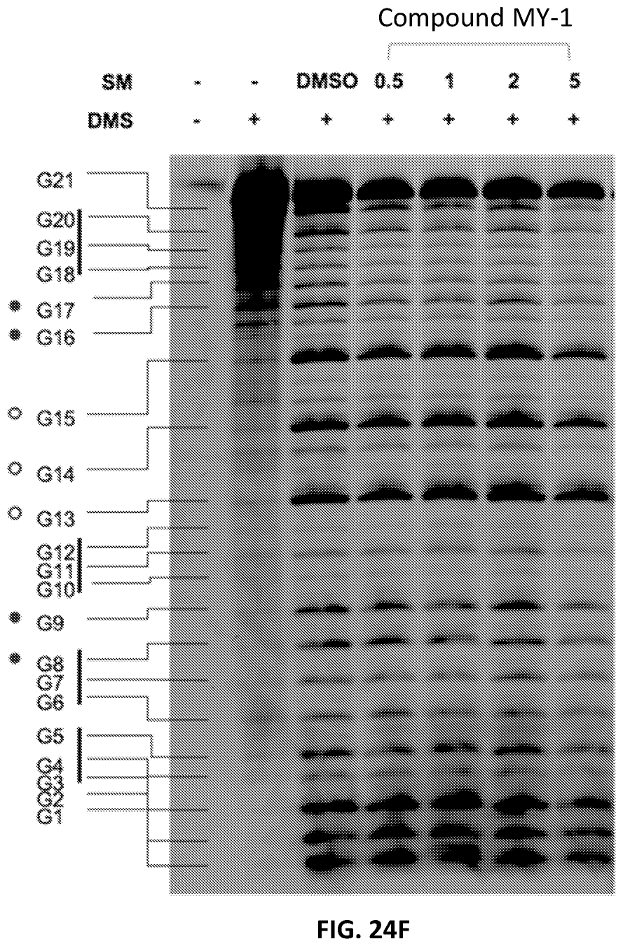

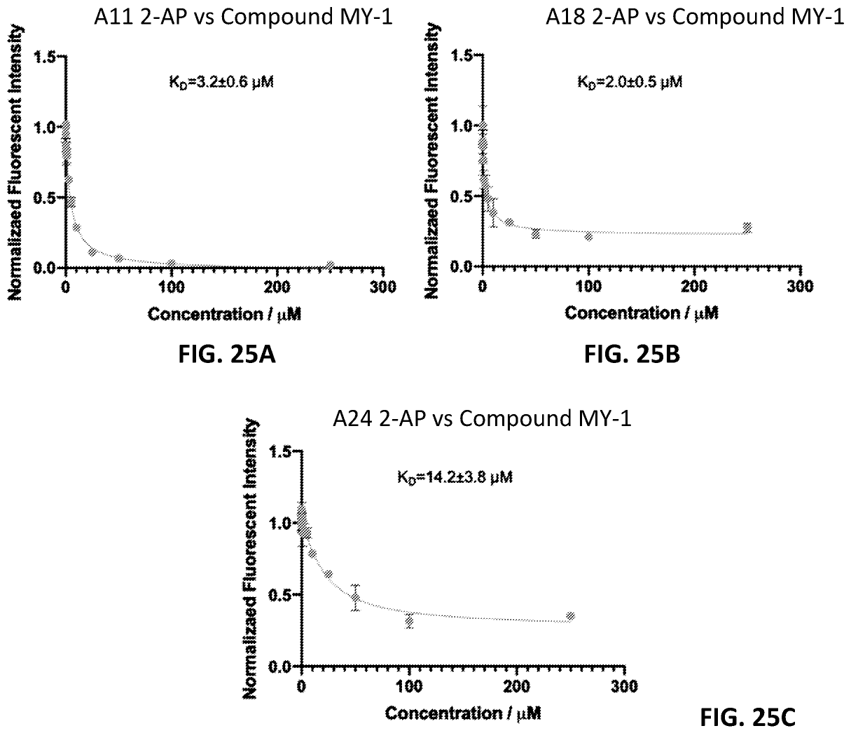

To further understand the binding details between compound MY-1 and MYCN G4, a series of FIA studies were designed and performed. Given that the hit compound bound to G4 with 1:1 stoichiometry, it was hypothesized that the unique binding behavior was related to the hairpin structure. The binding region was first investigated by introducing environment-sensitive 2-aminopurine (2-AP) fluorophores at distinct positions (All, A18, and A24) of the G4 (FIG. 24A). After incubating 100 μM MY-1 with labeled DNA samples, fluorescence intensities were measured and compared to DMSO controls. Fluorescence of the A11 2-AP DNA sample was quenched by almost 100%, while the quenching percentages were 80% and 70% for A18 and A24 2-AP DNAs, respectively (FIG. 24B, FIGS. 25A-25C). Based on the previous observation of different quenching behaviors between 5′ and 3′-Cy5 labeled oligos (15% and 0% quenching, FIG. 24C), it was hypothesized that MY-1 was binding to the region near the hairpin. Next, the necessity of hairpin in this binding event was explored by designing two DNA constructs including mutated long loop (could not form base-pairing) and truncated sequence (no hairpin) (see FIG. 2). By titrating MY-1 into the two DNAs, the binding affinity was decreased considerably to >100 μM micromolar (˜30 fold weaker than that of the wild type MYCN G4). In contrast, compound 1 was also tested against these G4s, and affinities were comparable to wild type (WT) constructs (FIG. 24D). These results indicated that the hairpin structure in MYCN G4 was important to maintain the binding with MY-1, but not with 1.

In addition, to explore whether the compound bound to minor groove of the hairpin, MY-1 was tested together with other two classical minor groove binders (Hoechst 33258 and netropsin) by fluorescence displacement assay (Alniss, J. Medicinal Chemistry 2019, 62:385-402). Since Hoechst 33258 has been reported as a fluorophore and widely used as DNA staining reagent, it was first incubated and titrated with different concentrations of unlabeled MYCN G4 DNAs. By utilizing excitation wavelength of 352 nm and an emission wavelength of 500 nm, the fluorescence intensity was enhanced with the increasing DNA concentration as expected (FIG. 24E). Next, either MY-1 or netropsin was added as a competitor into the solutions during DNA titration. Netropsin, as a well-known minor groove binder, showed a significant displacement of Hoechst 33258 as measured by corresponding decrease in fluorescence intensity. However, for MY-1, there was no measurable change observed at 5 μM, while partial fluorescence decrease was observed at 50 μM. Thus, although it currently is believed that the hairpin facilitates MY-1 binding, it does not appear to be a classical minor groove binder.

In order to better understand the mode of binding of compounds to the MYCN G4, a dimethyl sulfate (DMS) foot-printing assay was developed. This assay exploits the ability of DMS to methylate the free N7 within guanines (Gs), which can subsequently be cleaved using piperidine (43,44). In contrast, Hoogsteen bonded Gs in the quadruplex structure remain protected from modification. Further, changes in modification upon incubation with small molecules can be observed near binding sites. 5′Cy5-labeled MYCN G4 was annealed in the presence of 5 mM KCl, and subsequently compounds were added at concentrations according to observed K_{D}s. As shown in FIG. 24F, bands corresponding to G10-G12 were dim due to protection indicating that those Gs were incorporated within tetrads in the quadruplex. However, G13-G16 remained unprotected showing much brighter bands, due to hairpin formation via Watson-Crick base pairing. In the presence of compound MY-1, concentration-dependent protection of various Gs was observed, indicating that the compound stabilized the G4 structure. Upon the addition of MY-1, G8, G9, G16, G17 were protected considerably, while G13-G15 were slightly protected. This result suggested that MY-1 was binding to the junction between quadruplex and hairpin, in agreement with multiple results described above. (FIG. 24B).

### Example 5

### Structure-Activity Relationship (SAR) Study of Compound MY-1

To obtain a better binder for MYCN G4 target, a structure-activity relationship (SAR) study was performed by using a series of analogs of MY-1 (see Table 3). A focused library containing MY-1 and 13 derivatives (MY-2 to MY-14) was either purchased or synthesized. Each compound was evaluated by SPR and 2-AP titration as described above. In the preliminary study, it currently is believed that the pyrrolidine group in MY-1 played a role not only by maintaining the binding with G4, but also helping with the solubility. Meanwhile, it also currently is believed that the heterocyclic core of MY-1 (shown in Table 3) facilitates MYCN G4 recognition. Thus, most analogs contained altered side chain R groups.

TABLE 3
SAR study of compound (MY-1)

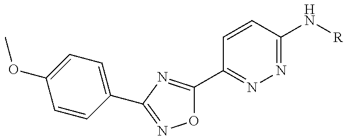

| | | SPR K_{D} | A11 2AP |
|---|---|---|---|
| Name | R | (μM) | K_{D} (μM) |
| MY-1 | 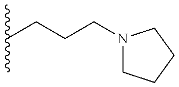 [SMILES: *CCCN1CCCC1] | 3.6 ± 2.4 | 3.2 ± 0.6 |
| MY-2 | 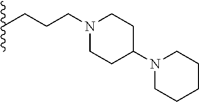 [SMILES: *CCCN1CCC(N2CCCCC2)CC1] | 2.6 ± 1.9 | 0.20 ± 0.04 |
| MY-3 | 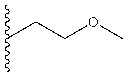 [SMILES: *CCOC] | 5.9 ± 2.7 | 1.2 ± 0.6 |
| MY-4 | 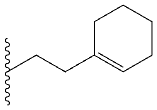 [SMILES: *CCC1=CCCCC1] | N/A | N/A |
| MY-5 | 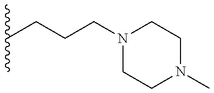 [SMILES: *CCCN1CCN(C)CC1] | 1.4 ± 0.2 | 1.0 ± 0.1 |
| MY-6 | 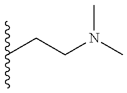 [SMILES: *CCN(C)C] | 4.9 ± 2.6 | 1.0 ± 0.2 |
| MY-7 | 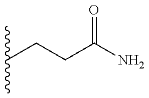 [SMILES: *CCC(N)=O] | 23.5 ± 4.7 | 13.2 ± 4.6 |
| MY-8 | 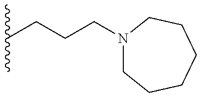 [SMILES: *CCCN1CCCCCC1] | 1.5 ± 0.3 | 0.6 ± 0.2 |
| MY-9 | 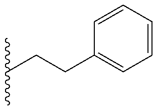 [SMILES: *CCc1ccccc1] | N/A | N/A |
| MY-10 | 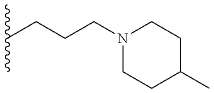 [SMILES: *CCCN1CCC(C)CC1] | 2.2 ± 0.6 | 0.7 ± 0.2 |
| MY-11 | 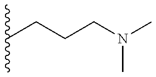 [SMILES: *CCCN(C)C] | 9.2 ± 1.9 | 1.2 ± 0.2 |
| MY-12 | 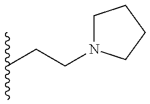 [SMILES: *CCN1CCCC1] | >100 | N/A |
| MY-13 | 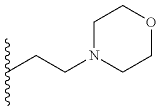 [SMILES: *CCN1CCOCC1] | 8.2 ± 2.4 | 3.2 ± 0.7 |
| MY-14 | 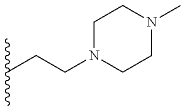 [SMILES: *CCN1CCN(C)CC1] | 3.6 ± 1.0 | 0.9 ± 0.2 |

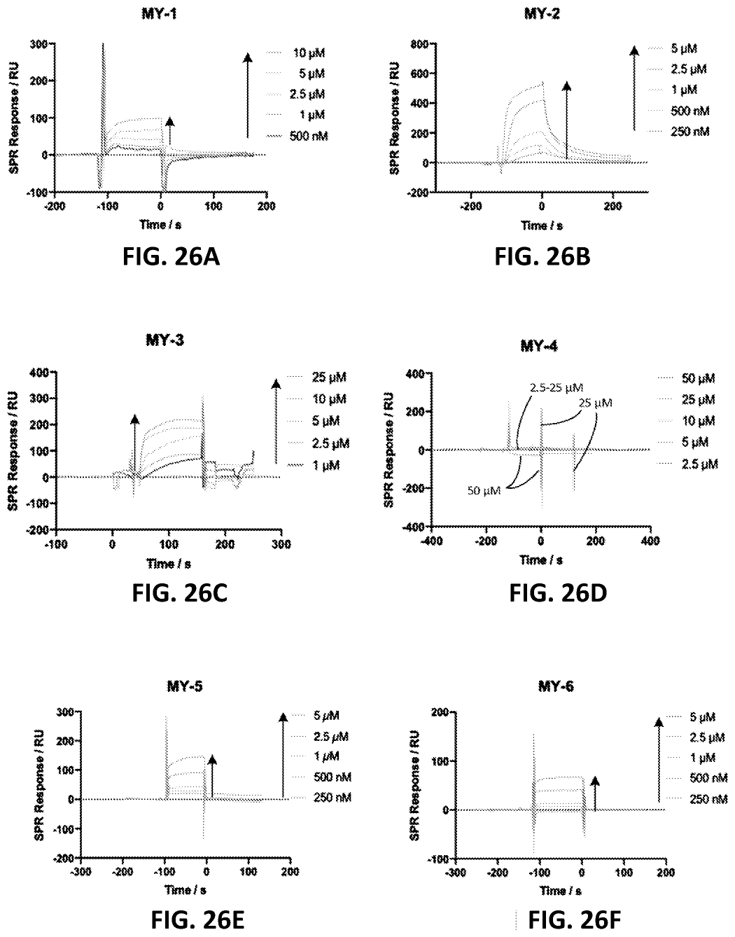

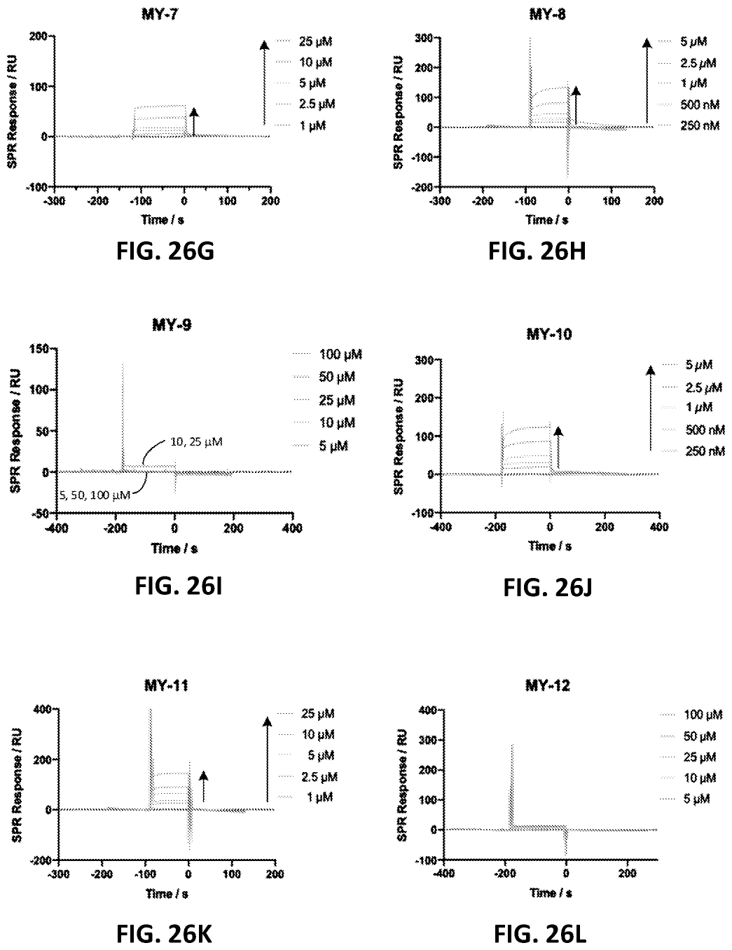

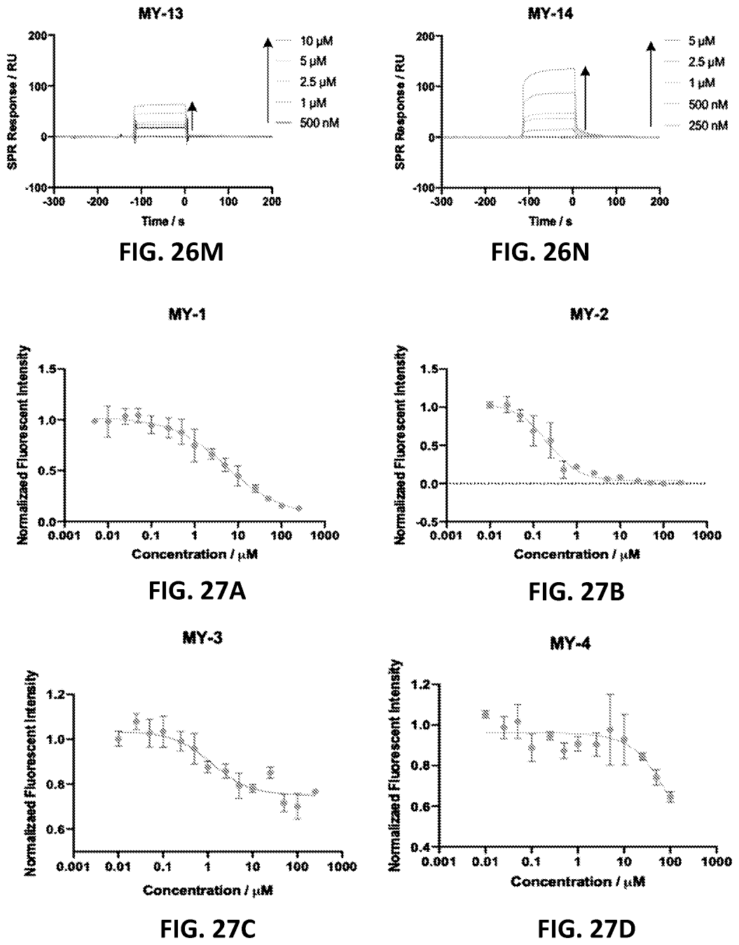

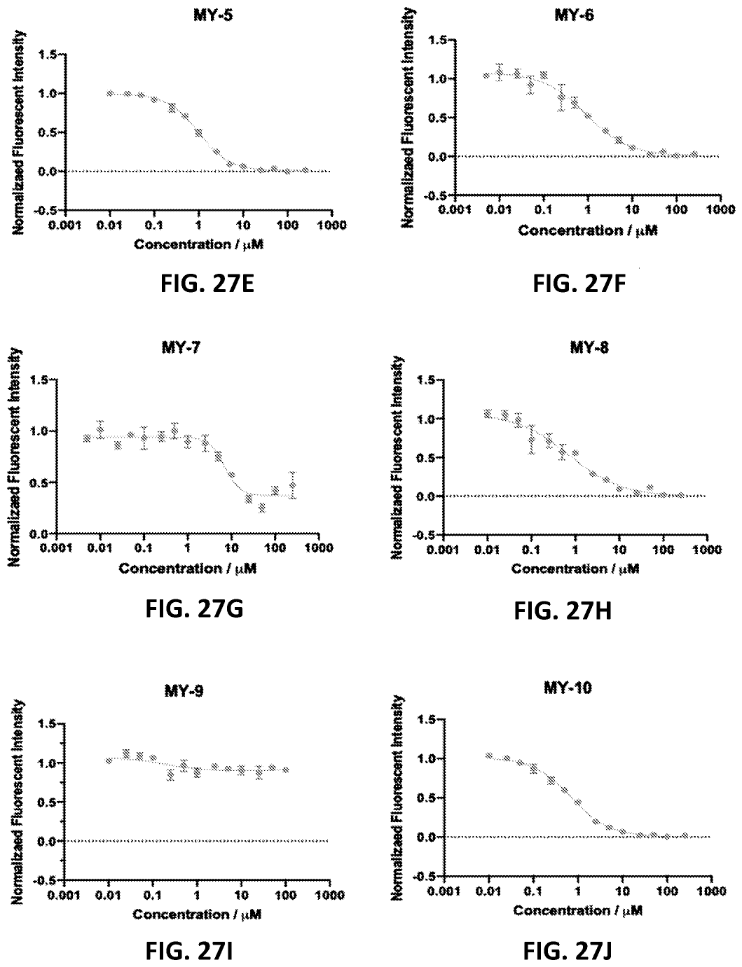

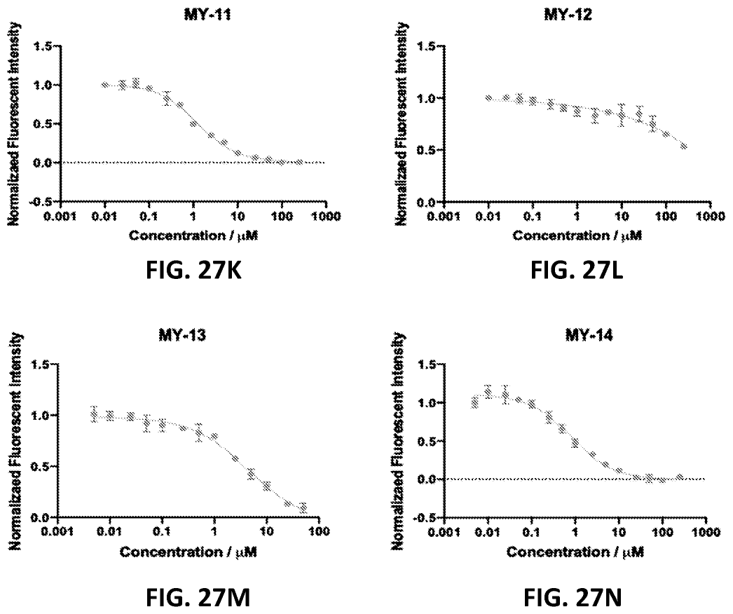

A systematic binding assay between 14 analogs and MYCN G4 was performed by SPR and 2-AP (A11) fluorescence titration (FIGS. 26A-26N, 27A-27N). As an overview, the observed binding affinities ranged from 1.4 to 23.5 μM (SPR), and 0.2 to 7.0 μM (2-AP titration). Most of the analogs exhibited good binding behavior toward the MYCN G4, except MY-4 and MY-9, both of which had solubility in suitable buffers. In SPR experiments, 4 analogs (MY-2, 5, 8, and 10) were identified as stronger binders than the parent compound, among which MY-5 and MY-8 displayed the tightest binding affinities of 1.4±0.2 μM and 1.5±0.3 μM, respectively. Compound MY-2 showed a high response in the SPR sensorgrams, suggesting that it was an aggregator. For 2-AP fluorescence titration assay (using an A11-substituted construct) observed K_{D} values aligned well with SPR experiments, although the values were slightly lower overall. Among the tested analogs, four compounds (MY-2, MY-8, MY-10, and MY-14) were discovered as sub-micromolar binders toward MYCN G4 target. Again, analog MY-2 showed the highest binding affinity with 0.2±0.03 μM, however, it was not chosen for further test due to aggregation potential/poor solubility. Taking all the SAR data into consideration, MY-8 was elected for cell-based assays. Results are tabulated in Table 3 above.

### Example 6

### Effect on MYCN-MYCNOS Expression after MY-8 Treatment

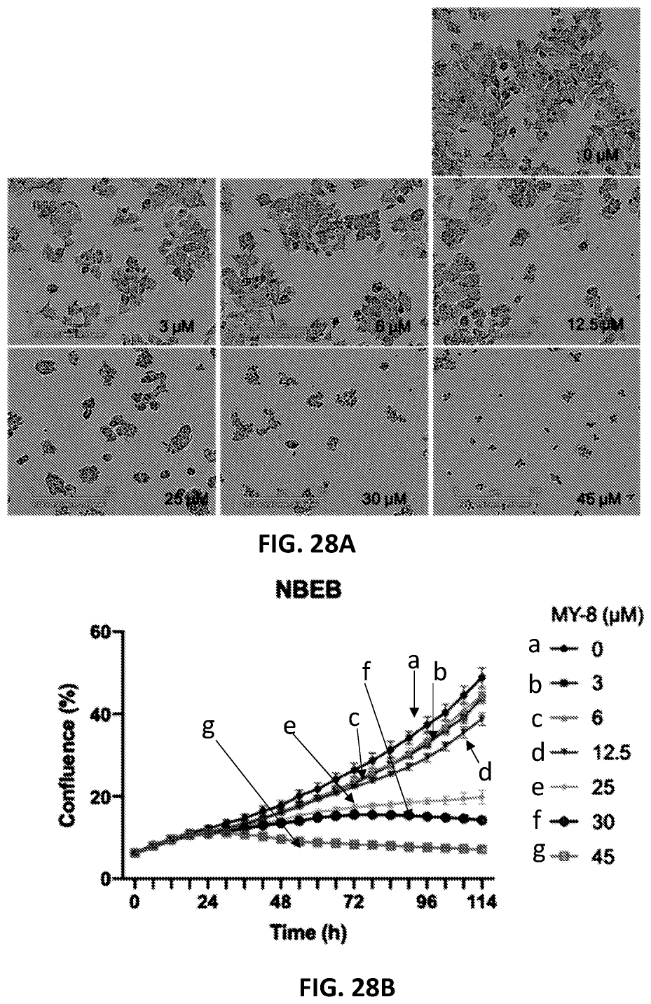

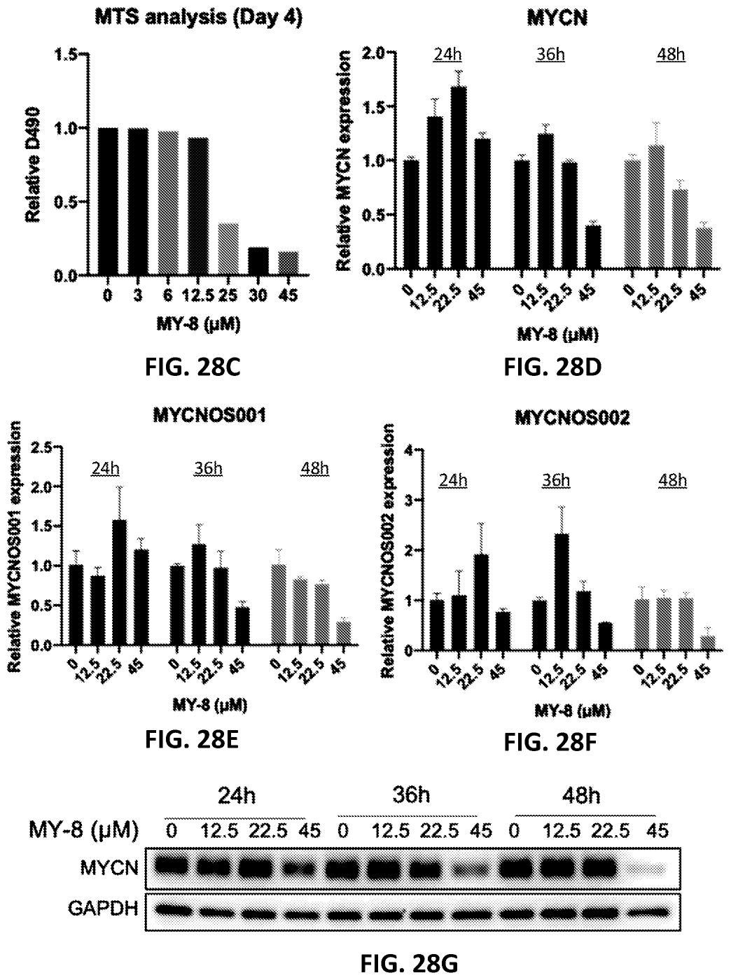

To evaluate the effects of MY-8 on MYCN gene expression, NBEB cells were cultured and treated with different concentrations of MY-8. The effects of MY-8 on cell viability were evaluated using an Incucyte live cell imaging system. Based on a 4-day measurement of cell confluence after single treatment of MY-8, a significant inhibition of NBEB cell growth was observed (FIGS. 28A and 28B). With the increase of compound concentration up to 45 μM, a CC₅₀ of 20.5 μM was confirmed by MTS assay (FIG. 28C). Next, mRNA levels of genes including MYCN, as well as two MYCNOS transcripts (MYCNOS001 and MYCNOS002), were measured at different time points (24, 36, and 48 h) after MY-8 treatment using qRT-PCR (FIGS. 28D-28F). At higher concentrations of MY-8, clear decreases of mRNA level were observed for all three transcripts, indicating that the treatment with MY-8 decreased expression levels of both MYCN and MYCNOS. Interestingly, MYCNOS002 levels decreased as early as 24 h, earlier than the other two transcripts. Levels of N-Myc protein were also measured using Western blotting (FIG. 28G). Here, results aligned with qPCR experiments, and levels of N-Myc protein were decreased upon treatment with MY-8 in a dose-dependent fashion.

As shown by data herein, the biophysical analyses using multiple orthogonal techniques described herein establish that compounds, such as compound MY-1 and MY-8, bind to the MYCN G4 in an atypical manner by interacting with the unique fold formed by the G-tetrad and hairpin. And, in some examples, biological evaluation of MY-8 revealed that it decreased levels of MYCN as well as MYCNOS, downregulating levels of both gene products at the RNA and protein level.

Data and information disclosed herein establish that targeting higher order structure within DNA G4s provides a new avenue for developing selective inhibitors that control the expression of undruggable oncogenes. Indeed, higher complexity RNA G4s have been successful frameworks for highly selective small molecule binding. While these examples are mostly evolved fluorescent RNA aptamers, they are an elegant demonstration that complex structures containing G4s can provide unique pockets for highly selective small molecule recognition.

In the broader sense, small molecule recognition of G4 elements in the presence of lncRNA promoters may be a way to control lncRNA expression. While lncRNAs represent important drivers in multiple cancer types, there are few examples of small molecules capable of controlling their expression or function. Data provided herein indicates that the unique folds formed by these complex structures could be valuable functional targets for small molecules as well, facilitating the targeting of disease-relevant genes including protein coding as well as non-coding gene products.

### Example 7

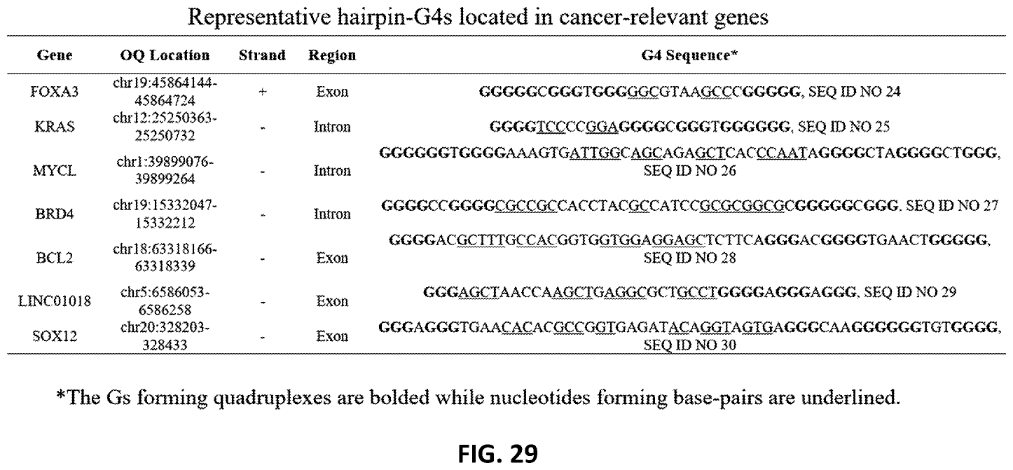

Given the potential for hybrid G4s containing hairpins as targets, the existence of other G4s with similar structures in human genome was further explored. Using data from genome-wide G4-seq, -2 million unique loops (>7 nt) were identified between G-tracts of G4s embedded in 407,491 regions of observed quadruplexes (OQs). After a hairpin-folding test using UNAfold (46) (hybrid-ss-min package at 37° C.), 33,912 of OQs were found to contain a hairpin structure (˜8.3% of the total OQs), in agreement with a previous computational prediction. Among the hairpin-G4 regions, 58% were associated with protein coding genes while 13% were associated with non-coding RNAs. Several of the hairpin-containing G4s were located in genes of cancer-relevant proteins (such as FOXA3, KRAS, MYCL, and BRD4), (see FIG. 29 for representative hairpin-G4s). This example shows that higher complexity G4s that contain embedded hairpins are prevalent throughout the genome and are often associated with oncogenes or lncRNAs.

### Example 8

In this example, SPR binding assays were performed to analyze binding activity of representative bivalent compounds of the present disclosure. The compounds of this example included a non-canonical binder component and a G4 stacker component. A BIAcore 3000 (GE Healthcare) instrument was used. A CM5 SPR biochip was used and primed with running buffer (10 mM Tris, pH7.0, 100 mM KCl, 0.005% Tween 20, 5% DMSO). To immobilize the compounds onto the chip surface, the flow rate was set as 5 μL/min. Then the carboxylated dextran on the chip surface was activated by EDC/NHS (0.4 M/0.1 M) aqueous solution for 15 min, followed with an injection of streptavidin (SA) solution (0.2 mg/mL in 10 mM sodium acetate buffer, pH 4.5) for 30 minutes. After the immobilization amount of SA, the surface was quenched by injecting 1 M ethanolamine (EA) aqueous solution (pH 8.5) for 10 min and regenerated with 10 mM NaOH for 2 min to remove the physical adsorption. Furthermore, biotinylated MYCN G4 DNA was prepared at 5 μM in the annealing buffer and heated up to 95° C. for 5 min followed with slowly cooling down to room temperature within 1 h. After annealing, a total of 150 μL solution was injected in the Fc 2 of SPR system for 30 min to immobilize DNA onto the chip surface. The compound solutions were then tested once the baseline was stable.

To detect the binding signal as well as the binding affinity, a higher flowing rate (25 μL/min) was used in both Fc 1 (blank) and Fc 2 (target). Each of the compound solutions was prepared at 20× designed the concentrations in DMSO, and then diluted into non-DMSO running buffer, resulting in a final concentration of 5% DMSO. Then, a total of 50 μL compound solution was injected in Fc 1-2 flow path for 120 s for association, followed with 200 s running of buffer for dissociation. An injection of 50 μL regeneration buffer (1 M KCl) could be performed between two samples if necessary. The final binding curve was obtained by referencing the blank channel (Fc 1). To determine the binding affinity (K_{D}), a series of diluted compound solutions were injected and K_{D} was calculated by BIAevaluation 4.0 software (GE Healthcare) using Langmuir 1:1 binding model.

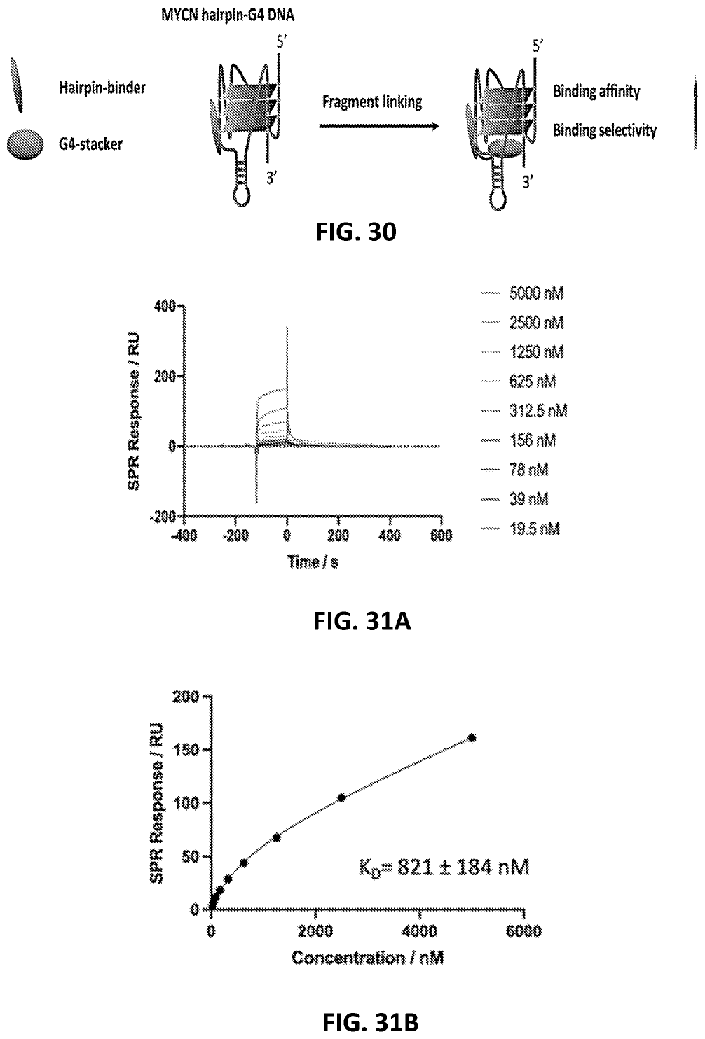

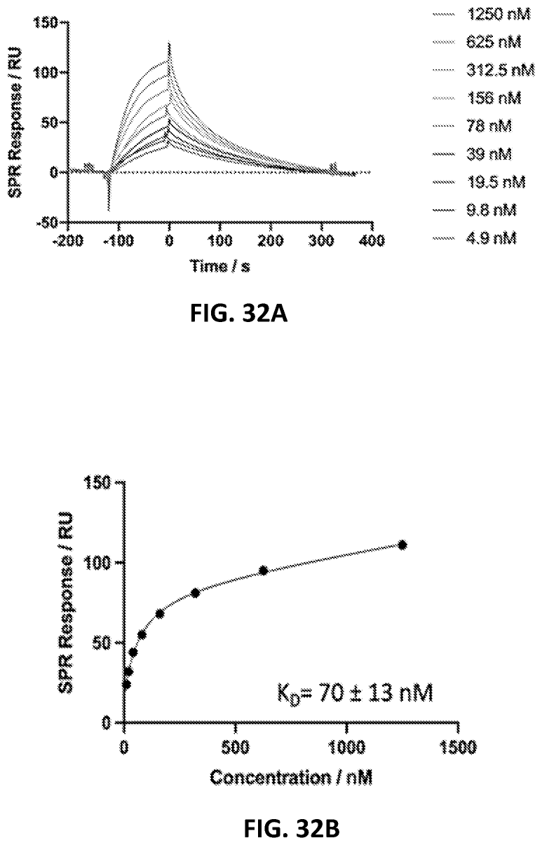

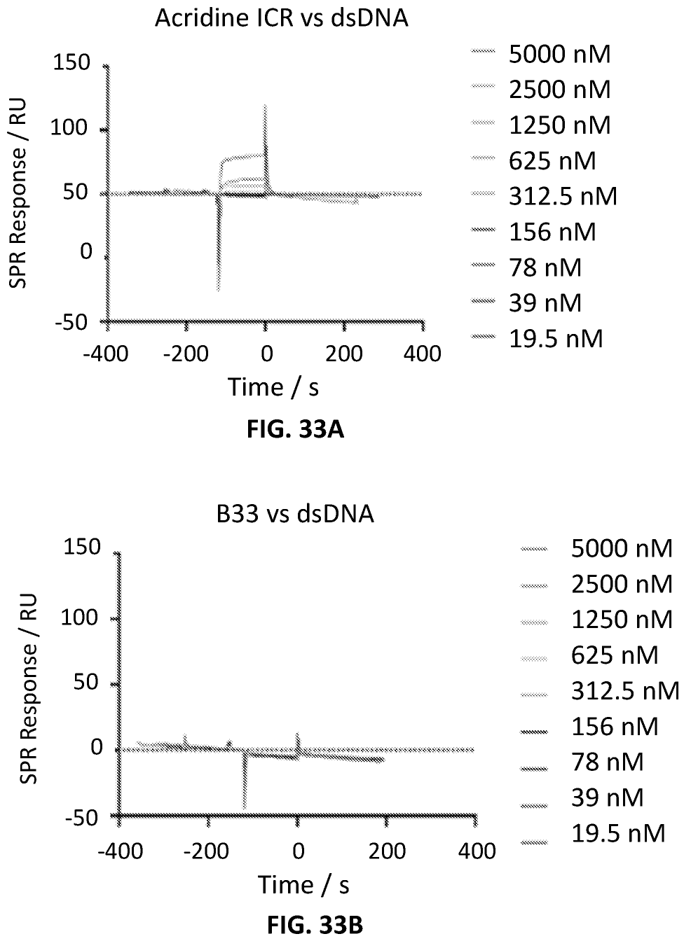

Results for particular implementations performed for this example are shown in FIGS. 30, 31A, 31B, 32A, and 32B. A schematic illustrating the binding of the bivalent compounds of the present example, as is currently understood, is provided by FIG. 30. Acridine ICR 191 had good binding signals during the titration (other acridinyl compounds that were evaluated included 9-aminoacridine, acridine Cl, and acridine NH₂), showing a K_{D} of 821±184 nM (FIGS. 31A and 31B). The binding between compound B33 and MYCN G4 was quantified by SPR titration. Compound B33 showed a great improvement in binding affinity (K_{D}=70±13 nM) compared to either reported MY-8 (1.5±0.3 μM by SPR) or acridine ICR 191 (FIGS. 32A and 32B). Besides, the binding curves of compound B33 showed different kinetics from that of acridine ICR 191 (fast-on/off). Moreover, the newly developed compound did not show any binding to dsDNA on SPR, even with 5 μM compound injection. In contrast, the acridine ICR showed dose-dependent binding signals to the dsDNA, due to the groove-binding and intercalation (FIGS. 33A and 33B). These results showed that compound B33 had an excellent binding preference toward G4 structure over B-DNA structure, which would lead to a better specificity in complex solutions.

### Example 9

In this study, FIA was used for validating the bindings between certain bivalent and MYCN G4 oligos. To measure the binding affinity, a solution of each compound was prepared in triplicate in a 96 well-plate (Costar, black side clear bottom), resulting in a final concentration of 100 nM (5% final DMSO in the working solution). Unlabeled MYCN G4 DNA was folded based on the above-mentioned annealing method, then added into the well plate resulting in different concentrations by a seral dilution. The plate was incubated at room temperature for 30 min, followed by centrifuging at 500 rpm for 2 minutes. The fluorescence intensity was then quantitatively recorded by a Synergy Mx microplate reader (BioTek) at Ex 340 nm/Em 500 nm. The fluorescence intensities were then normalized, and the binding affinity was calculated by fitting the curve using one-site total model in GraphPad Prism 8.3.1 software.

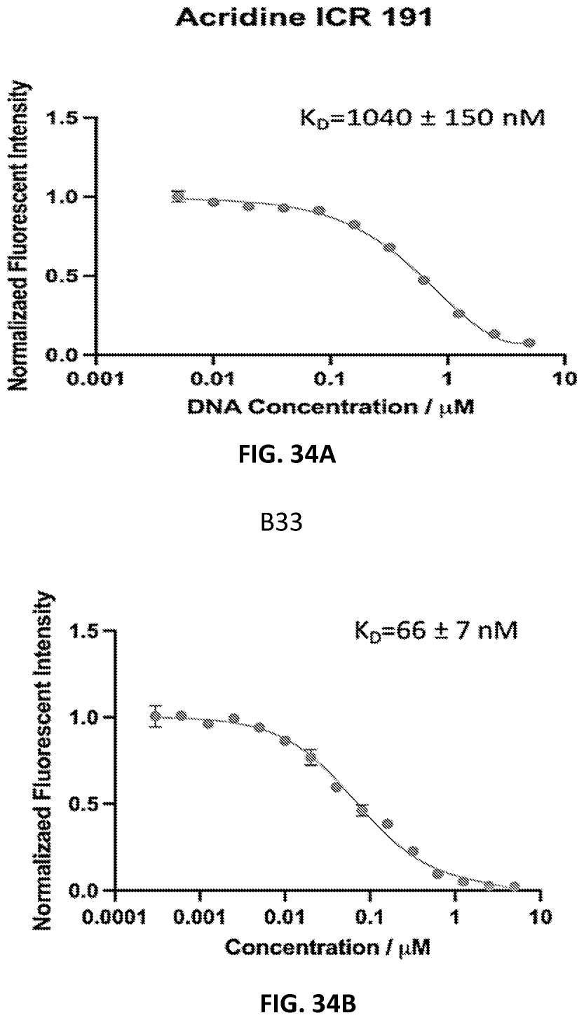

The FIA study was also conducted by taking advantage of the fluorescent property of acridine-derived molecules. Acridine ICR and compound B33 were excited at 340 nm, showing a fluorescent emission peak at ˜500 nm. By titrating the folded DNA G4 into 100 nM small molecule solution, the curve of dose-dependent response was obtained and fitted. As a result, acridine ICR 191 and compound B33 showed 1040±150 nM and 66±7 nM, respectively (FIGS. 34A and 34B). This result confirmed the tight binding between compound B33 and MYCN hairpin-G4.

### Example 10

In this example, a small-scale DNA G4 microarray containing 12 different G4 oligos as well as non-G4 oligos (dsDNA and ssDNA) was fabricated and tested (Table 4). To start with, a SA-coated glass surface was prepared. An amino-functionalized glass slide was firstly modified with a solution of N, N′-disuccinimidyl carbonate (DSC, 1.0 M) and N, N-diisopropylethylamine (DIPEA, 1.0 M) in DMF overnight at room temperature. After being washed successively with EtOH, Milli-Q water (5 min each) and dried with N₂ gas, the slide was incubated with 1 mg/mL of SA solution for 12 h (overnight) in a 4° C. fridge, followed by blocking the surface with EA buffer for 30 minutes. Then, the slide was rinsed with PBS buffer and Milli-Q water, and dried b centrifugation (1,700 g, 2 min).

TABLE 4

| | | | SEQ |
|---|---|---|---|
| | | | ID |
| No. | Name | Sequence | NO |
| 1 | MYCN | AGG GGG TGG GAG GGG GCA TGC | 7 |
| | | AGA TGC AGG GGG T | |
| 2 | hTERT | AGG GGG CTG GGC CGG GGA CCC | 31 |
| | | GGG AGG GGT CGG GAC GGG GCG | |
| | | GGG T | |
| 3 | BCL2 | CGG GCG GGA GCG CGG CGG GCG | 32 |
| | | GGC GGG CA | |
| 4 | RB1 | CGG GGG GTT TTG GGC GGC | 33 |
| 5 | VEGF | CGG GGC GGG CCG GGG GCG GGG | 34 |
| | | T | |
| 6 | KRAS | AGG GCG GTG TGG GAA GAG GGA | 3 |
| | | AGA GGG GGA GGC AG | |
| 7 | c-MYC | TGG GGA GGG TGG GGA GGG TGG | 35 |
| | | GGA AGG | |
| 8 | mTOR | GGG GAA GGC GGG CGG TGG GGC | 4 |
| | | AGG GGG | |
| 9 | Telomeric | TTA GGG TTA GGG TTA GGG TTA | 6 |
| | DNA | GGG TTA | |
| 10 | c-kit | AGG GAG GGC GCT GGG AGG AGG | 36 |
| | | G | |
| 11 | dsDNA | CAA TCG GAT CGA ATT CGA TCC | 37 |
| | | GAT TG | |
| 12 | HIF1-α | GGG AGG GAG AGG GGG CGG G | 38 |
| 13 | NRAS | UGU GGG AGG GGC GGG UCU GGG | 5 |
| 14 | ssDNA | TGT CCC CAC ACC CCT GTC CCC | 39 |
| | | ACA CCC CTG T | |

In parallel, biotin-labeled DNA/RNA oligos were prepared in annealing buffer resulting in 5 μM stock solutions, followed by annealing (according to above methods). Then all the oligo solutions were transferred into a 384-well plate. Next, the microarray printing was conducted by a robotic arrayer (Nanoprint, Arrayit, USA) with a humidity of 60%. After the microarray was fabricated, the slide was placed in a slide box with a piece of wet Kimwipe tissue and incubated at 4° C. for 2 h.

To incubate with the fluorescent compounds (compound B33, thiazole orange (“TO”), and amsacrine), the slide was thoroughly rinsed with PBST, PBS, and water to remove the unbounded oligos. Then, the slide was dried by centrifugation (1,700 g, 2 min) and quickly assembled with microarray gaskets (Agilent, USA), followed by loading the compound solutions. After 1 h incubation, the slide was washed and dried with the above-mentioned method. Finally, the microarray slide was imaged by a fluorescence scanner (Mapix) using green channel and the fluorescence intensities were quantified.

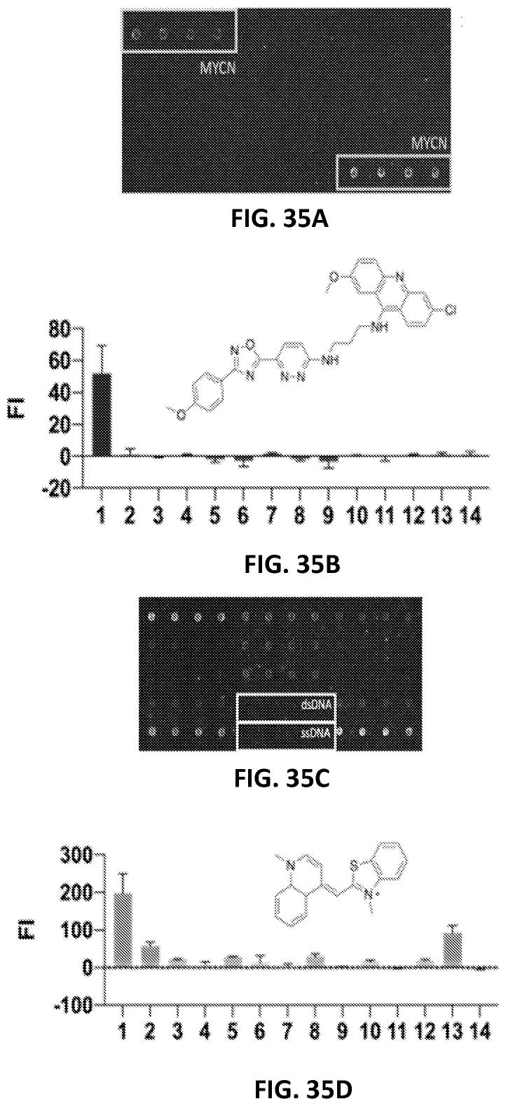

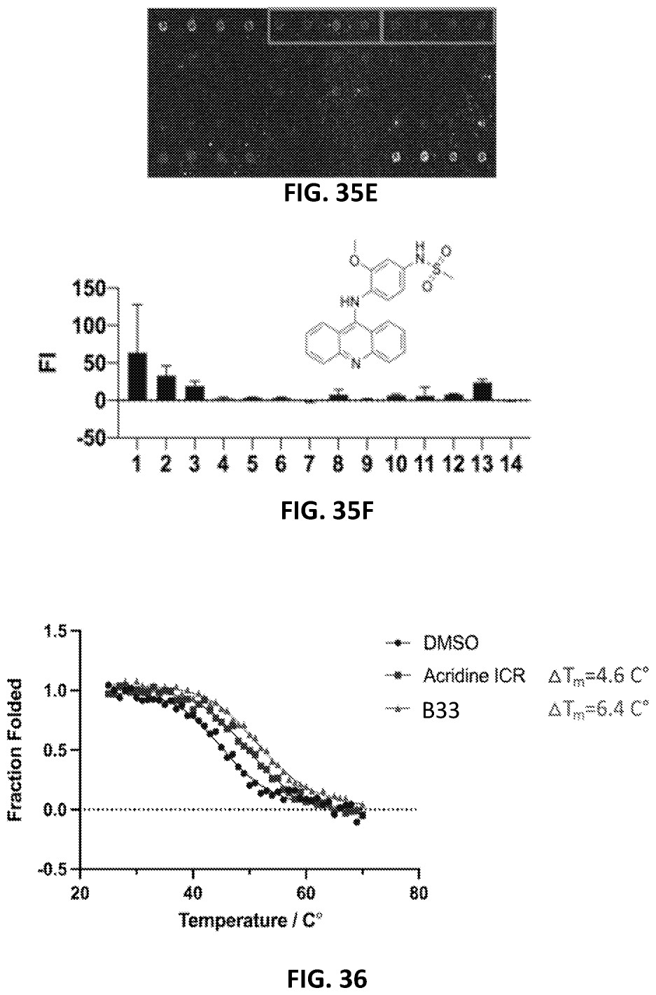

At 2.5 μM, compound B33 showed relative higher signal-to-noise ratio (SNR) and lower background, compared to other incubating concentrations (data not shown). As a result, only MYCN spots lighted up in the compound B33 treated microarray, indicating a promising binding selectivity against the corresponding target (FIGS. 35A-35F). Meanwhile, another two reported hairpin containing G4 DNAs (hTERT and BCL2) didn't show fluorescence, suggesting that the molecular recognition highly depended on the hairpin in MYCN G4. In addition, no binding to dsDNA/ssDNA was observed, which was consistent with the G4-binding behavior of compound B33. In contrast, TO, as a general G4 binder, exhibited the binding to most of the oligos except for dsDNA/ssDNA, suggesting a broad-spectrum G4 binding capability. Amsacrine, which shared the acridine core with compound B33 but had a shorter tail (benzylic sulfonamide), showed binding to several G4s in the microarray, especially NRAS RNA G4 and the three hairpin-G4s (MYCN, hTERT and BCL2). These results demonstrated the promising binding selectivity of compound B33 to MYCN hairpin-G4 over other G4 structures.

### Example 11

In this example, the folding of G-quadruplex structure was characterized by circular dichroism using a J-1500 circular dichroism spectrometer (Jasco). To optimize the buffer condition, unlabeled MYCN oligonucleotide was prepared in different annealing buffer conditions at 5 μM concentration. The annealing procedure was the same as mentioned above. CD spectra were recorded from 320 to 200 nm at 25° C. with an interval of 1 nm. Each spectrum was obtained by averaging the signals of three replicate scans.

For CD melting assay, MYCN oligonucleotides were prepared in KCl buffer (10 mM sodium phosphate, pH7.0, 5 mM KCl) at 5 μM concentration. To test the stabilization effect of the compounds, MYCN G4 DNA samples were mixed with/without the compound (final solution containing 5% DMSO) at designed concentration. Then a total of 300 μL solution was added in a cuvette and heated from 20 to 80° C. in a CD spectrometer with an interval of 1° C. To calculate the melting temperature (Tₘ), the peak of CD spectrum at 263 nm was tracked, and ellipsometry vs temperature was plotted and fitted using a nonlinear sigmoidal dose-response model with a variable slope in GraphPad Prism 8 software.

The thermal melting assay using CD was carried out to investigate the effect of the compound on MYCN G4 stability. Compared with acridine ICR 191 (ΔTₘ=4.6° C.), compound B33 showed a much higher ΔTₘ (6.4° C.), which could be attributed to the synergistic effect of the two fragments in compound B33 (FIG. 36). This result agreed to our hypothesis that the bivalent interaction could not only improve the binding affinity but also increase the thermal stability of the MYCN hairpin-G4, due to the introduction of more contacts between the small molecule and the oligo.

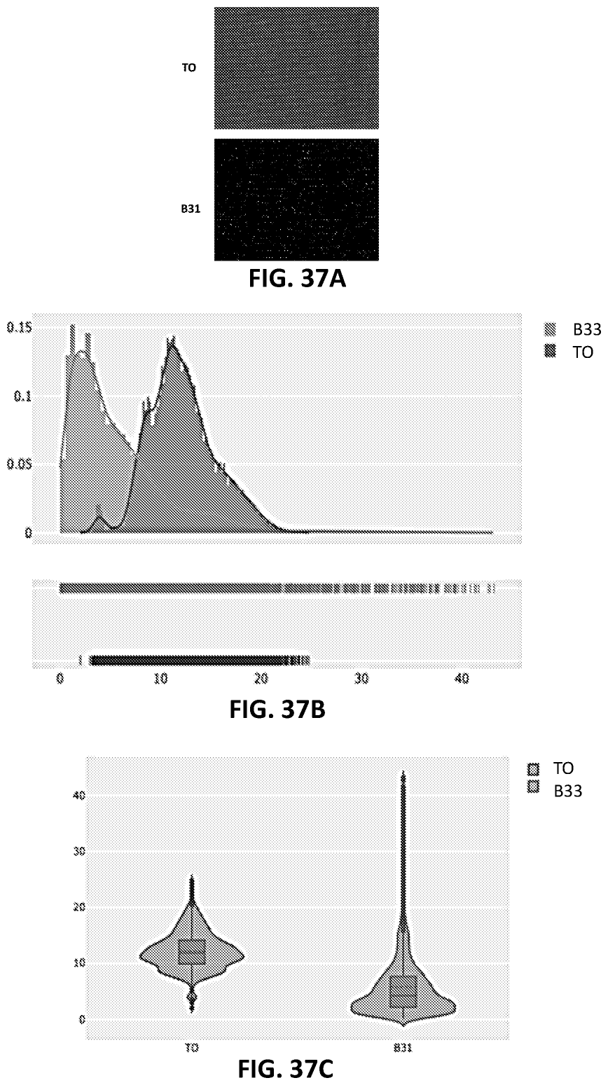

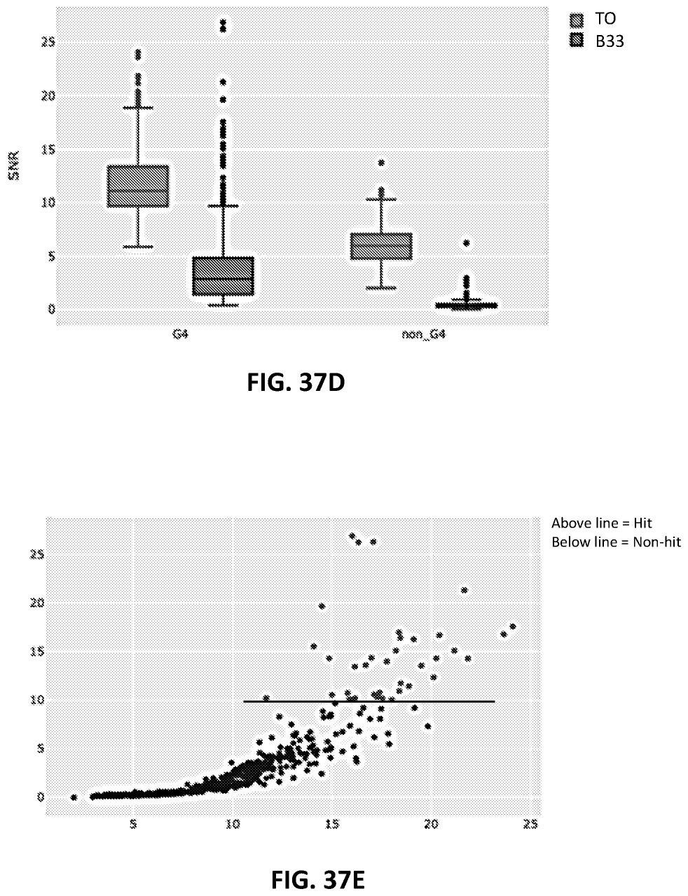

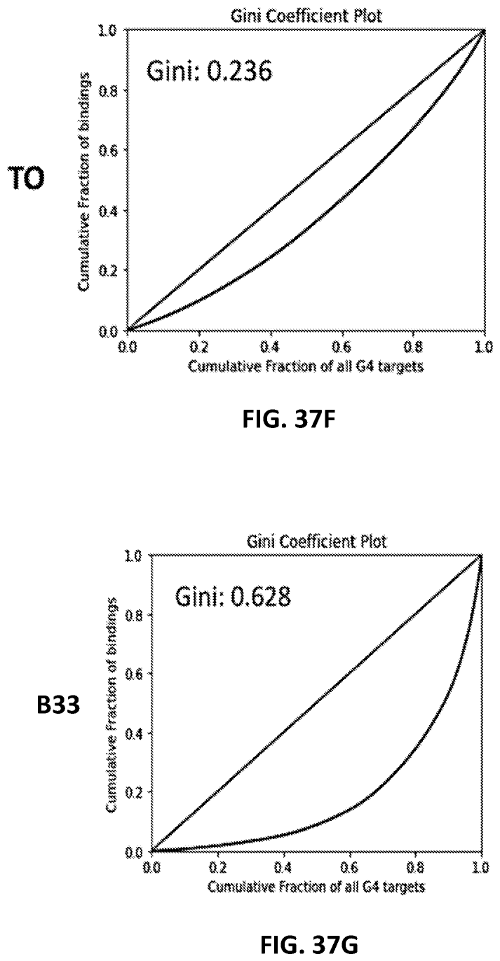

To further characterize the binding selectivity of compound B33, high-density and large-scale DNA microarrays that contained ˜19,000 different G4 sequences were fabricated and used. In this microarray, 340 reported G4s (G-rich sequences) as well as 280 non-G4s (C-rich sequences) were designed as positive and negative controls. Additionally, over 17,000 canonical G4 oligos with short loops (1˜7 nt) were also designed and synthesized by varying the loop sequences to meet the diversity of G4 structure. After folding the surface-grafted DNA oligos in high concentration of KCl buffer, TO and compound B33 solutions (500 nM) were incubated with the microarray, respectively. Then the slides were thoroughly washed, dried, and imaged by a fluorescent scanner using green channel. As a result, TO-treated microarray presented a global fluorescence and the fluorescent signal was evenly distributed (except for some negative control spots), which indicated that almost all the G4s were folded (FIG. 37A). However, compound B33 slide only showed a few bright spots while the other spots remained dark as background, suggesting that compound B33 had a strong preferential binding behavior. By quantifying the fluorescence intensity of each spot, the bindings of TO and compound B33 were further investigated. The kernel density estimate (KDE) plot and violin plot represented the distribution of the binding signals throughout the whole microarray. As shown in FIG. 37B, the two bands corresponding to the two small molecules were well separated. Compound B33 spectrum spanned a much wider range than that of TO. In the violin plot (FIG. 37C), the mean SNR value of TO group was 12, much higher than that of the compound B33 group. Besides, TO group had a smaller error bar which confirmed the evenly distributed binding signals in the microarray. The violin plot of the compound B33 group presented a needle-like shape at top part and a body at very bottom, indicating that there was a strongly biased binding event (FIG. 37C). By focusing on the 620 G4 and non-G4 controls, TO showed binding to both G4s and non-G4s, although the average SNR of the former was significantly higher than that of the latter (FIG. 37D). Compound B33, as a contrast, showed some binding to G4s while there was no binding observed in non-G4 group. The result showed that compound B33 had a preference to G4s over non-G4 structures, which was consistent with the global analysis of the microarray. By comparing the SNR values between TO and compound B33 groups, the scatter plot deviated from the 45-degree line (FIG. 37E), again indicating that the binding behavior of compound B33 was quite different from that of a non-selective binder (TO). Furthermore, the selectivity of the two compounds were quantitatively profiled by using Gini coefficient. Gini coefficient, initially used as an economic concept, has been applied in profiling the selectivity/promiscuity of kinase inhibitors as well as RNA binders. A higher Gini value represents a better selectivity of a compound (Gini coefficient>0.75 is considered an excellent selectivity). The Gini coefficient based on the SNRs in the microarray. As a result, TO and compound B33 showed Gini coefficient values of 0.236 and 0.628 (FIGS. 37F and 37G, respectively). The high Gini value confirmed that compound B33 had a relatively promising selectivity among G4s in a quantitative manner.

### Example 12

In this example, DMS-footprinting was conducted by using 5′-Cy5-labeled MYCN hairpin-G4 DNA. 5 μM of the oligo was annealed in optimized buffer condition (10 mM Tris, pH 7.0, 10 mM LiCl and 10 mM NaCl) by heating up to 95° C. followed with slow cooling. The folded DNA was subsequently incubated with compound B33 at different concentrations (0, 10, 25, 50, 100 nM) for 30 minutes. Then, the solutions were subjected to 1% DMS treatment for 10 min at room temperature and the reaction was stopped by 2.5 M NH₄OAc and 0.1 M β-mercaptoethanol. The treated DNA was purified by phenol/chloroform/isoamyl alcohol and ethanol precipitated. The DMS-modified DNA was then cleaved using 10% piperidine at 90° C. for 30 minutes. The solution was dried and washed twice with 100 μL water using SpeedVac vacuum concentrator and dissolved in nuclease-free water. The treated DNA samples were resolved on a 17% denaturing polyacrylamide gel and visualized on a Typhoon Imager (Amersham) followed by processing with ImageJ software.

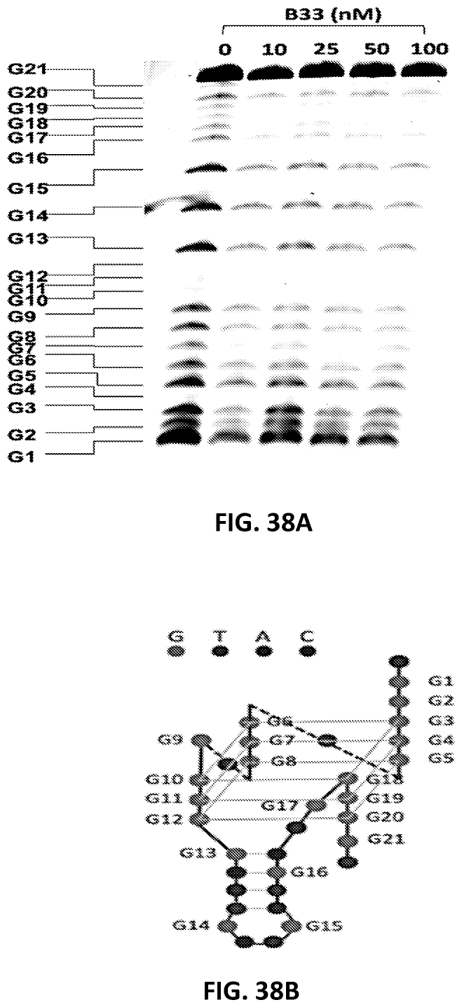

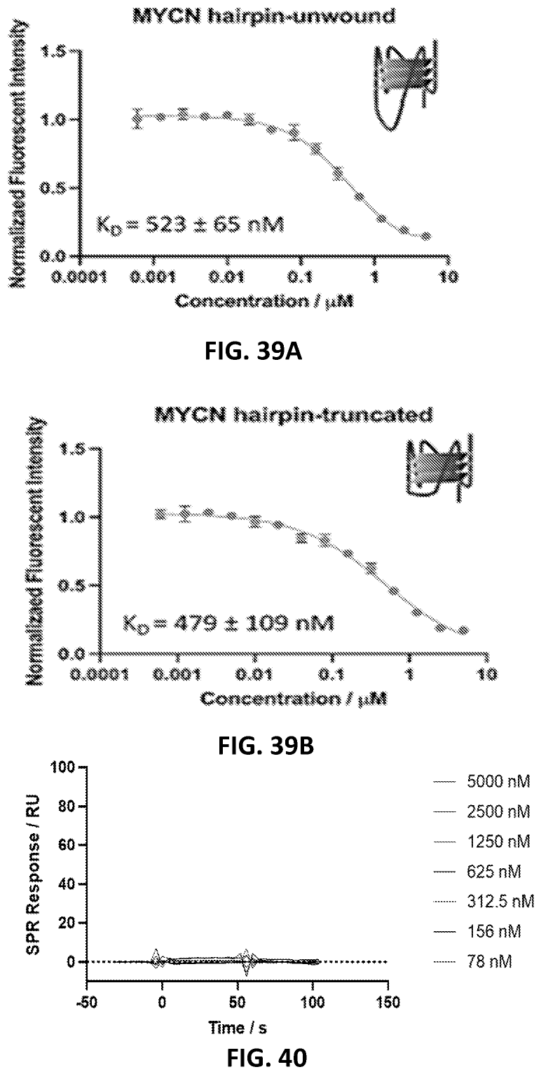

Buffer optimization was conducted to observe the small molecule induced G4 formation. By CD spectrum study, 10 mM LiCl+10 mM NaCl was selected as the salt condition in Tris-HCl buffer. After 5′-Cy5-labeled MYCN G4 was annealed and incubated with different concentrations of compound B33, the DMS-footprinting assay was carried out (FIG. 38A). In absence of the compound, bands of G13-15 were more intense corresponding to the hairpin region, while some bands of the Gs were dim because of the quadruplex formation (FIG. 38B). With the addition of compound B33, the oligo was induced to form G4 structure due to the protection of G2-9, G10-12, and G17-21. Besides, G13-15 were observed to be partially protected, indicating that during the formation of the quadruplex, this hairpin region was also contacting to the molecule. This result was consistent with the binding study using unwound and truncated MYCN G4s (FIGS. 39A and 39B, respectively), again confirming that the hairpin was involved in the binding. However, in another test, a duplex-containing RNA G4 (MANGO II) showed weak binding to compound B33 by SPR (FIG. 40), because the duplex and quadruplex structures were separated instead of forming a junction (PDB: 6C63). Without being bound to a single theory, it currently is believed that this result may indicate that the molecular recognition is facilitated by the existence of the tertiary structure formed by a hairpin-quadruplex.

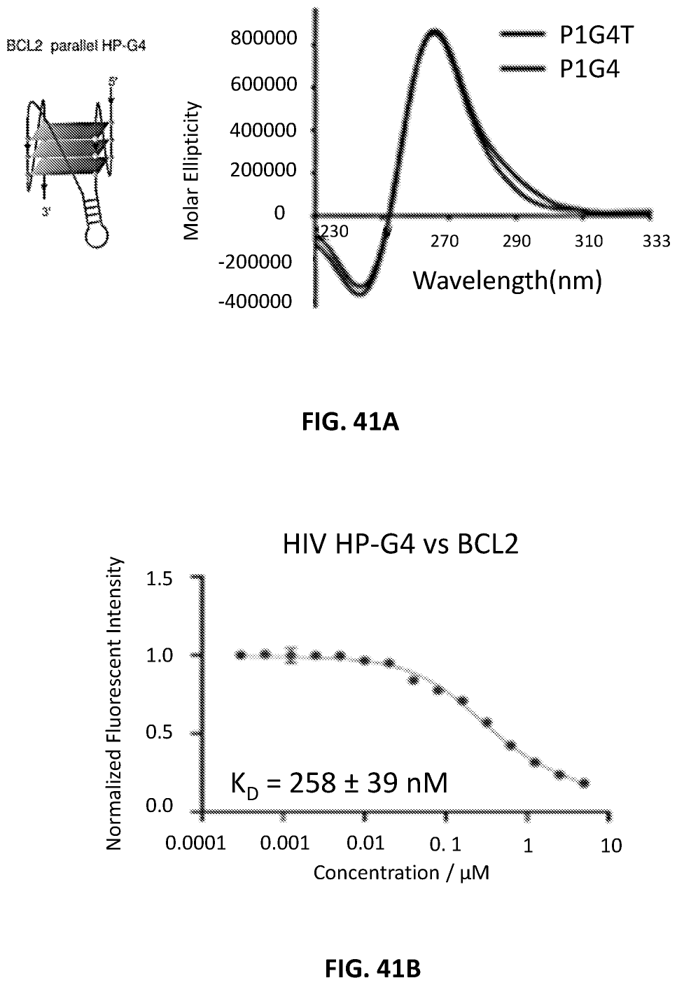

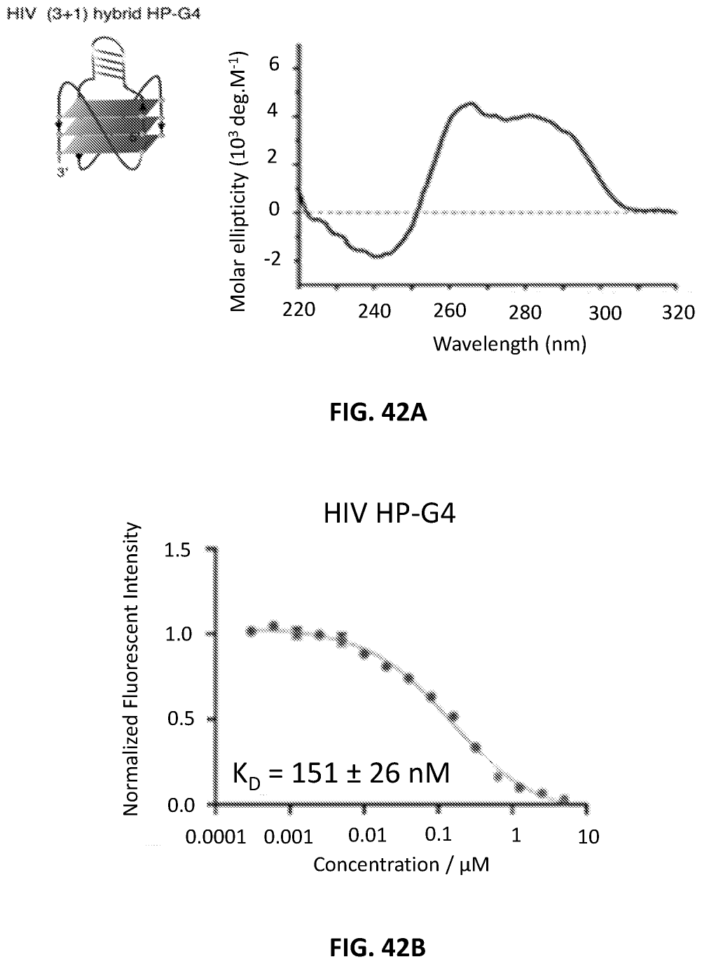

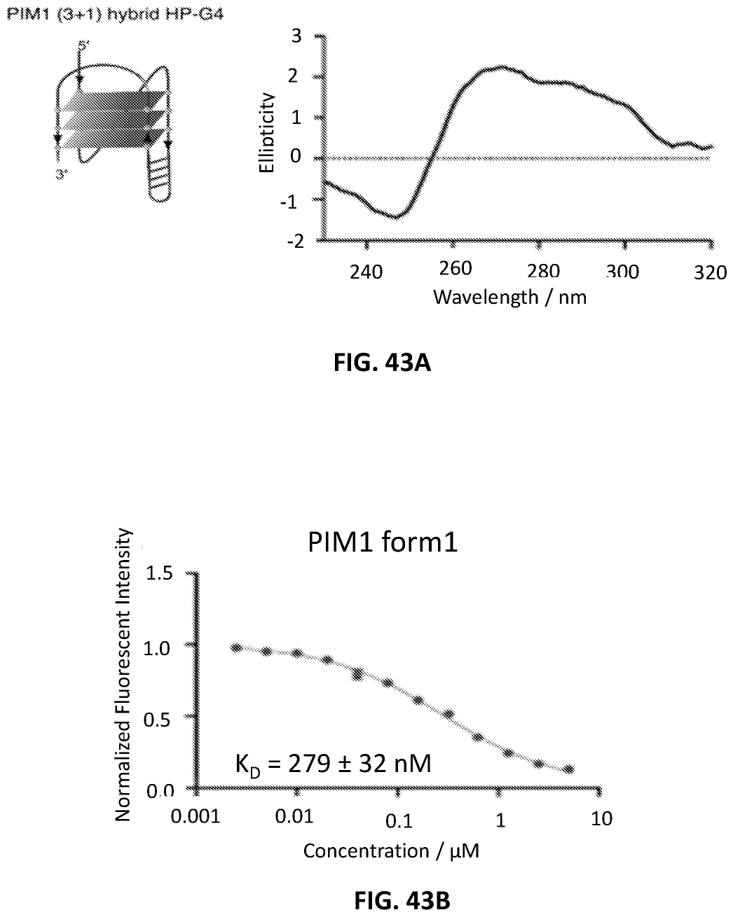

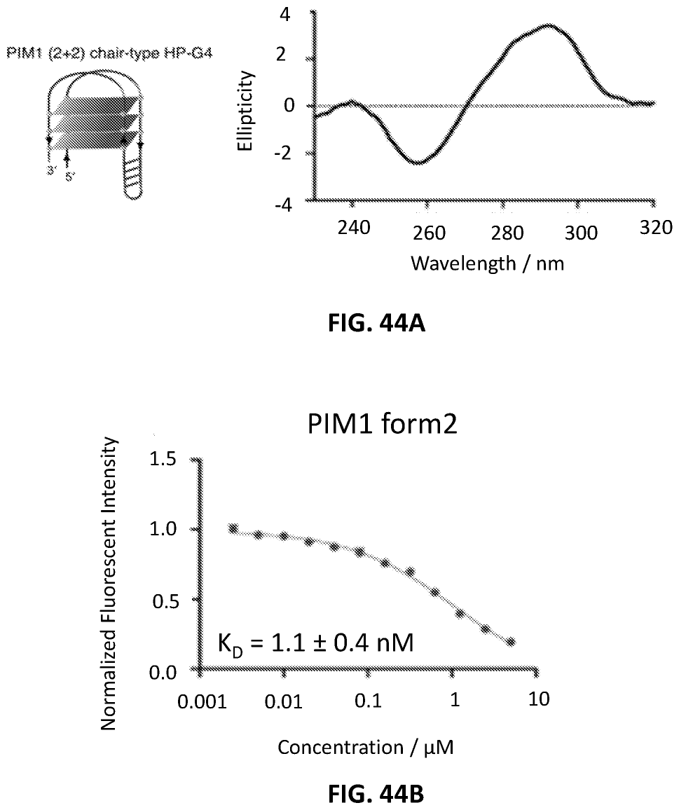

In addition to the DMS-footprinting study, the bivalent binding mode of compound B33 toward MYCN hairpin-G4 was also investigated by comparing HP-G4s with different folding types. Four reported HP-G4s with parallel structure (BCL2), (3+1) hybrid structure (HIV and PIM1-form1), and (2+2) chair-type structure (PIM1-form2) were examined by FIA binding test using compound B33 (FIGS. 41A, 41B, 42A, 42B, 43A, 43B, 44A, and 44B). As a result, BCL2 HP-G4 showed a K_{D} of 258±39 nM, which was almost 4 times weaker than that of MYCN HP-G4. For (3+1) hybrid G4s, HIV and PIM1-form1 HP-G4s showed 151±26 nM and 279±32 nM bindings to the small molecule. For anti-parallel structure, PIM1-form2 HP-G4 showed a much weaker binding affinity (1.1±0.4 μM) toward compound B33, which totally lost the synergistic effect of the two fragments. This observation might be attributed to the conformational difference from parallel MYCN G4 that separated the plain G-tetrad and the groove. These results indicated that the binding event between the developed molecule (compound B33) and the HP-G4 was highly related to the conformation of the hairpin-quadruplex junction, which would determine the shape of the binding site.

### Example 13

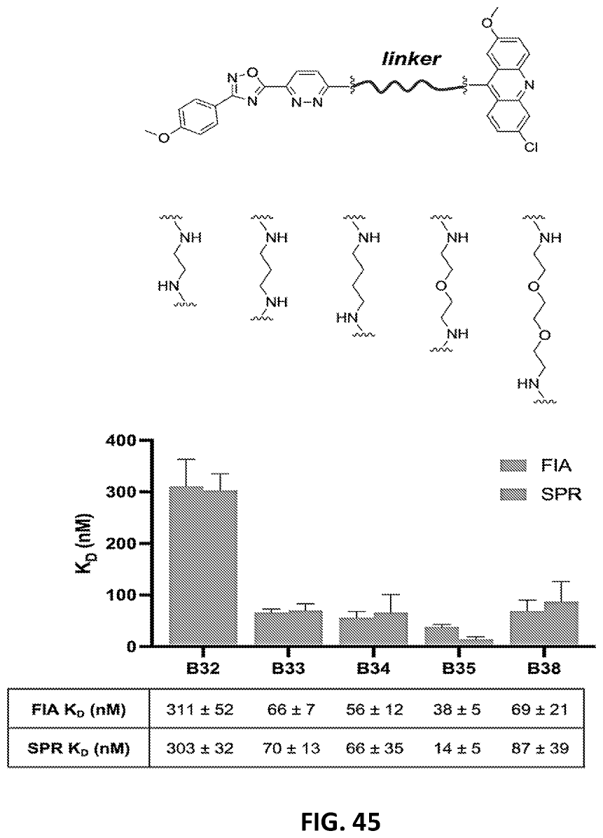

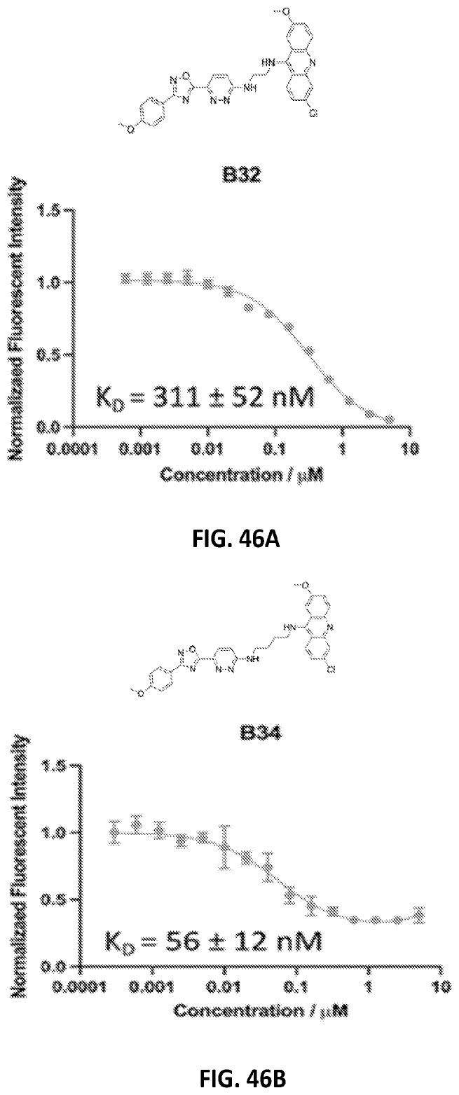

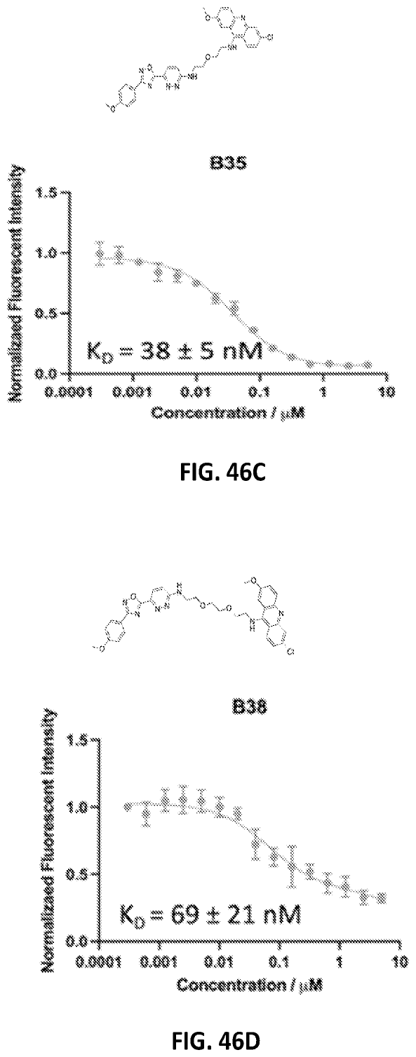

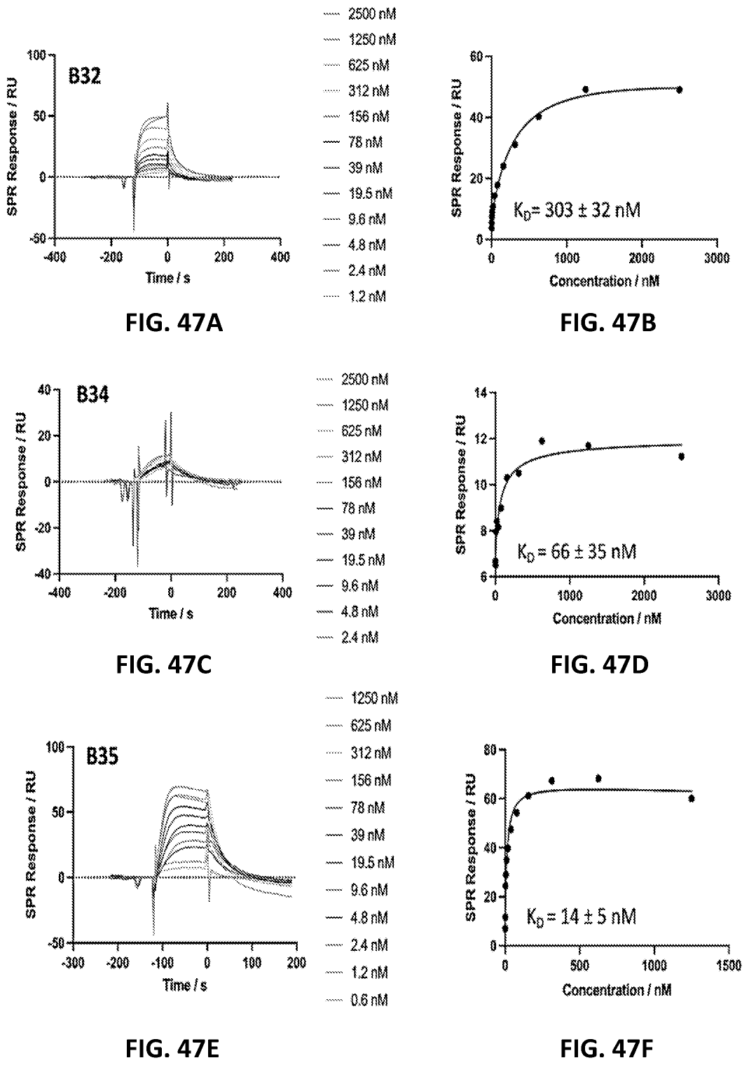

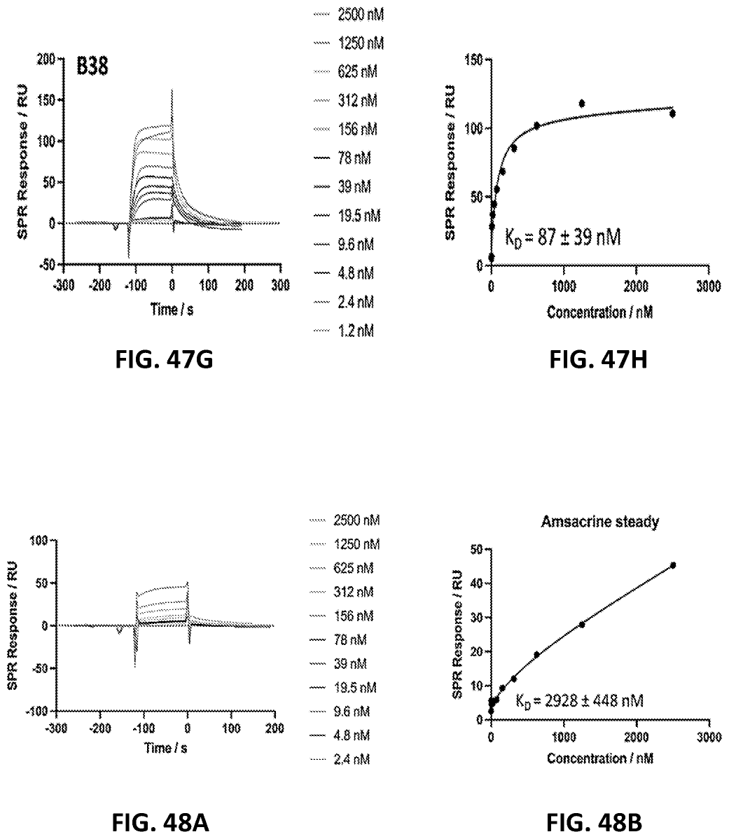

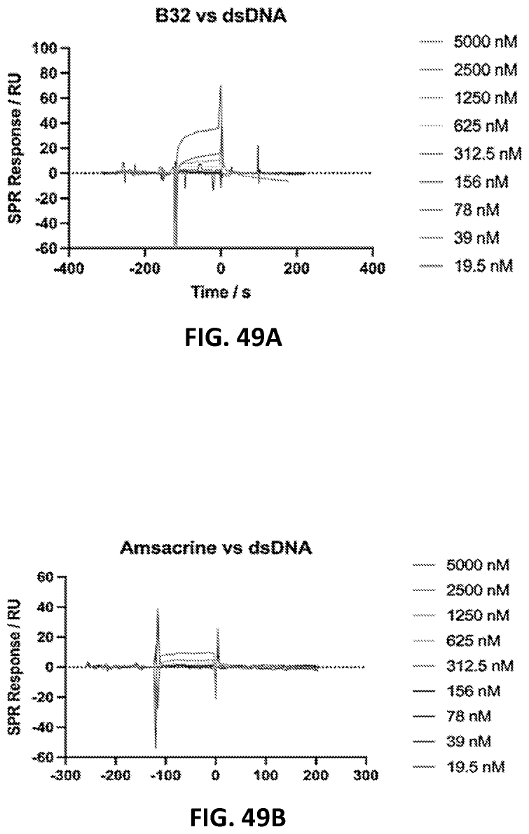

In this example, different linker groups for bivalent compounds were evaluated. Linkers having different lengths and/or functional groups were evaluated and results are summarized in FIG. 45. Specific results for K_{D} values for each compound as measured using FIA and SPR are provided by FIGS. 46A-46D and FIGS. 47A-47H, respectively. In this example, B35 showed the lowest K_{D} (38±5 nM by FIA, 14±6 nM by SPR), exhibiting an >10-fold improvement compared to B32 (311±52 nM by FIA, 303±32 nM by SPR). This dissociation constant was comparable with that of the reported antibodies (scFV: K_{D}±30 nM) that bound to parallel G4 structures. When the linker was further elongated (e.g., using a PEG2 group, such as in compound B38), a weaker binding was observed (69±21 nM by FIA, 87±39 nM by SPR), no tighter than that of B33. In addition, amsacrine, which had a short linker, showed much weaker binding (2.9±0.5 μM) to MYCN HP-G4 (see FIGS. 48A and 48B). And, compound B32 and amsacrine bound to dsDNA at low micromolar level (see FIGS. 49A and 49B). In particular example, the PEG linker in compound B35 not only facilitated fitting the two fragments into the binding sites with proper confirmation, but also contributed to the water solubility, which can facilitate cell assay evaluations.

### IX. Additional Sequences

Sequence for an exemplary genomic DNA sequence encoding human MYCN (SEQ ID NO: 1)

AGGCTGTGACAGTCATCTGTCTGGACGCGCTGGGTGGATGCGGGGGGCTCCTGGGAACTGTG
TTGGAGCCGAGCAAGCGCTAGCCAGGCGCAAGCGCGCACAGACTGTAGCCATCCGAGGACAC
CCCCGCCCCCCCGGCCCACCCGGAGACACCCGCGCAGAATCGCCTCCGGATCCCCTGCAGTC
GGCGGGAGGTAAGGAGCAGGGCTTGCAAACCGCCCGGCGCCCAGGGAAGCGACGAGCGCCGG
GGCAAGGCAAGCCCTGGACGGGATTGCGACGTGCGCACCGGGCGCCCTAATATGCCCGGGGG
ACTGTTTCTGCTTCCGAAACAAAACCATCTCTGGGTTTTCCCAGAAAAGCCAGTTCCAGCCC
CGAAGGCATCCTGGCTAGAGGAGACCCGCCCTAATCCTTTTGCAGCCCTTACCGGGGGGAGT
AATGGCTTCTGCGAAAAGAAATTCCCTCGGCTCTAGAAGATCTGTCTGTGTTTGAGCTGTCG
GAGAGCCGGTGCGTCCCCACCCCAGGCTGGGGTTCTTCTCCAAAGGGTGCCCCTGGAGGAAG
AAGAGGGGGGGATTAGGCAGGGCGAGGCCGCCGCGGTCGCAATCTGGGTCACGGCTGCTCCA
GCTTGGAGGAGAGGCGGCTCTCCCGGCGACCCTCCTCGCGCGGGCGCCCCTGCCATTCCCGG
GAACAGGGGCTCAGCCTCTCCCTCCCTGGAAGAGGACGTTGTCGTGGGTTTGGAAGAGCAGG
GGTGGGCTTAGAGAGCTTCCAATTAAGCTATTGGCAGGAGTATCCCTGCAGCGGGTGAATGC
CGAGGGGCGTTTGCTCAAATTTGGGGAGGGGAAGGATTTGTGGATATGGGTGTCTGTTGTTG
GTCTCTGTCTAGAGAAAGGCTTTTTTTTATTTGCAAAGTTTTCTAAATCCCCTGCTATCATT
TGCACTCCTGAGGTTGCATTTTTACAAAGGGGGTAGAAGGTACTCCAAATACCATTCCCGGT
AGCTGGGTCGGAGAGCCTGGGGCTTCCCCTGAGCAGCCGGCCCCACACCGCTGCGAGTGCGG
TTGTCTGCGTGCTCGTGAGAGCTAGAATTCTGCAGCCAGGAACAGCCCCCTCCCCCAGGCAG
TGCCTTGTGTGAATGAAATGGCAGTTTCCAAAGTTGCGGAGCCTCGCCACCACCCCCTGCAT
CTGCATGCCCCCTCCCACCCCCTGTCGTAGACAGCTTGTACACAAAAGGAGGGCGGGAGGGA
GGGAGCGAGAGGCACAACTTCCTCCACCTTCGGGAGCAGTGGGCAGAGTGGGGGGCTTGGAG
GGAAGATTGGGGAACCTGGTTAGAGGGGGCGCCCATTGCCTATCCCCTCGGTCTGCCCCGTT
TGCCCACCCTCTCCGGTGTGTCTGTCGGTTGCAGTGTTGGAGGTCGGCGCCGGCCCCCGCCT
TCCGCGCCCCCCACGGGAAGGAAGCACCCCCGGTATTAAAACGAACGGGGCGGAAAGAAGCC
CTCAGTCGCCGGCCGGGAGGCGAGCCGATGCCGAGCTGCTCCACGTCCACCATGCCGGGCAT
GATCTGCAAGAACCCAGACCTCGAGTTTGACTCGCTACAGCCCTGCTTCTACCCGGACGAAG
ATGACTTCTACTTCGGCGGCCCCGACTCGACCCCCCCGGGGGAGGACATCTGGAAGAAGTTT
GAGCTGCTGCCCACGCCCCCGCTGTCGCCCAGCCGTGGCTTCGCGGAGCACAGCTCCGAGCC
CCCGAGCTGGGTCACGGAGATGCTGCTTGAGAACGAGCTGTGGGGCAGCCCGGCCGAGGAGG
ACGCGTTCGGCCTGGGGGGACTGGGTGGCCTCACCCCCAACCCGGTCATCCTCCAGGACTGC
ATGTGGAGCGGCTTCTCCGCCCGCGAGAAGCTGGAGCGCGCCGTGAGCGAGAAGCTGCAGCA
CGGCCGCGGGCCGCCAACCGCCGGTTCCACCGCCCAGTCCCCGGGAGCCGGCGCCGCCAGCC
CTGCGGGTCGCGGGCACGGCGGGGCTGCGGGAGCCGGCCGCGCCGGGGCCGCCCTGCCCGCC
GAGCTCGCCCACCCGGCCGCCGAGTGCGTGGATCCCGCCGTGGTCTTCCCCTTTCCCGTGAA
CAAGCGCGAGCCAGCGCCCGTGCCCGCAGCCCCGGCCAGTGCCCCGGCGGCGGGCCCTGCGG
TCGCCTCGGGGGCGGGTATTGCCGCCCCAGCCGGGGCCCCGGGGGTCGCCCCTCCGCGCCCA
GGCGGCCGCCAGACCAGCGGCGGCGACCACAAGGCCCTCAGTACCTCCGGAGAGGACACCCT
GAGCGATTCAGGTAAAGACCGAACTCGGGTCCGGCTGCCTCCCTGGGGCACTGGACCCCGGG
TCGCGTCCCCTTTGTTAGTGCTCGTATGTCTTGGCCTGGGGAGCATTTTGGAGGCAGTGCTA
GGGGCAGAGAGGTCCTGTTTCCCCCAAGTCTCTCCTCGGGGTAAAGAGAAGGGGCTGAGAGA
ATGCCGTTGCAAAAGGGGTGCTCTCCAATTCTCGCCTTCACTAAAGTTCCTTCCACCCTCTC
CTGGGGAGCCCTCCTCTAGGCCATCACGGGCCCTCACCCGGTCCCCCACCTCTCTTTTGCAG
CGCAGTCTGAGGAATAAAATTGGAGAAAGTTGGTGGCTAAACCGGGTGGGGGTTTAGGGGGT
TGCTGGGTGCACTGCCTGGACAGAAACCTGTTAGCGCAGGGGTGAAAGGGACTCTCTGGCCC
AGGTCAGGGGAGGGAAAGACATCCCGAGAAGATTCAAGGGCTGTGCAAAGCCCTGTTTAAGG
CGCAGGAACTTATAGGAGGGTTGCACAGATGGCTAGAGCCGATTTTCTATTCTTTTTCTTTT
TCTTTTTTTTTTTTTTTCAAATGTCGGTACCTTTCCCTTCCCCCATCCTCGGTGGGTGGTGG
GCTATTTGCTCCTGGTGCGTGGCCAGCAGGCGGCGATATGCGAGGCCAGCAGGCGGGCCCGG
GATCTGAAAGGCTGGGGGTGGTGGGGGCACCCTCCCTCCCTCCATTCAGCAGCTGGCTGCAA
GTGCAACAGCAGTTGTGTACATTCTCAGGGGGCCTCCTCTTTCCAGTGTGCAGTGGAAACTG
GCTGTAGTTTTGTCTTCCAGCCTGAATTCCAGGCCTAATTTGAGATGTGAGTTGTATCTGTA
ACCCAGTGCCCTTGAAGGTGAGGGCAGGCACTCAGCAGCCTCTCCAGGAAGGCTCACATCCT
GGGAGGACTCACTGATTAGTTCTATTGTGTTCATTTGTCTGTGTCTTAAGCTGAAGGGAAGA
GTTAAAACCAAGCCTTTCCCTGGGGGTCTGGATGAACAGAACTCAACCCAAAGAGTGGCATT
GCCTTGTCCTTGGAGCAGGGAGCTGGGACCCCCCTTGGACTTTGAAAACCAGTGTTTTCAGA
ATGCAGGTGGATAACAAGCCTAAATTTACTTCTGGGCTGAGGAGAGATCTTTGAGGCTCCTG
GAAGGAAACTTGGTGATAAGCCTCCAGTTTGAAACGGCTCTGTCCCTTTAATGTCTGTGCCT
TGACAGCTTTTGGTGAGGAAGCACTTCCTTCCAACAGCTGTCTTCTTGGCAGAAAACCAAAA
CATTGGCTTAAAGGGACCCACAGACTGGAACAGCCTCACATTTCGGCTTTAGAACAAATCCC
ACAATTGTTCAGCTTTCCGGTCCCCTTCAGATCAAGCAGAAGATATGTTTTGATTTTCATGC
TTGTATTTTAAACAATAATTTTCTACCCCAGCGTGGTAGTCAATGAGGAGAGAGGGGAAGAA
TGCGCACATGATGCTACACGTTTCTGTTGTTGCTGTTATTATTGGTGGCTTTGAGGAGAGCT
GCTCCCATTTGGGGGTTTATACCAACTGTGGATTATGGCTTTGTCATTAAGATTTGATCTTT
GTTAAATGAAAAACTGTTTATTGTATAAAACTCAGGTTTGTGGACGAAAAGTTGTTTTTTTT
CTTCAGTTAATTAAATTGTTCCTCAAGTTTGTTTAAGGACTTAAAATCAAACACAACCATGT
GTAAACTGCTAAATGAGGCTCCTAAAATGAGAGGCCTCAACTCTTTAAGTGTGGAGCTAGAA
ATGTAAATAAGTCCACAGGGCAGACTGGTGATTATGATAAAAGCTACCATTTACTGAGCATC
TGTCTACTAGGCTCAGCTCTATGCTAAGTCTACATGTTATCTGTCAAAGTGGTATCATCCCC
ATTTAATAGCTGAGGAAACAGAGGCTTAGAAAGGCTGGGTAACTTGACCAGGGTCATGCAAC
TAGTCTGCGGTGGAGCCAGGATTCTGTCTGACCCTAAAGGCCAAGTTCTTTATATTTATTTC
TACCACCTGCTAAAGTCTTGAATGGAGGCTGAAAGCACAGTTGGGGTATGGGGAAGAAAAAT
ATATATACATACATATATGTATATGTATGTATGTATGTATGGGGGGTTGTTTTGTTTTTGTT
TTTGATAAGGAGTTTTGCTCTTGTTGCCCAGGCTGGAGTGCAGTGGTATGATCTGGGCTCAC
TGCAACCTCCGCCTCCCGGGTTCAAGTCATTCTCCTGCCTCAGCCTCCCGAGTAGCTGGGAT
TACCGGAGCATGCCACCACACCCAGCAAAGTTTTGTATTTTTAGTAGAGACAGGGTTTCACC
ATGTTGGCCAGGCTGATCTTGAACTCCTCATCTCAGGTGATCTGCCCGCCTCCGCTTCCCAA
AGTGCTGGGATTACAGGTGTGAGTCACCGCGTCCGGCCTACAGATATATTTAATTTAAAGAG
ATCTAAAACAAATACAAAACTGTCCACATCTATGTTGATGGACCCATAAAAATAGCAGTCTG
CCAGGGTCTGCCGGAAGAGACAGATAAGCATACATATTAACATGGATATATATGTGAATTTC
ATTCAAATGGTTCTCACATGAGAGTAACTAGCATCTTTCTCTCAGATGATGAAGATGATGAA
GAGGAAGATGAAGAGGAAGAAATCGACGTGGTCACTGTGGAGAAGCGGCGTTCCTCCTCCAA
CACCAAGGCTGTCACCACATTCACCATCACTGTGCGTCCCAAGAACGCAGCCCTGGGTCCCG
GGAGGGCTCAGTCCAGCGAGCTGATCCTCAAACGATGCCTTCCCATCCACCAGCAGCACAAC
TATGCCGCCCCCTCTCCCTACGTGGAGAGTGAGGATGCACCCCCACAGAAGAAGATAAAGAG
CGAGGCGTCCCCACGTCCGCTCAAGAGTGTCATCCCCCCAAAGGCTAAGAGCTTGAGCCCCC
GAAACTCTGACTCGGAGGACAGTGAGCGTCGCAGAAACCACAACATCCTGGAGCGCCAGCGC
CGCAACGACCTTCGGTCCAGCTTTCTCACGCTCAGGGACCACGTGCCGGAGTTGGTAAAGAA
TGAGAAGGCCGCCAAGGTGGTCATTTTGAAAAAGGCCACTGAGTATGTCCACTCCCTCCAGG
CCGAGGAGCACCAGCTTTTGCTGGAAAAGGAAAAATTGCAGGCAAGACAGCAGCAGTTGCTA
AAGAAAATTGAACACGCTCGGACTTGCTAGACGCTTCTCAAAACTGGACAGTCACTGCCACT
TTGCACATTTTGATTTTTTTTTTAAACAAACATTGTGTTGACATTAAGAATGTTGGTTTACT
TTCAAATCGGTCCCCTGTCGAGTTCGGCTCTGGGTGGGCAGTAGGACCACCAGTGTGGGGTT
CTGCTGGGACCTTGGAGAGCCTGCATCCCAGGATGCTGGGTGGCCCTGCAGCCTCCTCCACC
TCACCTCCATGACAGCGCTAAACGTTGGTGACGGTTGGGAGCCTCTGGGGCTGTTGAAGTCA
CCTTGTGTGTTCCAAGTTTCCAAACAACAGAAAGTCATTCCTTCTTTTTAAAATGGTGCTTA
AGTTCCAGCAGATGCCACATAAGGGGTTTGCCATTTGATACCCCTGGGGAACATTTCTGTAA
ATACCATTGACACATCCGCCTTTTGTATACATCCTGGGTAATGAGAGGTGGCTTTTGCGGCC
AGTATTAGACTGGAAGTTCATACCTAAGTACTGTAATAATACCTCAATGTTTGAGGAGCATG
TTTTGTATACAAATATATTGTTAATCTCTGTTATGTACTGTACTAATTCTTACACTGCCTGT
ATACTTTAGTATGACGCTGATACATAACTAAATTTGATACTTATATTTTCGTATGAAAATGA
GTTGTGAAAGTTTTGAGTAGATATTACTTTATCACTTTTTGAACTAAGAAACTTTTGTAAAG
AAATTTACTATATATATATGCCTTTTTCCTAGCCTGTTTCTTCCTGTTAATGTATTTGTTCA
TGTTTGGTGCATAGAACTGGGTAAATGCAAAGTTCTGTGTTTAATTTCTTCAAAATGTATAT
ATTTAGTGCTGCATCTTATAGCACTTTGAAATACCTCATGTTTATGAAAATAAATAGCTTAA
AATTAAA.

Sequence for an exemplary genomic DNA sequence encoding human MYCNOS (SEQ ID NO: 40):

AGGCTGTGACAGTCATCTGTCTGGACGCGCTGGGTGGATGCGGGGGGCTCCTGGGAACTGTGT
TGGAGCCGAGCAAGCGCTAGCCAGGCGCAAGCGCGCACAGACTGTAGCCATCCGAGGACACCC
CCGCCCCCCCGGCCCACCCGGAGACACCCGCGCAGAATCGCCTCCGGATCCCCTGCAGTCGGC
GGGAGGTAAGGAGCAGGGCTTGCAAACCGCCCGGCGCCCAGGGAAGCGACGAGCGCCGGGGCA
AGGCAAGCCCTGGACGGGATTGCGACGTGCGCACCGGGCGCCCTAATATGCCCGGGGGACTGT
TTCTGCTTCCGAAACAAAACCATCTCTGGGTTTTCCCAGAAAAGCCAGTTCCAGCCCCGAAGG
CATCCTGGCTAGAGGAGACCCGCCCTAATCCTTTTGCAGCCCTTACCGGGGGGAGTAATGGCT
TCTGCGAAAAGAAATTCCCTCGGCTCTAGAAGATCTGTCTGTGTTTGAGCTGTCGGAGAGCCG
GTGCGTCCCCACCCCAGGCTGGGGTTCTTCTCCAAAGGGTGCCCCTGGAGGAAGAAGAGGGGG
GGATTAGGCAGGGCGAGGCCGCCGCGGTCGCAATCTGGGTCACGGCTGCTCCAGCTTGGAGGA
GAGGCGGCTCTCCCGGCGACCCTCCTCGCGCGGGCGCCCCTGCCATTCCCGGGAACAGGGGCT
CAGCCTCTCCCTCCCTGGAAGAGGACGTTGTCGTGGGTTTGGAAGAGCAGGGGTGGGCTTAGA
GAGCTTCCAATTAAGCTATTGGCAGGAGTATCCCTGCAGCGGGTGAATGCCGAGGGGCGTTTG
CTCAAATTTGGGGAGGGGAAGGATTTGTGGATATGGGTGTCTGTTGTTGGTCTCTGTCTAGAG
AAAGGCTTTTTTTTATTTGCAAAGTTTTCTAAATCCCCTGCTATCATTTGCACTCCTGAGGTT
GCATTTTTACAAAGGGGGTAGAAGGTACTCCAAATACCATTCCCGGTAGCTGGGTCGGAGAGC
CTGGGGCTTCCCCTGAGCAGCCGGCCCCACACCGCTGCGAGTGCGGTTGTCTGCGTGCTCGTG
AGAGCTAGAATTCTGCAGCCAGGAACAGCCCCCTCCCCCAGGCAGTGCCTTGTGTGAATGAAA
TGGCAGTTTCCAAAGTTGCGGAGCCTCGCCACCACCCCCTGCATCTGCATGCCCCCTCCCACC
CCCTGTCGTAGACAGCTTGTACACAAAAGGAGGGCGGGAGGGAGGGAGCGAGAGGCACAACTT
CCTCCACCTTCGGGAGCAGTGGGCAGAGTGGGGGGCTTGGAGGGAAGATTGGGGAACCTGGTT
AGAGGGGGCGCCCATTGCCTATCCCCTCGGTCTGCCCCGTTTGCCCACCCTCTCCGGTGTGTC
TGTCGGTTGCAGTGTTGGAGGTCGGCGCCGGCCCCCGCCTTCCGCGCCCCCCACGGGAAGGAA
GCACCCCCGGTATTAAAACGAACGGGGCGGAAAGAAGCCCTCAGTCGCCGGCCGGGAGGCGAG
CCGATGCCGAGCTGCTCCACGTCCACCATGCCGGGCATGATCTGCAAGAACCCAGACCTCGAG
TTTGACTCGCTACAGCCCTGCTTCTACCCGGACGAAGATGACTTCTACTTCGGCGGCCCCGAC
TCGACCCCCCCGGGGGAGGACATCTGGAAGAAGTTTGAGCTGCTGCCCACGCCCCCGCTGTCG
CCCAGCCGTGGCTTCGCGGAGCACAGCTCCGAGCCCCCGAGCTGGGTCACGGAGATGCTGCTT
GAGAACGAGCTGTGGGGCAGCCCGGCCGAGGAGGACGCGTTCGGCCTGGGGGGACTGGGTGGC
CTCACCCCCAACCCGGTCATCCTCCAGGACTGCATGTGGAGCGGCTTCTCCGCCCGCGAGAAG
CTGGAGCGCGCCGTGAGCGAGAAGCTGCAGCACGGCCGCGGGCCGCCAACCGCCGGTTCCACC
GCCCAGTCCCCGGGAGCCGGCGCCGCCAGCCCTGCGGGTCGCGGGCACGGCGGGGCTGCGGGA
GCCGGCCGCGCCGGGGCCGCCCTGCCCGCCGAGCTCGCCCACCCGGCCGCCGAGTGCGTGGAT
CCCGCCGTGGTCTTCCCCTTTCCCGTGAACAAGCGCGAGCCAGCGCCCGTGCCCGCAGCCCCG
GCCAGTGCCCCGGCGGCGGGCCCTGCGGTCGCCTCGGGGGGGGTATTGCCGCCCCAGCCGGGG
CCCCGGGGGTCGCCCCTCCGCGCCCAGGCGGCCGCCAGACCAGCGGCGGCGACCACAAGGCCC
TCAGTACCTCCGGAGAGGACACCCTGAGCGATTCAGGTAAAGACCGAACTCGGGTCCGGCTGC
CTCCCTGGGGCACTGGACCCCGGGTCGCGTCCCCTTTGTTAGTGCTCGTATGTCTTGGCCTGG
GGAGCATTTTGGAGGCAGTGCTAGGGGCAGAGAGGTCCTGTTTCCCCCAAGTCTCTCCTCGGG
GTAAAGAGAAGGGGCTGAGAGAATGCCGTTGCAAAAGGGGTGCTCTCCAATTCTCGCCTTCAC
TAAAGTTCCTTCCACCCTCTCCTGGGGAGCCCTCCTCTAGGCCATCACGGGCCCTCACCCGGT
CCCCCACCTCTCTTTTGCAGCGCAGTCTGAGGAATAAAATTGGAGAAAGTTGGTGGCTAAACC
GGGTGGGGGTTTAGGGGGTTGCTGGGTGCACTGCCTGGACAGAAACCTGTTAGCGCAGGGGTG
AAAGGGACTCTCTGGCCCAGGTCAGGGGAGGGAAAGACATCCCGAGAAGATTCAAGGGCTGTG
CAAAGCCCTGTTTAAGGCGCAGGAACTTATAGGAGGGTTGCACAGATGGCTAGAGCCGATTTT
CTATTCTTTTTCTTTTTCTTTTTTTTTTTTTTTCAAATGTCGGTACCTTTCCCTTCCCCCATC
CTCGGTGGGTGGTGGGCTATTTGCTCCTGGTGCGTGGCCAGCAGGCGGCGATATGCGAGGCCA
GCAGGGGGCCCGGGATCTGAAAGGCTGGGGGTGGTGGGGGCACCCTCCCTCCCTCCATTCAGC
AGCTGGCTGCAAGTGCAACAGCAGTTGTGTACATTCTCAGGGGGCCTCCTCTTTCCAGTGTGC
AGTGGAAACTGGCTGTAGTTTTGTCTTCCAGCCTGAATTCCAGGCCTAATTTGAGATGTGAGT
TGTATCTGTAACCCAGTGCCCTTGAAGGTGAGGGCAGGCACTCAGCAGCCTCTCCAGGAAGGC
TCACATCCTGGGAGGACTCACTGATTAGTTCTATTGTGTTCATTTGTCTGTGTCTTAAGCTGA
AGGGAAGAGTTAAAACCAAGCCTTTCCCTGGGGGTCTGGATGAACAGAACTCAACCCAAAGAG
TGGCATTGCCTTGTCCTTGGAGCAGGGAGCTGGGACCCCCCTTGGACTTTGAAAACCAGTGTT
TTCAGAATGCAGGTGGATAACAAGCCTAAATTTACTTCTGGGCTGAGGAGAGATCTTTGAGGC
TCCTGGAAGGAAACTTGGTGATAAGCCTCCAGTTTGAAACGGCTCTGTCCCTTTAATGTCTGT
GCCTTGACAGCTTTTGGTGAGGAAGCACTTCCTTCCAACAGCTGTCTTCTTGGCAGAAAACCA
AAACATTGGCTTAAAGGGACCCACAGACTGGAACAGCCTCACATTTCGGCTTTAGAACAAATC
CCACAATTGTTCAGCTTTCCGGTCCCCTTCAGATCAAGCAGAAGATATGTTTTGATTTTCATG
CTTGTATTTTAAACAATAATTTTCTACCCCAGCGTGGTAGTCAATGAGGAGAGAGGGGAAGAA
TGCGCACATGATGCTACACGTTTCTGTTGTTGCTGTTATTATTGGTGGCTTTGAGGAGAGCTG
CTCCCATTTGGGGGTTTATACCAACTGTGGATTATGGCTTTGTCATTAAGATTTGATCTTTGT
TAAATGAAAAACTGTTTATTGTATAAAACTCAGGTTTGTGGACGAAAAGTTGTTTTTTTTCTT
CAGTTAATTAAATTGTTCCTCAAGTTTGTTTAAGGACTTAAAATCAAACACAACCATGTGTAA
ACTGCTAAATGAGGCTCCTAAAATGAGAGGCCTCAACTCTTTAAGTGTGGAGCTAGAAATGTA
AATAAGTCCACAGGGCAGACTGGTGATTATGATAAAAGCTACCATTTACTGAGCATCTGTCTA
CTAGGCTCAGCTCTATGCTAAGTCTACATGTTATCTGTCAAAGTGGTATCATCCCCATTTAAT
AGCTGAGGAAACAGAGGCTTAGAAAGGCTGGGTAACTTGACCAGGGTCATGCAACTAGTCTGC
GGTGGAGCCAGGATTCTGTCTGACCCTAAAGGCCAAGTTCTTTATATTTATTTCTACCACCTG
CTAAAGTCTTGAATGGAGGCTGAAAGCACAGTTGGGGTATGGGGAAGAAAAATATATATACAT
ACATATATGTATATGTATGTATGTATGTATGGGGGGTTGTTTTGTTTTTGTTTTTGATAAGGA
GTTTTGCTCTTGTTGCCCAGGCTGGAGTGCAGTGGTATGATCTGGGCTCACTGCAACCTCCGC
CTCCCGGGTTCAAGTCATTCTCCTGCCTCAGCCTCCCGAGTAGCTGGGATTACCGGAGCATGC
CACCACACCCAGCAAAGTTTTGTATTTTTAGTAGAGACAGGGTTTCACCATGTTGGCCAGGCT
GATCTTGAACTCCTCATCTCAGGTGATCTGCCCGCCTCCGCTTCCCAAAGTGCTGGGATTACA
GGTGTGAGTCACCGCGTCCGGCCTACAGATATATTTAATTTAAAGAGATCTAAAACAAATACA
AAACTGTCCACATCTATGTTGATGGACCCATAAAAATAGCAGTCTGCCAGGGTCTGCCGGAAG
AGACAGATAAGCATACATATTAACATGGATATATATGTGAATTTCATTCAAATGGTTCTCACA
TGAGAGTAACTAGCATCTTTCTCTCAGATGATGAAGATGATGAAGAGGAAGATGAAGAGGAAG
AAATCGACGTGGTCACTGTGGAGAAGCGGCGTTCCTCCTCCAACACCAAGGCTGTCACCACAT
TCACCATCACTGTGCGTCCCAAGAACGCAGCCCTGGGTCCCGGGAGGGCTCAGTCCAGCGAGC
TGATCCTCAAACGATGCCTTCCCATCCACCAGCAGCACAACTATGCCGCCCCCTCTCCCTACG
TGGAGAGTGAGGATGCACCCCCACAGAAGAAGATAAAGAGCGAGGCGTCCCCACGTCCGCTCA
AGAGTGTCATCCCCCCAAAGGCTAAGAGCTTGAGCCCCCGAAACTCTGACTCGGAGGACAGTG
AGCGTCGCAGAAACCACAACATCCTGGAGCGCCAGCGCCGCAACGACCTTCGGTCCAGCTTTC
TCACGCTCAGGGACCACGTGCCGGAGTTGGTAAAGAATGAGAAGGCCGCCAAGGTGGTCATTT
TGAAAAAGGCCACTGAGTATGTCCACTCCCTCCAGGCCGAGGAGCACCAGCTTTTGCTGGAAA
AGGAAAAATTGCAGGCAAGACAGCAGCAGTTGCTAAAGAAAATTGAACACGCTCGGACTTGCT
AGACGCTTCTCAAAACTGGACAGTCACTGCCACTTTGCACATTTTGATTTTTTTTTTAAACAA
ACATTGTGTTGACATTAAGAATGTTGGTTTACTTTCAAATCGGTCCCCTGTCGAGTTCGGCTC
TGGGTGGGCAGTAGGACCACCAGTGTGGGGTTCTGCTGGGACCTTGGAGAGCCTGCATCCCAG
GATGCTGGGTGGCCCTGCAGCCTCCTCCACCTCACCTCCATGACAGCGCTAAACGTTGGTGAC
GGTTGGGAGCCTCTGGGGCTGTTGAAGTCACCTTGTGTGTTCCAAGTTTCCAAACAACAGAAA
GTCATTCCTTCTTTTTAAAATGGTGCTTAAGTTCCAGCAGATGCCACATAAGGGGTTTGCCAT
TTGATACCCCTGGGGAACATTTCTGTAAATACCATTGACACATCCGCCTTTTGTATACATCCT
GGGTAATGAGAGGTGGCTTTTGCGGCCAGTATTAGACTGGAAGTTCATACCTAAGTACTGTAA
TAATACCTCAATGTTTGAGGAGCATGTTTTGTATACAAATATATTGTTAATCTCTGTTATGTA
CTGTACTAATTCTTACACTGCCTGTATACTTTAGTATGACGCTGATACATAACTAAATTTGAT
ACTTATATTTTCGTATGAAAATGAGTTGTGAAAGTTTTGAGTAGATATTACTTTATCACTTTT
TGAACTAAGAAACTTTTGTAAAGAAATTTACTATATATATATGCCTTTTTCCTAGCCTGTTTC
TTCCTGTTAATGTATTTGTTCATGTTTGGTGCATAGAACTGGGTAAATGCAAAGTTCTGTGTT
TAATTTCTTCAAAATGTATATATTTAGTGCTGCATCTTATAGCACTTTGAAATACCTCATGTT
TATGAAAATAAATAGCTTAAAATTAAA.

In view of the many possible implementations to which the principles of the present disclosure may be applied, it should be recognized that the illustrated implementations are only preferred examples and should not be taken as limiting the scope of the present disclosure. Rather, the scope is defined by the following claims. We therefore claim as our invention all that comes within the scope and spirit of these claims.

1. A compound, or a stereoisomer, tautomer, or pharmaceutically acceptable salt or ester thereof, according to Formula IA 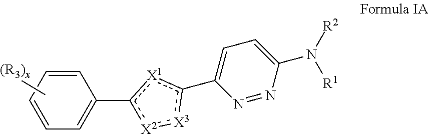
wherein
each of X¹, X², and X³ independently is N or O;
R¹ is -(linker)ₜ-R^{b} wherein R^{b} is aromatic, heteroaliphatic, or aliphatic; the linker is an aliphatic or heteroaliphatic group; and t is 0 or 1;
R² is H or aliphatic;
each R³ independently is heteroaliphatic, hydroxy, aliphatic, haloaliphatic, or halo;
x is an integer selected from 0 to 5; and
provided that the compound is not 6-(3-(4-methoxyphenyl)-1,2,4-oxadiazol-5-yl)-N-(3-(pyrrolidin-1-yl)propyl)pyridazin-3-amine, N-(3-([1,4′-bipiperidin]-1′-yl)propyl)-6-(3-(4-methoxyphenyl)-1,2,4-oxadiazol-5-yl)pyridazin-3-amine, 6-(3-(4-methoxyphenyl)-1,2,4-oxadiazol-5-yl)-N-(3-(4-methylpiperazin-1-yl)propyl)pyridazin-3-amine, N1-(6-(3-(4-methoxyphenyl)-1,2,4-oxadiazol-5-yl)pyridazin-3-yl)-N2,N2-dimethylethane-1,2-diamine, N-(3-(azepan-1-yl)propyl)-6-(3-(4-methoxyphenyl)-1,2,4-oxadiazol-5-yl)pyridazin-3-amine, 6-(3-(4-methoxyphenyl)-1,2,4-oxadiazol-5-yl)-N-(3-(4-methylpiperidin-1-yl)propyl)pyridazin-3-amine, or 6-(3-(4-methoxyphenyl)-1,2,4-oxadiazol-5-yl)-N-(2-morpholinoethyl)pyridazin-3-amine.

2-5. (canceled)

6. The compound of claim 1, wherein the ring bearing X¹, X², and X³ is

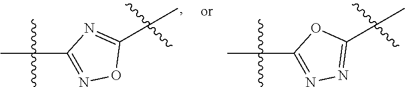 [SMILES: *c1noc(*)n1 ; *c1nnc(*)o1]

7-10. (canceled)

11. The compound of claim 1, wherein:
(i) x is 1, 2, or 3; or
(ii) one R³ is in a para position to the ring bearing X¹, X², and X³; or
(iii) both (i) and (ii).

12. (canceled)

13. The compound of claim 1, wherein each R³ independently is C₁-C₃ alkoxy or hydroxy.

14. The compound of claim 1, wherein x is 1 and R³ is methoxy.

15. The compound of claim 1, wherein the compound has a structure according to Formula II, Formula III, or Formula IV 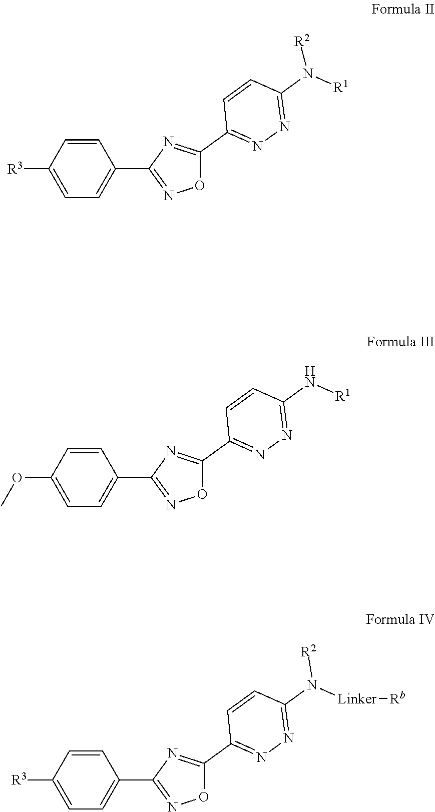
wherein, for Formula IV, the linker is —(CR^{a}₂)ₘ— or —[(CR^{a}₂)ₘO]ᵣ—(CR^{a}₂)ₛ— wherein each R^{a} independently is H, aliphatic, or halo: m is 1, 2, 3, 4, or 5; r is 1, 2, 3, 4, or 5; and s is 0 or 1.

16. The compound of claim 1, wherein R² is H and/or R¹ is —(CH₂)ₘ—R^{b}.

17-18. (canceled)

19. The compound of claim 16, wherein m is 2 or 3.

20. The compound of claim 16, wherein R^{b} is an N-containing cyclic group, —N(R^{C})₂, or —C(O)N(R^{c})₂ where each R^{c} is H or alkyl.

21. The compound of claim 1, wherein R¹ is:

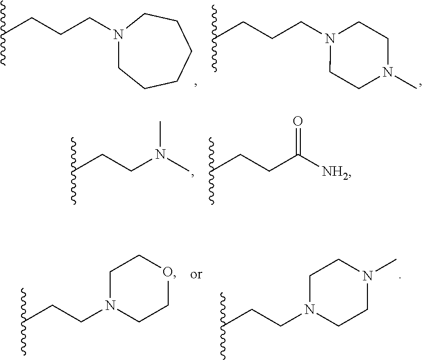

22. The compound of claim 1, wherein the compound is:

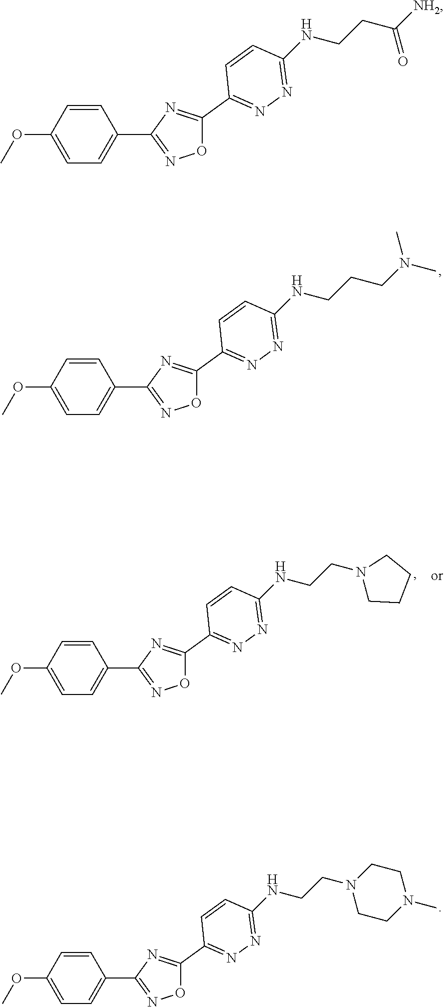

23. (canceled)

24. The compound of claim 15, wherein the compound has a structure according to Formula V 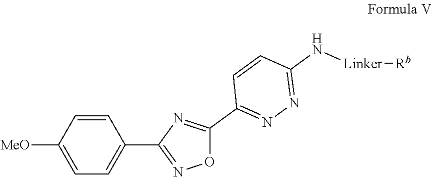
wherein the linker is —(CH₂)ₘ— or —[(CH₂)ₘO]ᵣ—(CH₂)ₛ— wherein m is 1, 2, 3, 4, or 5; r is 1, 2, 3, 4, or 5; and s is 0 or 1.

25. The compound of claim 1, wherein R¹ is:

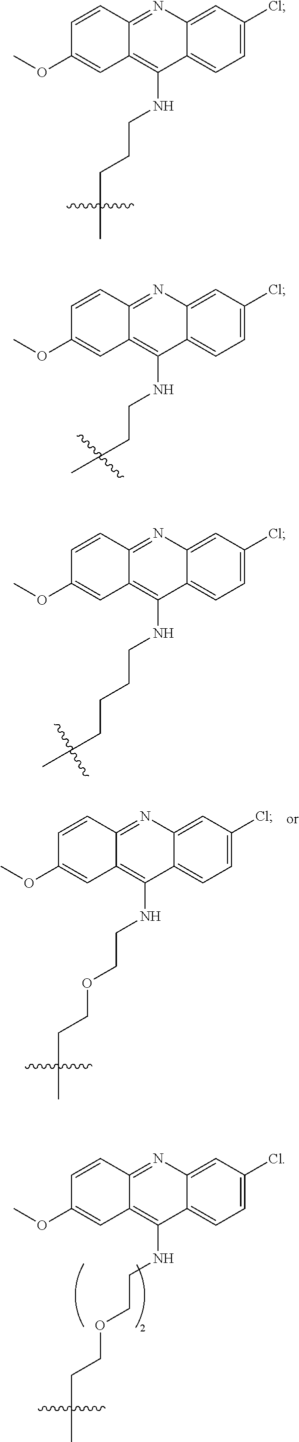

26. The compound of claim 1, wherein the compound is:

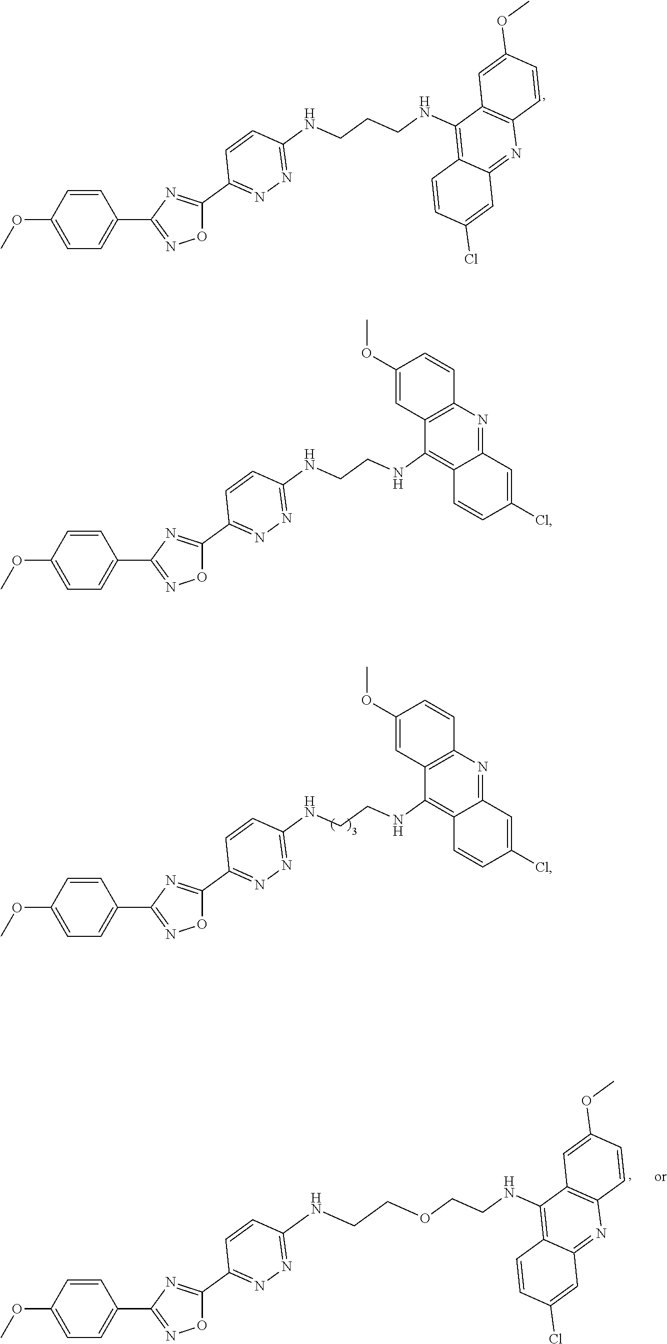

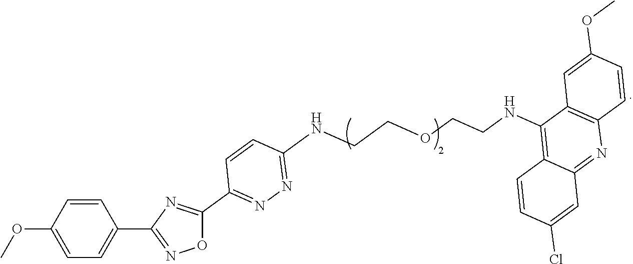

27. A pharmaceutical composition comprising a compound according to claim 1 and at least one pharmaceutically acceptable additive.

28. The pharmaceutical composition of claim 27, comprising a unit dosage form of a therapeutic amount of the compound and/or an anticancer agent.

29. (canceled)

30. A method of decreasing cancer-relevant protein expression in a cell, comprising contacting the cell with an effective amount of a compound, or a stereoisomer, tautomer, or pharmaceutically acceptable salt or ester thereof, according to claim 1.

31-38. (canceled)

39. The method of claim 30, wherein the cell is in vitro or the cell is in vivo.

40. (canceled)

41. The method of claim 30, wherein decreasing cancer-relevant protein expression in the cell decreases growth and/or proliferation of the cell and wherein the cell is a cell with overexpression of the MYCN gene.

42. (canceled)

43. The method of claim 30, wherein the compound selectively binds to a non-canonical G4 quadruplex nucleic acid region in the MYCN gene and wherein the non-canonical G4 quadruplex nucleic acid region comprises a hairpin structure.

44. (canceled)

45. The method of claim 30, wherein the cell is a cancer cell in a subject, and wherein the method further comprising treating or preventing cancer in the subject, comprising the step of administering to a subject in need thereof a therapeutically effective amount of the compound, or the pharmaceutically acceptable salt or ester thereof, to decrease N-Myc expression in the cancer cell, thereby treating or preventing the cancer in the subject.

46. The method of claim 45, wherein the cancer cell is a neurological cancer cell or a lung cancer cell and/or wherein the cancer is neuroblastoma, rhabdomyosarcoma, prostate cancer, or small cell lung cancer.

47-48. (canceled)

49. The method of claim 45, further comprising administering a therapeutically effective amount of an additional anticancer agent to the subject.

50-55. (canceled)
